(19) United States
(12) Patent Application Publication
(10) Pub. No.: US 2024/0285759 A1
(43) Pub. Date: Aug. 29, 2024

(54) T CELL RECEPTORS TARGETING Q61-COMPRISING RAS MUTATIONS AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Smita S. CHANDRAN, Long Island City, NY (US); Inaki Etxeberria URIZ, New York, NY (US); Christopher A. KLEBANOFF, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/660,551

(22) Filed: May 10, 2024

Related U.S. Application Data
(63) Continuation of application No. PCT/US22/49485, filed on Nov. 10, 2022.
(60) Provisional application No. 63/277,832, filed on Nov. 10, 2021.

(51) Int. Cl. A61K 39/00; A61P 35/00; C12N 15/64

(52) U.S. Cl. CPC A61K 39/4632; A61K 39/4611; A61K 39/4613; A61P 35/00; C12N 15/64

(57) ABSTRACT

The presently disclosed subject matter provides novel T cell receptors (TCRs) that target a mutated RAS protooncogene. The presently disclosed subject matter further provides cells comprising such TCRs. and methods of using such cells for treating cancers associated with RAS.

### CROSS-REFERENCE TO RELATED APPLICATIONS

This application is a Continuation of International Patent Application No. PCT/US22/49485, filed Nov. 10, 2022, which claims priority to U.S. Provisional Patent Application No. 63/277,832, filed on Nov. 10, 2021, the content of each of which is incorporated by reference in its entirety, and to each of which priority is claimed.

### SEQUENCE LISTING

The instant application contains a Sequence Listing which has been submitted electronically) in XML format and is hereby incorporated by reference in its entirety. Said XML copy, created on May 10, 2024, is named 0927870121.xml and is 372,736 bytes in size.

### 1. INTRODUCTION

The presently disclosed subject matter provides novel T cell receptors (TCRs) that target mutated RAS proto-oncogenes. The presently disclosed subject matter further provides cells comprising such TCRs, and methods of using such cells for treating cancers associated with mutated RAS.

### 2. BACKGROUND OF THE INVENTION

Cell-based immunotherapy is a therapy with curative potential for the treatment of cancers. Immunoresponsive cells (e.g., T cells) may be modified to target tumor antigens through the introduction of genetic material coding for T cell receptors (TCRs) that confer specificity to selected antigens. Targeted T cell therapy using specific TCRs has shown clinical success in treating multiple solid and hematologic malignancies.

Collectively, the RAS proteins are the most commonly mutated family of oncoproteins in human cancer. Patients with oncogenic mutations encoding a RAS protein (e.g., KRAS A/B, NRAS, and HRAS) typically respond poorly to standard therapies. Hotspot mutations at NRAS(Q61) are found in diverse cancer types of high unmet medical need. These include colorectal cancer, thyroid cancer, and about 20% of cutaneous melanomas. Most colorectal and thyroid cancers fail to respond to immune checkpoint inhibitors. Further, unlike other genomic variants of melanoma, those that harbor NRAS mutations respond poorly to current immunotherapies and targeted agents. New therapies with curative potential are therefore urgently needed for patients with cancers associated with NRAS(Q61) mutations. Accordingly, there remains a critical unmet need for novel therapeutic strategies to identify and generate TCRs targeting NRAS mutations, and for strategies capable of inducing potent cancer eradication with minimal risk of normal tissue toxicity.

### 3. SUMMARY OF THE INVENTION

The presently disclosed subject matter provides T cell receptors (TCR) that bind to a RAS peptide that comprises a mutation. In certain embodiments, the RAS peptide comprises a Q61 mutation. In certain embodiments, the RAS peptide comprises a Q61R mutation, a Q61K mutation, or a Q61L mutation. In certain embodiments, the RAS peptide comprises or consists of the amino acid sequence set forth in SEQ ID NO: 1, SEQ ID NO: 2, or SEQ ID NO: 3. In certain embodiments, the RAS peptide comprises or consists of the amino acid sequence set forth in SEQ ID NO: 1. In certain embodiments, the RAS peptide comprises or consists of the amino acid sequence set forth in SEQ ID NO: 2. In certain embodiments, the RAS peptide comprises or consists of the amino acid sequence set forth in SEQ ID NO: 3. In certain embodiments, the RAS peptide is associated with an HLA class I complex. In certain embodiments, the HLA class I complex is selected from an HLA-A, an HLA-B, and an HLA-C. In certain embodiments, the HLA class I complex is an HLA-A. In certain embodiments, the HLA-A is an HLA-A*01 superfamily member. In certain embodiments, the HLA-A*01 superfamily member is selected from the group consisting of HLA-A*01, HLA-A*25, HLA-A*26, HLA-A*30, HLA-A*31, HLA-A*32, HLA-A*36, HLA-A*74:10, and HLA-A*80. In certain embodiments, the HLA-A*01 superfamily member is HLA-A*01:01.

In certain embodiments, the TCR comprises an extracellular domain that binds to the RAS peptide, wherein the extracellular domain comprises an α chain and a β chain, wherein the α chain comprises an α chain variable region and α chain constant region, and the β chain comprises a β chain variable region and a β chain constant region. In certain embodiments,
a) the α chain variable region comprises a CDR3 comprising the amino acid sequence set forth in SEQ ID NO: 7 or a conservative modification thereof, and the β chain variable region comprises a CDR3 comprising the amino acid sequence set forth in SEQ ID NO: 10 or a conservative modification thereof;
b) the α chain variable region comprises a CDR3 comprising the amino acid sequence set forth in SEQ ID NO: 17 or a conservative modification thereof, and the β chain variable region comprises a CDR3 comprising the amino acid sequence set forth in SEQ ID NO: 19 or a conservative modification thereof;
c) the α chain variable region comprises a CDR3 comprising the amino acid sequence set forth in SEQ ID NO: 26 or a conservative modification thereof, and the β chain variable region comprises a CDR3 comprising the amino acid sequence set forth in SEQ ID NO: 29 or a conservative modification thereof;
d) the α chain variable region comprises a CDR3 comprising the amino acid sequence set forth in SEQ ID NO: 36 or a conservative modification thereof, and the β chain variable region comprises a CDR3 comprising the amino acid sequence set forth in SEQ ID NO: 39 or a conservative modification thereof;
e) the α chain variable region comprises a CDR3 comprising the amino acid sequence set forth in SEQ ID NO: 44 or a conservative modification thereof, and the β chain variable region comprises a CDR3 comprising the amino acid sequence set forth in SEQ ID NO: 39 or a conservative modification thereof;
f) the α chain variable region comprises a CDR3 comprising the amino acid sequence set forth in SEQ ID NO: 47 or a conservative modification thereof, and the β chain variable region comprises a CDR3 comprising the amino acid sequence set forth in SEQ ID NO: 50 or a conservative modification thereof;
g) the α chain variable region comprises a CDR3 comprising the amino acid sequence set forth in SEQ ID NO: 55 or a conservative modification thereof, and the β chain variable region comprises a CDR3 comprising the amino acid sequence set forth in SEQ ID NO: 56 or a conservative modification thereof;
h) the α chain variable region comprises a CDR3 comprising the amino acid sequence set forth in SEQ ID NO: 55 or a conservative modification thereof, and the β chain variable region comprises a CDR3 comprising the amino acid sequence set forth in SEQ ID NO: 61 or a conservative modification thereof;
i) the α chain variable region comprises a CDR3 comprising the amino acid sequence set forth in SEQ ID NO: 66 or a conservative modification thereof, and the β chain variable region comprises a CDR3 comprising the amino acid sequence set forth in SEQ ID NO: 67 or a conservative modification thereof;
j) the α chain variable region comprises a CDR3 comprising the amino acid sequence set forth in SEQ ID NO: 74 or a conservative modification thereof, and the β chain variable region comprises a CDR3 comprising the amino acid sequence set forth in SEQ ID NO: 67 or a conservative modification thereof;
k) the α chain variable region comprises a CDR3 comprising the amino acid sequence set forth in SEQ ID NO: 77 or a conservative modification thereof, and the β chain variable region comprises a CDR3 comprising the amino acid sequence set forth in SEQ ID NO: 80 or a conservative modification thereof;
l) the α chain variable region comprises a CDR3 comprising the amino acid sequence set forth in SEQ ID NO: 87 or a conservative modification thereof, and the β chain variable region comprises a CDR3 comprising the amino acid sequence set forth in SEQ ID NO: 88 or a conservative modification thereof;
m) the α chain variable region comprises a CDR3 comprising the amino acid sequence set forth in SEQ ID NO: 95 or a conservative modification thereof, and the β chain variable region comprises a CDR3 comprising the amino acid sequence set forth in SEQ ID NO: 96 or a conservative modification thereof;
n) the α chain variable region comprises a CDR3 comprising the amino acid sequence set forth in SEQ ID NO: 101 or a conservative modification thereof, and the β chain variable region comprises a CDR3 comprising the amino acid sequence set forth in SEQ ID NO: 104 or a conservative modification thereof;
o) the α chain variable region comprises a CDR3 comprising the amino acid sequence set forth in SEQ ID NO: 111 or a conservative modification thereof, and the β chain variable region comprises a CDR3 comprising the amino acid sequence set forth in SEQ ID NO: 114 or a conservative modification thereof;
p) the α chain variable region comprises a CDR3 comprising the amino acid sequence set forth in SEQ ID NO: 121 or a conservative modification thereof, and the β chain variable region comprises a CDR3 comprising the amino acid sequence set forth in SEQ ID NO: 123 or a conservative modification thereof;
q) the α chain variable region comprises a CDR3 comprising the amino acid sequence set forth in SEQ ID NO: 130 or a conservative modification thereof, and the β chain variable region comprises a CDR3 comprising the amino acid sequence set forth in SEQ ID NO: 133 or a conservative modification thereof;
r) the α chain variable region comprises a CDR3 comprising the amino acid sequence set forth in SEQ ID NO: 139 or a conservative modification thereof, and the β chain variable region comprises a CDR3 comprising the amino acid sequence set forth in SEQ ID NO: 140 or a conservative modification thereof;
s) the α chain variable region comprises a CDR3 comprising the amino acid sequence set forth in SEQ ID NO: 147 or a conservative modification thereof, and the β chain variable region comprises a CDR3 comprising the amino acid sequence set forth in SEQ ID NO: 140 or a conservative modification thereof;
t) the α chain variable region comprises a CDR3 comprising the amino acid sequence set forth in SEQ ID NO: 150 or a conservative modification thereof, and the β chain variable region comprises a CDR3 comprising the amino acid sequence set forth in SEQ ID NO: 151 or a conservative modification thereof;
u) the α chain variable region comprises a CDR3 comprising the amino acid sequence set forth in SEQ ID NO: 158 or a conservative modification thereof, and the β chain variable region comprises a CDR3 comprising the amino acid sequence set forth in SEQ ID NO: 161 or a conservative modification thereof;
v) the α chain variable region comprises a CDR3 comprising the amino acid sequence set forth in SEQ ID NO: 173 or a conservative modification thereof, and the β chain variable region comprises a CDR3 comprising the amino acid sequence set forth in SEQ ID NO: 174 or a conservative modification thereof;
w) the α chain variable region comprises a CDR3 comprising the amino acid sequence set forth in SEQ ID NO: 181 or a conservative modification thereof, and the β chain variable region comprises a CDR3 comprising the amino acid sequence set forth in SEQ ID NO: 184 or a conservative modification thereof;
x) the α chain variable region comprises a CDR3 comprising the amino acid sequence set forth in SEQ ID NO: 189 or a conservative modification thereof, and the β chain variable region comprises a CDR3 comprising the amino acid sequence set forth in SEQ ID NO: 190 or a conservative modification thereof;
y) the α chain variable region comprises a CDR3 comprising the amino acid sequence set forth in SEQ ID NO: 197 or a conservative modification thereof, and the β chain variable region comprises a CDR3 comprising the amino acid sequence set forth in SEQ ID NO: 198 or a conservative modification thereof;
z) the α chain variable region comprises a CDR3 comprising the amino acid sequence set forth in SEQ ID NO: 205 or a conservative modification thereof, and the β chain variable region comprises a CDR3 comprising the amino acid sequence set forth in SEQ ID NO: 206 or a conservative modification thereof;
aa) the α chain variable region comprises a CDR3 comprising the amino acid sequence set forth in SEQ ID NO: 213 or a conservative modification thereof, and the β chain variable region comprises a CDR3 comprising the amino acid sequence set forth in SEQ ID NO: 216 or a conservative modification thereof;
ab) the α chain variable region comprises a CDR3 comprising the amino acid sequence set forth in SEQ ID NO: 221 or a conservative modification thereof, and the β chain variable region comprises a CDR3 comprising the amino acid sequence set forth in SEQ ID NO: 222 or a conservative modification thereof;
ac) the α chain variable region comprises a CDR3 comprising the amino acid sequence set forth in SEQ ID NO: 229 or a conservative modification thereof, and the β chain variable region comprises a CDR3 comprising the amino acid sequence set forth in SEQ ID NO: 232 or a conservative modification thereof;
ad) the α chain variable region comprises a CDR3 comprising the amino acid sequence set forth in SEQ ID NO: 237 or a conservative modification thereof, and the β chain variable region comprises a CDR3 comprising the amino acid sequence set forth in SEQ ID NO: 240 or a conservative modification thereof;
ae) the α chain variable region comprises a CDR3 comprising the amino acid sequence set forth in SEQ ID NO: 245 or a conservative modification thereof, and the β chain variable region comprises a CDR3 comprising the amino acid sequence set forth in SEQ ID NO: 248 or a conservative modification thereof;
af) the α chain variable region comprises a CDR3 comprising the amino acid sequence set forth in SEQ ID NO: 254 or a conservative modification thereof, and the β chain variable region comprises a CDR3 comprising the amino acid sequence set forth in SEQ ID NO: 255 or a conservative modification thereof;
ag) the α chain variable region comprises a CDR3 comprising the amino acid sequence set forth in SEQ ID NO: 262 or a conservative modification thereof, and the β chain variable region comprises a CDR3 comprising the amino acid sequence set forth in SEQ ID NO: 265 or a conservative modification thereof;
ah) the α chain variable region comprises a CDR3 comprising the amino acid sequence set forth in SEQ ID NO: 270 or a conservative modification thereof, and the β chain variable region comprises a CDR3 comprising the amino acid sequence set forth in SEQ ID NO: 273 or a conservative modification thereof;
ai) the α chain variable region comprises a CDR3 comprising the amino acid sequence set forth in SEQ ID NO: 278 or a conservative modification thereof, and the β chain variable region comprises a CDR3 comprising the amino acid sequence set forth in SEQ ID NO: 280 or a conservative modification thereof;
aj) the α chain variable region comprises a CDR3 comprising the amino acid sequence set forth in SEQ ID NO: 287 or a conservative modification thereof, and the β chain variable region comprises a CDR3 comprising the amino acid sequence set forth in SEQ ID NO: 290 or a conservative modification thereof;
ak) the α chain variable region comprises a CDR3 comprising the amino acid sequence set forth in SEQ ID NO: 295 or a conservative modification thereof, and the β chain variable region comprises a CDR3 comprising the amino acid sequence set forth in SEQ ID NO: 298 or a conservative modification thereof;
al) the α chain variable region comprises a CDR3 comprising the amino acid sequence set forth in SEQ ID NO: 303 or a conservative modification thereof, and the β chain variable region comprises a CDR3 comprising the amino acid sequence set forth in SEQ ID NO: 306 or a conservative modification thereof;
am) the α chain variable region comprises a CDR3 comprising the amino acid sequence set forth in SEQ ID NO: 311 or a conservative modification thereof, and the β chain variable region comprises a CDR3 comprising the amino acid sequence set forth in SEQ ID NO: 312 or a conservative modification thereof;
an) the α chain variable region comprises a CDR3 comprising the amino acid sequence set forth in SEQ ID NO: 319 or a conservative modification thereof, and the β chain variable region comprises a CDR3 comprising the amino acid sequence set forth in SEQ ID NO: 320 or a conservative modification thereof;
ao) the α chain variable region comprises a CDR3 comprising the amino acid sequence set forth in SEQ ID NO: 325 or a conservative modification thereof, and the β chain variable region comprises a CDR3 comprising the amino acid sequence set forth in SEQ ID NO: 328 or a conservative modification thereof;
ap) the α chain variable region comprises a CDR3 comprising the amino acid sequence set forth in SEQ ID NO: 333 or a conservative modification thereof, and the β chain variable region comprises a CDR3 comprising the amino acid sequence set forth in SEQ ID NO: 334 or a conservative modification thereof;
aq) the α chain variable region comprises a CDR3 comprising the amino acid sequence set forth in SEQ ID NO: 339 or a conservative modification thereof, and the β chain variable region comprises a CDR3 comprising the amino acid sequence set forth in SEQ ID NO: 340 or a conservative modification thereof;
ar) the α chain variable region comprises a CDR3 comprising the amino acid sequence set forth in SEQ ID NO: 345 or a conservative modification thereof, and the β chain variable region comprises a CDR3 comprising the amino acid sequence set forth in SEQ ID NO: 340 or a conservative modification thereof;
as) the α chain variable region comprises a CDR3 comprising the amino acid sequence set forth in SEQ ID NO: 349 or a conservative modification thereof, and the β chain variable region comprises a CDR3 comprising the amino acid sequence set forth in SEQ ID NO: 351 or a conservative modification thereof;
at) the α chain variable region comprises a CDR3 comprising the amino acid sequence set forth in SEQ ID NO: 356 or a conservative modification thereof, and the β chain variable region comprises a CDR3 comprising the amino acid sequence set forth in SEQ ID NO: 351 or a conservative modification thereof;
au) the α chain variable region comprises a CDR3 comprising the amino acid sequence set forth in SEQ ID NO: 360 or a conservative modification thereof, and the β chain variable region comprises a CDR3 comprising the amino acid sequence set forth in SEQ ID NO: 361 or a conservative modification thereof;
av) the α chain variable region comprises a CDR3 comprising the amino acid sequence set forth in SEQ ID NO: 325 or a conservative modification thereof, and the β chain variable region comprises a CDR3 comprising the amino acid sequence set forth in SEQ ID NO: 290 or a conservative modification thereof;
aw) the α chain variable region comprises a CDR3 comprising the amino acid sequence set forth in SEQ ID NO: 371 or a conservative modification thereof, and the β chain variable region comprises a CDR3 comprising the amino acid sequence set forth in SEQ ID NO: 290 or a conservative modification thereof;
ax) the α chain variable region comprises a CDR3 comprising the amino acid sequence set forth in SEQ ID NO: 377 or a conservative modification thereof, and the β chain variable region comprises a CDR3 comprising the amino acid sequence set forth in SEQ ID NO: 378 or a conservative modification thereof; or
ay) the α chain variable region comprises a CDR3 comprising the amino acid sequence set forth in SEQ ID NO: 383 or a conservative modification thereof, and the β chain variable region comprises a CDR3 comprising the amino acid sequence set forth in SEQ ID NO: 378 or a conservative modification thereof.

In certain embodiments,
a) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 9 or a conservative modification thereof;
b) the α chain variable region comprises a CDR2 comprising the amino acid sequence set forth in SEQ ID NO: 16 or a conservative modification thereof, and the β chain variable region comprises a CDR2 comprising the amino acid sequence set forth in SEQ ID NO: 18 or a conservative modification thereof;
c) the α chain variable region comprises a CDR2 comprising the amino acid sequence set forth in SEQ ID NO: 25 or a conservative modification thereof, and the β chain variable region comprises a CDR2 comprising the amino acid sequence set forth in SEQ ID NO: 28 or a conservative modification thereof;
d) the α chain variable region comprises a CDR2 comprising the amino acid sequence set forth in SEQ ID NO: 35 or a conservative modification thereof, and the β chain variable region comprises a CDR2 comprising the amino acid sequence set forth in SEQ ID NO: 38 or a conservative modification thereof;
e) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 49 or a conservative modification thereof;
f) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 38 or a conservative modification thereof;
g) the α chain variable region comprises a CDR2 comprising the amino acid sequence set forth in SEQ ID NO: 65 or a conservative modification thereof, and the β chain variable region comprises a CDR2 comprising the amino acid sequence set forth in SEQ ID NO: 49 or a conservative modification thereof;
h) the α chain variable region comprises a CDR2 comprising the amino acid sequence set forth in SEQ ID NO: 73 or a conservative modification thereof, and the β chain variable region comprises a CDR2 comprising the amino acid sequence set forth in SEQ ID NO: 49 or a conservative modification thereof;
i) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 79 or a conservative modification thereof;
j) the α chain variable region comprises a CDR2 comprising the amino acid sequence set forth in SEQ ID NO: 86 or a conservative modification thereof, and the β chain variable region comprises a CDR2 comprising the amino acid sequence set forth in SEQ ID NO: 38 or a conservative modification thereof;
k) the α chain variable region comprises a CDR2 comprising the amino acid sequence set forth in SEQ ID NO: 94 or a conservative modification thereof, and the β chain variable region comprises a CDR2 comprising the amino acid sequence set forth in SEQ ID NO: 49 or a conservative modification thereof;
l) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 103 or a conservative modification thereof;
m) the α chain variable region comprises a CDR2 comprising the amino acid sequence set forth in SEQ ID NO: 110 or a conservative modification thereof, and the β chain variable region comprises a CDR2 comprising the amino acid sequence set forth in SEQ ID NO: 113 or a conservative modification thereof;
n) the α chain variable region comprises a CDR2 comprising the amino acid sequence set forth in SEQ ID NO: 120 or a conservative modification thereof, and the β chain variable region comprises a CDR2 comprising the amino acid sequence set forth in SEQ ID NO: 113 or a conservative modification thereof;
o) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 132 or a conservative modification thereof;
p) the α chain variable region comprises a CDR2 comprising the amino acid sequence set forth in SEQ ID NO: 35 or a conservative modification thereof, and the β chain variable region comprises a CDR2 comprising the amino acid sequence set forth in SEQ ID NO: 49 or a conservative modification thereof;
q) the α chain variable region comprises a CDR2 comprising the amino acid sequence set forth in SEQ ID NO: 146 or a conservative modification thereof, and the β chain variable region comprises a CDR2 comprising the amino acid sequence set forth in SEQ ID NO: 49 or a conservative modification thereof;
r) the α chain variable region comprises a CDR2 comprising the amino acid sequence set forth in SEQ ID NO: 157 or a conservative modification thereof, and the β chain variable region comprises a CDR2 comprising the amino acid sequence set forth in SEQ ID NO: 160 or a conservative modification thereof;
s) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 183 or a conservative modification thereof;
t) the α chain variable region comprises a CDR2 comprising the amino acid sequence set forth in SEQ ID NO: 120 or a conservative modification thereof, and the β chain variable region comprises a CDR2 comprising the amino acid sequence set forth in SEQ ID NO: 38 or a conservative modification thereof;
u) the α chain variable region comprises a CDR2 comprising the amino acid sequence set forth in SEQ ID NO: 196 or a conservative modification thereof, and the β chain variable region comprises a CDR2 comprising the amino acid sequence set forth in SEQ ID NO: 160 or a conservative modification thereof;
v) the α chain variable region comprises a CDR2 comprising the amino acid sequence set forth in SEQ ID NO: 204 or a conservative modification thereof, and the β chain variable region comprises a CDR2 comprising the amino acid sequence set forth in SEQ ID NO: 113 or a conservative modification thereof;
w) the α chain variable region comprises a CDR2 comprising the amino acid sequence set forth in SEQ ID NO: 212 or a conservative modification thereof, and the β chain variable region comprises a CDR2 comprising the amino acid sequence set forth in SEQ ID NO: 215 or a conservative modification thereof;
x) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 160 or a conservative modification thereof;
y) the α chain variable region comprises a CDR2 comprising the amino acid sequence set forth in SEQ ID NO: 228 or a conservative modification thereof, and the β chain variable region comprises a CDR2 comprising the amino acid sequence set forth in SEQ ID NO: 231 or a conservative modification thereof;
z) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 239 or a conservative modification thereof;
aa) the α chain variable region comprises a CDR2 comprising the amino acid sequence set forth in SEQ ID NO: 73 or a conservative modification thereof, and the β chain variable region comprises a CDR2 comprising the amino acid sequence set forth in SEQ ID NO: 247 or a conservative modification thereof;
ab) the α chain variable region comprises a CDR2 comprising the amino acid sequence set forth in SEQ ID NO: 253 or a conservative modification thereof, and the β chain variable region comprises a CDR2 comprising the amino acid sequence set forth in SEQ ID NO: 248 or a conservative modification thereof;
ac) the α chain variable region comprises a CDR2 comprising the amino acid sequence set forth in SEQ ID NO: 254 or a conservative modification thereof, and the β chain variable region comprises a CDR2 comprising the amino acid sequence set forth in SEQ ID NO: 38 or a conservative modification thereof;
ad) the α chain variable region comprises a CDR2 comprising the amino acid sequence set forth in SEQ ID NO: 261 or a conservative modification thereof, and the β chain variable region comprises a CDR2 comprising the amino acid sequence set forth in SEQ ID NO: 264 or a conservative modification thereof;
ae) the α chain variable region comprises a CDR2 comprising the amino acid sequence set forth in SEQ ID NO: 204 or a conservative modification thereof, and the β chain variable region comprises a CDR2 comprising the amino acid sequence set forth in SEQ ID NO: 272 or a conservative modification thereof;
af) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 279 or a conservative modification thereof;
ag) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 289 or a conservative modification thereof;
ah) the α chain variable region comprises a CDR2 comprising the amino acid sequence set forth in SEQ ID NO: 228 or a conservative modification thereof, and the β chain variable region comprises a CDR2 comprising the amino acid sequence set forth in SEQ ID NO: 297 or a conservative modification thereof;
ai) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 305 or a conservative modification thereof;
aj) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 28 or a conservative modification thereof;
ak) the α chain variable region comprises a CDR2 comprising the amino acid sequence set forth in SEQ ID NO: 318 or a conservative modification thereof, and the β chain variable region comprises a CDR2 comprising the amino acid sequence set forth in SEQ ID NO: 279 or a conservative modification thereof;
al) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 327 or a conservative modification thereof;
am) the α chain variable region comprises a CDR2 comprising the amino acid sequence set forth in SEQ ID NO: 94 or a conservative modification thereof, and the β chain variable region comprises a CDR2 comprising the amino acid sequence set forth in SEQ ID NO: 350 or a conservative modification thereof;
an) the α chain variable region comprises a CDR2 comprising the amino acid sequence set forth in SEQ ID NO: 196 or a conservative modification thereof, and the β chain variable region comprises a CDR2 comprising the amino acid sequence set forth in SEQ ID NO: 350 or a conservative modification thereof;
ao) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 38 or a conservative modification thereof;
ap) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 327 or a conservative modification thereof;
aq) the α chain variable region comprises a CDR2 comprising the amino acid sequence set forth in SEQ ID NO: 370 or a conservative modification thereof, and the β chain variable region comprises a CDR2 comprising the amino acid sequence set forth in SEQ ID NO: 327 or a conservative modification thereof; or ar) the α chain variable region comprises a CDR2 comprising the amino acid sequence set forth in SEQ ID NO: 376 or a conservative modification thereof, and the β chain variable region comprises a CDR2 comprising the amino acid sequence set forth in SEQ ID NO: 38 or a conservative modification thereof.

In certain embodiments,
a) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 8 or a conservative modification thereof;
b) the α chain variable region comprises a CDR1 comprising the amino acid sequence set forth in SEQ ID NO: 15 or a conservative modification thereof, and the β chain variable region comprises a CDR1 comprising the amino acid sequence set forth in SEQ ID NO: 8 or a conservative modification thereof;
c) the α chain variable region comprises a CDR1 comprising the amino acid sequence set forth in SEQ ID NO: 24 or a conservative modification thereof, and the β chain variable region comprises a CDR1 comprising the amino acid sequence set forth in SEQ ID NO: 27 or a conservative modification thereof;
d) the α chain variable region comprises a CDR1 comprising the amino acid sequence set forth in SEQ ID NO: 34 or a conservative modification thereof, and the β chain variable region comprises a CDR1 comprising the amino acid sequence set forth in SEQ ID NO: 37 or a conservative modification thereof;
e) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 48 or a conservative modification thereof
f) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 37 or a conservative modification thereof;
g) the α chain variable region comprises a CDR1 comprising the amino acid sequence set forth in SEQ ID NO: 64 or a conservative modification thereof, and the β chain variable region comprises a CDR1 comprising the amino acid sequence set forth in SEQ ID NO: 48 or a conservative modification thereof;
h) the α chain variable region comprises a CDR1 comprising the amino acid sequence set forth in SEQ ID NO: 72 or a conservative modification thereof, and the β chain variable region comprises a CDR1 comprising the amino acid sequence set forth in SEQ ID NO: 48 or a conservative modification thereof;
i) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 78 or a conservative modification thereof;
j) the α chain variable region comprises a CDR1 comprising the amino acid sequence set forth in SEQ ID NO: 85 or a conservative modification thereof, and the β chain variable region comprises a CDR1 comprising the amino acid sequence set forth in SEQ ID NO: 37 or a conservative modification thereof;
k) the α chain variable region comprises a CDR1 comprising the amino acid sequence set forth in SEQ ID NO: 93 or a conservative modification thereof, and the β chain variable region comprises a CDR1 comprising the amino acid sequence set forth in SEQ ID NO: 48 or a conservative modification thereof;
l) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 102 or a conservative modification thereof;
m) the α chain variable region comprises a CDR1 comprising the amino acid sequence set forth in SEQ ID NO: 109 or a conservative modification thereof, and the β chain variable region comprises a CDR1 comprising the amino acid sequence set forth in SEQ ID NO: 112 or a conservative modification thereof;
n) the α chain variable region comprises a CDR1 comprising the amino acid sequence set forth in SEQ ID NO: 119 or a conservative modification thereof, and the β chain variable region comprises a CDR1 comprising the amino acid sequence set forth in SEQ ID NO: 122 or a conservative modification thereof;
o) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 131 or a conservative modification thereof;
p) the α chain variable region comprises a CDR1 comprising the amino acid sequence set forth in SEQ ID NO: 138 or a conservative modification thereof, and the β chain variable region comprises a CDR1 comprising the amino acid sequence set forth in SEQ ID NO: 102 or a conservative modification thereof;
q) the α chain variable region comprises a CDR1 comprising the amino acid sequence set forth in SEQ ID NO: 145 or a conservative modification thereof, and the β chain variable region comprises a CDR1 comprising the amino acid sequence set forth in SEQ ID NO: 102 or a conservative modification thereof;
r) the α chain variable region comprises a CDR1 comprising the amino acid sequence set forth in SEQ ID NO: 156 or a conservative modification thereof, and the β chain variable region comprises a CDR1 comprising the amino acid sequence set forth in SEQ ID NO: 159 or a conservative modification thereof;
s) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 182 or a conservative modification thereof;
t) the α chain variable region comprises a CDR1 comprising the amino acid sequence set forth in SEQ ID NO: 119 or a conservative modification thereof, and the β chain variable region comprises a CDR1 comprising the amino acid sequence set forth in SEQ ID NO: 37 or a conservative modification thereof;
u) the α chain variable region comprises a CDR1 comprising the amino acid sequence set forth in SEQ ID NO: 195 or a conservative modification thereof, and the β chain variable region comprises a CDR1 comprising the amino acid sequence set forth in SEQ ID NO: 159 or a conservative modification thereof;
v) the α chain variable region comprises a CDR1 comprising the amino acid sequence set forth in SEQ ID NO: 203 or a conservative modification thereof, and the β chain variable region comprises a CDR1 comprising the amino acid sequence set forth in SEQ ID NO: 122 or a conservative modification thereof;
w) the α chain variable region comprises a CDR1 comprising the amino acid sequence set forth in SEQ ID NO: 211 or a conservative modification thereof, and the β chain variable region comprises a CDR1 comprising the amino acid sequence set forth in SEQ ID NO: 214 or a conservative modification thereof;
x) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 159 or a conservative modification thereof;
y) the α chain variable region comprises a CDR1 comprising the amino acid sequence set forth in SEQ ID NO: 227 or a conservative modification thereof, and the β chain variable region comprises a CDR1 comprising the amino acid sequence set forth in SEQ ID NO: 230 or a conservative modification thereof;
z) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 238 or a conservative modification thereof;
aa) the α chain variable region comprises a CDR1 comprising the amino acid sequence set forth in SEQ ID NO: 72 or a conservative modification thereof, and the β chain variable region comprises a CDR1 comprising the amino acid sequence set forth in SEQ ID NO: 246 or a conservative modification thereof;
ab) the α chain variable region comprises a CDR1 comprising the amino acid sequence set forth in SEQ ID NO: 156 or a conservative modification thereof, and the β chain variable region comprises a CDR1 comprising the amino acid sequence set forth in SEQ ID NO: 37 or a conservative modification thereof;
ac) the α chain variable region comprises a CDR1 comprising the amino acid sequence set forth in SEQ ID NO: 260 or a conservative modification thereof, and the β chain variable region comprises a CDR1 comprising the amino acid sequence set forth in SEQ ID NO: 263 or a conservative modification thereof;
ad) the α chain variable region comprises a CDR1 comprising the amino acid sequence set forth in SEQ ID NO: 203 or a conservative modification thereof, and the β chain variable region comprises a CDR1 comprising the amino acid sequence set forth in SEQ ID NO: 271 or a conservative modification thereof;
ae) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 263 or a conservative modification thereof;
af) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 288 or a conservative modification thereof;
ag) the α chain variable region comprises a CDR1 comprising the amino acid sequence set forth in SEQ ID NO: 227 or a conservative modification thereof, and the β chain variable region comprises a CDR1 comprising the amino acid sequence set forth in SEQ ID NO: 296 or a conservative modification thereof;
ah) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 304 or a conservative modification thereof;
ai) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 27 or a conservative modification thereof;
aj) the α chain variable region comprises a CDR1 comprising the amino acid sequence set forth in SEQ ID NO: 317 or a conservative modification thereof, and the β chain variable region comprises a CDR1 comprising the amino acid sequence set forth in SEQ ID NO: 263 or a conservative modification thereof;
ak) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 326 or a conservative modification thereof;
al) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 288 or a conservative modification thereof;
am) the α chain variable region comprises a CDR1 comprising the amino acid sequence set forth in SEQ ID NO: 93 or a conservative modification thereof, and the β chain variable region comprises a CDR1 comprising the amino acid sequence set forth in SEQ ID NO: 182 or a conservative modification thereof;
an) the α chain variable region comprises a CDR1 comprising the amino acid sequence set forth in SEQ ID NO: 195 or a conservative modification thereof, and the β chain variable region comprises a CDR1 comprising the amino acid sequence set forth in SEQ ID NO: 182 or a conservative modification thereof;
ao) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 37 or a conservative modification thereof;
ap) the α chain variable region comprises a CDR1 comprising the amino acid sequence set forth in SEQ ID NO: 369 or a conservative modification thereof, and the β chain variable region comprises a CDR1 comprising the amino acid sequence set forth in SEQ ID NO: 326 or a conservative modification thereof; or
aq) the α chain variable region comprises a CDR1 comprising the amino acid sequence set forth in SEQ ID NO: 375 or a conservative modification thereof, and the β chain variable region comprises a CDR1 comprising the amino acid sequence set forth in SEQ ID NO: 37 or a conservative modification thereof.

In certain embodiments,
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
c) the α chain variable region comprises a CDR1 comprising the amino acid sequence set forth in SEQ ID NO: 24; a CDR2 comprising the amino acid sequence set forth in SEQ ID NO: 25; and a CDR3 comprising the amino acid sequence set forth in SEQ ID NO: 26;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55;
h) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66;
i) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
j) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77;
k) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87;
l) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95;
m) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101;
n) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111;
o) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121;
p) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130;
q) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139;
r) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147;
s) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150;
t) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158;
u) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173;
v) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181;
w) the α chain variable region comprises a CDR1 comprising the amino acid sequence set forth in SEQ ID NO: 119; a CDR2 comprising the amino acid sequence set forth in SEQ ID NO: 120; and a CDR3 comprising the amino acid sequence set forth in SEQ ID NO: 189;
x) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197;
y) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205;
z) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213;
aa) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221;
ab) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229;
ac) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237;
ad) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245;
ac) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254;
af) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262;
ag) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270;
ah) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278;
ai) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287;
aj) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295;
ak) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303;
al) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311;
am) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319;
an) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325;
ao) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333;
ap) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345;
ar) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349;
as) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356;
at) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360;
au) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371;
av) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; or
aw) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383.

In certain embodiments,
a) the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
f) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 55;
g) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
h) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
i) the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
j) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
k) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
l) the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
m) the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
n) the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
o) the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
p) the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
q) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
r) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
s) the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
t) the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
u) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
v) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
w) the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
x) the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
y) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
z) the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
aa) the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ab) the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
ac) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ad) the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ac) the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
af) the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
ag) the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ah) the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
ai) the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
aj) the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
ak) the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
al) the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
am) the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
an) the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ao) the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
ap) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
aq) the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290; or
ar) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments,
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56;
h) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
i) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
j) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
k) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
l) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
m) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
n) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
o) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
p) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
q) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
r) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
s) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
t) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
u) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
v) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
w) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
x) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
y) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
z) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
aa) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
ab) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
ac) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
ad) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ae) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
af) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ag) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ah) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
ai) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
aj) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ak) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
al) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
am) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
an) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
ao) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
ap) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ar) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
as) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
at) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
au) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
av) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
aw) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
ax) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378; or
ay) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments,
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; or
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19.

In certain embodiments, the α chain variable region comprises an amino acid sequence that is at least about 80% homologous or identical to the amino acid sequence set forth in SEQ ID NO: 11, SEQ ID NO: 20, SEQ ID NO: 30, SEQ ID NO: 40, SEQ ID NO: 45, SEQ ID NO: 51, SEQ ID NO: 57, SEQ ID NO: 68, SEQ ID NO: 75, SEQ ID NO: 81, SEQ ID NO: 89, SEQ ID NO: 97, SEQ ID NO: 105, SEQ ID NO: 115, SEQ ID NO: 124, SEQ ID NO: 134, SEQ ID NO: 141, SEQ ID NO: 148, SEQ ID NO: 152, SEQ ID NO: 162, SEQ ID NO: 175, SEQ ID NO: 185, SEQ ID NO: 191, SEQ ID NO: 199, SEQ ID NO: 207, SEQ ID NO: 217, SEQ ID NO: 223, SEQ ID NO: 233, SEQ ID NO: 241, SEQ ID NO: 249, SEQ ID NO: 256, SEQ ID NO: 266, SEQ ID NO: 274, SEQ ID NO: 281, SEQ ID NO: 291, SEQ ID NO: 299, SEQ ID NO: 307, SEQ ID NO: 313, SEQ ID NO: 321, SEQ ID NO: 329, SEQ ID NO: 335, SEQ ID NO: 341, SEQ ID NO: 346, SEQ ID NO: 352, SEQ ID NO: 357, SEQ ID NO: 362, SEQ ID NO: 375, SEQ ID NO: 379, or SEQ ID NO: 384. In certain embodiments, the α chain variable region comprises the amino acid sequence set forth in SEQ ID NO: 11, SEQ ID NO: 20, SEQ ID NO: 30, SEQ ID NO: 40, SEQ ID NO: 45, SEQ ID NO: 51, SEQ ID NO: 57, SEQ ID NO: 68, SEQ ID NO: 75, SEQ ID NO: 81, SEQ ID NO: 89, SEQ ID NO: 97, SEQ ID NO: 105, SEQ ID NO: 115, SEQ ID NO: 124, SEQ ID NO: 134, SEQ ID NO: 141, SEQ ID NO: 148, SEQ ID NO: 152, SEQ ID NO: 162, SEQ ID NO: 175, SEQ ID NO: 185, SEQ ID NO: 191, SEQ ID NO: 199, SEQ ID NO: 207, SEQ ID NO: 217, SEQ ID NO: 223, SEQ ID NO: 233, SEQ ID NO: 241, SEQ ID NO: 249, SEQ ID NO: 256, SEQ ID NO: 266, SEQ ID NO: 274, SEQ ID NO: 281, SEQ ID NO: 291, SEQ ID NO: 299, SEQ ID NO: 307, SEQ ID NO: 313, SEQ ID NO: 321, SEQ ID NO: 329, SEQ ID NO: 335, SEQ ID NO: 341, SEQ ID NO: 346, SEQ ID NO: 352, SEQ ID NO: 357, SEQ ID NO: 362, SEQ ID NO: 375, SEQ ID NO: 379, or SEQ ID NO: 384. In certain embodiments, the α chain variable region comprises the amino acid sequence set forth in SEQ ID NO: 11 or SEQ ID NO: 20.

In certain embodiments, the β chain variable region comprises an amino acid sequence that is at least about 80% homologous or identical to the amino acid sequence set forth in SEQ ID NO: 12, SEQ ID NO: 21, SEQ ID NO: 31, SEQ ID NO: 41, SEQ ID NO: 52, SEQ ID NO: 58, SEQ ID NO: 62, SEQ ID NO: 69, SEQ ID NO: 82, SEQ ID NO: 90, SEQ ID NO: 98, SEQ ID NO: 106, SEQ ID NO: 125, SEQ ID NO: 135, SEQ ID NO: 142, SEQ ID NO: 153, SEQ ID NO: 162, SEQ ID NO: 176, SEQ ID NO: 186, SEQ ID NO: 192, SEQ ID NO: 200, SEQ ID NO: 208, SEQ ID NO: 218, SEQ ID NO: 224, SEQ ID NO: 234, SEQ ID NO: 242, SEQ ID NO: 250, SEQ ID NO: 257, SEQ ID NO: 267, SEQ ID NO: 275, SEQ ID NO: 282, SEQ ID NO: 292, SEQ ID NO: 300, SEQ ID NO: 308, SEQ ID NO: 314, SEQ ID NO: 322, SEQ ID NO: 330, SEQ ID NO: 336, SEQ ID NO: 342, SEQ ID NO: 353, SEQ ID NO: 363, SEQ ID NO: 366, or SEQ ID NO: 380. In certain embodiments, the β chain variable region comprises the amino acid sequence set forth in SEQ ID NO: 12, SEQ ID NO: 21, SEQ ID NO: 31, SEQ ID NO: 41, SEQ ID NO: 52, SEQ ID NO: 58, SEQ ID NO: 62, SEQ ID NO: 69, SEQ ID NO: 82, SEQ ID NO: 90, SEQ ID NO: 98, SEQ ID NO: 106, SEQ ID NO: 125, SEQ ID NO: 135, SEQ ID NO: 142, SEQ ID NO: 153, SEQ ID NO: 162, SEQ ID NO: 176, SEQ ID NO: 186, SEQ ID NO: 192, SEQ ID NO: 200, SEQ ID NO: 208, SEQ ID NO: 218, SEQ ID NO: 224, SEQ ID NO: 234, SEQ ID NO: 242, SEQ ID NO: 250, SEQ ID NO: 257, SEQ ID NO: 267, SEQ ID NO: 275, SEQ ID NO: 282, SEQ ID NO: 292, SEQ ID NO: 300, SEQ ID NO: 308, SEQ ID NO: 314, SEQ ID NO: 322, SEQ ID NO: 330, SEQ ID NO: 336, SEQ ID NO: 342, SEQ ID NO: 353, SEQ ID NO: 363, SEQ ID NO: 366, or SEQ ID NO: 380. In certain embodiments, the β chain variable region comprises the amino acid sequence set forth in SEQ ID NO: 12 or SEQ ID NO: 21.

In certain embodiments,
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 20, and the β chain variable region comprises an amino acid sequence that is at least about 80% homologous or identical to the amino acid sequence set forth in SEQ ID NO: 21;
c) the α chain variable region comprises an amino acid sequence that is at least about 80% homologous or identical to the amino acid sequence set forth in SEQ ID NO: 30, and the β chain variable region comprises an amino acid sequence that is at least about 80% homologous or identical to the amino acid sequence set forth in SEQ ID NO: 31;
d) the α chain variable region comprises an amino acid sequence that is at least about 80% homologous or identical to the amino acid sequence set forth in SEQ ID NO: 40, and the β chain variable region comprises an amino acid sequence that is at least about 80% homologous or identical to the amino acid sequence set forth in SEQ ID NO: 41;
e) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 41;
f) the α chain variable region comprises an amino acid sequence that is at least about 80% homologous or identical to the amino acid sequence set forth in SEQ ID NO: 51, and the β chain variable region comprises an amino acid sequence that is at least about 80% homologous or identical to the amino acid sequence set forth in SEQ ID NO: 52;
g) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 58;
h) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 62;
i) the α chain variable region comprises an amino acid sequence that is at least about 80% homologous or identical to the amino acid sequence set forth in SEQ ID NO: 68, and the β chain variable region comprises an amino acid sequence that is at least about 80% homologous or identical to the amino acid sequence set forth in SEQ ID NO: 69;
j) the α chain variable region comprises an amino acid sequence that is at least about 80% homologous or identical to the amino acid sequence set forth in SEQ ID NO: 75, and the β chain variable region comprises an amino acid sequence that is at least about 80% homologous or identical to the amino acid sequence set forth in SEQ ID NO: 69;
k) the α chain variable region comprises an amino acid sequence that is at least about 80% homologous or identical to the amino acid sequence set forth in SEQ ID NO: 81, and the β chain variable region comprises an amino acid sequence that is at least about 80% homologous or identical to the amino acid sequence set forth in SEQ ID NO: 82;
l) the α chain variable region comprises an amino acid sequence that is at least about 80% homologous or identical to the amino acid sequence set forth in SEQ ID NO: 89, and the β chain variable region comprises an amino acid sequence that is at least about 80% homologous or identical to the amino acid sequence set forth in SEQ ID NO: 90;
m) the α chain variable region comprises an amino acid sequence that is at least about 80% homologous or identical to the amino acid sequence set forth in SEQ ID NO: 97, and the β chain variable region comprises an amino acid sequence that is at least about 80% homologous or identical to the amino acid sequence set forth in SEQ ID NO: 98;
n) the α chain variable region comprises an amino acid sequence that is at least about 80% homologous or identical to the amino acid sequence set forth in SEQ ID NO: 105, and the β chain variable region comprises an amino acid sequence that is at least about 80% homologous or identical to the amino acid sequence set forth in SEQ ID NO: 106;
o) the α chain variable region comprises an amino acid sequence that is at least about 80% homologous or identical to the amino acid sequence set forth in SEQ ID NO: 115, and the β chain variable region comprises an amino acid sequence that is at least about 80% homologous or identical to the amino acid sequence set forth in SEQ ID NO: 116;
p) the α chain variable region comprises an amino acid sequence that is at least about 80% homologous or identical to the amino acid sequence set forth in SEQ ID NO: 124, and the β chain variable region comprises an amino acid sequence that is at least about 80% homologous or identical to the amino acid sequence set forth in SEQ ID NO: 125;
q) the α chain variable region comprises an amino acid sequence that is at least about 80% homologous or identical to the amino acid sequence set forth in SEQ ID NO: 134, and the β chain variable region comprises an amino acid sequence that is at least about 80% homologous or identical to the amino acid sequence set forth in SEQ ID NO: 135;
r) the α chain variable region comprises an amino acid sequence that is at least about 80% homologous or identical to the amino acid sequence set forth in SEQ ID NO: 141, and the β chain variable region comprises an amino acid sequence that is at least about 80% homologous or identical to the amino acid sequence set forth in SEQ ID NO: 142;
s) the α chain variable region comprises an amino acid sequence that is at least about 80% homologous or identical to the amino acid sequence set forth in SEQ ID NO: 148, and the β chain variable region comprises an amino acid sequence that is at least about 80% homologous or identical to the amino acid sequence set forth in SEQ ID NO: 142;
t) the α chain variable region comprises an amino acid sequence that is at least about 80% homologous or identical to the amino acid sequence set forth in SEQ ID NO: 152, and the β chain variable region comprises an amino acid sequence that is at least about 80% homologous or identical to the amino acid sequence set forth in SEQ ID NO: 153;
u) the α chain variable region comprises an amino acid sequence that is at least about 80% homologous or identical to the amino acid sequence set forth in SEQ ID NO: 162, and the β chain variable region comprises the amino acid sequence set forth in SEQ ID NO: 163;
v) the α chain variable region comprises an amino acid sequence that is at least about 80% homologous or identical to the amino acid sequence set forth in SEQ ID NO: 175, and the β chain variable region comprises an amino acid sequence that is at least about 80% homologous or identical to the amino acid sequence set forth in SEQ ID NO: 176;
w) the α chain variable region comprises an amino acid sequence that is at least about 80% homologous or identical to the amino acid sequence set forth in SEQ ID NO: 185, and the β chain variable region comprises an amino acid sequence that is at least about 80% homologous or identical to the amino acid sequence set forth in SEQ ID NO: 186;
x) the α chain variable region comprises an amino acid sequence that is at least about 80% homologous or identical to the amino acid sequence set forth in SEQ ID NO: 191, and the β chain variable region comprises an amino acid sequence that is at least about 80% homologous or identical to the amino acid sequence set forth in SEQ ID NO: 192;
y) the α chain variable region comprises an amino acid sequence that is at least about 80% homologous or identical to the amino acid sequence set forth in SEQ ID NO: 199, and the β chain variable region comprises an amino acid sequence that is at least about 80% homologous or identical to the amino acid sequence set forth in SEQ ID NO: 200;
z) the α chain variable region comprises an amino acid sequence that is at least about 80% homologous or identical to the amino acid sequence set forth in SEQ ID NO: 207, and the β chain variable region comprises an amino acid sequence that is at least about 80% homologous or identical to the amino acid sequence set forth in SEQ ID NO: 208;
aa) the α chain variable region comprises an amino acid sequence that is at least about 80% homologous or identical to the amino acid sequence set forth in SEQ ID NO: 217, and the β chain variable region comprises an amino acid sequence that is at least about 80% homologous or identical to the amino acid sequence set forth in SEQ ID NO: 218;
ab) the α chain variable region comprises an amino acid sequence that is at least about 80% homologous or identical to the amino acid sequence set forth in SEQ ID NO: 223, and the β chain variable region comprises an amino acid sequence that is at least about 80% homologous or identical to the amino acid sequence set forth in SEQ ID NO: 224;
ac) the α chain variable region comprises an amino acid sequence that is at least about 80% homologous or identical to the amino acid sequence set forth in SEQ ID NO: 233, and the β chain variable region comprises an amino acid sequence that is at least about 80% homologous or identical to the amino acid sequence set forth in SEQ ID NO: 234;
ad) the α chain variable region comprises an amino acid sequence that is at least about 80% homologous or identical to the amino acid sequence set forth in SEQ ID NO: 241, and the β chain variable region comprises an amino acid sequence that is at least about 80% homologous or identical to the amino acid sequence set forth in SEQ ID NO: 242;
ac) the α chain variable region comprises an amino acid sequence that is at least about 80% homologous or identical to the amino acid sequence set forth in SEQ ID NO: 249, and the β chain variable region comprises an amino acid sequence that is at least about 80% homologous or identical to the amino acid sequence set forth in SEQ ID NO: 250;
af) the α chain variable region comprises an amino acid sequence that is at least about 80% homologous or identical to the amino acid sequence set forth in SEQ ID NO: 256, and the β chain variable region comprises an amino acid sequence that is at least about 80% homologous or identical to the amino acid sequence set forth in SEQ ID NO: 257;
ag) the α chain variable region comprises an amino acid sequence that is at least about 80% homologous or identical to the amino acid sequence set forth in SEQ ID NO: 266, and the β chain variable region comprises an amino acid sequence that is at least about 80% homologous or identical to the amino acid sequence set forth in SEQ ID NO: 267;
ah) the α chain variable region comprises an amino acid sequence that is at least about 80% homologous or identical to the amino acid sequence set forth in SEQ ID NO: 274, and the β chain variable region comprises an amino acid sequence that is at least about 80% homologous or identical to the amino acid sequence set forth in SEQ ID NO: 275;
ai) the α chain variable region comprises an amino acid sequence that is at least about 80% homologous or identical to the amino acid sequence set forth in SEQ ID NO: 281, and the β chain variable region comprises an amino acid sequence that is at least about 80% homologous or identical to the amino acid sequence set forth in SEQ ID NO: 282;
aj) the α chain variable region comprises an amino acid sequence that is at least about 80% homologous or identical to the amino acid sequence set forth in SEQ ID NO: 291, and the β chain variable region comprises an amino acid sequence that is at least about 80% homologous or identical to the amino acid sequence set forth in SEQ ID NO: 292;
ak) the α chain variable region comprises an amino acid sequence that is at least about 80% homologous or identical to the amino acid sequence set forth in SEQ ID NO: 299, and the β chain variable region comprises an amino acid sequence that is at least about 80% homologous or identical to the amino acid sequence set forth in SEQ ID NO: 300;
al) the α chain variable region comprises an amino acid sequence that is at least about 80% homologous or identical to the amino acid sequence set forth in SEQ ID NO: 307, and the β chain variable region comprises an amino acid sequence that is at least about 80% homologous or identical to the amino acid sequence set forth in SEQ ID NO: 308;
am) the α chain variable region comprises an amino acid sequence that is at least about 80% homologous or identical to the amino acid sequence set forth in SEQ ID NO: 313, and the β chain variable region comprises an amino acid sequence that is at least about 80% homologous or identical to the amino acid sequence set forth in SEQ ID NO: 314;
an) the α chain variable region comprises an amino acid sequence that is at least about 80% homologous or identical to the amino acid sequence set forth in SEQ ID NO: 321, and the β chain variable region comprises an amino acid sequence that is at least about 80% homologous or identical to the amino acid sequence set forth in SEQ ID NO: 322;
ao) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 330;
ap) the α chain variable region comprises an amino acid sequence that is at least about 80% homologous or identical to the amino acid sequence set forth in SEQ ID NO: 335, and the β chain variable region comprises an amino acid sequence that is at least about 80% homologous or identical to the amino acid sequence set forth in SEQ ID NO: 336;
aq) the α chain variable region comprises an amino acid sequence that is at least about 80% homologous or identical to the amino acid sequence set forth in SEQ ID NO: 341, and the β chain variable region comprises an amino acid sequence that is at least about 80% homologous or identical to the amino acid sequence set forth in SEQ ID NO: 342;
ar) the α chain variable region comprises an amino acid sequence that is at least about 80% homologous or identical to the amino acid sequence set forth in SEQ ID NO: 346, and the β chain variable region comprises an amino acid sequence that is at least about 80% homologous or identical to the amino acid sequence set forth in SEQ ID NO: 342;
as) the α chain variable region comprises an amino acid sequence that is at least about 80% homologous or identical to the amino acid sequence set forth in SEQ ID NO: 352, and the β chain variable region comprises an amino acid sequence that is at least about 80% homologous or identical to the amino acid sequence set forth in SEQ ID NO: 353;
at) the α chain variable region comprises an amino acid sequence that is at least about 80% homologous or identical to the amino acid sequence set forth in SEQ ID NO: 357, and the β chain variable region comprises an amino acid sequence that is at least about 80% homologous or identical to the amino acid sequence set forth in SEQ ID NO: 353;
au) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
av) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
aw) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 366;
ax) the α chain variable region comprises an amino acid sequence that is at least about 80% homologous or identical to the amino acid sequence set forth in SEQ ID NO: 372, and the β chain variable region comprises an amino acid sequence that is at least about 80% homologous or identical to the amino acid sequence set forth in SEQ ID NO: 366;
ay) the α chain variable region comprises an amino acid sequence that is at least about 80% homologous or identical to the amino acid sequence set forth in SEQ ID NO: 379, and the β chain variable region comprises an amino acid sequence that is at least about 80% homologous or identical to the amino acid sequence set forth in SEQ ID NO: 380; or
az) the α chain variable region comprises an amino acid sequence that is at least about 80% homologous or identical to the amino acid sequence set forth in SEQ ID NO: 384, and the β chain variable region comprises an amino acid sequence that is at least about 80% homologous or identical to the amino acid sequence set forth in SEQ ID NO: 380.

In certain embodiments,
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 20, and the β chain variable region comprises the amino acid sequence set forth in SEQ ID NO: 21;
c) the α chain variable region comprises the amino acid sequence set forth in SEQ ID NO: 30, and the β chain variable region comprises the amino acid sequence set forth in SEQ ID NO: 31;
d) the α chain variable region comprises the amino acid sequence set forth in SEQ ID NO: 40, and the β chain variable region comprises the amino acid sequence set forth in SEQ ID NO: 41;
e) the α chain variable region comprises the amino acid sequence set forth in SEQ ID NO: 45, and the β chain variable region comprises the amino acid sequence set forth in SEQ ID NO: 41;
f) the α chain variable region comprises the amino acid sequence set forth in SEQ ID NO: 51, and the β chain variable region comprises the amino acid sequence set forth in SEQ ID NO: 52;
g) the α chain variable region comprises the amino acid sequence set forth in SEQ ID NO: 57, and the β chain variable region comprises the amino acid sequence set forth in SEQ ID NO: 58;
h) the α chain variable region comprises the amino acid sequence set forth in SEQ ID NO: 57, and the β chain variable region comprises the amino acid sequence set forth in SEQ ID NO: 62;
i) the α chain variable region comprises the amino acid sequence set forth in SEQ ID NO: 68, and the β chain variable region comprises the amino acid sequence set forth in SEQ ID NO: 69;
j) the α chain variable region comprises the amino acid sequence set forth in SEQ ID NO: 75, and the β chain variable region comprises the amino acid sequence set forth in SEQ ID NO: 69;
k) the α chain variable region comprises the amino acid sequence set forth in SEQ ID NO: 81, and the β chain variable region comprises the amino acid sequence set forth in SEQ ID NO: 82;
l) the α chain variable region comprises the amino acid sequence set forth in SEQ ID NO: 89, and the β chain variable region comprises the amino acid sequence set forth in SEQ ID NO: 90;
m) the α chain variable region comprises the amino acid sequence set forth in SEQ ID NO: 97, and the β chain variable region comprises the amino acid sequence set forth in SEQ ID NO: 98;
n) the α chain variable region comprises the amino acid sequence set forth in SEQ ID NO: 105, and the β chain variable region comprises the amino acid sequence set forth in SEQ ID NO: 106;
o) the α chain variable region comprises the amino acid sequence set forth in SEQ ID NO: 115, and the β chain variable region comprises the amino acid sequence set forth in SEQ ID NO: 116;
p) the α chain variable region comprises the amino acid sequence set forth in SEQ ID NO: 124, and the β chain variable region comprises the amino acid sequence set forth in SEQ ID NO: 125;
q) the α chain variable region comprises the amino acid sequence set forth in SEQ ID NO: 134, and the β chain variable region comprises the amino acid sequence set forth in SEQ ID NO: 135;
r) the α chain variable region comprises the amino acid sequence set forth in SEQ ID NO: 141, and the β chain variable region comprises the amino acid sequence set forth in SEQ ID NO: 142;
s) the α chain variable region comprises the amino acid sequence set forth in SEQ ID NO: 148, and the β chain variable region comprises the amino acid sequence set forth in SEQ ID NO: 142;
t) the α chain variable region comprises the amino acid sequence set forth in SEQ ID NO: 152, and the β chain variable region comprises the amino acid sequence set forth in SEQ ID NO: 153;
u) the α chain variable region comprises the amino acid sequence set forth in SEQ ID NO: 162, and the β chain variable region comprises an amino acid sequence that is at least about 80% homologous or identical to the amino acid sequence set forth in SEQ ID NO: 163;
v) the α chain variable region comprises the amino acid sequence set forth in SEQ ID NO: 175, and the β chain variable region comprises the amino acid sequence set forth in SEQ ID NO: 176;
w) the α chain variable region comprises the amino acid sequence set forth in SEQ ID NO: 185, and the β chain variable region comprises the amino acid sequence set forth in SEQ ID NO: 186;
x) the α chain variable region comprises the amino acid sequence set forth in SEQ ID NO: 191, and the β chain variable region comprises the amino acid sequence set forth in SEQ ID NO: 192;
y) the α chain variable region comprises the amino acid sequence set forth in SEQ ID NO: 199, and the β chain variable region comprises the amino acid sequence set forth in SEQ ID NO: 200;
z) the α chain variable region comprises the amino acid sequence set forth in SEQ ID NO: 207, and the β chain variable region comprises the amino acid sequence set forth in SEQ ID NO: 208;
aa) the α chain variable region comprises the amino acid sequence set forth in SEQ ID NO: 217, and the β chain variable region comprises the amino acid sequence set forth in SEQ ID NO: 218;
ab) the α chain variable region comprises the amino acid sequence set forth in SEQ ID NO: 223, and the β chain variable region comprises the amino acid sequence set forth in SEQ ID NO: 224;
ac) the α chain variable region comprises the amino acid sequence set forth in SEQ ID NO: 233, and the β chain variable region comprises the amino acid sequence set forth in SEQ ID NO: 234;
ad) the α chain variable region comprises the amino acid sequence set forth in SEQ ID NO: 241, and the β chain variable region comprises the amino acid sequence set forth in SEQ ID NO: 242;
ac) the α chain variable region comprises the amino acid sequence set forth in SEQ ID NO: 249, and the β chain variable region comprises the amino acid sequence set forth in SEQ ID NO: 250;
af) the α chain variable region comprises the amino acid sequence set forth in SEQ ID NO: 256, and the β chain variable region comprises the amino acid sequence set forth in SEQ ID NO: 257;
ag) the α chain variable region comprises the amino acid sequence set forth in SEQ ID NO: 266, and the β chain variable region comprises the amino acid sequence set forth in SEQ ID NO: 267;
ah) the α chain variable region comprises the amino acid sequence set forth in SEQ ID NO: 274, and the β chain variable region comprises the amino acid sequence set forth in SEQ ID NO: 275;
ai) the α chain variable region comprises the amino acid sequence set forth in SEQ ID NO: 281, and the β chain variable region comprises the amino acid sequence set forth in SEQ ID NO: 282;
aj) the α chain variable region comprises the amino acid sequence set forth in SEQ ID NO: 291, and the β chain variable region comprises the amino acid sequence set forth in SEQ ID NO: 292;
ak) the α chain variable region comprises the amino acid sequence set forth in SEQ ID NO: 299, and the β chain variable region comprises the amino acid sequence set forth in SEQ ID NO: 300;
al) the α chain variable region comprises the amino acid sequence set forth in SEQ ID NO: 307, and the β chain variable region comprises the amino acid sequence set forth in SEQ ID NO: 308;
am) the α chain variable region comprises the amino acid sequence set forth in SEQ ID NO: 313, and the β chain variable region comprises the amino acid sequence set forth in SEQ ID NO: 314;
an) the α chain variable region comprises the amino acid sequence set forth in SEQ ID NO: 321, and the β chain variable region comprises the amino acid sequence set forth in SEQ ID NO: 322;
ao) the α chain variable region comprises the amino acid sequence set forth in SEQ ID NO: 329, and the β chain variable region comprises the amino acid sequence set forth in SEQ ID NO: 330;
ap) the α chain variable region comprises the amino acid sequence set forth in SEQ ID NO: 335, and the β chain variable region comprises the amino acid sequence set forth in SEQ ID NO: 336;
aq) the α chain variable region comprises the amino acid sequence set forth in SEQ ID NO: 341, and the β chain variable region comprises the amino acid sequence set forth in SEQ ID NO: 342;
ar) the α chain variable region comprises the amino acid sequence set forth in SEQ ID NO: 346, and the β chain variable region comprises the amino acid sequence set forth in SEQ ID NO: 342;
as) the α chain variable region comprises the amino acid sequence set forth in SEQ ID NO: 352, and the β chain variable region comprises the amino acid sequence set forth in SEQ ID NO: 353;
at) the α chain variable region comprises the amino acid sequence set forth in SEQ ID NO: 357, and the β chain variable region comprises the amino acid sequence set forth in SEQ ID NO: 353;
au) the α chain variable region comprises the amino acid sequence set forth in SEQ ID NO: 362, and the β chain variable region comprises the amino acid sequence set forth in SEQ ID NO: 363;
av) the α chain variable region comprises the amino acid sequence set forth in SEQ ID NO: 362, and the β chain variable region comprises the amino acid sequence set forth in SEQ ID NO: 363;
aw) the α chain variable region comprises the amino acid sequence set forth in SEQ ID NO: 329, and the β chain variable region comprises the amino acid sequence set forth in SEQ ID NO: 366;
ax) the α chain variable region comprises the amino acid sequence set forth in SEQ ID NO: 372, and the β chain variable region comprises the amino acid sequence set forth in SEQ ID NO: 366;
ay) the α chain variable region comprises the amino acid sequence set forth in SEQ ID NO: 379, and the β chain variable region comprises the amino acid sequence set forth in SEQ ID NO: 380; or
az) the α chain variable region comprises the amino acid sequence set forth in SEQ ID NO: 384, and the β chain variable region comprises the amino acid sequence set forth in SEQ ID NO: 380.

In certain embodiments,
a) the α chain variable region comprises the amino acid sequence set forth in SEQ ID NO: 11, and the β chain variable region comprises the amino acid sequence set forth in SEQ ID NO: 12; or
b) the α chain variable region comprises the amino acid sequence set forth in SEQ ID NO: 20, and the β chain variable region comprises the amino acid sequence set forth in SEQ ID NO: 21.

In certain embodiments,
a) the α chain comprises the amino acid sequence set forth in SEQ ID NO: 13, and the β chain comprises the amino acid sequence set forth in SEQ ID NO: 14;
b) the α chain comprises the amino acid sequence set forth in SEQ ID NO: 22, and the β chain comprises the amino acid sequence set forth in SEQ ID NO: 23;
c) the α chain comprises the amino acid sequence set forth in SEQ ID NO: 32, and the β chain comprises the amino acid sequence set forth in SEQ ID NO: 33;
d) the α chain comprises the amino acid sequence set forth in SEQ ID NO: 42, and the β chain comprising the amino acid sequence set forth in SEQ ID NO: 43;
e) the α chain comprises the amino acid sequence set forth in SEQ ID NO: 46, and the β chain comprises the amino acid sequence set forth in SEQ ID NO: 43;
f) the α chain comprises the amino acid sequence set forth in SEQ ID NO: 53, and the β chain comprises the amino acid sequence set forth in SEQ ID NO: 54;
g) the α chain comprises the amino acid sequence set forth in SEQ ID NO: 59, and the β chain comprises the amino acid sequence set forth in SEQ ID NO: 60;
h) the α chain comprises the amino acid sequence set forth in SEQ ID NO: 59, and the β chain comprises the amino acid sequence set forth in SEQ ID NO: 63;
i) the α chain comprises the amino acid sequence set forth in SEQ ID NO: 70, and the β chain comprises the amino acid sequence set forth in SEQ ID NO: 71;
j) the α chain comprises the amino acid sequence set forth in SEQ ID NO: 76, and the β chain comprises the amino acid sequence set forth in SEQ ID NO: 71;
k) the α chain comprises the amino acid sequence set forth in SEQ ID NO: 83, and the β chain comprises the amino acid sequence set forth in SEQ ID NO: 84;
l) the α chain comprises the amino acid sequence set forth in SEQ ID NO: 91, and the β chain comprises the amino acid sequence set forth in SEQ ID NO: 92;
m) the α chain comprises the amino acid sequence set forth in SEQ ID NO: 99, and the β chain comprises the amino acid sequence set forth in SEQ ID NO: 100;
n) the α chain comprises the amino acid sequence set forth in SEQ ID NO: 107, and the β chain comprises the amino acid sequence set forth in SEQ ID NO: 108;
o) the α chain comprises the amino acid sequence set forth in SEQ ID NO: 117, and the β chain comprises the amino acid sequence set forth in SEQ ID NO: 118;
p) the α chain comprises the amino acid sequence set forth in SEQ ID NO: 126, and the β chain comprises the amino acid sequence set forth in SEQ ID NO: 127;
q) the α chain comprises the amino acid sequence set forth in SEQ ID NO: 136, and the β chain comprises the amino acid sequence set forth in SEQ ID NO: 137;
r) the α chain comprises the amino acid sequence set forth in SEQ ID NO: 143, and the β chain comprises the amino acid sequence set forth in SEQ ID NO: 144;
s) the α chain comprises the amino acid sequence set forth in SEQ ID NO: 149, and the β chain comprises the amino acid sequence set forth in SEQ ID NO: 144;
t) the α chain comprises the amino acid sequence set forth in SEQ ID NO: 154, and the β chain comprises the amino acid sequence set forth in SEQ ID NO: 155;
u) the α chain comprises the amino acid sequence set forth in SEQ ID NO: 164, and the β chain comprises the amino acid sequence set forth in SEQ ID NO: 165;
v) the α chain comprises the amino acid sequence set forth in SEQ ID NO: 177, and the β chain comprises the amino acid sequence set forth in SEQ ID NO: 178;
w) the α chain comprises the amino acid sequence set forth in SEQ ID NO: 187, and the β chain comprises the amino acid sequence set forth in SEQ ID NO: 188;
x) the α chain comprises the amino acid sequence set forth in SEQ ID NO: 193, and the β chain comprises the amino acid sequence set forth in SEQ ID NO: 194;
y) the α chain comprises the amino acid sequence set forth in SEQ ID NO: 201, and the β chain comprising the amino acid sequence set forth in SEQ ID NO: 202;
z) the α chain comprises the amino acid sequence set forth in SEQ ID NO: 209, and the β chain comprises the amino acid sequence set forth in SEQ ID NO: 210;
aa) the α chain comprises the amino acid sequence set forth in SEQ ID NO: 219, and the β chain comprises the amino acid sequence set forth in SEQ ID NO: 220;
ab) the α chain comprises the amino acid sequence set forth in SEQ ID NO: 225, and the β chain comprises the amino acid sequence set forth in SEQ ID NO: 226;
ac) the α chain comprises the amino acid sequence set forth in SEQ ID NO: 235, and the β chain comprises the amino acid sequence set forth in SEQ ID NO: 236;
ad) the α chain comprises the amino acid sequence set forth in SEQ ID NO: 243, and the β chain comprises the amino acid sequence set forth in SEQ ID NO: 244;
ac) the α chain comprises the amino acid sequence set forth in SEQ ID NO: 251, and the β chain comprises the amino acid sequence set forth in SEQ ID NO: 252;
af) the α chain comprises the amino acid sequence set forth in SEQ ID NO: 258, and the β chain comprises the amino acid sequence set forth in SEQ ID NO: 259;
ag) the α chain comprises the amino acid sequence set forth in SEQ ID NO: 268, and the β chain comprises the amino acid sequence set forth in SEQ ID NO: 269;
ah) the α chain comprises the amino acid sequence set forth in SEQ ID NO: 276, and the β chain comprises the amino acid sequence set forth in SEQ ID NO: 277;
ai) the α chain comprises the amino acid sequence set forth in SEQ ID NO: 283, and the β chain comprises the amino acid sequence set forth in SEQ ID NO: 284;
aj) the α chain comprises the amino acid sequence set forth in SEQ ID NO: 293, and the β chain comprises the amino acid sequence set forth in SEQ ID NO: 294;
ak) the α chain comprises the amino acid sequence set forth in SEQ ID NO: 301, and the β chain comprises the amino acid sequence set forth in SEQ ID NO: 302;
al) the α chain comprises the amino acid sequence set forth in SEQ ID NO: 309, and the β chain comprises the amino acid sequence set forth in SEQ ID NO: 310;
am) the α chain comprises the amino acid sequence set forth in SEQ ID NO: 315, and the β chain comprises the amino acid sequence set forth in SEQ ID NO: 316;
an) the α chain comprises the amino acid sequence set forth in SEQ ID NO: 323, and the β chain comprises the amino acid sequence set forth in SEQ ID NO: 324;
ao) the α chain comprises the amino acid sequence set forth in SEQ ID NO: 331, and the β chain comprises the amino acid sequence set forth in SEQ ID NO: 332;
ap) the α chain comprises the amino acid sequence set forth in SEQ ID NO: 337, and the β chain comprises the amino acid sequence set forth in SEQ ID NO: 338;
aq) the α chain comprises the amino acid sequence set forth in SEQ ID NO: 343, and the β chain comprises the amino acid sequence set forth in SEQ ID NO: 344;
ar) the α chain comprises the amino acid sequence set forth in SEQ ID NO: 347, and the β chain comprising the amino acid sequence set forth in SEQ ID NO: 348;
as) the α chain comprises the amino acid sequence set forth in SEQ ID NO: 354, and the β chain comprises the amino acid sequence set forth in SEQ ID NO: 355;
at) the α chain comprises the amino acid sequence set forth in SEQ ID NO: 358, and the β chain comprises the amino acid sequence set forth in SEQ ID NO: 359;
au) the α chain comprises the amino acid sequence set forth in SEQ ID NO: 364, and the β chain comprises the amino acid sequence set forth in SEQ ID NO: 365;
av) the α chain comprises the amino acid sequence set forth in SEQ ID NO: 367, and the β chain comprises the amino acid sequence set forth in SEQ ID NO: 368;
aw) the α chain comprises the amino acid sequence set forth in SEQ ID NO: 373, and the β chain comprises the amino acid sequence set forth in SEQ ID NO: 374;
ax) the α chain comprises the amino acid sequence set forth in SEQ ID NO: 381, and the β chain comprises the amino acid sequence set forth in SEQ ID NO: 382; or
ay) the α chain comprises the amino acid sequence set forth in SEQ ID NO: 385, and the β chain comprises the amino acid sequence set forth in SEQ ID NO: 386.

In certain embodiments,
a) the α chain comprises the amino acid sequence set forth in SEQ ID NO: 13, and the β chain comprises the amino acid sequence set forth in SEQ ID NO: 14; or
b) the α chain comprises the amino acid sequence set forth in SEQ ID NO: 22, and the β chain comprises the amino acid sequence set forth in SEQ ID NO: 23.

In certain embodiments, the extracellular domain binds to the same RAS peptide as a reference TCR or a functional fragment thereof, wherein the reference TCR or functional fragment thereof comprises an α chain variable region and a β chain variable region, wherein:
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56;
h) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
i) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; and the β chain variable comprising the amino acid sequence set forth in SEQ ID NO: 49, and a CDR3 comprising the amino acid sequence set forth in SEQ ID NO: 67;
j) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
k) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
l) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
m) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
n) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
o) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
p) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
q) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
r) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
s) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
t) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
u) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
v) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
w) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
x) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
y) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
z) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
aa) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
ab) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
ac) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
ad) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ac) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
af) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ag) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ah) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
ai) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
aj) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ak) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
al) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
am) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
an) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
ao) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
ap) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ar) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
as) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
at) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
au) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
av) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
aw) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
ax) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378; or
ay) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments, the TCR is recombinantly expressed, and/or expressed from a vector. In certain embodiments, the TCR does not bind to a RAS peptide comprising or consisting of the amino acid sequence set forth in SEQ ID NO: 4.

In certain embodiments, the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 166 or SEQ ID NO: 167. In certain embodiments, the α chain constant region comprises the amino acid sequence set forth in SEQ ID NO: 166 or SEQ ID NO: 167.

In certain embodiments, the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 168, SEQ ID NO: 169, SEQ ID NO: 170, or SEQ ID NO: 171. In certain embodiments, the β chain constant region comprises the amino acid sequence set forth in SEQ ID NO: 168, SEQ ID NO: 169, SEQ ID NO: 170, or SEQ ID NO: 171.

The presently disclosed subject matter provides nucleic acids encoding the TRCs disclosed herein. The presently disclosed subject matter further provides cells comprising the TRCs or nucleic acids disclosed herein. In certain embodiments, the cell is transduced with the TCR. In certain embodiments, the TCR is constitutively expressed on the surface of the cell.

In certain embodiments, the cell is an immunoresponsive cell. In certain embodiments, the cell is selected from the group consisting of a T cell, a Natural Killer (NK) cell, and a pluripotent stem cell from which a lymphoid cell may be differentiated. In certain embodiments, the cell is a T cell. In certain embodiments, the cell is a Natural Killer (NK) cell. In certain embodiments, the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell. In certain embodiments, the T cell is a γδ T cell. In certain embodiments, the T cell is a NK-T cell.

In certain embodiments, the TCR or the nucleic acid is integrated at a locus within the genome of the cell. In certain embodiments, the locus is selected from the group consisting of a TRAC locus, a TRBC locus, a TRDC locus, and a TRGC locus. In certain embodiments, the locus is a TRAC locus or a TRBC locus.

The presently disclosed subject matter also provides compositions comprising the cells disclosed herein. In certain embodiments, the composition is a pharmaceutical composition further comprising a pharmaceutically acceptable carrier.

Further, the presently disclosed subject matter also provides vectors comprising the nucleic acids disclosed herein. In certain embodiments, the vector is a γ-retroviral vector.

Additionally, the presently disclosed subject matter provides methods for producing a cell that binds to a RAS peptide that comprises a Q61 mutation. In certain embodiments, the method comprises introducing into the cell a nucleic acid or a vector disclosed herein.

Moreover, the presently disclosed subject matter provides polypeptides comprising a TCR (or functional fragment thereof) disclosed herein and a first binding molecule. In certain embodiments, the first binding molecule is an antibody, an antibody fragment, a peptide, or a binding mimetic. In certain embodiments, the polypeptide further comprises a second binding molecule. In certain embodiments, the second binding molecule is an antibody, an antibody fragment, a peptide, or a binding mimetic.

Furthermore, the presently disclosed subject matter provides methods of treating and/or preventing a tumor associated with RAS in a subject. In certain embodiments, the method comprises administering to the subject the cell, the composition, or the polypeptide disclosed herein. In certain embodiments, the tumor is associated with a RAS mutation. In certain embodiments, the RAS mutation is a Q61R mutation, a Q61K mutation, or a Q61L mutation. In certain embodiments, the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer (also known as “stomach cancer”), neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

In certain embodiments, the tumor is colorectal cancer. In certain embodiments, the tumor is thyroid cancer. In certain embodiments, the tumor is cutaneous melanoma. In certain embodiments, the subject is a human.

Finally, the presently disclosed subject matter provides cells, compositions, or polypeptides disclosed herein for use in treating and/or preventing a tumor associated with RAS in a subject. In certain embodiments, wherein the tumor is associated with a RAS mutation. In certain embodiments, the RAS mutation is a Q61R mutation, a Q61K mutation, or a Q61L mutation. In certain embodiments, the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma. In certain embodiments, the tumor is colorectal cancer, thyroid cancer, or cutaneous melanoma. In certain embodiments, the subject is human.

### 4. BRIEF DESCRIPTION OF THE FIGURES

The following Detailed Description, given by way of example but not intended to limit the invention to specific embodiments described, may be understood in conjunction with the accompanying drawings.

FIGS. 1A-1D illustrate validation of NRAS Q61mut public neoantigen specific TCRs. FIG. 1A shows a drawing illustrating the experimental design to validate the functionality of NRAS Q61mut public neoantigen TCRs. FIG. 1B shows the functional avidity of CD8⁺ (left) or CD4⁺ (right) T cells individually transduced with TCR 1 or TCR 2. Transduced T cells were co-cultured with COS7 cells electroporated with HLA-A*01:01 and pulsed with the indicated concentrations of WT or Q61R mut NRAS peptides. FIG. 1C shows the physiological recognition of the cognate antigen by the T cells individually transduced with TCR 1 or TCR2 assessed by co-culturing the T cells with human primary melanoma cell lines of molecularly defined NRAS Q61 mutation status and HLA-A*01 status. The cell lines were pre-incubated with IFNγ when indicated. FIG. 1D shows cross-protection towards the NRAS Q61K neoantigen by T cells transduced with TCR 1. The primary melanoma cell lines were electroporated with HLA-A*01:01 mRNA where indicated.

FIG. 2 shows validation of RAS Q61mut public neoantigen specific TCRs. T cells expressing the indicated TCR were labeled with dual fluorophore-conjugated dextramers including HLA-A multimers bound to RAS wild-type or Q61 mut.

FIG. 3 shows functional avidity of T cells individually transduced with inducated TCR. Transduced T cells were co-cultured with COS7 cells electroporated with HLA-A*01:01 and pulsed with the indicated concentrations of WT or Q61mut RAS peptides.

FIG. 4 shows specific cytolysis of fluorescently-labeled primary melanoma cell lines expressing RAS wild-type or Q61mut by T cells transduced with TCRs. Real time tumor cell tracking was performed using IncuCyte imaging system.

### 5. DETAILED DESCRIPTION OF THE INVENTION

The presently disclosed subject matter provides TCRs, targeting RAS comprising a mutation, e.g., a Q61R or Q61K mutation. Furthermore, the presently disclosed subject matter provides cells (e.g., T cells) comprising the RAS-targeted TCRs, and methods of using such cells for treating tumors associated with RAS mutation(s).

For purposes of clarity of disclosure and not by way of limitation, the detailed description is divided into the following subsections:
5.1. Definitions;
5.2. RAS;
5.3. TCRs;
5.4. Cells;
5.5. Nucleic Acids and Genetic Modifications of Cell;
5.6. Formulations and Administration;
5.7. Methods of Treatments;
5.8. Diagnostic and Prognostic Methods;
5.9. Kits; and
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and β-chain. Generally, a TCR comprises three CDRs in the α-chain variable region and three CDRs in the β-chain variable region. CDRs provide the majority of contact residues for the binding of the TCR to the antigen or epitope. CDRs regions can be delineated using the Kabat system (Kabat, E. A., et al. (1991) Sequences of Proteins of Immunological Interest, Fifth Edition, U.S. Department of Health and Human Services, NIH Publication No. 91-3242), the Chothia numbering system (Chothia et al., J Mol Biol. (1987) 196:901-17), the AbM numbering system (Abhinandan et al., Mol. Immunol. 2008, 45, 3832-3839), or the IMGT numbering system (accessible athttp://www.imgt.org/IMGTScientificChart/Numbering/IMGTIGVLsuperfamily.html #table1, http://www.imgt.org/IMGTScientificChart/Nomenclature/IMGT-FRCDRdefinition.html). In certain embodiments, the CDRs regions are delineated using the IMGT numbering system.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparaginc, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “disease” refers to any condition or disorder that damages or interferes with the normal function of a cell, tissue, or organ. Examples of diseases include neoplasm or pathogen infection of a cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a tumor), prevent or delay the recurrence of a tumor, or otherwise reduce the pathological consequences of the disease (e.g., a tumor). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “antigen” refers to any substance that the body (e.g., human body) regards as foreign and that therefore elicits an immune response (e.g., formation of specific antibodies capable of binding to it).

As used herein, the term “neoantigen” or “NeoAg” refers to aberrant antigens, which can be recognized by immune cells and result from mutations in cancer cell genes (e.g., caused by genetic instability during carcinogenesis). Neoantigens can arise through diverse mechanisms including, but not limited to, gene fusions, missense mutations, alternative splicing and indels. Neoantigens can be presented on the cell surface and subsequently recognized by T cells under the action of major histocompatibility complex (MHC) molecules.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses; and non-human primates such as apes and monkeys.

### 5.2. RAS

RAS is a family of oncoproteins encoding small GTPases involved in regulating cell growth, differentiation and survival of cells. In humans, the RAS family includes HRAS, NRAS, and KRAS. The KRAS gene has two splice variants, KRAS4A and KRAS4B. The expression of all isoforms is nearly ubiquitous, although they show quantitative and qualitative differences in expression depending on the tissue and/or developmental stage.

RAS proteins contain two domains: a G domain that binds guanosine nucleotides, and a C-terminal hypervariable region. The G domain is highly conserved between HRAS, NRAS, KRAS4A and KRAS4B and is responsible for binding and hydrolysis of guanine nucleotides. The hypervariable regions undergo differential post-translational modifications that in turn direct isoform-specific subcellular organization. RAS proteins act as binary molecular switches and cycle between an inactive GDP-bound and active GTP-bound state. Upon activation, RAS proteins recruit and activate proteins like c-Raf and PI3-kinase that result in cell proliferation, migration and protection from apoptosis.

RAS mutations play a critical role in driving some of the most common and deadly carcinomas, including pancreatic, lung, and colorectal cancers, among numerous others. The conserved G domain includes several locations for hotspot mutations including G12, G13, and Q61. One of the most frequent mutations of RAS genes occur at codon 61 (i.e., Q61R/K/L). Across cancers, the most common NRAS mutation is Q61R, which is a single point mutation with a glutamine-to-arginine substitution at codon 61. The second most common NRAS mutation is Q61K, which is a single point mutation with a glutamine-to-lysine substitution at codon 61. The third most common NRAS mutation is Q61L, which is a single point mutation with a glutamine-to-leucine substitution at codon 61.

### 5.3. T-Cell Receptor (TCR)

A TCR is a disulfide-linked heterodimeric protein consisting of two variable chains expressed as part of a non-covalent complex with the invariant CD3 chain molecules (CD3δ, CD3ε, CD3γ, CD3ζ). A TCR is found on the surface of T cells, and is responsible for recognizing antigens bound to major histocompatibility complex (MHC) molecules. In certain embodiments, a TCR comprises an α chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its cognate peptide antigen/MHC (peptide/MHC), the T cell expressing the TCR complex is activated.

The presently disclosed subject matter provides recombinant TCRs. In certain embodiments, the recombinant TCR differs from any naturally occurring TCR by at least one amino acid residue. In certain embodiments, the recombinant TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the recombinant TCR is modified from a naturally occurring TCR by at least one amino acid residue. In certain embodiments, the recombinant TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the presently disclosed TCR targets or binds to a RAS peptide that comprises a mutation (“a mutant RAS peptide”). In certain embodiments, the mutation is a point mutation. In certain embodiments, the mutation is a Q61 mutation. In certain embodiments, the RAS peptide comprises or consists of the amino acid sequence set forth in SEQ ID NO: 1. In certain embodiments, the RAS peptide comprises or consists of the amino acid sequence set forth in SEQ ID NO: 2. In certain embodiments, the RAS peptide comprises or consists of the amino acid sequence set forth in SEQ ID NO: 3. In certain embodiments, the presently disclosed TCR does not bind to a wildtype RAS. In certain embodiments, the presently disclosed TCR does not bind to a RAS peptide comprising or consisting of the amino acid sequence set forth in SEQ ID NO: 4. SEQ ID NOs: 1-4 are provided below.

[SEQ ID NO: 1]
ILDTAGREEY
[SEQ ID NO: 2]
ILDTAGKEEY
[SEQ ID NO: 3]
ILDTAGLEEY
[SEQ ID NO: 4]
ILDTAGQEEY

In certain embodiments, the presently disclosed TCR targets or binds to NRAS comprising a RAS peptide comprising or consisting of the amino acid sequence set forth in SEQ ID NO: 1. In certain embodiments, the presently disclosed TCR targets or binds to NRAS comprising a RAS peptide comprising or consisting of the amino acid sequence set forth in SEQ ID NO: 2. In certain embodiments, the presently disclosed TCR targets or binds to NRAS comprising a RAS peptide comprising or consisting of the amino acid sequence set forth in SEQ ID NO: 3.

In certain embodiments, the presently disclosed TCR targets or binds to KRAS comprising a RAS peptide comprising or consisting of the amino acid sequence set forth in SEQ ID NO: 1. In certain embodiments, the presently disclosed TCR targets or binds to KRAS comprising a RAS peptide comprising or consisting of the amino acid sequence set forth in SEQ ID NO: 2. In certain embodiments, the presently disclosed TCR targets or binds to KRAS comprising a RAS peptide comprising or consisting of the amino acid sequence set forth in SEQ ID NO: 3.

In certain embodiments, the presently disclosed TCR targets or binds to HRAS comprising a RAS peptide comprising or consisting of the amino acid sequence set forth in SEQ ID NO: 1. In certain embodiments, the presently disclosed TCR targets or binds to HRAS comprising a RAS peptide comprising or consisting of the amino acid sequence set forth in SEQ ID NO: 2. In certain embodiments, the presently disclosed TCR targets or binds to HRAS comprising a RAS peptide comprising or consisting of the amino acid sequence set forth in SEQ ID NO: 3. In certain embodiments, the presently disclosed TCR targets or binds to a RAS peptide associated with an HLA class I complex, e.g., HLA-A, HLA-B and HLA-C.

In certain embodiments, the presently disclosed TCR targets or binds to a RAS peptide associated with an HLA-A*01 superfamily (e.g., in an HLA-A*01 superfamily dependent manner). In certain embodiments, the HLA-A*01 superfamily member is selected from the group consisting of HLA-A*01, HLA-A*25, HLA-A*26, HLA-A*30, HLA-A*31, HLA-A*32, HLA-A*36, HLA-A*74:10, and HLA-A*80. In certain embodiments, the presently disclosed TCR targets or binds to a RAS peptide associated with an HLA-A*01 molecule.

### 5.3.1. TCRs

### Variable Regions 5.3.1.1.

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof. SEQ ID NOS: 5-7 are disclosed in Table 1. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7.

In certain embodiments, the extracellular domain of the TCR comprises a β chain variable region comprising a CDR1 comprising the amino acid sequence set forth in SEQ ID NO: 8 or a conservative modification thereof, a CDR2 comprising the amino acid sequence set forth in SEQ ID NO: 9 or a conservative modification thereof, and a CDR3 comprising the amino acid sequence set forth in SEQ ID NO: 10 or a conservative modification thereof. SEQ ID NOS: 8-10 are disclosed in Table 1. In certain embodiments, the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 7 or a conservative modification thereof; and the β chain variable region comprises a CDR1 comprising the amino acid sequence set forth in SEQ ID NO: 8 or a conservative modification thereof, a CDR2 comprising the amino acid sequence set forth in SEQ ID NO: 9 or a conservative modification thereof, and a CDR3 comprising the amino acid sequence set forth in SEQ ID NO: 10 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 11. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11. In certain embodiments, the α chain variable region comprises the amino acid sequence set forth in SEQ ID NO: 11. SEQ ID NO: 11 is provided in Table 1.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 12. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 12. In certain embodiments, the β chain variable region comprises the amino acid sequence set forth in SEQ ID NO: 12. SEQ ID NO: 12 is provided in Table 1.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 11; and the β chain variable region comprises an amino acid sequence that is at least about 80% (e.g., at least about 85%, at least about 90%, or at least about 95%) homologous or identical to the amino acid sequence set forth in SEQ ID NO: 12. In certain embodiments, the α chain variable region comprises the amino acid sequence set forth in SEQ ID NO: 11; and the β chain variable region comprises the amino acid sequence set forth in SEQ ID NO: 12.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 13. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 13. In certain embodiments, the α chain comprises the amino acid sequence set forth in SEQ ID NO: 13.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 14. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 14. In certain embodiments, the β chain comprises the amino acid sequence set forth in SEQ ID NO: 14.

In certain embodiments, the α chain comprises an amino acid sequence that is at least about 80% (e.g., at least about 85%, at least about 90%, or at least about 95%) homologous or identical to the amino acid sequence set forth in SEQ ID NO: 13; and the β chain comprises an amino acid sequence that is at least about 80% (e.g., at least about 85%, at least about 90%, or at least about 95%) homologous or identical to the amino acid sequence set forth in SEQ ID NO: 14. In certain embodiments, the α chain comprises the amino acid sequence set forth in SEQ ID NO: 13; and the β chain comprises the amino acid sequence set forth in SEQ ID NO: 14. In certain embodiments, the TCR is designated as “TCR1”. In certain embodiments, the TCR1 binds to a RAS peptide comprising or consisting of the amino acid sequence set forth in SEQ ID NO: 1. In certain embodiments, the TCR1 binds to a RAS peptide comprising or consisting of the amino acid sequence set forth in SEQ ID NO: 2.

In certain embodiments, the CDRs sequences described above including Table 1 are delineated using the IMGT numbering system.

TABLE 1
(TCR1)

| CDRs | 1 | 2 | 3 |
|---|---|---|---|
| α-chain | SIFNT | LYKAGEL | CAGRDDAGNMLTF |
| | [SEQ ID | [SEQ ID | [SEQ ID NO: 7] |
| | NO: 5] | NO: 6] | |
| β-chain | DFQATT | SNEGSKA | CSASVRMNTEAFF |
| | [SEQ ID | [SEQ ID | [SEQ ID NO: 10] |
| | NO: 8] | NO: 9] | |

| | |
|---|---|
| α-chain | MLLEHLLIILWMQLTWVSGQQLNQSPQSMFIQEGEDV |
| variable | SMNCTSSSIFNTWLWYKQEPGEGPVLLIALYKAGELT |
| | SNGRLTAQFGITRKDSFLNISASIPSDVGIYFCAGRD |
| | DAGNMLTFGGGTRLMVKP |
| | [SEQ ID NO: 11] |
| β-chain | MLLLLLLLGPGSGLGAVVSQHPSRVICKSGTSVKIEC |
| variable | RSLDFQATTMFWYRQFPKKSLMLMATSNEGSKATYEQ |
| | GVEKDKELINHASLTLSTLTVTSAHPEDSSFYICSAS |
| | VRMNTEAFFGQGTRLTVV |
| | [SEQ ID NO: 12] |
| Full α- | MLLEHLLIILWMQLTWVSGQQLNQSPQSMFIQEGEDV |
| chain | SMNCTSSSIFNTWLWYKQEPGEGPVLLIALYKAGELT |
| | SNGRLTAQFGITRKDSFLNISASIPSDVGIYFCAGRD |
| | DAGNMLTFGGGTRLMVKPNIQNPDPAVYQLRDSKSSD |
| | KSVCLFTDFDSQTNVSQSKDSDVYITDKTVLDMRSMD |
| | FKSNSAVAWSNKSDFACANAFNNSIIPEDTFFPSPES |
| | SCDVKLVEKSFETDTNLNFQNLSVIGFRILLLKVAGF |
| | NLLMTLRLWSS |
| | [SEQ ID NO: 13] |
| Full β- | MLLLLLLLGPGSGLGAVVSQHPSRVICKSGTSVKIEC |
| chain | RSLDFQATTMFWYRQFPKKSLMLMATSNEGSKATYEQ |
| | GVEKDKFLINHASLTLSTLTVTSAHPEDSSFYICSAS |
| | VRMNTEAFFGQGTRLTVVEDLNKVFPPEVAVFEPSEA |
| | EISHTQKATLVCLATGFFPDHVELSWWVNGKEVHSGV |
| | STDPQPLKEQPALNDSRYCLSSRLRVSATFWQNPRNH |
| | FRCQVQFYGLSENDEWTQDRAKPVTQIVSAEAWGRAD |
| | CGFTSVSYQQGVLSATILYEILLGKATLYAVLVSALV |
| | LMAMVKRKDF |
| | [SEQ ID NO: 14] |

In certain embodiments, the extracellular domain of the TCR comprises an α chain variable region comprising a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOS: 15-17 are disclosed in Table 2. In certain embodiments, the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

In certain embodiments, the extracellular domain of the TCR comprises a β chain variable region comprising a CDR1 comprising the amino acid sequence set forth in SEQ ID NO: 8 or a conservative modification thereof, a CDR2 comprising the amino acid sequence set forth in SEQ ID NO: 18 or a conservative modification thereof, and a CDR3 comprising the amino acid sequence set forth in SEQ ID NO: 19 or a conservative modification thereof. SEQ ID NOS: 8, 18, and 19 are disclosed in Table 2. In certain embodiments, the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19.

In certain embodiments, the α chain variable region comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof; and the β chain variable region comprises a CDR1 comprising the amino acid sequence set forth in SEQ ID NO: 8 or a conservative modification thereof, a CDR2 comprising the amino acid sequence set forth in SEQ ID NO: 18 or a conservative modification thereof, and a CDR3 comprising the amino acid sequence set forth in SEQ ID NO: 19 or a conservative modification thereof. In certain embodiments, the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 20. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 20. In certain embodiments, the α chain variable region comprises the amino acid sequence set forth in SEQ ID NO: 20. SEQ ID NO: 20 is provided in Table 2.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 21. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21. In certain embodiments, the β chain variable region comprises the amino acid sequence set forth in SEQ ID NO: 21. SEQ ID NO: 21 is provided in Table 2.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 20; and the β chain variable region comprises an amino acid sequence that is at least about 80% (e.g., at least about 85%, at least about 90%, or at least about 95%) homologous or identical to the amino acid sequence set forth in SEQ ID NO: 21. In certain embodiments, the α chain variable region comprises the amino acid sequence set forth in SEQ ID NO: 20; and the β chain variable region comprises the amino acid sequence set forth in SEQ ID NO: 21.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 22. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 22. In certain embodiments, the α chain comprises the amino acid sequence set forth in SEQ ID NO: 22.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 23. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23. In certain embodiments, the β chain comprises the amino acid sequence set forth in SEQ ID NO: 23.

In certain embodiments, the α chain comprises an amino acid sequence that is at least about 80% (e.g., at least about 85%, at least about 90%, or at least about 95%) homologous or identical to the amino acid sequence set forth in SEQ ID NO: 21; and the β chain comprises an amino acid sequence that is at least about 80% (e.g., at least about 85%, at least about 90%, or at least about 95%) homologous or identical to the amino acid sequence set forth in SEQ ID NO: 23. In certain embodiments, the α chain comprises the amino acid sequence set forth in SEQ ID NO: 22; and the β chain comprises the amino acid sequence set forth in SEQ ID NO: 23. In certain embodiments, the TCR is designated as “TCR2”. In certain embodiments, the TCR2 binds to a RAS peptide comprising or consisting of the amino acid sequence set forth in SEQ ID NO: 1.

In certain embodiments, the CDRs sequences described above including Table 2 are delineated using the IMGT numbering system.

TABLE 2
(TCR2)

| CDRs | 1 | 2 | 3 |
|---|---|---|---|
| α-chain | NSASDY | IRSNMDK | CAENIRLTGANNLFF |
| | [SEQ ID | [SEQ ID | [SEQ ID NO: 17] |
| | NO: 15] | NO: 16] | |
| β-chain | DFQATT | SNEGSKAT | CSARLLSYEQYF |
| | [SEQ ID | [SEQ ID | [SEQ ID NO: 19] |
| | NO: 8] | NO: 18] | |

| | |
|---|---|
| α-chain | MAGIRALFMYLWLQLDWVSRGESVGLHLPTLSVQEGD |
| variable | NSIINCAYSNSASDYFIWYKQESGKGPQFIIDIRSNM |
| | DKRQGQRVTVLLNKTVKHLSLQIAATQPGDSAVYFCA |
| | ENIRLTGANNLFFGTGTRLTVIP |
| | [SEQ ID NO: 20] |
| β-chain | MLLLLLLLGPGSGLGAVVSQHPSWVICKSGTSVKIEC |
| variable | RSLDFQATTMFWYRQFPKQSLMLMATSNEGSKATYEQ |
| | GVEKDKFLINHASLTLSTLTVTSAHPEDSSFYICSAR |
| | LLSYEQYFGPGTRLTVT |
| | [SEQ ID NO: 21] |
| Full α- | MAGIRALFMYLWLQLDWVSRGESVGLHLPTLSVQEGD |
| chain | NSIINCAYSNSASDYFIWYKQESGKGPQFIIDIRSNM |
| | DKRQGQRVTVLLNKTVKHLSLQIAATQPGDSAVYFCA |
| | ENIRLTGANNLFFGTGTRLTVIPNIQNPDPAVYQLRD |
| | SKSSDKSVCLFTDFDSQTNVSQSKDSDVYITDKTVLD |
| | MRSMDFKSNSAVAWSNKSDFACANAENNSIIPEDTFF |
| | PSPESSCDVKLVEKSFETDINLNFQNLSVIGFRILLL |
| | KVAGENLLMTLRLWSS |
| | [SEQ ID NO: 22] |
| Full β- | MLLLLLLLGPGSGLGAVVSQHPSWVICKSGTSVKIEC |
| chain | RSLDFQATTMFWYRQFPKQSLMLMATSNEGSKATYEQ |
| | GVEKDKFLINHASLTLSTLTVTSAHPEDSSFYICSAR |
| | LLSYEQYFGPGTRLTVTEDLKNVEPPKVAVFEPSEAE |
| | ISHTQKATLVCLATGFYPDHVELSWWVNGKEVHSGVS |
| | TDPQPLKEQPALNDSRYCLSSRLRVSATFWQNPRNHF |
| | RCQVQFYGLSENDEWTQDRAKPVTQIVSAEAWGRADC |
| | GFTSESYQQGVLSATILYEILLGKATLYAVLVSALVL |
| | MAMVKRKDSRG |
| | [SEQ ID NO: 23] |

In certain embodiments, the extracellular domain of the TCR comprises an α chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set Active 117586966.1 forth in SEQ ID NO: 26 or a conservative modification thereof. SEQ ID NOS: 24-26 are disclosed in Table 3. In certain embodiments, the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the extracellular domain of the TCR comprises a β chain variable region comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof. SEQ ID NOS: 27-29 are disclosed in Table 3. In certain embodiments, the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

In certain embodiments, the α chain variable region comprises a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof; and the β chain variable region comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof. In certain embodiments, the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 30. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 30. In certain embodiments, the α chain variable region comprises the amino acid sequence set forth in SEQ ID NO: 30. SEQ ID NO: 30 is provided in Table 3.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 31. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 31. In certain embodiments, the β chain variable region comprises the amino acid sequence set forth in SEQ ID NO: 31. SEQ ID NO: 31 is provided in Table 3.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 30; and the β chain variable region comprises an amino acid sequence that is at least about 80% (e.g., at least about 85%, at least about 90%, or at least about 95%) homologous or identical to the amino acid sequence set forth in SEQ ID NO: 31. In certain embodiments, the α chain variable region comprises the amino acid sequence set forth in SEQ ID NO: 30; and the β chain variable region comprises the amino acid sequence set forth in SEQ ID NO: 31.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 32. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 32. In certain embodiments, the α chain comprises the amino acid sequence set forth in SEQ ID NO: 32.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 33. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 33. In certain embodiments, the β chain comprises the amino acid sequence set forth in SEQ ID NO: 33.

In certain embodiments, the α chain comprises an amino acid sequence that is at least about 80% (e.g., at least about 85%, at least about 90%, or at least about 95%) homologous or identical to the amino acid sequence set forth in SEQ ID NO: 32; and the β chain comprises an amino acid sequence that is at least about 80% (e.g., at least about 85%, at least about 90%, or at least about 95%) homologous or identical to the amino acid sequence set forth in SEQ ID NO: 33. In certain embodiments, the α chain comprises the amino acid sequence set forth in SEQ ID NO: 32; and the β chain comprises the amino acid sequence set forth in SEQ ID NO: 33. In certain embodiments, the TCR is designated as “TCR3”. In certain embodiments, the TCR3 binds to a RAS peptide comprising or consisting of the amino acid sequence set forth in SEQ ID NO: 1.

In certain embodiments, the CDRs sequences described above including Table 3 are delineated using the IMGT numbering system.

TABLE 3
(TCR3)

| CDRs | 1 | 2 | 3 |
|---|---|---|---|
| α-chain | SVFSS | VVTGGEV | CAGGWLISNFGNEKLTF |
| | [SEQ ID | [SEQ ID | [SEQ ID NO: 26] |
| | NO: 24] | NO: 25] | |
| β-chain | SGHNT | YYREEE | CASSLGTAGNEKLFF |
| | [SEQ ID | [SEQ ID | [SEQ ID NO: 29] |
| | NO: 27] | NO: 28] | |

| | |
|---|---|
| α-chain | MVLKFSVSILWIQLAWVSTQLLEQSPQFLSIQEGENL |
| variable | TVYCNSSSVFSSLQWYRQEPGEGPVLLVTVVTGGEVK |
| | KLKRLTFQFGDARKDSSLHITAAQPGDTGLYLCAGGW |
| | LISNFGNEKLTFGTGTRLTIIP |
| | [SEQ ID NO: 30] |
| β-chain | MGPGLLCWALLCLLGAGSVETGVTQSPTHLIKTRGQQ |
| variable | VTLRCSSQSGHNTVSWYQQALGQGPQFIFQYYREEEN |
| | GRGNFPPRESGLQFPNYSSELNVNALELDDSALYLCA |
| | SSLGTAGNEKLFFGSGTQLSVL |
| | [SEQ ID NO: 31] |
| Full α- | MVLKFSVSILWIQLAWVSTQLLEQSPQFLSIQEGENL |
| chain | TVYCNSSSVFSSLQWYRQEPGEGPVLLVTVVTGGEVK |
| | KLKRLTFQFGDARKDSSLHITAAQPGDTGLYLCAGGW |
| | LISNFGNEKLTFGTGTRLTIIPNIQNPDPAVYQLRDS |
| | KSSDKSVCLFTDFDSQTNVSQSKDSDVYITDKTVLDM |
| | RSMDFKSNSAVAWSNKSDFACANAENNSIIPEDTFFP |
| | SPESSCDVKLVEKSFETDTNLNFQNLSVIGFRILLLK |
| | VAGFNLLMTLRLWSS |
| | [SEQ ID NO: 32] |
| Full β- | MGPGLLCWALLCLLGAGSVETGVTQSPTHLIKTRGQQ |
| chain | VTLRCSSQSGHNTVSWYQQALGQGPQFIFQYYREEEN |
| | GRGNFPPRFSGLQFPNYSSELNVNALELDDSALYLCA |
| | SSLGTAGNEKLFFGSGTQLSVL |
| | [SEQ ID NO: 33] |

In certain embodiments, the extracellular domain of the TCR comprises an α chain variable region comprising a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 36 or a conservative modification thereof. SEQ ID NOS: 34-36 are disclosed in Table 4. In certain embodiments, the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOS: 37-39 are disclosed in Table 4. In certain embodiments, the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39.

In certain embodiments, the α chain variable region comprises a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 36 or a conservative modification thereof; and the β chain variable region CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. In certain embodiments, the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and CDR3 comprising the amino acid sequence set forth in SEQ ID NO: 39.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 40. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 40. In certain embodiments, the α chain variable region comprises the amino acid sequence set forth in SEQ ID NO: 40. SEQ ID NO: 40 is provided in Table 4.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 41. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 41. In certain embodiments, the β chain variable region comprises the amino acid sequence set forth in SEQ ID NO: 41. SEQ ID NO: 41 is provided in Table 4.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 40; and the β chain variable region comprises an amino acid sequence that is at least about 80% (e.g., at least about 85%, at least about 90%, or at least about 95%) homologous or identical to the amino acid sequence set forth in SEQ ID NO: 41. In certain embodiments, the α chain variable region comprises the amino acid sequence set forth in SEQ ID NO: 40; and the β chain variable region comprises the amino acid sequence set forth in SEQ ID NO: 41.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 42. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 42. In certain embodiments, the α chain comprises the amino acid sequence set forth in SEQ ID NO: 42.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 43. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 43. In certain embodiments, the β chain comprises the amino acid sequence set forth in SEQ ID NO: 43.

In certain embodiments, the α chain comprises an amino acid sequence that is at least about 80% (e.g., at least about 85%, at least about 90%, or at least about 95%) homologous or identical to the amino acid sequence set forth in SEQ ID NO: 42; and the β chain comprises an amino acid sequence that is at least about 80% (e.g., at least about 85%, at least about 90%, or at least about 95%) homologous or identical to the amino acid sequence set forth in SEQ ID NO: 43. In certain embodiments, the α chain comprises the amino acid sequence set forth in SEQ ID NO: 42; and the β chain comprises the amino acid sequence set forth in SEQ ID NO: 43. In certain embodiments, the TCR is designated as “TCR4”. In certain embodiments, the TCR4 binds to a RAS peptide comprising or consisting of the amino acid sequence set forth in SEQ ID NO: 1.

In certain embodiments, the CDRs sequences described above including Table 4 are delineated using the IMGT numbering system.

TABLE 4
(TCR4)

| CDRs | 1 | 2 | 3 |
|---|---|---|---|
| α-chain | TSESDYY | QEAYKQQN | CAYREYGNKLVF |
| | [SEQ ID | [SEQ ID | [SEQ ID NO: 36] |
| | NO: 34] | NO: 35] | |
| β-chain | SGDLS | YYNGEE | CASSAGRGGFHEQYE |
| | [SEQ ID | [SEQ ID | [SEQ ID NO: 39] |
| | NO: 37] | NO: 38] | |

| | |
|---|---|
| α-chain | MACPGFLWALVISTCLEFSMAQTVTQSQPEMSVQEAE |
| variable | TVTLSCTYDTSESDYYLFWYKQPPSRQMILVIRQEAY |
| | KQQNATENRFSVNFQKAAKSFSLKISDSQLGDAAMYF |
| | CAYREYGNKLVFGAGTILRVKS |
| | [SEQ ID NO: 40] |
| β-chain | MGFRLLCCVAFCLLGAGPVDSGVTQTPKHLITATGQR |
| variable | VTLRCSPRSGDLSVYWYQQSLDQGLQFLIQYYNGEER |
| | AKGNILERFSAQQFPDLHSELNLSSLELGDSALYFCA |
| | SSAGRGGFHEQYFGPGTRLTVT |
| | [SEQ ID NO: 41] |
| Full α- | MACPGFLWALVISTCLEFSMAQTVTQSQPEMSVQEAE |
| chain | TVTLSCTYDTSESDYYLFWYKQPPSRQMILVIRQEAY |
| | KQQNATENRFSVNFQKAAKSFSLKISDSQLGDAAMYF |
| | CAYREYGNKLVFGAGTILRVKSNIQNPDPAVYQLRDS |
| | KSSDKSVCLFTDFDSQTNVSQSKDSDVYITDKTVLDM |
| | RSMDFKSNSAVAWSNKSDFACANAENNSIIPEDTFFP |
| | SPESSCDVKLVEKSFETDINLNFQNLSVIGFRILLLK |
| | VAGENLLMTLRLWSS |
| | [SEQ ID NO: 42] |
| Full β- | MGFRLLCCVAFCLLGAGPVDSGVTQTPKHLITATGQR |
| chain | VTLRCSPRSGDLSVYWYQQSLDQGLQFLIQYYNGEER |
| | AKGNILERFSAQQFPDLHSELNLSSLELGDSALYFCA |
| | SSAGRGGFHEQYFGPGTRLTVTEDLNKVFPPEVAVFE |
| | PSEAEISHTQKATLVCLATGFFPDHVELSWWVNGKEV |
| | HSGVSTDPQPLKEQPALNDSRYCLSSRLRVSATFWQN |
| | PRNHFRCQVQFYGLSENDEWTQDRAKPVTQIVSAEAW |
| | GRADCGFTSVSYQQGVLSATILYEILLGKATLYAVLV |
| | SALVLMAMVKRKDF |
| | [SEQ ID NO: 43] |

In certain embodiments, the extracellular domain of the TCR comprises an α chain variable region comprising a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 44 or a conservative modification thereof. SEQ ID NOS: 34, 35, and 44 are disclosed in Table 5. In certain embodiments, the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOS: 37-39 are disclosed in Table 5. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39.

In certain embodiments, the α chain variable region comprises a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 44 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. In certain embodiments, the α chain variable region comprises a CDR1 comprising the amino acid sequence set forth in SEQ ID NO: 34, a CDR2 comprising the amino acid sequence set forth in SEQ ID NO: 35, a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 45. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 45. In certain embodiments, the α chain variable region comprises the amino acid sequence set forth in SEQ ID NO: 45. SEQ ID NO: 45 is provided in Table 5.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 41. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 41. In certain embodiments, the β chain variable region comprises the amino acid sequence set forth in SEQ ID NO: 41. SEQ ID NO: 41 is provided in Table 5.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 45; and the β chain variable region comprises an amino acid sequence that is at least about 80% (e.g., at least about 85%, at least about 90%, or at least about 95%) homologous or identical to the amino acid sequence set forth in SEQ ID NO: 41. In certain embodiments, the α chain variable region comprises the amino acid sequence set forth in SEQ ID NO: 45; and the β chain variable region comprises the amino acid sequence set forth in SEQ ID NO: 41.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 46. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 46. In certain embodiments, the α chain comprises the amino acid sequence set forth in SEQ ID NO: 46.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 43. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 43. In certain embodiments, the β chain comprises the amino acid sequence set forth in SEQ ID NO: 43.

In certain embodiments, the α chain comprises an amino acid sequence that is at least about 80% (e.g., at least about 85%, at least about 90%, or at least about 95%) homologous or identical to the amino acid sequence set forth in SEQ ID NO: 46; and the β chain comprises an amino acid sequence that is at least about 80% (e.g., at least about 85%, at least about 90%, or at least about 95%) homologous or identical to the amino acid sequence set forth in SEQ ID NO: 43. In certain embodiments, the α chain comprises the amino acid sequence set forth in SEQ ID NO: 46; and the β chain comprises the amino acid sequence set forth in SEQ ID NO: 43. In certain embodiments, the TCR is designated as “TCR5”. In certain embodiments, the TCR5 binds to a RAS peptide comprising or consisting of the amino acid sequence set forth in SEQ ID NO: 1.

In certain embodiments, the CDRs sequences described above including Table 5 are delineated using the IMGT numbering system.

TABLE 5
(TCR5)

| CDRs | 1 | 2 | 3 |
|---|---|---|---|
| α-chain | TSESDYY | QEAYKQQN | CAYSNDNYGQNFVF |
| | [SEQ ID | [SEQ ID | [SEQ ID NO: 44] |
| | NO: 34] | NO: 35] | |
| β-chain | SGDLS | YYNGEE | CASSAGRGGFHEQYF |
| | [SEQ ID | [SEQ ID | [SEQ ID NO: 39] |
| | NO: 37] | NO: 38] | |

| | |
|---|---|
| α-chain | MACPGFLWALVISTCLEFSMAQTVTQSQPEMSVQEAE |
| variable | TVTLSCTYDTSESDYYLFWYKQPPSRQMILVIRQEAY |
| | KQQNATENRFSVNFQKAAKSFSLKISDSQLGDAAMYF |
| | CAYSNDNYGQNFVFGPGTRLSVLP |
| | [SEQ ID NO: 45] |
| β-chain | MGFRLLCCVAFCLLGAGPVDSGVTQTPKHLITATGQR |
| variable | VTLRCSPRSGDLSVYWYQQSLDQGLQFLIQYYNGEER |
| | AKGNILERFSAQQFPDLHSELNLSSLELGDSALYFCA |
| | SSAGRGGFHEQYFGPGTRLTVT |
| | [SEQ ID NO: 41] |
| Full α- | MACPGFLWALVISTCLEFSMAQTVTQSQPEMSVQEAE |
| chain | TVTLSCTYDTSESDYYLFWYKQPPSRQMILVIRQEAY |
| | KQQNATENRFSVNFQKAAKSFSLKISDSQLGDAAMYF |
| | CAYSNDNYGQNFVFGPGTRLSVLPNIQNPDPAVYQLR |
| | DSKSSDKSVCLFTDFDSQTNVSQSKDSDVYITDKTVL |
| | DMRSMDFKSNSAVAWSNKSDFACANAFNNSIIPEDTF |
| | FPSPESSCDVKLVEKSFETDTNLNFQNLSVIGFRILL |
| | LKVAGFNLLMTLRLWSS |
| | [SEQ ID NO: 46] |
| Full β- | MGFRLLCCVAFCLLGAGPVDSGVTQTPKHLITATGQR |
| chain | VTLRCSPRSGDLSVYWYQQSLDQGLQFLIQYYNGEER |
| | AKGNILERFSAQQFPDLHSELNLSSLELGDSALYFCA |
| | SSAGRGGFHEQYFGPGTRLTVTEDLNKVFPPEVAVFE |
| | PSEAEISHTQKATLVCLATGFFPDHVELSWWVNGKEV |
| | HSGVSTDPQPLKEQPALNDSRYCLSSRLRVSATFWQN |
| | PRNHFRCQVQFYGLSENDEWTQDRAKPVTQIVSAEAW |
| | GRADCGFTSVSYQQGVLSATILYEILLGKATLYAVLV |
| | SALVLMAMVKRKDF |
| | [SEQ ID NO: 43] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 47 or a conservative modification thereof. SEQ ID NOS: 5, 6, and 47 are disclosed in Table 6. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47.

In certain embodiments, the extracellular domain of the TCR comprises a β chain variable region comprising a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 50 or a conservative modification thereof. SEQ ID NOS: 48-50 are disclosed in Table 6. In certain embodiments, the β chain variable region CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 47 or a conservative modification thereof; and the β chain variable region comprises a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 50 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 51. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 51. In certain embodiments, the α chain variable region comprises the amino acid sequence set forth in SEQ ID NO: 51. SEQ ID NO: 51 is provided in Table 6.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 52. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 52. In certain embodiments, the β chain variable region comprises the amino acid sequence set forth in SEQ ID NO: 52. SEQ ID NO: 52 is provided in Table 6.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 51; and the β chain variable region comprises an amino acid sequence that is at least about 80% (e.g., at least about 85%, at least about 90%, or at least about 95%) homologous or identical to the amino acid sequence set forth in SEQ ID NO: 52. In certain embodiments, the α chain variable region comprises the amino acid sequence set forth in SEQ ID NO: 51; and the β chain variable region comprises the amino acid sequence set forth in SEQ ID NO: 52.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 53. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 53. In certain embodiments, the α chain comprises the amino acid sequence set forth in SEQ ID NO: 53.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 54. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 54. In certain embodiments, the β chain comprises the amino acid sequence set forth in SEQ ID NO: 54.

In certain embodiments, the α chain comprises an amino acid sequence that is at least about 80% (e.g., at least about 85%, at least about 90%, or at least about 95%) homologous or identical to the amino acid sequence set forth in SEQ ID NO: 53; and the β chain comprises an amino acid sequence that is at least about 80% (e.g., at least about 85%, at least about 90%, or at least about 95%) homologous or identical to the amino acid sequence set forth in SEQ ID NO: 54. In certain embodiments, the α chain comprises the amino acid sequence set forth in SEQ ID NO: 53; and the β chain comprises the amino acid sequence set forth in SEQ ID NO: 54. In certain embodiments, the TCR is designated as “TCR6”. In certain embodiments, the TCR6 binds to a RAS peptide comprising or consisting of the amino acid sequence set forth in SEQ ID NO: 1.

In certain embodiments, the CDRs sequences described above including Table 6 are delineated using the IMGT numbering system.

TABLE 6
(TCR6)

| CDRs | 1 | 2 | 3 |
|---|---|---|---|
| α-chain | SIFNT | LYKAGEL | CAGHEVSDGQKLLF |
| | [SEQ ID | [SEQ ID | [SEQ ID NO: 47] |
| | NO: 5] | NO: 6] | |
| β-chain | LNHNV | YYDKDF | CATSRTHSYEQYF |
| | [SEQ ID | [SEQ ID | [SEQ ID NO: 50] |
| | NO: 48] | NO: 49] | |

| | |
|---|---|
| α-chain | MLLEHLLIILWMQLTWVSGQQLNQSPQSMFIQEGEDV |
| variable | SMNCTSSSIFNTWLWYKQDPGEGPVLLIALYKAGELT |
| | RSNGLTAQFGITRKDSFLNISASIPSDVGIYFCAGHE |
| | VSDGQKLLFARGTMLKVDL |
| | [SEQ ID NO: 51] |
| β-chain | MGPGLLHWMALCLLGTGHGDAMVIQNPRYQVTQFGKP |
| variable | VTLSCSQTLNHNVMYWYQQKSSQAPKLLFHYYDKDFN |
| | NEADTPDNFQSRRPNTSFCFLDIRSPGLGDAAMYLCA |
| | TSRTHSYEQYFGPGTRLTVT |
| | [SEQ ID NO: 52] |
| Full α- | MLLEHLLIILWMQLTWVSGQQLNQSPQSMFIQEGEDV |
| chain | SMNCTSSSIFNTWLWYKQDPGEGPVLLIALYKAGELT |
| | SNGRLTAQFGITRKDSFLNISASIPSDVGIYFCAGHE |
| | VSDGQKLLFARGTMLKVDLNIQNPDPAVYQLRDSKSS |
| | DKSVCLFTDFDSQTNVSQSKDSDVYITDKTVLDMRSM |
| | DFKSNSAVAWSNKSDFACANAFNNSIIPEDTFFPSPE |
| | SSCDVKLVEKSFETDTNLNFQNLSVIGFRILLLKVAG |
| | FNLLMTLRLWSS |
| | [SEQ ID NO: 53] |
| Full β- | MGPGLLHWMALCLLGTGHGDAMVIQNPRYQVTQFGKP |
| chain | VTLSCSQTLNHNVMYWYQQKSSQAPKLLFHYYDKDFN |
| | NEADTPDNFQSRRPNTSFCFLDIRSPGLGDAAMYLCA |
| | TSRTHSYEQYFGPGTRLTVTEDLNKVFPPEVAVFEPS |
| | EAEISHTQKATLVCLATGFFPDHVELSWWVNGKEVHS |
| | GVSTDPQPLKEQPALNDSRYCLSSRLRVSATFWQNPR |
| | NHFRCQVQFYGLSENDEWTQDRAKPVTQIVSAEAWGR |
| | ADCGFTSVSYQQGVLSATILYEILLGKATLYAVLVSA |
| | LVLMAMVKRKDF |
| | [SEQ ID NO: 54] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 55 or a conservative modification thereof. SEQ ID NOS: 5, 6, and 55 are disclosed in Table 7. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 56 or a conservative modification thereof. SEQ ID NOS: 36, 37, and 56 are disclosed in Table 7. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 55 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 56 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 57. In certain embodiments, the α chain variable region comprises the amino acid sequence set forth in SEQ ID NO: 57. SEQ ID NO: 57 is provided in Table 7.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 58. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 58. In certain embodiments, the β chain variable region comprises the amino acid sequence set forth in SEQ ID NO: 58. SEQ ID NO: 58 is provided in Table 7.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57; and the β chain variable region comprises an amino acid sequence that is at least about 80% (e.g., at least about 85%, at least about 90%, or at least about 95%) homologous or identical to the amino acid sequence set forth in SEQ ID NO: 58. In certain embodiments, the α chain variable region comprises the amino acid sequence set forth in SEQ ID NO: 57; and the β chain variable region comprises the amino acid sequence set forth in SEQ ID NO: 58.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 59. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 59. In certain embodiments, the α chain comprises the amino acid sequence set forth in SEQ ID NO: 59.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 60. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 60. In certain embodiments, the β chain comprises the amino acid sequence set forth in SEQ ID NO: 60.

In certain embodiments, the α chain comprises an amino acid sequence that is at least about 80% (e.g., at least about 85%, at least about 90%, or at least about 95%) homologous or identical to the amino acid sequence set forth in SEQ ID NO: 59; and the β chain comprises an amino acid sequence that is at least about 80% (e.g., at least about 85%, at least about 90%, or at least about 95%) homologous or identical to the amino acid sequence set forth in SEQ ID NO: 60. In certain embodiments, the α chain comprises the amino acid sequence set forth in SEQ ID NO: 59; and the β chain comprises the amino acid sequence set forth in SEQ ID NO: 60. In certain embodiments, the TCR is designated as “TCR7”. In certain embodiments, the TCR7 binds to a RAS peptide comprising or consisting of the amino acid sequence set forth in SEQ ID NO: 1.

In certain embodiments, the CDRs sequences described above including Table 7 are delineated using the IMGT numbering system.

TABLE 7
(TCR7)

| CDRs | 1 | 2 | 3 |
|---|---|---|---|
| α-chain | SIFNT | LYKAGEL | CAGYTGANSKLTF |
| | [SEQ ID | [SEQ ID | [SEQ ID NO: 55] |
| | NO: 5] | NO: 6] | |
| β-chain | SGDLS | YYNGEE | CASSVESGDYEQYF |
| | [SEQ ID | [SEQ ID | [SEQ ID NO: 56] |
| | NO: 37] | NO: 38] | |

| | |
|---|---|
| α-chain | MLLEHLLIILWMQLTWVSGQQLNQSPQSMFIQEGEDV |
| variable | SMNCTSSSIFNTWLWYKQDPGEGPVLLIALYKAGELT |
| | SNGRLTAQFGITRKDSFLNISASIPSDVGIYFCAGYT |
| | GANSKLTFGKGITLSVRP |
| | [SEQ ID NO: 57] |
| β-chain | MGFRLLCCVAFCLLGAGPVDSGVTQTPKHLITATGQR |
| variable | VTLRCSPRSGDLSVYWYQQSLDQGLQFLIQYYNGEER |
| | AKGNILERFSAQQFPDLHSELNLSSLELGDSALYFCA |
| | SSVESGDYEQYFGPGTRLTVT |
| | [SEQ ID NO: 58] |
| Full α- | MLLEHLLIILWMQLTWVSGQQLNQSPQSMFIQEGEDV |
| chain | SMNCTSSSIFNTWLWYKQDPGEGPVLLIALYKAGELT |
| | SNGRLTAQFGITRKDSFLNISASIPSDVGIYFCAGYT |
| | GANSKLTFGKGITLSVRPNIQNPDPAVYQLRDSKSSD |
| | KSVCLFTDFDSQTNVSQSKDSDVYITDKTVLDMRSMD |
| | FKSNSAVAWSNKSDFACANAFNNSIIPEDTFFPSPES |
| | SCDVKLVEKSFETDTNLNFQNLSVIGFRILLLKVAGF |
| | NLLMTLRLWSS |
| | [SEQ ID NO: 59] |
| Full β- | MGFRLLCCVAFCLLGAGPVDSGVTQTPKHLITATGQR |
| chain | VTLRCSPRSGDLSVYWYQQSLDQGLQFLIQYYNGEER |
| | AKGNILERFSAQQFPDLHSELNLSSLELGDSALYFCA |
| | SSVESGDYEQYFGPGTRLTVTEDLNKVFPPEVAVFEP |
| | SEAEISHTQKATLVCLATGFFPDHVELSWWVNGKEVH |
| | SGVSTDPQPLKEQPALNDSRYCLSSRLRVSATFWQNP |
| | RNHFRCQVQFYGLSENDEWTQDRAKPVTQIVSAEAWG |
| | RADCGFTSVSYQQGVLSATILYEILLGKATLYAVLVS |
| | ALVLMAMVKRKDF |
| | [SEQ ID NO: 60] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 55 or a conservative modification thereof. SEQ ID NOS: 5, 6, and 55 are disclosed in Table 8. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 37 or a conservative modification thereof, and a CDR3 comprising the amino acid sequence set forth in SEQ ID NO: 61 or a conservative modification thereof. SEQ ID NOS: 38, 38, and 61 are disclosed in Table 8. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 55 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 61 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 57. In certain embodiments, the α chain variable region comprises the amino acid sequence set forth in SEQ ID NO: 57. SEQ ID NO: 57 is provided in Table 8.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 62. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 62. In certain embodiments, the β chain variable region comprises the amino acid sequence set forth in SEQ ID NO: 62. SEQ ID NO: 62 is provided in Table 8.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57; and the β chain variable region comprises an amino acid sequence that is at least about 80% (e.g., at least about 85%, at least about 90%, or at least about 95%) homologous or identical to the amino acid sequence set forth in SEQ ID NO: 62. In certain embodiments, the α chain variable region comprises the amino acid sequence set forth in SEQ ID NO: 57; and the β chain variable region comprises the amino acid sequence set forth in SEQ ID NO: 62.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 59. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 59. In certain embodiments, the α chain comprises the amino acid sequence set forth in SEQ ID NO: 59.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 63. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 63. In certain embodiments, the β chain comprises the amino acid sequence set forth in SEQ ID NO: 63.

In certain embodiments, the α chain comprises an amino acid sequence that is at least about 80% (e.g., at least about 85%, at least about 90%, or at least about 95%) homologous or identical to the amino acid sequence set forth in SEQ ID NO: 59; and the β chain comprises an amino acid sequence that is at least about 80% (e.g., at least about 85%, at least about 90%, or at least about 95%) homologous or identical to the amino acid sequence set forth in SEQ ID NO: 63. In certain embodiments, the α chain comprises the amino acid sequence set forth in SEQ ID NO: 59; and the β chain comprises the amino acid sequence set forth in SEQ ID NO: 63. In certain embodiments, the TCR is designated as “TCR8”. In certain embodiments, the TCR8 binds to a RAS peptide comprising or consisting of the amino acid sequence set forth in SEQ ID NO: 1.

In certain embodiments, the CDRs sequences described above including Table 8 are delineated using the IMGT numbering system.

TABLE 8
(TCR8)

| CDRs | 1 | 2 | 3 |
|---|---|---|---|
| α-chain | SIFNT | LYKAGEL | CAGYTGANSKLTF |
| | [SEQ ID NO: 5] | [SEQ ID NO: 6] | [SEQ ID NO: 55] |
| ß-chain | SGDLS | YYNGEE | CASSVGATYEQYF |
| | [SEQ ID NO: 37] | [SEQ ID NO: 38] | [SEQ ID NO: 61] |

| | |
|---|---|
| α-chain | MLLEHLLIILWMQLTWVSGQQLNQSPQSMFIQEGEDVSMNCTSSSIFNTWLWYKQDPGEGPV |
| variable | LLIALYKAGELTSNGRLTAQFGITRKDSFLNISASIPSDVGIYFCAGYTGANSKLTFGKGIT |
| | LSVRP |
| | [SEQ ID NO: 57] |
| ß-chain | MGFRLLCCVAFCLLGAGPVDSGVTQTPKHLITATGQRVTLRCSPRSGDLSVYWYQQSLDQGL |
| variable | QFLIQYYNGEERAKGNILERFSAQQFPDLHSELNLSSLELGDSALYFCASSVGATYEQYFGP |
| | GTRLTVT |
| | [SEQ ID NO: 62] |
| Full α- | MLLEHLLIILWMQLTWVSGQQLNQSPQSMFIQEGEDVSMNCTSSSIFNTWLWYKQDPGEGPV |
| chain | LLIALYKAGELTSNGRLTAQFGITRKDSFLNISASIPSDVGIYFCAGYTGANSKLTFGKGIT |
| | LSVRPNIQNPDPAVYQLRDSKSSDKSVCLFTDFDSQTNVSQSKDSDVYITDKTVLDMRSMDF |
| | KSNSAVAWSNKSDFACANAFNNSIIPEDTFFPSPESSCDVKLVEKSFETDTNLNFQNLSVIG |
| | FRILLLKVAGFNLLMTLRLWSS |
| | [SEQ ID NO: 59] |
| Full ß- | MGFRLLCCVAFCLLGAGPVDSGVTQTPKHLITATGQRVTLRCSPRSGDLSVYWYQQSLDQGL |
| chain | QFLIQYYNGEERAKGNILERFSAQQFPDLHSELNLSSLELGDSALYFCASSVGATYEQYFGP |
| | GTRLTVTEDLNKVEPPEVAVFEPSEAEISHTQKATLVCLATGFFPDHVELSWWVNGKEVHSG |
| | VSTDPQPLKEQPALNDSRYCLSSRLRVSATFWQNPRNHERCQVQFYGLSENDEWTQDRAKPV |
| | TQIVSAEAWGRADCGFTSVSYQQGVLSATILYEILLGKATLYAVLVSALVLMAMVKRKDF |
| | [SEQ ID NO: 63] |

In certain embodiments, the extracellular domain of the TCR comprises an α chain variable region comprising a CDR1 comprising the amino acid sequence set forth in SEQ ID NO: 64 or a conservative modification thereof, a CDR2 comprising the amino acid sequence set forth in SEQ ID NO: 65 or a conservative modification thereof, and a CDR3 comprising the amino acid sequence set forth in SEQ ID NO: 66 or a conservative modification thereof. SEQ ID NOS: 64-66 are disclosed in Table 9. In certain embodiments, the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66.

In certain embodiments, the extracellular domain of the TCR comprises a β chain variable region comprising a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 67 or a conservative modification thereof. SEQ ID NOS: 48, 49, and 67 are disclosed in Table 9. In certain embodiments, the β chain variable region CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67.

In certain embodiments, the α chain variable region comprises a CDR1 comprising the amino acid sequence set forth in SEQ ID NO: 64 or a conservative modification thereof, a CDR2 comprising the amino acid sequence set forth in SEQ ID NO: 65 or a conservative modification thereof, and a CDR3 comprising the amino acid sequence set forth in SEQ ID NO: 66 or a conservative modification thereof; and the β chain variable region comprises a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 67 or a conservative modification thereof. In certain embodiments, the α chain variable region comprises a CDR1 comprising the amino acid sequence set forth in SEQ ID NO: 64, a CDR2 comprising the amino acid sequence set forth in SEQ ID NO: 65, a CDR3 comprising the amino acid sequence set forth in SEQ ID NO: 66; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 68. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 68. In certain embodiments, the α chain variable region comprises the amino acid sequence set forth in SEQ ID NO: 68. SEQ ID NO: 68 is provided in Table 9.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 69. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 69. In certain embodiments, the β chain variable region comprises the amino acid sequence set forth in SEQ ID NO: 69. SEQ ID NO: 69 is provided in Table 9.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 68; and the β chain variable region comprises an amino acid sequence that is at least about 80% (e.g., at least about 85%, at least about 90%, or at least about 95%) homologous or identical to the amino acid sequence set forth in SEQ ID NO: 69. In certain embodiments, the α chain variable region comprises the amino acid sequence set forth in SEQ ID NO: 68; and the β chain variable region comprises the amino acid sequence set forth in SEQ ID NO: 69.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 70. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 70. In certain embodiments, the α chain comprises the amino acid sequence set forth in SEQ ID NO: 70.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 71. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 71. In certain embodiments, the β chain comprises the amino acid sequence set forth in SEQ ID NO: 71.

In certain embodiments, the α chain comprises an amino acid sequence that is at least about 80% (e.g., at least about 85%, at least about 90%, or at least about 95%) homologous or identical to the amino acid sequence set forth in SEQ ID NO: 70; and the β chain comprises an amino acid sequence that is at least about 80% (e.g., at least about 85%, at least about 90%, or at least about 95%) homologous or identical to the amino acid sequence set forth in SEQ ID NO: 71. In certain embodiments, the α chain comprises the amino acid sequence set forth in SEQ ID NO: 70; and the β chain comprises the amino acid sequence set forth in SEQ ID NO: 71. In certain embodiments, the TCR is designated as “TCR9”. In certain embodiments, the TCR9 binds to a RAS peptide comprising or consisting of the amino acid sequence set forth in SEQ ID NO: 1.

In certain embodiments, the CDRs sequences described above including Table 9 are delineated using the IMGT numbering system.

TABLE 9
(TCR9)

| CDRs | 1 | 2 | 3 |
|---|---|---|---|
| α-chain | VSNAYN | GSKP | CAAPASSASKIIF |
| | [SEQ ID NO: 64] | [SEQ ID NO: 65] | [SEQ ID NO: 66] |
| ß-chain | LNHNV | YYDKDF | CATSRLGGPTPNEKLFF |
| | [SEQ ID NO: 48] | [SEQ ID NO: 49] | [SEQ ID NO: 67] |

| | |
|---|---|
| α-chain | MALQSTLGAVWLGLLLNSLWKVAESKDQVFQPSTVASSEGAVVEIFCNHSVSNAYN |
| variable | FFWYLHFPGCAPRLLVKGSKPSQQGRYNMTYERFSSSLLILQVREADAAVYYCAAP |
| | ASSASKIIFGSGTRLSIRP |
| | [SEQ ID NO: 68] |
| ß-chain | MGPGLLHWMALCLLGTGHGDAMVIQNPRYQVTQFGKPVTLSCSQTLNHNVMYWYQQ |
| variable | KSSQAPKLLFHYYDKDFNNEADTPDNFQSRRPNTSFCFLDIRSPGLGDAAMYLCAT |
| | SRLGGPTPNEKLFFGSGTQLSVL |
| | [SEQ ID NO: 69] |
| Full α- | MALQSTLGAVWLGLLLNSLWKVAESKDQVFQPSTVASSEGAVVEIFCNHSVSNAYN |
| chain | FFWYLHFPGCAPRLLVKGSKPSQQGRYNMTYERFSSSLLILQVREADAAVYYCAAP |
| | ASSASKIIFGSGTRLSIRPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 70] |
| Full ß- | MGPGLLHWMALCLLGTGHGDAMVIQNPRYQVTQFGKPVTLSCSQTLNHNVMYWYQQ |
| chain | KSSQAPKLLFHYYDKDFNNEADTPDNFQSRRPNTSFCELDIRSPGLGDAAMYLCAT |
| | SRLGGPTPNEKLFFGSGTQLSVLEDLKNVFPPKVAVFEPSEAEISHTQKATLVCLA |
| | TGFYPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSESYQQGVLSA |
| | TILYEILLGKATLYAVLVSALVLMAMVKRKDSRG |
| | [SEQ ID NO: 71] |

In certain embodiments, the extracellular domain of the TCR comprises an α chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof. SEQ ID NOS: 72-74 are disclosed in Table 10. In certain embodiments, the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the extracellular domain of the TCR comprises a β chain variable region comprising a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 67 or a conservative modification thereof. SEQ ID NOS: 48, 49, 67 are disclosed in Table 10. In certain embodiments, the β chain variable region CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67.

In certain embodiments, the α chain variable region comprises a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof; and the β chain variable region comprises a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 67 or a conservative modification thereof. In certain embodiments, the α chain variable region comprises a CDR1 comprising the amino acid sequence set forth in SEQ ID NO: 72, a CDR2 comprising the amino acid sequence set forth in SEQ ID NO: 73, a CDR3 comprising the amino acid sequence set forth in SEQ ID NO: 74; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 75. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 75. In certain embodiments, the α chain variable region comprises the amino acid sequence set forth in SEQ ID NO: 75. SEQ ID NO: 75 is provided in Table 10.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 69. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 69. In certain embodiments, the β chain variable region comprises the amino acid sequence set forth in SEQ ID NO: 69. SEQ ID NO: 69 is provided in Table 10.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 75; and the β chain variable region comprises an amino acid sequence that is at least about 80% (e.g., at least about 85%, at least about 90%, or at least about 95%) homologous or identical to the amino acid sequence set forth in SEQ ID NO: 69. In certain embodiments, the α chain variable region comprises the amino acid sequence set forth in SEQ ID NO: 75; and the β chain variable region comprises the amino acid sequence set forth in SEQ ID NO: 69.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 76. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 76. In certain embodiments, the α chain comprises the amino acid sequence set forth in SEQ ID NO: 76.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 71. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 71. In certain embodiments, the β chain comprises the amino acid sequence set forth in SEQ ID NO: 71.

In certain embodiments, the α chain comprises an amino acid sequence that is at least about 80% (e.g., at least about 85%, at least about 90%, or at least about 95%) homologous or identical to the amino acid sequence set forth in SEQ ID NO: 76; and the β chain comprises an amino acid sequence that is at least about 80% (e.g., at least about 85%, at least about 90%, or at least about 95%) homologous or identical to the amino acid sequence set forth in SEQ ID NO: 71. In certain embodiments, the α chain comprises the amino acid sequence set forth in SEQ ID NO: 76; and the β chain comprises the amino acid sequence set forth in SEQ ID NO: 71. In certain embodiments, the TCR is designated as “TCR10”. In certain embodiments, the TCR10 binds to a RAS peptide comprising or consisting of the amino acid sequence set forth in SEQ ID NO: 1.

In certain embodiments, the CDRs sequences described above including Table 10 are delineated using the IMGT numbering system.

TABLE 10
(TCR10)

| CDRs | 1 | 2 | 3 |
|---|---|---|---|
| α-chain | YGATPY | YFSGDTLV | CAVGAAGGSYIPTF |
| | [SEQ ID NO: 72] | [SEQ ID NO: 73] | [SEQ ID NO: 74] |
| ß-chain | LNHNV | YYDKDF | CATSRLGGPTPNEKLFF |
| | [SEQ ID NO: 48] | [SEQ ID NO: 49] | [SEQ ID NO: 67] |

| | |
|---|---|
| α-chain | MLLELIPLLGIHFVLRTARAQSVTQPDIHITVSEGASLELRCNYSYGATPYLFWYV |
| variable | QSPGQGLQLLLKYFSGDTLVQGIKGFEAEFKRSQSSENLRKPSVHWSDAAEYFCAV |
| | GAAGGSYIPTFGRGTSLIVHP |
| | [SEQ ID NO: 75] |
| ß-chain | MGPGLLHWMALCLLGTGHGDAMVIQNPRYQVTQFGKPVTLSCSQTLNHNVMYWYQQ |
| variable | KSSQAPKLLFHYYDKDFNNEADTPDNFQSRRPNTSFCFLDIRSPGLGDAAMYLCAT |
| | SRLGGPTPNEKLFFGSGTQLSVL |
| | [SEQ ID NO: 69] |
| Full α- | MLLELIPLLGIHFVLRTARAQSVTQPDIHITVSEGASLELRCNYSYGATPYLFWYV |
| chain | QSPGQGLQLLLKYFSGDTLVQGIKGFEAEFKRSQSSENLRKPSVHWSDAAEYFCAV |
| | GAAGGSYIPTFGRGTSLIVHPNIQNPDPAVYQLRDSKSSDKSVCLFTDFDSQTNVS |
| | QSKDSDVYITDKTVLDMRSMDFKSNSAVAWSNKSDFACANAFNNSIIPEDTFFPSP |
| | ESSCDVKLVEKSFETDTNLNFQNLSVIGFRILLLKVAGFNLLMTLRLWSS |
| | [SEQ ID NO: 76] |
| Full ß- | MGPGLLHWMALCLLGTGHGDAMVIQNPRYQVTQFGKPVTLSCSQTLNHNVMYWYQQ |
| chain | KSSQAPKLLFHYYDKDFNNEADTPDNFQSRRPNTSFCFLDIRSPGLGDAAMYLCAT |
| | SRLGGPTPNEKLFFGSGTQLSVLEDLKNVEPPKVAVFEPSEAEISHTQKATLVCLA |
| | TGFYPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSESYQQGVLSA |
| | TILYEILLGKATLYAVLVSALVLMAMVKRKDSRG |
| | [SEQ ID NO: 71] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 77 or a conservative modification thereof. SEQ ID NOS: 5, 6, and 77 are disclosed in Table 11. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77.

In certain embodiments, the extracellular domain of the TCR comprises a β chain variable region comprising a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof. SEQ ID NOS: 78-80 are disclosed in Table 11. In certain embodiments, the β chain variable region CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 77 or a conservative modification thereof; and the β chain variable region comprises a CDR1 comprising the amino acid sequence set forth in SEQ ID NO: 78 or a conservative modification thereof, a CDR2 comprising the amino acid sequence set forth in SEQ ID NO: 79 or a conservative modification thereof, and a CDR3 comprising the amino acid sequence set forth in SEQ ID NO: 80 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 81. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 81. In certain embodiments, the α chain variable region comprises the amino acid sequence set forth in SEQ ID NO: 81. SEQ ID NO: 81 is provided in Table 11.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 82. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82. In certain embodiments, the β chain variable region comprises the amino acid sequence set forth in SEQ ID NO: 82. SEQ ID NO: 82 is provided in Table 11.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 81; and the β chain variable region comprises an amino acid sequence that is at least about 80% (e.g., at least about 85%, at least about 90%, or at least about 95%) homologous or identical to the amino acid sequence set forth in SEQ ID NO: 82. In certain embodiments, the α chain variable region comprises the amino acid sequence set forth in SEQ ID NO: 81; and the β chain variable region comprises the amino acid sequence set forth in SEQ ID NO: 82.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 83. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 83. In certain embodiments, the α chain comprises the amino acid sequence set forth in SEQ ID NO: 83.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 84. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 84. In certain embodiments, the β chain comprises the amino acid sequence set forth in SEQ ID NO: 84.

In certain embodiments, the α chain comprises an amino acid sequence that is at least about 80% (e.g., at least about 85%, at least about 90%, or at least about 95%) homologous or identical to the amino acid sequence set forth in SEQ ID NO: 83; and the β chain comprises an amino acid sequence that is at least about 80% (e.g., at least about 85%, at least about 90%, or at least about 95%) homologous or identical to the amino acid sequence set forth in SEQ ID NO: 84. In certain embodiments, the α chain comprises the amino acid sequence set forth in SEQ ID NO: 83; and the β chain comprises the amino acid sequence set forth in SEQ ID NO: 84. In certain embodiments, the TCR is designated as “TCR11”. In certain embodiments, the TCR11 binds to a RAS peptide comprising or consisting of the amino acid sequence set forth in SEQ ID NO: 1.

In certain embodiments, the CDRs sequences described above including Table 11 are delineated using the IMGT numbering system.

TABLE 11
(TCR11)

| CDRs | 1 | 2 | 3 |
|---|---|---|---|
| α-chain | SIFNT | LYKAGEL | CAGRRTTDSWGKLQF |
| | [SEQ ID NO: 5] | [SEQ ID NO: 6] | [SEQ ID NO: 77] |
| ß-chain | MDHEN | SYDVKM | CASSLFGGSNTGELFF |
| | [SEQ ID NO: 78] | [SEQ ID NO: 79] | [SEQ ID NO: 80] |

| | |
|---|---|
| α-chain | MLLEHLLIILWMQLTWVSGQQLNQSPQSMFIQEGEDVSMNCTSSSIFNTWLWYKQD |
| variable | PGEGPVLLIALYKAGELTSNGRLTAQFGITRKDSELNISASIPSDVGIYFCAGRRT |
| | TDSWGKLQFGAGTQVVVTP |
| | [SEQ ID NO: 81] |
| ß-chain | MGIRLLCRVAFCFLAVGLVDVKVTQSSRYLVKRTGEKVFLECVQDMDHENMFWYRQ |
| variable | DPGLGLRLIYFSYDVKMKEKGDIPEGYSVSREKKERESLILESASTNQTSMYLCAS |
| | SLFGGSNTGELFFGEGSRLTVL |
| | [SEQ ID NO: 82] |
| Full α- | MLLEHLLIILWMQLTWVSGQQLNQSPQSMFIQEGEDVSMNCTSSSIFNTWLWYKQD |
| chain | PGEGPVLLIALYKAGELTSNGRLTAQFGITRKDSELNISASIPSDVGIYFCAGRRT |
| | TDSWGKLQFGAGTQVVVTPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 83] |
| Full ß- | MGIRLLCRVAFCFLAVGLVDVKVTQSSRYLVKRTGEKVFLECVQDMDHENMFWYRQ |
| chain | DPGLGLRLIYFSYDVKMKEKGDIPEGYSVSREKKERFSLILESASTNQTSMYLCAS |
| | SLFGGSNTGELFFGEGSRLTVLEDLNKVFPPEVAVFEPSEAEISHTQKATLVCLAT |
| | GFFPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSVSYQQGVLSAT |
| | ILYEILLGKATLYAVLVSALVLMAMVKRKDF |
| | [SEQ ID NO: 84] |

In certain embodiments, the extracellular domain of the TCR comprises an α chain variable region comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof. SEQ ID NOS: 85-87 are disclosed in Table 12. In certain embodiments, the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 88 or a conservative modification thereof. SEQ ID NOS: 37, 38, and 88 are disclosed in Table 12. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88.

In certain embodiments, the α chain variable region comprises a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 88 or a conservative modification thereof. In certain embodiments, the α chain variable region comprises a CDR1 comprising the amino acid sequence set forth in SEQ ID NO: 85, a CDR2 comprising the amino acid sequence set forth in SEQ ID NO: 86, a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 89. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 89. In certain embodiments, the α chain variable region comprises the amino acid sequence set forth in SEQ ID NO: 89. SEQ ID NO: 89 is provided in Table 12.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 90. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 90. In certain embodiments, the β chain variable region comprises the amino acid sequence set forth in SEQ ID NO: 90. SEQ ID NO: 90 is provided in Table 12.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 89; and the β chain variable region comprises an amino acid sequence that is at least about 80% (e.g., at least about 85%, at least about 90%, or at least about 95%) homologous or identical to the amino acid sequence set forth in SEQ ID NO: 90. In certain embodiments, the α chain variable region comprises the amino acid sequence set forth in SEQ ID NO: 89; and the β chain variable region comprises the amino acid sequence set forth in SEQ ID NO: 90.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 91. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 91. In certain embodiments, the α chain comprises the amino acid sequence set forth in SEQ ID NO: 91.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 92. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 92. In certain embodiments, the β chain comprises the amino acid sequence set forth in SEQ ID NO: 92.

In certain embodiments, the α chain comprises an amino acid sequence that is at least about 80% (e.g., at least about 85%, at least about 90%, or at least about 95%) homologous or identical to the amino acid sequence set forth in SEQ ID NO: 91; and the β chain comprises an amino acid sequence that is at least about 80% (e.g., at least about 85%, at least about 90%, or at least about 95%) homologous or identical to the amino acid sequence set forth in SEQ ID NO: 92. In certain embodiments, the α chain comprises the amino acid sequence set forth in SEQ ID NO: 91; and the β chain comprises the amino acid sequence set forth in SEQ ID NO: 92. In certain embodiments, the TCR is designated as “TCR12”. In certain embodiments, the TCR12 binds to a RAS peptide comprising or consisting of the amino acid sequence set forth in SEQ ID NO: 1.

In certain embodiments, the CDRs sequences described above including Table 11 are delineated using the IMGT numbering system.

TABLE 12
(TCR12)

| CDRs | 1 | 2 | 3 |
|---|---|---|---|
| α-chain | SSNFYA | MTLNGDE | CASPDNNARLMF |
| | [SEQ ID NO: 85] | [SEQ ID NO: 86] | [SEQ ID NO: 87] |
| ß-chain | SGDLS | YYNGEE | CASSPNLSWELFF |
| | [SEQ ID NO: 37] | [SEQ ID NO: 38] | [SEQ ID NO: 88] |

| | |
|---|---|
| α-chain | MEKNPLAAPLLILWFHLDCVSSILNVEQSPQSLHVQEGDSTNFTCSFPSSNFYALH |
| variable | WYRWETAKSPEALFVMTLNGDEKKKGRISATLNTKEGYSYLYIKGSQPEDSATYLC |
| | ASPDNNARLMFGDGTQLVVKP |
| | [SEQ ID NO: 89] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SPNLSWELFFGEGSRLTVL |
| | [SEQ ID NO: 90] |
| Full α- | MEKNPLAAPLLILWFHLDCVSSILNVEQSPQSLHVQEGDSTNFTCSFPSSNFYALH |
| chain | WYRWETAKSPEALFVMTLNGDEKKKGRISATLNTKEGYSYLYIKGSQPEDSATYLC |
| | ASPDNNARLMFGDGTQLVVKPNIQNPDPAVYQLRDSKSSDKSVCLFTDFDSQTNVS |
| | QSKDSDVYITDKTVLDMRSMDFKSNSAVAWSNKSDFACANAFNNSIIPEDTFFPSP |
| | ESSCDVKLVEKSFETDTNLNFQNLSVIGFRILLLKVAGFNLLMTLRLWSS |
| | [SEQ ID NO: 91] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SPNLSWELFFGEGSRLTVLEDLNKVEPPEVAVFEPSEAEISHTQKATLVCLATGFF |
| | PDHVELSWWVNGKEVHSGVSTDPQPLKEQPALNDSRYCLSSRLRVSATFWQNPRNH |
| | FRCQVQFYGLSENDEWTQDRAKPVTQIVSAEAWGRADCGFTSVSYQQGVLSATILY |
| | EILLGKATLYAVLVSALVLMAMVKRKDF |
| | [SEQ ID NO: 92] |

In certain embodiments, the extracellular domain of the TCR comprises an α chain variable region comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof. SEQ ID NOS: 93-95 are disclosed in Table 13. In certain embodiments, the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95.

In certain embodiments, the extracellular domain of the TCR comprises a β chain variable region comprising a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 96 or a conservative modification thereof. SEQ ID NOS: 48, 49, and 96 are disclosed in Table 13. In certain embodiments, the β chain variable region CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96.

In certain embodiments, the α chain variable region comprises a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 95 or a conservative modification thereof; and the β chain variable region comprises a CDR1 comprising the amino acid sequence set forth in SEQ ID NO: 48 or a conservative modification thereof, a CDR2 comprising the amino acid sequence set forth in SEQ ID NO: 49 or a conservative modification thereof, and a CDR3 comprising the amino acid sequence set forth in SEQ ID NO: 96 or a conservative modification thereof. In certain embodiments, the α chain variable region comprises a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 95; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 97. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 97. In certain embodiments, the α chain variable region comprises the amino acid sequence set forth in SEQ ID NO: 97. SEQ ID NO: 97 is provided in Table 13.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 98. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 98. In certain embodiments, the β chain variable region comprises the amino acid sequence set forth in SEQ ID NO: 98. SEQ ID NO: 98 is provided in Table 13.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 97; and the β chain variable region comprises an amino acid sequence that is at least about 80% (e.g., at least about 85%, at least about 90%, or at least about 95%) homologous or identical to the amino acid sequence set forth in SEQ ID NO: 98. In certain embodiments, the α chain variable region comprises the amino acid sequence set forth in SEQ ID NO: 97; and the β chain variable region comprises the amino acid sequence set forth in SEQ ID NO: 98.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 99. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 99. In certain embodiments, the α chain comprises the amino acid sequence set forth in SEQ ID NO: 99.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 100. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 100. In certain embodiments, the β chain comprises the amino acid sequence set forth in SEQ ID NO: 100.

In certain embodiments, the α chain comprises an amino acid sequence that is at least about 80% (e.g., at least about 85%, at least about 90%, or at least about 95%) homologous or identical to the amino acid sequence set forth in SEQ ID NO: 99; and the β chain comprises an amino acid sequence that is at least about 80% (e.g., at least about 85%, at least about 90%, or at least about 95%) homologous or identical to the amino acid sequence set forth in SEQ ID NO: 100. In certain embodiments, the α chain comprises the amino acid sequence set forth in SEQ ID NO: 99; and the β chain comprises the amino acid sequence set forth in SEQ ID NO: 100. In certain embodiments, the TCR is designated as “TCR13”. In certain embodiments, the TCR13 binds to a RAS peptide comprising or consisting of the amino acid sequence set forth in SEQ ID NO: 1.

In certain embodiments, the CDRs sequences described above including Table 13 are delineated using the IMGT numbering system.

TABLE 13
(TCR13)

| CDRs | 1 | 2 | 3 |
|---|---|---|---|
| α-chain | ATGYPS | ATKADDK | CALLNNNNDMRF |
| | [SEQ ID NO: 93] | [SEQ ID NO: 94] | [SEQ ID NO: 95] |
| ß-chain | LNHNV | YYDKDF | CATSRDKGTEAHYGYTF |
| | [SEQ ID NO: 48] | [SEQ ID NO: 49] | [SEQ ID NO: 96] |

| | |
|---|---|
| α-chain | MNYSPGLVSLILLLLGRTRGDSVTQMEGPVTLSEEAFLTINCTYTATGYPSLFWYV |
| variable | QYPGEGLQLLLKATKADDKGSNKGFEATYRKETTSFHLEKGSVQVSDSAVYFCALL |
| | NNNNDMRFGAGTRLTVKP |
| | [SEQ ID NO: 97] |
| ß-chain | MGPGLLHWMALCLLGTGHGDAMVIQNPRYQVTQFGKPVTLSCSQTLNHNVMYWYQQ |
| variable | KSSQAPKLLFHYYDKDFNNEADTPDNFQSRRPNTSFCFLDIRSPGLGDAAMYLCAT |
| | SRDKGTEAHYGYTFGSGTRLTVV |
| | [SEQ ID NO: 98] |
| Full α- | MNYSPGLVSLILLLLGRTRGDSVTQMEGPVTLSEEAFLTINCTYTATGYPSLFWYV |
| chain | QYPGEGLQLLLKATKADDKGSNKGFEATYRKETTSFHLEKGSVQVSDSAVYFCALL |
| | NNNNDMRFGAGTRLTVKPNIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESS |
| | CDVKLVEKSFETDTNLNFQNLSVIGFRILLLKVAGFNLLMTLRLWSS |
| | [SEQ ID NO: 99] |
| Full ß- | MGPGLLHWMALCLLGTGHGDAMVIQNPRYQVTQFGKPVTLSCSQTLNHNVMYWYQQ |
| chain | KSSQAPKLLFHYYDKDFNNEADTPDNFQSRRPNTSFCFLDIRSPGLGDAAMYLCAT |
| | SRDKGTEAHYGYTFGSGTRLTVVEDLKNVEPPKVAVFEPSEAEISHTQKATLVCLA |
| | TGFYPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSESYQQGVLSA |
| | TILYEILLGKATLYAVLVSALVLMAMVKRKDSRG |
| | [SEQ ID NO: 100] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 101 or a conservative modification thereof. SEQ ID NOS: 5, 6, and 101 are disclosed in Table 14. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101.

In certain embodiments, the extracellular domain of the TCR comprises a β chain variable region comprising a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 104 or a conservative modification thereof. SEQ ID NOS: 102-104 are disclosed in Table 14. In certain embodiments, the β chain variable region CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 101 or a conservative modification thereof; and the β chain variable region comprises a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 104 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 105. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 105. In certain embodiments, the α chain variable region comprises the amino acid sequence set forth in SEQ ID NO: 105. SEQ ID NO: 105 is provided in Table 14.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 106. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 106. In certain embodiments, the β chain variable region comprises the amino acid sequence set forth in SEQ ID NO: 106. SEQ ID NO: 106 is provided in Table 14.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 105; and the β chain variable region comprises an amino acid sequence that is at least about 80% (e.g., at least about 85%, at least about 90%, or at least about 95%) homologous or identical to the amino acid sequence set forth in SEQ ID NO: 106. In certain embodiments, the α chain variable region comprises the amino acid sequence set forth in SEQ ID NO: 105; and the β chain variable region comprises the amino acid sequence set forth in SEQ ID NO: 106.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 107. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 107. In certain embodiments, the α chain comprises the amino acid sequence set forth in SEQ ID NO: 107.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 108. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 108. In certain embodiments, the β chain comprises the amino acid sequence set forth in SEQ ID NO: 108.

In certain embodiments, the α chain comprises an amino acid sequence that is at least about 80% (e.g., at least about 85%, at least about 90%, or at least about 95%) homologous or identical to the amino acid sequence set forth in SEQ ID NO: 107; and the β chain comprises an amino acid sequence that is at least about 80% (e.g., at least about 85%, at least about 90%, or at least about 95%) homologous or identical to the amino acid sequence set forth in SEQ ID NO: 108. In certain embodiments, the α chain comprises the amino acid sequence set forth in SEQ ID NO: 107; and the β chain comprises the amino acid sequence set forth in SEQ ID NO: 108. In certain embodiments, the TCR is designated as “TCR14”. In certain embodiments, the TCR14 binds to a RAS peptide comprising or consisting of the amino acid sequence set forth in SEQ ID NO: 1.

In certain embodiments, the CDRs sequences described above including Table 14 are delineated using the IMGT numbering system.

TABLE 14
(TCR14)

| CDRs | 1 | 2 | 3 |
|---|---|---|---|
| α-chain | SIFNT | LYKAGEL | CAGHRGDNYGQNEVF |
| | [SEQ ID NO: 5] | [SEQ ID NO: 6] | [SEQ ID NO: 101] |
| ß-chain | MGHRA | YSYEKL | CASSQDRLTYNEQFF |
| | [SEQ ID NO: 102] | [SEQ ID NO: 103] | [SEQ ID NO: 104] |

| | |
|---|---|
| α-chain | MLLEHLLIILWMQLTWVSGQQLNQSPQSMFIQEGEDVSMNCTSSSIFNTWLWYKQD |
| variable | PGEGPVLLIALYKAGELTSNGRLTAQFGITRKDSELNISASIPSDVGIYFCAGHRG |
| | DNYGQNFVFGPGTRLSVLP |
| | [SEQ ID NO: 105] |
| ß-chain | MGCRLLCCAVLCLLGAVPIDTEVTQTPKHLVMGMTNKKSLKCEQHMGHRAMYWYKQ |
| variable | KAKKPPELMFVYSYEKLSINESVPSRFSPECPNSSLLNLHLHALQPEDSALYLCAS |
| | SQDRLTYNEQFFGPGTRLTVL |
| | [SEQ ID NO: 106] |
| Full α- | MLLEHLLIILWMQLTWVSGQQLNQSPQSMFIQEGEDVSMNCTSSSIFNTWLWYKQD |
| chain | PGEGPVLLIALYKAGELTSNGRLTAQFGITRKDSFLNISASIPSDVGIYFCAGHRG |
| | DNYGQNFVFGPGTRLSVLPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 107] |
| Full ß- | MGCRLLCCAVLCLLGAVPIDTEVTQTPKHLVMGMTNKKSLKCEQHMGHRAMYWYKQ |
| chain | KAKKPPELMFVYSYEKLSINESVPSRFSPECPNSSLLNLHLHALQPEDSALYLCAS |
| | SQDRLTYNEQFFGPGTRLTVLEDLNKVEPPEVAVFEPSEAEISHTQKATLVCLATG |
| | FFPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSVSYQQGVLSATI |
| | LYEILLGKATLYAVLVSALVLMAMVKRKDF |
| | [SEQ ID NO: 108] |

In certain embodiments, the extracellular domain of the TCR comprises an α chain variable region comprising a CDR1 comprising the amino acid sequence set forth in SEQ ID NO: 109 or a conservative modification thereof, a CDR2 comprising the amino acid sequence set forth in SEQ ID NO: 110 or a conservative modification thereof, and a CDR3 comprising the amino acid sequence set forth in SEQ ID NO: 111 or a conservative modification thereof. SEQ ID NOS: 109-111 are disclosed in Table 15. In certain embodiments, the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111.

In certain embodiments, the extracellular domain of the TCR comprises a β chain variable region comprising a CDR1 comprising the amino acid sequence set forth in SEQ ID NO: 112 or a conservative modification thereof, a CDR2 comprising the amino acid sequence set forth in SEQ ID NO: 113 or a conservative modification thereof, and a CDR3 comprising the amino acid sequence set forth in SEQ ID NO: 114 or a conservative modification thereof. SEQ ID NOS: 112-114 are disclosed in Table 15. In certain embodiments, the β chain variable region CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114.

In certain embodiments, the α chain variable region comprises a CDR1 comprising the amino acid sequence set forth in SEQ ID NO: 109 or a conservative modification thereof, a CDR2 comprising the amino acid sequence set forth in SEQ ID NO: 110 or a conservative modification thereof, and a CDR3 comprising the amino acid sequence set forth in SEQ ID NO: 111 or a conservative modification thereof; and the β chain variable region comprises a CDR1 comprising the amino acid sequence set forth in SEQ ID NO: 112 or a conservative modification thereof, a CDR2 comprising the amino acid sequence set forth in SEQ ID NO: 113 or a conservative modification thereof, and a CDR3 comprising the amino acid sequence set forth in SEQ ID NO: 114 or a conservative modification thereof. In certain embodiments, the α chain variable region comprises a CDR1 comprising the amino acid sequence set forth in SEQ ID NO: 109, a CDR2 comprising the amino acid sequence set forth in SEQ ID NO: 110, a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 115. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 115. In certain embodiments, the α chain variable region comprises the amino acid sequence set forth in SEQ ID NO: 115. SEQ ID NO: 115 is provided in Table 15.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 116. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 116. In certain embodiments, the β chain variable region comprises the amino acid sequence set forth in SEQ ID NO: 116. SEQ ID NO: 116 is provided in Table 15.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 115; and the β chain variable region comprises an amino acid sequence that is at least about 80% (e.g., at least about 85%, at least about 90%, or at least about 95%) homologous or identical to the amino acid sequence set forth in SEQ ID NO: 116. In certain embodiments, the α chain variable region comprises the amino acid sequence set forth in SEQ ID NO: 115; and the β chain variable region comprises the amino acid sequence set forth in SEQ ID NO: 116.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 117. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 117. In certain embodiments, the α chain comprises the amino acid sequence set forth in SEQ ID NO: 117.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 118. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 118. In certain embodiments, the β chain comprises the amino acid sequence set forth in SEQ ID NO: 118.

In certain embodiments, the α chain comprises an amino acid sequence that is at least about 80% (e.g., at least about 85%, at least about 90%, or at least about 95%) homologous or identical to the amino acid sequence set forth in SEQ ID NO: 117; and the β chain comprises an amino acid sequence that is at least about 80% (e.g., at least about 85%, at least about 90%, or at least about 95%) homologous or identical to the amino acid sequence set forth in SEQ ID NO: 118. In certain embodiments, the α chain comprises the amino acid sequence set forth in SEQ ID NO: 117; and the β chain comprises the amino acid sequence set forth in SEQ ID NO: 118. In certain embodiments, the TCR is designated as “TCR15”. In certain embodiments, the TCR15 binds to a RAS peptide comprising or consisting of the amino acid sequence set forth in SEQ ID NO: 3.

In certain embodiments, the CDRs sequences described above including Table 15 are delineated using the IMGT numbering system.

TABLE 15
(TCR15)

| CDRs | 1 | 2 | 3 |
|---|---|---|---|
| α-chain | TSGFYG | NALDGL | CAVHSWGKLQF |
| | [SEQ ID NO: 109] | [SEQ ID NO: 110] | [SEQ ID NO: 111] |
| ß-chain | SEHNR | FQNEAQ | CASSPLGGSYEQYF |
| | [SEQ ID NO: 112] | [SEQ ID NO: 113] | [SEQ ID NO: 114] |

| | |
|---|---|
| α-chain | MWGAFLLYVSMKMGGTAGQSLEQPSEVTAVEGAIVQINCTYQTSGFYGLSWYQQHD |
| variable | GGAPTFLSYNALDGLEETGRFSSFLSRSDSYGYLLLQELQMKDSASYFCAVHSWGK |
| | LQFGAGTQVVVTP |
| | [SEQ ID NO: 115] |
| ß-chain | MGTSLLCWMALCLLGADHADTGVSQDPRHKITKRGQNVTFRCDPISEHNRLYWYRQ |
| variable | TLGQGPEFLTYFQNEAQLEKSRLLSDRFSAERPKGSESTLEIQRTEQGDSAMYLCA |
| | SSPLGGSYEQYFGPGTRLTVT |
| | [SEQ ID NO: 116] |
| Full α- | MWGAFLLYVSMKMGGTAGQSLEQPSEVTAVEGAIVQINCTYQTSGFYGLSWYQQHD |
| chain | GGAPTFLSYNALDGLEETGRFSSFLSRSDSYGYLLLQELQMKDSASYFCAVHSWGK |
| | LQFGAGTQVVVTPNIQNPDPAVYQLRDSKSSDKSVCLFTDFDSQTNVSQSKDSDVY |
| | ITDKTVLDMRSMDFKSNSAVAWSNKSDFACANAFNNSIIPEDTFFPSPESSCDVKL |
| | VEKSFETDTNLNFQNLSVIGFRILLLKVAGFNLLMTLRLWSS |
| | [SEQ ID NO: 117] |
| Full ß- | MGTSLLCWMALCLLGADHADTGVSQDPRHKITKRGQNVTFRCDPISEHNRLYWYRQ |
| chain | TLGQGPEFLTYFQNEAQLEKSRLLSDRFSAERPKGSESTLEIQRTEQGDSAMYLCA |
| | SSPLGGSYEQYFGPGTRLTVTEDLNKVEPPEVAVFEPSEAEISHTQKATLVCLATG |
| | FFPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSVSYQQGVLSATI |
| | LYEILLGKATLYAVLVSALVLMAMVKRKDF |
| | [SEQ ID NO: 118] |

In certain embodiments, the extracellular domain of the TCR comprises an α chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. SEQ ID NOS: 119-121 are disclosed in Table 16. In certain embodiments, the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO:

121.

In certain embodiments, the extracellular domain of the TCR comprises a β chain variable region comprising a CDR1 comprising the amino acid sequence set forth in SEQ ID NO: 122 or a conservative modification thereof, a CDR2 comprising the amino acid sequence set forth in SEQ ID NO: 113 or a conservative modification thereof, and a CDR3 comprising the amino acid sequence set forth in SEQ ID NO: 123 or a conservative modification thereof. SEQ ID NOS: 113, 121, and 123 are disclosed in Table 16. In certain embodiments, the β chain variable region CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123.

In certain embodiments, the α chain variable region comprises a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof; and the β chain variable region comprises a CDR1 comprising the amino acid sequence set forth in SEQ ID NO: 122 or a conservative modification thereof, a CDR2 comprising the amino acid sequence set forth in SEQ ID NO: 113 or a conservative modification thereof, and a CDR3 comprising the amino acid sequence set forth in SEQ ID NO: 123 or a conservative modification thereof. In certain embodiments, the α chain variable region comprises a CDR1 comprising the amino acid sequence set forth in SEQ ID NO: 119, a CDR2 comprising the amino acid sequence set forth in SEQ ID NO: 120, a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 124. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 124. In certain embodiments, the α chain variable region comprises the amino acid sequence set forth in SEQ ID NO: 124. SEQ ID NO: 124 is provided in Table 16.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 125. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 125. In certain embodiments, the β chain variable region comprises the amino acid sequence set forth in SEQ ID NO: 125. SEQ ID NO: 125 is provided in Table 16.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 124; and the β chain variable region comprises an amino acid sequence that is at least about 80% (e.g., at least about 85%, at least about 90%, or at least about 95%) homologous or identical to the amino acid sequence set forth in SEQ ID NO: 125. In certain embodiments, the α chain variable region comprises the amino acid sequence set forth in SEQ ID NO: 124; and the β chain variable region comprises the amino acid sequence set forth in SEQ ID NO: 125.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 126. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 126. In certain embodiments, the α chain comprises the amino acid sequence set forth in SEQ ID NO: 126.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 127. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 127. In certain embodiments, the β chain comprises the amino acid sequence set forth in SEQ ID NO: 127.

In certain embodiments, the α chain comprises an amino acid sequence that is at least about 80% (e.g., at least about 85%, at least about 90%, or at least about 95%) homologous or identical to the amino acid sequence set forth in SEQ ID NO: 126; and the β chain comprises an amino acid sequence that is at least about 80% (e.g., at least about 85%, at least about 90%, or at least about 95%) homologous or identical to the amino acid sequence set forth in SEQ ID NO: 127. In certain embodiments, the α chain comprises the amino acid sequence set forth in SEQ ID NO: 126; and the β chain comprises the amino acid sequence set forth in SEQ ID NO: 127. In certain embodiments, the TCR is designated as “TCR16”. In certain embodiments, the TCR16 binds to a RAS peptide comprising or consisting of the amino acid sequence set forth in SEQ ID NO: 3.

In certain embodiments, the CDRs sequences described above including Table 16 are delineated using the IMGT numbering system.

TABLE 16
(TCR16)

| CDRs | 1 | 2 | 3 |
|---|---|---|---|
| α-chain | TSINN | IRSNERE | CATDAGGGNKLVF |
| | [SEQ ID NO: 119] | [SEQ ID NO: 120] | [SEQ ID NO: 121] |
| ß-chain | SGHVS | FQNEAQ | CASSELGGSNEKLFF |
| | [SEQ ID NO: 122] | [SEQ ID NO: 113] | [SEQ ID NO: 123] |

| | |
|---|---|
| α-chain | METLLGVSLVILWLQLARVNSQQGEEDPQALSIQEGENATMNCSYKTSINNLQWYR |
| variable | QNSGRGLVHLILIRSNEREKHSGRLRVTLDTSKKSSSLLITASRAADTASYFCATD |
| | AGGGNKLVFGAGTILRVKS |
| | [SEQ ID NO: 124] |
| ß-chain | MGTRLLCWVVLGFLGTDHTGAGVSQSPRYKVAKRGQDVALRCDPISGHVSLEWYQQ |
| variable | ALGQGPEFLTYFQNEAQLDKSGLPSDRFFAERPEGSVSTLKIQRTQQEDSAVYLCA |
| | SSFLGGSNEKLFFGSGTQLSVL |
| | [SEQ ID NO: 125] |
| Full α- | METLLGVSLVILWLQLARVNSQQGEEDPQALSIQEGENATMNCSYKTSINNLQWYR |
| chain | QNSGRGLVHLILIRSNEREKHSGRLRVTLDTSKKSSSLLITASRAADTASYFCATD |
| | AGGGNKLVFGAGTILRVKSNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 126] |
| Full ß- | MGTRLLCWVVLGFLGTDHTGAGVSQSPRYKVAKRGQDVALRCDPISGHVSLFWYQQ |
| chain | ALGQGPEFLTYFQNEAQLDKSGLPSDRFFAERPEGSVSTLKIQRTQQEDSAVYLCA |
| | SSFLGGSNEKLFFGSGTQLSVLEDLKNVEPPKVAVFEPSEAEISHTQKATLVCLAT |
| | GFYPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSESYQQGVLSAT |
| | ILYEILLGKATLYAVLVSALVLMAMVKRKDSRG |
| | [SEQ ID NO: 127] |

In certain embodiments, the extracellular domain of the TCR comprises an α chain variable region comprising a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 130 or a conservative modification thereof. SEQ ID NOS: 128-130 are disclosed in Table 17. In certain embodiments, the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130.

In certain embodiments, the extracellular domain of the TCR comprises a β chain variable region comprising a CDR1 comprising the amino acid sequence set forth in SEQ ID NO: 131 or a conservative modification thereof, a CDR2 comprising the amino acid sequence set forth in SEQ ID NO: 132 or a conservative modification thereof, and a CDR3 comprising the amino acid sequence set forth in SEQ ID NO: 133 or a conservative modification thereof. SEQ ID NOS: 131-133 are disclosed in Table 17. In certain embodiments, the β chain variable region CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133.

In certain embodiments, the α chain variable region comprises a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 130 or a conservative modification thereof; and the β chain variable region comprises a CDR1 comprising the amino acid sequence set forth in SEQ ID NO: 131 or a conservative modification thereof, a CDR2 comprising the amino acid sequence set forth in SEQ ID NO: 132 or a conservative modification thereof, and a CDR3 comprising the amino acid sequence set forth in SEQ ID NO: 133 or a conservative modification thereof. In certain embodiments, the α chain variable region comprises a CDR1 comprising the amino acid sequence set forth in SEQ ID NO: 128, a CDR2 comprising the amino acid sequence set forth in SEQ ID NO: 129, a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 134. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 134. In certain embodiments, the α chain variable region comprises the amino acid sequence set forth in SEQ ID NO: 134. SEQ ID NO: 134 is provided in Table 17.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 135. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 135. In certain embodiments, the β chain variable region comprises the amino acid sequence set forth in SEQ ID NO: 135. SEQ ID NO: 135 is provided in Table 17.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 134; and the β chain variable region comprises an amino acid sequence that is at least about 80% (e.g., at least about 85%, at least about 90%, or at least about 95%) homologous or identical to the amino acid sequence set forth in SEQ ID NO: 135. In certain embodiments, the α chain variable region comprises the amino acid sequence set forth in SEQ ID NO: 134; and the β chain variable region comprises the amino acid sequence set forth in SEQ ID NO: 135.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 136. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 136. In certain embodiments, the α chain comprises the amino acid sequence set forth in SEQ ID NO: 136.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 137. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 137. In certain embodiments, the β chain comprises the amino acid sequence set forth in SEQ ID NO: 137.

In certain embodiments, the α chain comprises an amino acid sequence that is at least about 80% (e.g., at least about 85%, at least about 90%, or at least about 95%) homologous or identical to the amino acid sequence set forth in SEQ ID NO: 136; and the β chain comprises an amino acid sequence that is at least about 80% (e.g., at least about 85%, at least about 90%, or at least about 95%) homologous or identical to the amino acid sequence set forth in SEQ ID NO: 137. In certain embodiments, the α chain comprises the amino acid sequence set forth in SEQ ID NO: 136; and the β chain comprises the amino acid sequence set forth in SEQ ID NO: 137. In certain embodiments, the TCR is designated as “TCR17”. In certain embodiments, the TCR17 binds to a RAS peptide comprising or consisting of the amino acid sequence set forth in SEQ ID NO: 3.

In certain embodiments, the CDRs sequences described above including Table 17 are delineated using the IMGT numbering system.

TABLE 17
(TCR17)

| CDRs | 1 | 2 | 3 |
|---|---|---|---|
| α-chain | NSMFDY | ISSIKDK | CAASDQAGTALIF |
| | [SEQ ID NO: 128] | [SEQ ID NO: 129] | [SEQ ID NO: 130] |
| ß-chain | KGHSY | FQNENV | CASSPPGQGTNTIYF |
| | [SEQ ID NO: 131] | [SEQ ID NO: 132] | [SEQ ID NO: 133] |

| | |
|---|---|
| α-chain | MAMLLGASVLILWLQPDWVNSQQKNDDQQVKQNSPSLSVQEGRISILNCDYTNSMF |
| variable | DYFLWYKKYPAEGPTFLISISSIKDKNEDGRFTVFLNKSAKHLSLHIVPSQPGDSA |
| | VYFCAASDQAGTALIFGKGTTLSVSS |
| | [SEQ ID NO: 134] |
| ß-chain | MSPIFTCITILCLLAAGSPGEEVAQTPKHLVRGEGQKAKLYCAPIKGHSYVFWYQQ |
| variable | VLKNEFKELISFQNENVEDETGMPKERFSAKCLPNSPCSLEIQATKLEDSAVYFCA |
| | SSPPGQGTNTIYFGEGSWLTVV |
| | [SEQ ID NO: 135] |
| Full α- | MAMLLGASVLILWLQPDWVNSQQKNDDQQVKQNSPSLSVQEGRISILNCDYTNSMF |
| chain | DYFLWYKKYPAEGPTFLISISSIKDKNEDGRFTVELNKSAKHLSLHIVPSQPGDSA |
| | VYFCAASDQAGTALIFGKGTTLSVSSNIQNPDPAVYQLRDSKSSDKSVCLFTDFDS |
| | QTNVSQSKDSDVYITDKTVLDMRSMDFKSNSAVAWSNKSDFACANAFNNSIIPEDT |
| | FFPSPESSCDVKLVEKSFETDTNLNFQNLSVIGFRILLLKVAGFNLLMTLRLWSS |
| | [SEQ ID NO: 136] |
| Full ß- | MSPIFTCITILCLLAAGSPGEEVAQTPKHLVRGEGQKAKLYCAPIKGHSYVFWYQQ |
| chain | VLKNEFKFLISFQNENVEDETGMPKERFSAKCLPNSPCSLEIQATKLEDSAVYFCA |
| | SSPPGQGTNTIYFGEGSWLTVVEDLKNVFPPKVAVFEPSEAEISHTQKATLVCLAT |
| | GFYPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSESYQQGVLSAT |
| | ILYEILLGKATLYAVLVSALVLMAMVKRKDSRG |
| | [SEQ ID NO: 137] |

In certain embodiments, the extracellular domain of the TCR comprises an α chain variable region comprising a CDR1 comprising the amino acid sequence set forth in SEQ ID NO: 138 or a conservative modification thereof, a CDR2 comprising the amino acid sequence set forth in SEQ ID NO: 35 or a conservative modification thereof, and a CDR3 comprising the amino acid sequence set forth in SEQ ID NO: 139 or a conservative modification thereof. SEQ ID NOS: 35, 139, and 139 are disclosed in Table 18. In certain embodiments, the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139.

In certain embodiments, the extracellular domain of the TCR comprises a β chain variable region comprising a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 140 or a conservative modification thereof. SEQ ID NOS: 102, 103, 140 are disclosed in Table 18. In certain embodiments, the β chain variable region CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 140.

In certain embodiments, the α chain variable region comprises a CDR1 comprising the amino acid sequence set forth in SEQ ID NO: 138 or a conservative modification thereof, a CDR2 comprising the amino acid sequence set forth in SEQ ID NO: 35 or a conservative modification thereof, and a CDR3 comprising the amino acid sequence set forth in SEQ ID NO: 139 or a conservative modification thereof; and the β chain variable region comprises a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 140 or a conservative modification thereof. In certain embodiments, the α chain variable region comprises a CDR1 comprising the amino acid sequence set forth in SEQ ID NO: 138, a CDR2 comprising the amino acid sequence set forth in SEQ ID NO: 35, a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 140.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 141. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 141. In certain embodiments, the α chain variable region comprises the amino acid sequence set forth in SEQ ID NO: 141. SEQ ID NO: 141 is provided in Table 18.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 142. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 142. In certain embodiments, the β chain variable region comprises the amino acid sequence set forth in SEQ ID NO: 142. SEQ ID NO: 142 is provided in Table 18.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 141; and the β chain variable region comprises an amino acid sequence that is at least about 80% (e.g., at least about 85%, at least about 90%, or at least about 95%) homologous or identical to the amino acid sequence set forth in SEQ ID NO: 142. In certain embodiments, the α chain variable region comprises the amino acid sequence set forth in SEQ ID NO: 141; and the β chain variable region comprises the amino acid sequence set forth in SEQ ID NO: 142.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 143. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 143. In certain embodiments, the α chain comprises the amino acid sequence set forth in SEQ ID NO: 143.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 144. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 144. In certain embodiments, the β chain comprises the amino acid sequence set forth in SEQ ID NO: 144.

In certain embodiments, the α chain comprises an amino acid sequence that is at least about 80% (e.g., at least about 85%, at least about 90%, or at least about 95%) homologous or identical to the amino acid sequence set forth in SEQ ID NO: 143; and the β chain comprises an amino acid sequence that is at least about 80% (e.g., at least about 85%, at least about 90%, or at least about 95%) homologous or identical to the amino acid sequence set forth in SEQ ID NO: 144. In certain embodiments, the α chain comprises the amino acid sequence set forth in SEQ ID NO: 143; and the β chain comprises the amino acid sequence set forth in SEQ ID NO: 144. In certain embodiments, the TCR is designated as “TCR18”. In certain embodiments, the TCR18 binds to a RAS peptide comprising or consisting of the amino acid sequence set forth in SEQ ID NO: 3.

In certain embodiments, the CDRs sequences described above including Table 18 are delineated using the IMGT numbering system.

TABLE 18
(TCR18)

| CDRs | 1 | 2 | 3 |
|---|---|---|---|
| α-chain | TSENNYY | QEAYKQQN | CAFLNARLMF |
| | [SEQ ID NO: 138] | [SEQ ID NO: 35] | [SEQ ID NO: 139] |
| ß-chain | MGHRA | YSYEKL | CASSQDPQGSYNEQFF |
| | [SEQ ID NO: 102] | [SEQ ID NO: 103] | [SEQ ID NO: 140] |

| | |
|---|---|
| α-chain | MTRVSLLWAVVVSTCLESGMAQTVTQSQPEMSVQEAETVTLSCTYDTSENNYYLFW |
| variable | YKQPPSRQMILVIRQEAYKQQNATENRFSVNFQKAAKSFSLKISDSQLGDTAMYFC |
| | AFLNARLMFGDGTQLVVKP |
| | [SEQ ID NO: 141] |
| ß-chain | MGCRLLCCAVLCLLGAVPIDTEVTQTPKHLVMGMTNKKSLKCEQHMGHRAMYWYKQ |
| variable | KAKKPPELMFVYSYEKLSINESVPSRFSPECPNSSLLNLHLHALQPEDSALYLCAS |
| | SQDPQGSYNEQFFGPGTRLTVL |
| | [SEQ ID NO: 142] |
| Full α- | MTRVSLLWAVVVSTCLESGMAQTVTQSQPEMSVQEAETVTLSCTYDTSENNYYLFW |
| chain | YKQPPSRQMILVIRQEAYKQQNATENRFSVNFQKAAKSFSLKISDSQLGDTAMYFC |
| | AFLNARLMFGDGTQLVVKPNIQNPDPAVYQLRDSKSSDKSVCLFTDEDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 143] |
| Full ß- | MGCRLLCCAVLCLLGAVPIDTEVTQTPKHLVMGMTNKKSLKCEQHMGHRAMYWYKQ |
| chain | KAKKPPELMFVYSYEKLSINESVPSRESPECPNSSLLNLHLHALQPEDSALYLCAS |
| | SQDPQGSYNEQFFGPGTRLTVLEDLNKVEPPEVAVFEPSEAEISHTQKATLVCLAT |
| | GFFPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSVSYQQGVLSAT |
| | ILYEILLGKATLYAVLVSALVLMAMVKRKDF |
| | [SEQ ID NO: 144] |

In certain embodiments, the extracellular domain of the TCR comprises an α chain variable region comprising a CDR1 comprising the amino acid sequence set forth in SEQ ID NO: 145 or a conservative modification thereof, a CDR2 comprising the amino acid sequence set forth in SEQ ID NO: 146 or a conservative modification thereof, and a CDR3 comprising the amino acid sequence set forth in SEQ ID NO: 147 or a conservative modification thereof. SEQ ID NOS: 145-147 are disclosed in Table 19. In certain embodiments, the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147.

In certain embodiments, the extracellular domain of the TCR comprises a β chain variable region comprising a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 140 or a conservative modification thereof. SEQ ID NOS: 102, 103, and 140 are disclosed in Table 19. In certain embodiments, the β chain variable region CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 140.

In certain embodiments, the α chain variable region comprises a CDR1 comprising the amino acid sequence set forth in SEQ ID NO: 145 or a conservative modification thereof, a CDR2 comprising the amino acid sequence set forth in SEQ ID NO: 146 or a conservative modification thereof, and a CDR3 comprising the amino acid sequence set forth in SEQ ID NO: 147 or a conservative modification thereof; and the β chain variable region comprises a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 140 or a conservative modification thereof. In certain embodiments, the α chain variable region comprises a CDR1 comprising the amino acid sequence set forth in SEQ ID NO: 145, a CDR2 comprising the amino acid sequence set forth in SEQ ID NO: 146, a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 140.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 148. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 148. In certain embodiments, the α chain variable region comprises the amino acid sequence set forth in SEQ ID NO: 148. SEQ ID NO: 148 is provided in Table 19.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 142. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 142. In certain embodiments, the β chain variable region comprises the amino acid sequence set forth in SEQ ID NO: 142. SEQ ID NO: 142 is provided in Table 19.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 148; and the β chain variable region comprises an amino acid sequence that is at least about 80% (e.g., at least about 85%, at least about 90%, or at least about 95%) homologous or identical to the amino acid sequence set forth in SEQ ID NO: 142. In certain embodiments, the α chain variable region comprises the amino acid sequence set forth in SEQ ID NO: 148; and the β chain variable region comprises the amino acid sequence set forth in SEQ ID NO: 142.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 149. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 149. In certain embodiments, the α chain comprises the amino acid sequence set forth in SEQ ID NO: 149.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 144. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 144. In certain embodiments, the β chain comprises the amino acid sequence set forth in SEQ ID NO: 144.

In certain embodiments, the α chain comprises an amino acid sequence that is at least about 80% (e.g., at least about 85%, at least about 90%, or at least about 95%) homologous or identical to the amino acid sequence set forth in SEQ ID NO: 149; and the β chain comprises an amino acid sequence that is at least about 80% (e.g., at least about 85%, at least about 90%, or at least about 95%) homologous or identical to the amino acid sequence set forth in SEQ ID NO: 144. In certain embodiments, the α chain comprises the amino acid sequence set forth in SEQ ID NO: 149; and the β chain comprises the amino acid sequence set forth in SEQ ID NO: 144. In certain embodiments, the TCR is designated as “TCR19”. In certain embodiments, the TCR19 binds to a RAS peptide comprising or consisting of the amino acid sequence set forth in SEQ ID NO: 3.

In certain embodiments, the CDRs sequences described above including Table 19 are delineated using the IMGT numbering system.

TABLE 19
(TCR19)

| CDRs | 1 | 2 | 3 |
|---|---|---|---|
| α-chain | TISGTDY | GLTSN | CILRGGSEKLVF |
| | [SEQ ID NO: 145] | [SEQ ID NO: 146] | [SEQ ID NO: 147] |
| ß-chain | MGHRA | YSYEKL | CASSQDPQGSYNEQFF |
| | [SEQ ID NO: 102] | [SEQ ID NO: 103] | [SEQ ID NO: 140] |

| | |
|---|---|
| α-chain | MKLVTSITVLLSLGIMGDAKTTQPNSMESNEEEPVHLPCNHSTISGTDYIHWYRQL |
| variable | PSQGPEYVIHGLTSNVNNRMASLAIAEDRKSSTLILHRATLRDAAVYYCILRGGSE |
| | KLVFGKGTKLTVNP |
| | [SEQ ID NO: 148] |
| ß-chain | MGCRLLCCAVLCLLGAVPIDTEVTQTPKHLVMGMTNKKSLKCEQHMGHRAMYWYKQ |
| variable | KAKKPPELMFVYSYEKLSINESVPSRFSPECPNSSLLNLHLHALQPEDSALYLCAS |
| | SQDPQGSYNEQFFGPGTRLTVL |
| | [SEQ ID NO: 142] |
| Full α- | MKLVTSITVLLSLGIMGDAKTTQPNSMESNEEEPVHLPCNHSTISGTDYIHWYRQL |
| chain | PSQGPEYVIHGLTSNVNNRMASLAIAEDRKSSTLILHRATLRDAAVYYCILRGGSE |
| | KLVFGKGTKLTVNPNIQNPDPAVYQLRDSKSSDKSVCLFTDFDSQTNVSQSKDSDV |
| | YITDKTVLDMRSMDFKSNSAVAWSNKSDFACANAFNNSIIPEDTFFPSPESSCDVK |
| | LVEKSFETDTNLNFQNLSVIGFRILLLKVAGFNLLMTLRLWSS |
| | [SEQ ID NO: 149] |
| Full ß- | MGCRLLCCAVLCLLGAVPIDTEVTQTPKHLVMGMTNKKSLKCEQHMGHRAMYWYKQ |
| chain | KAKKPPELMFVYSYEKLSINESVPSRFSPECPNSSLLNLHLHALQPEDSALYLCAS |
| | SQDPQGSYNEQFFGPGTRLTVLEDLNKVFPPEVAVFEPSEAEISHTQKATLVCLAT |
| | GFFPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSVSYQQGVLSAT |
| | ILYEILLGKATLYAVLVSALVLMAMVKRKDF |
| | [SEQ ID NO: 144] |

In certain embodiments, the extracellular domain of the TCR comprises an α chain variable region comprising a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 150 or a conservative modification thereof. SEQ ID NOS: 34, 35, and 150 are disclosed in Table 20. In certain embodiments, the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 151 or a conservative modification thereof. SEQ ID NOS: 37, 38, and 151 are disclosed in Table 20. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151.

In certain embodiments, the α chain variable region comprises a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 150 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 151 or a conservative modification thereof. In certain embodiments, the α chain variable region comprises a CDR1 comprising the amino acid sequence set forth in SEQ ID NO: 34, a CDR2 comprising the amino acid sequence set forth in SEQ ID NO: 35, a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 152. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 152. In certain embodiments, the α chain variable region comprises the amino acid sequence set forth in SEQ ID NO: 152. SEQ ID NO: 152 is provided in Table 20.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 153. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 153. In certain embodiments, the β chain variable region comprises the amino acid sequence set forth in SEQ ID NO: 153. SEQ ID NO: 153 is provided in Table 20.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 152; and the β chain variable region comprises an amino acid sequence that is at least about 80% (e.g., at least about 85%, at least about 90%, or at least about 95%) homologous or identical to the amino acid sequence set forth in SEQ ID NO: 153. In certain embodiments, the α chain variable region comprises the amino acid sequence set forth in SEQ ID NO: 152; and the β chain variable region comprises the amino acid sequence set forth in SEQ ID NO: 153.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 154. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 154. In certain embodiments, the α chain comprises the amino acid sequence set forth in SEQ ID NO: 154.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 155. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 155. In certain embodiments, the β chain comprises the amino acid sequence set forth in SEQ ID NO: 155.

In certain embodiments, the α chain comprises an amino acid sequence that is at least about 80% (e.g., at least about 85%, at least about 90%, or at least about 95%) homologous or identical to the amino acid sequence set forth in SEQ ID NO: 154; and the β chain comprises an amino acid sequence that is at least about 80% (e.g., at least about 85%, at least about 90%, or at least about 95%) homologous or identical to the amino acid sequence set forth in SEQ ID NO: 155. In certain embodiments, the α chain comprises the amino acid sequence set forth in SEQ ID NO: 154; and the β chain comprises the amino acid sequence set forth in SEQ ID NO: 155. In certain embodiments, the TCR is designated as “TCR20”. In certain embodiments, the TCR20 binds to a RAS peptide comprising or consisting of the amino acid sequence set forth in SEQ ID NO: 3.

In certain embodiments, the CDRs sequences described above including Table 20 are delineated using the IMGT numbering system.

TABLE 20
(TCR20)

| CDRs | 1 | 2 | 3 |
|---|---|---|---|
| α-chain | TSESDYY | QEAYKQQN | CAYRSPNTPLVF |
| | [SEQ ID NO: 34] | [SEQ ID NO: 35] | [SEQ ID NO: 150] |
| ß-chain | SGDLS | YYNGEE | CASSAWTSGELFF |
| | [SEQ ID NO: 37] | [SEQ ID NO: 38] | [SEQ ID NO: 151] |

| | |
|---|---|
| α-chain | MACPGFLWALVISTCLEFSMAQTVTQSQPEMSVQEAETVTLSCTYDTSESDYYLFW |
| variable | YKQPPSRQMILVIRQEAYKQQNATENRFSVNFQKAAKSFSLKISDSQLGDAAMYFC |
| | AYRSPNTPLVFGKGTRLSVIA |
| | [SEQ ID NO: 152] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SAWTSGELFFGEGSRLTVL |
| | [SEQ ID NO: 153] |
| Full α- | MACPGELWALVISTCLEFSMAQTVTQSQPEMSVQEAETVTLSCTYDTSESDYYLFW |
| chain | YKQPPSRQMILVIRQEAYKQQNATENRFSVNFQKAAKSFSLKISDSQLGDAAMYFC |
| | AYRSPNTPLVFGKGTRLSVIANIQNPDPAVYQLRDSKSSDKSVCLFTDFDSQTNVS |
| | QSKDSDVYITDKTVLDMRSMDFKSNSAVAWSNKSDFACANAFNNSIIPEDTFFPSP |
| | ESSCDVKLVEKSFETDTNLNFQNLSVIGFRILLLKVAGFNLLMTLRLWSS |
| | [SEQ ID NO: 154] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SAWTSGELFFGEGSRLTVLEDLNKVEPPEVAVFEPSEAEISHTQKATLVCLATGFF |
| | PDHVELSWWVNGKEVHSGVSTDPQPLKEQPALNDSRYCLSSRLRVSATFWQNPRNH |
| | FRCQVQFYGLSENDEWTQDRAKPVTQIVSAEAWGRADCGFTSVSYQQGVLSATILY |
| | EILLGKATLYAVLVSALVLMAMVKRKDF |
| | [SEQ ID NO: 155] |

In certain embodiments, the extracellular domain of the TCR comprises an α chain variable region comprising a CDR1 comprising the amino acid sequence set forth in SEQ ID NO: 156 or a conservative modification thereof, a CDR2 comprising the amino acid sequence set forth in SEQ ID NO: 157 or a conservative modification thereof, and a CDR3 comprising the amino acid sequence set forth in SEQ ID NO: 158 or a conservative modification thereof. SEQ ID NOS: 156-158 are disclosed in Table 21. In certain embodiments, the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158.

In certain embodiments, the extracellular domain of the TCR comprises a β chain variable region comprising a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 161 or a conservative modification thereof. SEQ ID NOS: 159-161 are disclosed in Table 20. In certain embodiments, the β chain variable region CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161.

In certain embodiments, the α chain variable region comprises a CDR1 comprising the amino acid sequence set forth in SEQ ID NO: 156 or a conservative modification thereof, a CDR2 comprising the amino acid sequence set forth in SEQ ID NO: 157 or a conservative modification thereof, and a CDR3 comprising the amino acid sequence set forth in SEQ ID NO: 158 or a conservative modification thereof; and the β chain variable region comprises a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 161 or a conservative modification thereof. In certain embodiments, the α chain variable region comprises a CDR1 comprising the amino acid sequence set forth in SEQ ID NO: 156, a CDR2 comprising the amino acid sequence set forth in SEQ ID NO: 157, a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 162. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 162. In certain embodiments, the α chain variable region comprises the amino acid sequence set forth in SEQ ID NO: 162. SEQ ID NO: 162 is provided in Table 21.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 163. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 163. In certain embodiments, the β chain variable region comprises the amino acid sequence set forth in SEQ ID NO: 163. SEQ ID NO: 163 is provided in Table 21.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 162; and the β chain variable region comprises an amino acid sequence that is at least about 80% (e.g., at least about 85%, at least about 90%, or at least about 95%) homologous or identical to the amino acid sequence set forth in SEQ ID NO: 163. In certain embodiments, the α chain variable region comprises the amino acid sequence set forth in SEQ ID NO: 162; and the β chain variable region comprises the amino acid sequence set forth in SEQ ID NO: 163.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 164. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 164. In certain embodiments, the α chain comprises the amino acid sequence set forth in SEQ ID NO: 164.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 165. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 165. In certain embodiments, the β chain comprises the amino acid sequence set forth in SEQ ID NO: 165.

In certain embodiments, the α chain comprises an amino acid sequence that is at least about 80% (e.g., at least about 85%, at least about 90%, or at least about 95%) homologous or identical to the amino acid sequence set forth in SEQ ID NO: 164; and the β chain comprises an amino acid sequence that is at least about 80% (e.g., at least about 85%, at least about 90%, or at least about 95%) homologous or identical to the amino acid sequence set forth in SEQ ID NO: 165. In certain embodiments, the α chain comprises the amino acid sequence set forth in SEQ ID NO: 164; and the β chain comprises the amino acid sequence set forth in SEQ ID NO: 165. In certain embodiments, the TCR is designated as “TCR21”. In certain embodiments, the TCR21 binds to a RAS peptide comprising or consisting of the amino acid sequence set forth in SEQ ID NO: 3.

In certain embodiments, the CDRs sequences described above including Table 21 are delineated using the IMGT numbering system.

TABLE 21
(TCR21)

| CDRs | 1 | 2 | 3 |
|---|---|---|---|
| α-chain | NIATNDY | GYKTK | CLVGDSLANFGNEKLTF |
| | [SEQ ID NO: 156] | [SEQ ID NO: 157] | [SEQ ID NO: 158] |
| ß-chain | SNHLY | FYNNEI | CASSSRTAQDGYTF |
| | [SEQ ID NO: 159] | [SEQ ID NO: 160] | [SEQ ID NO: 161] |

| | |
|---|---|
| α-chain | MRQVARVIVELTLSTLSLAKTTQPISMDSYEGQEVNITCSHNNIATNDYITWYQQF |
| variable | PSQGPRFIIQGYKTKVTNEVASLFIPADRKSSTLSLPRVSLSDTAVYYCLVGDSLA |
| | NFGNEKLTFGTGTRLTIIP |
| | [SEQ ID NO: 162] |
| ß-chain | MDTWLVCWAIFSLLKAGLTEPEVTQTPSHQVTQMGQEVILRCVPISNHLYFYWYRQ |
| variable | ILGQKVEFLVSFYNNEISEKSEIFDDQFSVERPDGSNFTLKIRSTKLEDSAMYFCA |
| | SSSRTAQDGYTFGSGTRLTVV |
| | [SEQ ID NO: 163] |
| Full α- | MRQVARVIVELTLSTLSLAKTTQPISMDSYEGQEVNITCSHNNIATNDYITWYQQF |
| chain | PSQGPRFIIQGYKTKVTNEVASLFIPADRKSSTLSLPRVSLSDTAVYYCLVGDSLA |
| | NFGNEKLTFGTGTRLTIIPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 164] |
| Full ß- | MDTWLVCWAIFSLLKAGLTEPEVTQTPSHQVTQMGQEVILRCVPISNHLYFYWYRQ |
| chain | ILGQKVEFLVSFYNNEISEKSEIFDDQFSVERPDGSNFTLKIRSTKLEDSAMYFCA |
| | SSSRTAQDGYTFGSGTRLTVVEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 165] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 173 or a conservative modification thereof. SEQ ID NOS: 5, 6 and 173 are disclosed in Table 22. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173.

In certain embodiments, the extracellular domain of the TCR comprises a β chain variable region comprising a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 174 or a conservative modification thereof. SEQ ID NOS: 102, 103, and 174 are disclosed in Table 22. In certain embodiments, the β chain variable region CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 173 or a conservative modification thereof; and the β chain variable region comprises a CDR1 comprising the amino acid sequence set forth in SEQ ID NO: 102 or a conservative modification thereof, a CDR2 comprising the amino acid sequence set forth in SEQ ID NO: 103 or a conservative modification thereof, and a CDR3 comprising the amino acid sequence set forth in SEQ ID NO: 174 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 175. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 175. In certain embodiments, the α chain variable region comprises the amino acid sequence set forth in SEQ ID NO: 175. SEQ ID NO: 175 is provided in Table 22.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 176. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 176. In certain embodiments, the β chain variable region comprises the amino acid sequence set forth in SEQ ID NO: 176. SEQ ID NO: 176 is provided in Table 22.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 175; and the β chain variable region comprises an amino acid sequence that is at least about 80% (e.g., at least about 85%, at least about 90%, or at least about 95%) homologous or identical to the amino acid sequence set forth in SEQ ID NO: 176. In certain embodiments, the α chain variable region comprises the amino acid sequence set forth in SEQ ID NO: 175; and the β chain variable region comprises the amino acid sequence set forth in SEQ ID NO: 176.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 177. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 177. In certain embodiments, the α chain comprises the amino acid sequence set forth in SEQ ID NO: 177.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 178. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 178. In certain embodiments, the β chain comprises the amino acid sequence set forth in SEQ ID NO: 178.

In certain embodiments, the α chain comprises an amino acid sequence that is at least about 80% (e.g., at least about 85%, at least about 90%, or at least about 95%) homologous or identical to the amino acid sequence set forth in SEQ ID NO: 177; and the β chain comprises an amino acid sequence that is at least about 80% (e.g., at least about 85%, at least about 90%, or at least about 95%) homologous or identical to the amino acid sequence set forth in SEQ ID NO: 178. In certain embodiments, the α chain comprises the amino acid sequence set forth in SEQ ID NO: 177; and the β chain comprises the amino acid sequence set forth in SEQ ID NO: 178. In certain embodiments, the TCR is designated as “TCR22”. In certain embodiments, the TCR22 binds to a RAS peptide comprising or consisting of the amino acid sequence set forth in SEQ ID NO: 1.

In certain embodiments, the CDRs sequences described above including Table 22 are delineated using the IMGT numbering system.

TABLE 22
(TCR22)

| CDRs | 1 | 2 | 3 |
|---|---|---|---|
| α-chain | SIFNT | LYKAGEL | AGHSGDNYGQNFV |
| | [SEQ ID NO: 5] | [SEQ ID NO: 6] | [SEQ ID NO: 173] |
| ß-chain | MGHRA | YSYEKL | ASSPDRLTYNEQF |
| | [SEQ ID NO: 102] | [SEQ ID NO: 103] | [SEQ ID NO: 174] |

| | |
|---|---|
| α-chain | MLLEHLLIILWMQLTWVSGQQLNQSPQSMFIQEGEDVSMNCTSSSIFNTWLWYKQD |
| variable | PGEGPVLLIALYKAGELTSNGRLTAQFGITRKDSFLNISASIPSDVGIYFCAGHSG |
| | DNYGQNFVFGPGTRLSVLP |
| | [SEQ ID NO: 175] |
| ß-chain | MGCRLLCCAVLCLLGAVPIDTEVTQTPKHLVMGMTNKKSLKCEQHMGHRAMYWYKQ |
| variable | KAKKPPELMFVYSYEKLSINESVPSRFSPECPNSSLLNLHLHALQPEDSALYLCAS |
| | SPDRLTYNEQFFGPGTRLTVL |
| | [SEQ ID NO: 176] |
| Full α- | MLLEHLLIILWMQLTWVSGQQLNQSPQSMFIQEGEDVSMNCTSSSIFNTWLWYKQD |
| chain | PGEGPVLLIALYKAGELTSNGRLTAQFGITRKDSELNISASIPSDVGIYFCAGHSG |
| | DNYGQNFVFGPGTRLSVLPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 177] |
| Full ß- | MGCRLLCCAVLCLLGAVPIDTEVTQTPKHLVMGMTNKKSLKCEQHMGHRAMYWYKQ |
| chain | KAKKPPELMFVYSYEKLSINESVPSRFSPECPNSSLLNLHLHALQPEDSALYLCAS |
| | SPDRLTYNEQFFGPGTRLTVLEDLKNVEPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 178] |

In certain embodiments, the extracellular domain of the TCR comprises an α chain variable region comprising a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 181 or a conservative modification thereof. SEQ ID NOS: 179-181 are disclosed in Table 23. In certain embodiments, the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181.

In certain embodiments, the extracellular domain of the TCR comprises a β chain variable region comprising a CDR1 comprising the amino acid sequence set forth in SEQ ID NO: 182 or a conservative modification thereof, a CDR2 comprising the amino acid sequence set forth in SEQ ID NO: 183 or a conservative modification thereof, and a CDR3 comprising the amino acid sequence set forth in SEQ ID NO: 184 or a conservative modification thereof. SEQ ID NOS: 182-184 are disclosed in Table 23. In certain embodiments, the β chain variable region CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184.

In certain embodiments, the α chain variable region comprises a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 181 or a conservative modification thereof; and the β chain variable region comprises a CDR1 comprising the amino acid sequence set forth in SEQ ID NO: 182 or a conservative modification thereof, a CDR2 comprising the amino acid sequence set forth in SEQ ID NO: 183 or a conservative modification thereof, and a CDR3 comprising the amino acid sequence set forth in SEQ ID NO: 184 or a conservative modification thereof. In certain embodiments, the α chain variable region comprises a CDR1 comprising the amino acid sequence set forth in SEQ ID NO: 179, a CDR2 comprising the amino acid sequence set forth in SEQ ID NO: 180, a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 185. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 185. In certain embodiments, the α chain variable region comprises the amino acid sequence set forth in SEQ ID NO: 185. SEQ ID NO: 185 is provided in Table 23.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 186. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 186. In certain embodiments, the β chain variable region comprises the amino acid sequence set forth in SEQ ID NO: 186. SEQ ID NO: 186 is provided in Table 23.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 185; and the β chain variable region comprises an amino acid sequence that is at least about 80% (e.g., at least about 85%, at least about 90%, or at least about 95%) homologous or identical to the amino acid sequence set forth in SEQ ID NO: 186. In certain embodiments, the α chain variable region comprises the amino acid sequence set forth in SEQ ID NO: 185; and the β chain variable region comprises the amino acid sequence set forth in SEQ ID NO: 186.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 187. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 187. In certain embodiments, the α chain comprises the amino acid sequence set forth in SEQ ID NO: 187.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 188. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 188. In certain embodiments, the β chain comprises the amino acid sequence set forth in SEQ ID NO: 188.

In certain embodiments, the α chain comprises an amino acid sequence that is at least about 80% (e.g., at least about 85%, at least about 90%, or at least about 95%) homologous or identical to the amino acid sequence set forth in SEQ ID NO: 187; and the β chain comprises an amino acid sequence that is at least about 80% (e.g., at least about 85%, at least about 90%, or at least about 95%) homologous or identical to the amino acid sequence set forth in SEQ ID NO: 188. In certain embodiments, the α chain comprises the amino acid sequence set forth in SEQ ID NO: 187; and the β chain comprises the amino acid sequence set forth in SEQ ID NO: 188. In certain embodiments, the TCR is designated as “TCR23”. In certain embodiments, the TCR23 binds to a RAS peptide comprising or consisting of the amino acid sequence set forth in SEQ ID NO: 1.

In certain embodiments, the CDRs sequences described above including Table 23 are delineated using the IMGT numbering system.

TABLE 23
(TCR23)

| CDRs | 1 | 2 | 3 |
|---|---|---|---|
| α-chain | DRGSQS | IYSNGD | AVNRGFGNVLH |
| | [SEQ ID NO: 179] | [SEQ ID NO: 180] | [SEQ ID NO: 181] |
| ß-chain | MNHEY | SMNVEV | ASSLGGILVTEAF |
| | [SEQ ID NO: 182] | [SEQ ID NO: 183] | [SEQ ID NO: 184] |

| | |
|---|---|
| α-chain | MKSLRVLLVILWLQLSWVWSQQKEVEQNSGPLSVPEGAIASLNCTYSDRGSQSFFW |
| variable | YRQYSGKSPELIMFIYSNGDKEDGRETAQLNKASQYVSLLIRDSQPSDSATYLCAV |
| | NRGFGNVLHCGSGTQVIVLP |
| | [SEQ ID NO: 185] |
| ß-chain | MGPQLLGYVVLCLLGAGPLEAQVTQNPRYLITVTGKKLTVTCSQNMNHEYMSWYRQ |
| variable | DPGLGLRQIYYSMNVEVTDKGDVPEGYKVSRKEKRNFPLILESPSPNQTSLYFCAS |
| | SLGGILVTEAFFGQGTRLTVV |
| | [SEQ ID NO: 186] |
| Full α- | MKSLRVLLVILWLQLSWVWSQQKEVEQNSGPLSVPEGAIASLNCTYSDRGSQSFFW |
| chain | YRQYSGKSPELIMFIYSNGDKEDGRFTAQLNKASQYVSLLIRDSQPSDSATYLCAV |
| | NRGFGNVLHCGSGTQVIVLPNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 187] |
| Full ß- | MGPQLLGYVVLCLLGAGPLEAQVTQNPRYLITVTGKKLTVTCSQNMNHEYMSWYRQ |
| chain | DPGLGLRQIYYSMNVEVTDKGDVPEGYKVSRKEKRNFPLILESPSPNQTSLYFCAS |
| | SLGGILVTEAFFGQGTRLTVVEDLNKVEPPEVAVFEPSEAEISHTQKATLVCLATG |
| | FFPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSVSYQQGVLSATI |
| | LYEILLGKATLYAVLVSALVLMAMVKRKDF |
| | [SEQ ID NO: 188] |

In certain embodiments, the extracellular domain of the TCR comprises an α chain variable region comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 189 or a conservative modification thereof. SEQ ID NOS: 119, 120, 189 are disclosed in Table 24. In certain embodiments, the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 190 or a conservative modification thereof. SEQ ID NOS: 37, 38, and 190 are disclosed in Table 24. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190.

In certain embodiments, the α chain variable region comprises a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 189 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 190 or a conservative modification thereof. In certain embodiments, the α chain variable region comprises a CDR1 comprising the amino acid sequence set forth in SEQ ID NO: 119, a CDR2 comprising the amino acid sequence set forth in SEQ ID NO: 120, a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 191. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 191. In certain embodiments, the α chain variable region comprises the amino acid sequence set forth in SEQ ID NO: 191. SEQ ID NO: 191 is provided in Table 24.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 192. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 192. In certain embodiments, the β chain variable region comprises the amino acid sequence set forth in SEQ ID NO: 192. SEQ ID NO: 192 is provided in Table 24.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 191; and the β chain variable region comprises an amino acid sequence that is at least about 80% (e.g., at least about 85%, at least about 90%, or at least about 95%) homologous or identical to the amino acid sequence set forth in SEQ ID NO: 192. In certain embodiments, the α chain variable region comprises the amino acid sequence set forth in SEQ ID NO: 191; and the β chain variable region comprises the amino acid sequence set forth in SEQ ID NO: 192.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 193. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 193. In certain embodiments, the α chain comprises the amino acid sequence set forth in SEQ ID NO: 193.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 194. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 194. In certain embodiments, the β chain comprises the amino acid sequence set forth in SEQ ID NO: 194.

In certain embodiments, the α chain comprises an amino acid sequence that is at least about 80% (e.g., at least about 85%, at least about 90%, or at least about 95%) homologous or identical to the amino acid sequence set forth in SEQ ID NO: 193; and the β chain comprises an amino acid sequence that is at least about 80% (e.g., at least about 85%, at least about 90%, or at least about 95%) homologous or identical to the amino acid sequence set forth in SEQ ID NO: 194. In certain embodiments, the α chain comprises the amino acid sequence set forth in SEQ ID NO: 193; and the β chain comprises the amino acid sequence set forth in SEQ ID NO: 194. In certain embodiments, the TCR is designated as “TCR24”. In certain embodiments, the TCR24 binds to a RAS peptide comprising or consisting of the amino acid sequence set forth in SEQ ID NO: 1.

In certain embodiments, the CDRs sequences described above including Table 24 are delineated using the IMGT numbering system.

TABLE 24
(TCR24)

| CDRs | 1 | 2 | 3 |
|---|---|---|---|
| α-chain | TSINN | IRSNERE | ATEPDSNYQLI |
| | [SEQ ID NO: 119] | [SEQ ID NO: 120] | [SEQ ID NO: 189] |
| ß-chain | SGDLS | YYNGEE | ASSGDRGKLF |
| | [SEQ ID NO: 37] | [SEQ ID NO: 38] | [SEQ ID NO: 190] |

| | |
|---|---|
| α-chain | METLLGVSLVILWLQLARVNSQQGEEDPQALSIQEGENATMNCSYKTSINNLQWYR |
| variable | QNSGRGLVHLILIRSNEREKHSGRLRVTLDTSKKSSSLLITASRAADTASYFCATE |
| | PDSNYQLIWGAGTKLIIKP |
| | [SEQ ID NO: 191] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SGDRGKLFFGSGTQLSVL |
| | [SEQ ID NO: 192] |
| Full α- | METLLGVSLVILWLQLARVNSQQGEEDPQALSIQEGENATMNCSYKTSINNLQWYR |
| chain | QNSGRGLVHLILIRSNEREKHSGRLRVTLDTSKKSSSLLITASRAADTASYFCATE |
| | PDSNYQLIWGAGTKLIIKPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 193] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SGDRGKLFFGSGTQLSVLEDLNKVEPPEVAVFEPSEAEISHTQKATLVCLATGFFP |
| | DHVELSWWVNGKEVHSGVSTDPQPLKEQPALNDSRYCLSSRLRVSATFWQNPRNHF |
| | RCQVQFYGLSENDEWTQDRAKPVTQIVSAEAWGRADCGFTSVSYQQGVLSATILYE |
| | ILLGKATLYAVLVSALVLMAMVKRKDF |
| | [SEQ ID NO: 194] |

In certain embodiments, the extracellular domain of the TCR comprises an α chain variable region comprising a CDR1 comprising the amino acid sequence set forth in SEQ ID NO: 195 or a conservative modification thereof, a CDR2 comprising the amino acid sequence set forth in SEQ ID NO: 196 or a conservative modification thereof, and a CDR3 comprising the amino acid sequence set forth in SEQ ID NO: 197 or a conservative modification thereof. SEQ ID NOS: 195-197 are disclosed in Table 25. In certain embodiments, the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197.

In certain embodiments, the extracellular domain of the TCR comprises a β chain variable region comprising a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 198 or a conservative modification thereof. SEQ ID NOS: 159, 160, and 198 are disclosed in Table 25. In certain embodiments, the β chain variable region CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198.

In certain embodiments, the α chain variable region comprises a CDR1 comprising the amino acid sequence set forth in SEQ ID NO: 195 or a conservative modification thereof, a CDR2 comprising the amino acid sequence set forth in SEQ ID NO: 196 or a conservative modification thereof, and a CDR3 comprising the amino acid sequence set forth in SEQ ID NO: 197 or a conservative modification thereof; and the β chain variable region comprises a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 198 or a conservative modification thereof. In certain embodiments, the α chain variable region comprises a CDR1 comprising the amino acid sequence set forth in SEQ ID NO: 195, a CDR2 comprising the amino acid sequence set forth in SEQ ID NO: 196, a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 199. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 199. In certain embodiments, the α chain variable region comprises the amino acid sequence set forth in SEQ ID NO: 199. SEQ ID NO: 199 is provided in Table 25.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 200. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 200. In certain embodiments, the β chain variable region comprises the amino acid sequence set forth in SEQ ID NO: 200. SEQ ID NO: 200 is provided in Table 25.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 199; and the β chain variable region comprises an amino acid sequence that is at least about 80% (e.g., at least about 85%, at least about 90%, or at least about 95%) homologous or identical to the amino acid sequence set forth in SEQ ID NO: 200. In certain embodiments, the α chain variable region comprises the amino acid sequence set forth in SEQ ID NO: 199; and the β chain variable region comprises the amino acid sequence set forth in SEQ ID NO: 200.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 201. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 201. In certain embodiments, the α chain comprises the amino acid sequence set forth in SEQ ID NO: 201.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 202. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 202. In certain embodiments, the β chain comprises the amino acid sequence set forth in SEQ ID NO: 202.

In certain embodiments, the α chain comprises an amino acid sequence that is at least about 80% (e.g., at least about 85%, at least about 90%, or at least about 95%) homologous or identical to the amino acid sequence set forth in SEQ ID NO: 201; and the β chain comprises an amino acid sequence that is at least about 80% (e.g., at least about 85%, at least about 90%, or at least about 95%) homologous or identical to the amino acid sequence set forth in SEQ ID NO: 202. In certain embodiments, the α chain comprises the amino acid sequence set forth in SEQ ID NO: 201; and the β chain comprises the amino acid sequence set forth in SEQ ID NO: 202. In certain embodiments, the TCR is designated as “TCR25”. In certain embodiments, the TCR25 binds to a RAS peptide comprising or consisting of the amino acid sequence set forth in SEQ ID NO: 1. In certain embodiments, the TCR25 binds to a RAS peptide comprising or consisting of the amino acid sequence set forth in SEQ ID NO: 2. In certain embodiments, the TCR25 binds to a RAS peptide comprising or consisting of the amino acid sequence set forth in SEQ ID NO: 3.

In certain embodiments, the CDRs sequences described above including Table 25 are delineated using the IMGT numbering system.

TABLE 25
(TCR25)

| CDRs | 1 | 2 | 3 |
|---|---|---|---|
| α-chain | TTLSN | LVKSGEV | GNQAGTALI |
| | [SEQ ID NO: 195] | [SEQ ID NO: 196] | [SEQ ID NO: 197] |
| ß-chain | SNHLY | FYNNEI | ASSETFRDSIDGYT |
| | [SEQ ID NO: 159] | [SEQ ID NO: 160] | [SEQ ID NO: 198] |

| | |
|---|---|
| α-chain | MLLITSMLVLWMQLSQVNGQQVMQIPQYQHVQEGEDFTTYCNSSTTLSNIQWYKQR |
| variable | PGGHPVFLIQLVKSGEVKKQKRLTFQFGEAKKNSSLHITATQTTDVGTYFCGNQAG |
| | TALIFGKGTTLSVSS |
| | [SEQ ID NO: 199] |
| ß-chain | MDTWLVCWAIFSLLKAGLTEPEVTQTPSHQVTQMGQEVILRCVPISNHLYFYWYRQ |
| variable | ILGQKVEFLVSFYNNEISEKSEIFDDQFSVERPDGSNFTLKIRSTKLEDSAMYFCA |
| | SSETFRDSIDGYTFGSGTRLTVV |
| | [SEQ ID NO: 200] |
| Full α- | MLLITSMLVLWMQLSQVNGQQVMQIPQYQHVQEGEDFTTYCNSSTTLSNIQWYKQR |
| chain | PGGHPVFLIQLVKSGEVKKQKRLTFQFGEAKKNSSLHITATQTTDVGTYFCGNQAG |
| | TALIFGKGTTLSVSSNIQNPDPAVYQLRDSKSSDKSVCLFTDFDSQTNVSQSKDSD |
| | VYITDKTVLDMRSMDFKSNSAVAWSNKSDFACANAFNNSIIPEDTFFPSPESSCDV |
| | KLVEKSFETDTNLNFQNLSVIGFRILLLKVAGFNLLMTLRLWSS |
| | [SEQ ID NO: 201] |
| Full ß- | MDTWLVCWAIFSLLKAGLTEPEVTQTPSHQVTQMGQEVILRCVPISNHLYFYWYRQ |
| chain | ILGQKVEFLVSFYNNEISEKSEIFDDQFSVERPDGSNFTLKIRSTKLEDSAMYFCA |
| | SSETFRDSIDGYTFGSGTRLTVVEDLNKVEPPEVAVFEPSEAEISHTQKATLVCLA |
| | TGFFPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSVSYQQGVLSA |
| | TILYEILLGKATLYAVLVSALVLMAMVKRKDF |
| | [SEQ ID NO: 202] |

In certain embodiments, the extracellular domain of the TCR comprises an α chain variable region comprising a CDR1 comprising the amino acid sequence set forth in SEQ ID NO: 203 or a conservative modification thereof, a CDR2 comprising the amino acid sequence set forth in SEQ ID NO: 204 or a conservative modification thereof, and a CDR3 comprising the amino acid sequence set forth in SEQ ID NO: 205 or a conservative modification thereof. SEQ ID NOS: 203-205 are disclosed in Table 26. In certain embodiments, the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205.

In certain embodiments, the extracellular domain of the TCR comprises a β chain variable region comprising a CDR1 comprising the amino acid sequence set forth in SEQ ID NO: 122 or a conservative modification thereof, a CDR2 comprising the amino acid sequence set forth in SEQ ID NO: 113 or a conservative modification thereof, and a CDR3 comprising the amino acid sequence set forth in SEQ ID NO: 206 or a conservative modification thereof. SEQ ID NOS: 113, 122, and 206 are disclosed in Table 26. In certain embodiments, the β chain variable region CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206.

In certain embodiments, the α chain variable region comprises a CDR1 comprising the amino acid sequence set forth in SEQ ID NO: 203 or a conservative modification thereof, a CDR2 comprising the amino acid sequence set forth in SEQ ID NO: 204 or a conservative modification thereof, and a CDR3 comprising the amino acid sequence set forth in SEQ ID NO: 205 or a conservative modification thereof; and the β chain variable region comprises a CDR1 comprising the amino acid sequence set forth in SEQ ID NO: 122 or a conservative modification thereof, a CDR2 comprising the amino acid sequence set forth in SEQ ID NO: 113 or a conservative modification thereof, and a CDR3 comprising the amino acid sequence set forth in SEQ ID NO: 206 or a conservative modification thereof. In certain embodiments, the α chain variable region comprises a CDR1 comprising the amino acid sequence set forth in SEQ ID NO: 203, a CDR2 comprising the amino acid sequence set forth in SEQ ID NO: 204, a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 207. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 207. In certain embodiments, the α chain variable region comprises the amino acid sequence set forth in SEQ ID NO: 207. SEQ ID NO: 207 is provided in Table 26.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 208. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 208. In certain embodiments, the β chain variable region comprises the amino acid sequence set forth in SEQ ID NO: 208. SEQ ID NO: 208 is provided in Table 26.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 207; and the β chain variable region comprises an amino acid sequence that is at least about 80% (e.g., at least about 85%, at least about 90%, or at least about 95%) homologous or identical to the amino acid sequence set forth in SEQ ID NO: 208. In certain embodiments, the α chain variable region comprises the amino acid sequence set forth in SEQ ID NO: 207; and the β chain variable region comprises the amino acid sequence set forth in SEQ ID NO: 208.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 209. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 209. In certain embodiments, the α chain comprises the amino acid sequence set forth in SEQ ID NO: 209.

In certain embodiments, the extracellular domain of the TCR comprises a β chain that comprises a β chain variable region and a β chain constant region. In certain embodiments, the chain comprises an amino acid sequence that is at least about 80% (e.g., at least about 85%, at least about 90%, or at least about 95%) homologous or identical to the amino acid sequence set forth in SEQ ID NO: 210. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 210. In certain embodiments, the β chain comprises the amino acid sequence set forth in SEQ ID NO: 210.

In certain embodiments, the α chain comprises an amino acid sequence that is at least about 80% (e.g., at least about 85%, at least about 90%, or at least about 95%) homologous or identical to the amino acid sequence set forth in SEQ ID NO: 209; and the β chain comprises an amino acid sequence that is at least about 80% (e.g., at least about 85%, at least about 90%, or at least about 95%) homologous or identical to the amino acid sequence set forth in SEQ ID NO: 210. In certain embodiments, the α chain comprises the amino acid sequence set forth in SEQ ID NO: 209; and the β chain comprises the amino acid sequence set forth in SEQ ID NO: 210. In certain embodiments, the TCR is designated as “TCR26”. In certain embodiments, the TCR26 binds to a RAS peptide comprising or consisting of the amino acid sequence set forth in SEQ ID NO: 1.

In certain embodiments, the CDRs sequences described above including Table 26 are delineated using the IMGT numbering system.

TABLE 26
(TCR26)

| CDRs | 1 | 2 | 3 |
|---|---|---|---|
| α-chain | DSAIYN | IQSSQRE | AVMAMDSSYKLI |
| | [SEQ ID NO: 203] | [SEQ ID NO: 204] | [SEQ ID NO: 205] |
| ß-chain | SEHNR | FQNEAQ | ASAGGLDPDTQY |
| | [SEQ ID NO: 122] | [SEQ ID NO: 113] | [SEQ ID NO: 206] |

| | |
|---|---|
| α-chain | METLLGLLILWLQLQWVSSKQEVTQIPAALSVPEGENLVLNCSFTDSAIYNLQWFR |
| variable | QDPGKGLTSLLLIQSSQREQTSGRLNASLDKSSGRSTLYIAASQPGDSATYLCAVM |
| | AMDSSYKLIFGSGTRLLVRP |
| | [SEQ ID NO: 207] |
| ß-chain | MGTSLLCWMALCLLGADHADTGVSQDPRHKITKRGQNVTFRCDPISEHNRLYWYRQ |
| variable | TLGQGPEFLTYFQNEAQLEKSRLLSDRFSAERPKGSFSTLEIQRTEQGDSAMYLCA |
| | SAGGLDPDTQYFGPGTRLTVL |
| | [SEQ ID NO: 208] |
| Full α- | METLLGLLILWLQLQWVSSKQEVTQIPAALSVPEGENLVLNCSFTDSAIYNLQWFR |
| chain | QDPGKGLTSLLLIQSSQREQTSGRLNASLDKSSGRSTLYIAASQPGDSATYLCAVM |
| | AMDSSYKLIFGSGTRLLVRPNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 209] |
| Full ß- | MGTSLLCWMALCLLGADHADTGVSQDPRHKITKRGQNVTFRCDPISEHNRLYWYRQ |
| chain | TLGQGPEFLTYFQNEAQLEKSRLLSDRFSAERPKGSESTLEIQRTEQGDSAMYLCA |
| | SAGGLDPDTQYFGPGTRLTVLEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 210] |

In certain embodiments, the extracellular domain of the TCR comprises an α chain variable region comprising a CDR1 comprising the amino acid sequence set forth in SEQ ID NO: 211 or a conservative modification thereof, a CDR2 comprising the amino acid sequence set forth in SEQ ID NO: 212 or a conservative modification thereof, and a CDR3 comprising the amino acid sequence set forth in SEQ ID NO: 213 or a conservative modification thereof. SEQ ID NOS: 211-213 are disclosed in Table 27. In certain embodiments, the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213.

In certain embodiments, the extracellular domain of the TCR comprises a β chain variable region comprising a CDR1 comprising the amino acid sequence set forth in SEQ ID NO: 214 or a conservative modification thereof, a CDR2 comprising the amino acid sequence set forth in SEQ ID NO: 215 or a conservative modification thereof, and a CDR3 comprising the amino acid sequence set forth in SEQ ID NO: 216 or a conservative modification thereof. SEQ ID NOS: 214-216 are disclosed in Table 27. In certain embodiments, the β chain variable region CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216.

In certain embodiments, the α chain variable region comprises a CDR1 comprising the amino acid sequence set forth in SEQ ID NO: 211 or a conservative modification thereof, a CDR2 comprising the amino acid sequence set forth in SEQ ID NO: 212 or a conservative modification thereof, and a CDR3 comprising the amino acid sequence set forth in SEQ ID NO: 213 or a conservative modification thereof; and the β chain variable region comprises a CDR1 comprising the amino acid sequence set forth in SEQ ID NO: 214 or a conservative modification thereof, a CDR2 comprising the amino acid sequence set forth in SEQ ID NO: 215 or a conservative modification thereof, and a CDR3 comprising the amino acid sequence set forth in SEQ ID NO: 216 or a conservative modification thereof. In certain embodiments, the α chain variable region comprises a CDR1 comprising the amino acid sequence set forth in SEQ ID NO: 211, a CDR2 comprising the amino acid sequence set forth in SEQ ID NO: 212, a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 217. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 217. In certain embodiments, the α chain variable region comprises the amino acid sequence set forth in SEQ ID NO: 217. SEQ ID NO: 217 is provided in Table 27.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 218. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 218. In certain embodiments, the β chain variable region comprises the amino acid sequence set forth in SEQ ID NO: 218. SEQ ID NO: 218 is provided in Table 27.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 217; and the β chain variable region comprises an amino acid sequence that is at least about 80% (e.g., at least about 85%, at least about 90%, or at least about 95%) homologous or identical to the amino acid sequence set forth in SEQ ID NO: 218. In certain embodiments, the α chain variable region comprises the amino acid sequence set forth in SEQ ID NO: 217; and the β chain variable region comprises the amino acid sequence set forth in SEQ ID NO: 218.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 219. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 219. In certain embodiments, the α chain comprises the amino acid sequence set forth in SEQ ID NO: 219.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 220. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 220. In certain embodiments, the β chain comprises the amino acid sequence set forth in SEQ ID NO: 220.

In certain embodiments, the α chain comprises an amino acid sequence that is at least about 80% (e.g., at least about 85%, at least about 90%, or at least about 95%) homologous or identical to the amino acid sequence set forth in SEQ ID NO: 219; and the β chain comprises an amino acid sequence that is at least about 80% (e.g., at least about 85%, at least about 90%, or at least about 95%) homologous or identical to the amino acid sequence set forth in SEQ ID NO: 220. In certain embodiments, the α chain comprises the amino acid sequence set forth in SEQ ID NO: 219; and the β chain comprises the amino acid sequence set forth in SEQ ID NO: 220. In certain embodiments, the TCR is designated as “TCR27”. In certain embodiments, the TCR27 binds to a RAS peptide comprising or consisting of the amino acid sequence set forth in SEQ ID NO: 1.

In certain embodiments, the CDRs sequences described above including Table 27 are delineated using the IMGT numbering system.

TABLE 27
(TCR27)

| CDRs | 1 | 2 | 3 |
|---|---|---|---|
| α-chain | SSVSVY | YLSGSTLV | AVSSRGGNTDKLI |
| | [SEQ ID NO: 211] | [SEQ ID NO: 212] | [SEQ ID NO: 213] |
| ß-chain | GTSNPN | SVGIG | AWSRQGLGQPQH |
| | [SEQ ID NO: 214] | [SEQ ID NO: 215] | [SEQ ID NO: 216] |

| | |
|---|---|
| α-chain | MLLLLVPAFQVIFTLGGTRAQSVTQLDSQVPVFEEAPVELRCNYSSSVSVYLFWYV |
| variable | QYPNQGLQLLLKYLSGSTLVKGINGFEAEFNKSQTSFHLRKPSVHISDTAEYFCAV |
| | SSRGGNTDKLIFGTGTRLQVFP |
| | [SEQ ID NO: 217] |
| ß-chain | MLCSLLALLLGTFFGVRSQTIHQWPATLVQPVGSPLSLECTVEGTSNPNLYWYRQA |
| variable | AGRGLQLLFYSVGIGQISSEVPQNLSASRPQDRQFILSSKKLLLSDSGFYLCAWSR |
| | QGLGQPQHFGDGIRLSIL |
| | [SEQ ID NO: 218] |
| Full α- | MLLLLVPAFQVIFTLGGTRAQSVTQLDSQVPVFEEAPVELRCNYSSSVSVYLFWYV |
| chain | QYPNQGLQLLLKYLSGSTLVKGINGFEAEENKSQTSFHLRKPSVHISDTAEYFCAV |
| | SSRGGNTDKLIFGTGTRLQVFPNIQNPDPAVYQLRDSKSSDKSVCLFTDFDSQTNV |
| | SQSKDSDVYITDKTVLDMRSMDFKSNSAVAWSNKSDFACANAFNNSIIPEDTFFPS |
| | PESSCDVKLVEKSFETDTNLNFQNLSVIGFRILLLKVAGFNLLMTLRLWSS |
| | [SEQ ID NO: 219] |
| Full ß- | MLCSLLALLLGTFFGVRSQTIHQWPATLVQPVGSPLSLECTVEGTSNPNLYWYRQA |
| chain | AGRGLQLLFYSVGIGQISSEVPQNLSASRPQDRQFILSSKKLLLSDSGFYLCAWSR |
| | QGLGQPQHFGDGIRLSILEDLNKVFPPEVAVFEPSEAEISHTQKATLVCLATGFFP |
| | DHVELSWWVNGKEVHSGVSTDPQPLKEQPALNDSRYCLSSRLRVSATFWQNPRNHF |
| | RCQVQFYGLSENDEWTQDRAKPVTQIVSAEAWGRADCGFTSVSYQQGVLSATILYE |
| | ILLGKATLYAVLVSALVLMAMVKRKDF |
| | [SEQ ID NO: 220] |

In certain embodiments, the extracellular domain of the TCR comprises an α chain variable region comprising a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 221 or a conservative modification thereof. SEQ ID NOS: 179, 180, and 221 are disclosed in Table 28. In certain embodiments, the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221.

In certain embodiments, the extracellular domain of the TCR comprises a β chain variable region comprising a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 222 or a conservative modification thereof. SEQ ID NOS: 159, 160, and 222 are disclosed in Table 28. In certain embodiments, the β chain variable region CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222.

In certain embodiments, the α chain variable region comprises a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 221 or a conservative modification thereof; and the β chain variable region comprises a CDR1 comprising the amino acid sequence set forth in SEQ ID NO: 159 or a conservative modification thereof, a CDR2 comprising the amino acid sequence set forth in SEQ ID NO: 160 or a conservative modification thereof, and a CDR3 comprising the amino acid sequence set forth in SEQ ID NO: 222 or a conservative modification thereof. In certain embodiments, the α chain variable region comprises a CDR1 comprising the amino acid sequence set forth in SEQ ID NO: 179, a CDR2 comprising the amino acid sequence set forth in SEQ ID NO: 180, a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 223. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 223. In certain embodiments, the α chain variable region comprises the amino acid sequence set forth in SEQ ID NO: 223. SEQ ID NO: 223 is provided in Table 28.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 224. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 224. In certain embodiments, the β chain variable region comprises the amino acid sequence set forth in SEQ ID NO: 224. SEQ ID NO: 224 is provided in Table 28.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 223; and the β chain variable region comprises an amino acid sequence that is at least about 80% (e.g., at least about 85%, at least about 90%, or at least about 95%) homologous or identical to the amino acid sequence set forth in SEQ ID NO: 224. In certain embodiments, the α chain variable region comprises the amino acid sequence set forth in SEQ ID NO: 223; and the β chain variable region comprises the amino acid sequence set forth in SEQ ID NO: 224.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 225. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 225. In certain embodiments, the α chain comprises the amino acid sequence set forth in SEQ ID NO: 225.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 226. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 226. In certain embodiments, the β chain comprises the amino acid sequence set forth in SEQ ID NO: 226.

In certain embodiments, the α chain comprises an amino acid sequence that is at least about 80% (e.g., at least about 85%, at least about 90%, or at least about 95%) homologous or identical to the amino acid sequence set forth in SEQ ID NO: 225; and the β chain comprises an amino acid sequence that is at least about 80% (e.g., at least about 85%, at least about 90%, or at least about 95%) homologous or identical to the amino acid sequence set forth in SEQ ID NO: 226. In certain embodiments, the α chain comprises the amino acid sequence set forth in SEQ ID NO: 225; and the β chain comprises the amino acid sequence set forth in SEQ ID NO: 226. In certain embodiments, the TCR is designated as “TCR28”. In certain embodiments, the TCR28 binds to a RAS peptide comprising or consisting of the amino acid sequence set forth in SEQ ID NO: 1.

In certain embodiments, the CDRs sequences described above including Table 28 are delineated using the IMGT numbering system.

TABLE 28
(TCR28)

| CDRs | 1 | 2 | 3 |
|---|---|---|---|
| α-chain | DRGSQS | IYSNGD | AVPSGNTPLV |
| | [SEQ ID NO: 179] | [SEQ ID NO: 180] | [SEQ ID NO: 221] |
| ß-chain | SNHLY | FYNNEI | ASESDEKLF |
| | [SEQ ID NO: 159] | [SEQ ID NO: 160] | [SEQ ID NO: 222] |

| | |
|---|---|
| α-chain | MKSLRVLLVILWLQLSWVWSQQKEVEQNSGPLSVPEGAIASLNCTYSDRGSQSFFW |
| variable | YRQYSGKSPELIMSIYSNGDKEDGRFTAQLNKASQYVSLLIRDSQPSDSATYLCAV |
| | PSGNTPLVFGKGTRLSVIAN |
| | [SEQ ID NO: 223] |
| ß-chain | MDTWLVCWAIFSLLKAGLTEPEVTQTPSHQVTQMGQEVILRCVPISNHLYFYWYRQ |
| variable | ILGQKVEFLVSFYNNEISEKSEIFDDQFSVERPDGSNFTLKIRSTKLEDSAMYFCA |
| | SESDEKLFFGSGTQLSVL |
| | [SEQ ID NO: 224] |
| Full α- | MKSLRVLLVILWLQLSWVWSQQKEVEQNSGPLSVPEGAIASLNCTYSDRGSQSFFW |
| chain | YRQYSGKSPELIMSIYSNGDKEDGRFTAQLNKASQYVSLLIRDSQPSDSATYLCAV |
| | PSGNTPLVFGKGTRLSVIANNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 225] |
| Full ß- | MDTWLVCWAIFSLLKAGLTEPEVTQTPSHQVTQMGQEVILRCVPISNHLYFYWYRQ |
| chain | ILGQKVEFLVSFYNNEISEKSEIFDDQFSVERPDGSNFTLKIRSTKLEDSAMYFCA |
| | SESDEKLFFGSGTQLSVLEDLNKVEPPEVAVFEPSEAEISHTQKATLVCLATGFFP |
| | DHVELSWWVNGKEVHSGVSTDPQPLKEQPALNDSRYCLSSRLRVSATFWQNPRNHF |
| | RCQVQFYGLSENDEWTQDRAKPVTQIVSAEAWGRADCGFTSVSYQQGVLSATILYE |
| | ILLGKATLYAVLVSALVLMAMVKRKDF |
| | [SEQ ID NO: 226] |

In certain embodiments, the extracellular domain of the TCR comprises an α chain variable region comprising a CDR1 comprising the amino acid sequence set forth in SEQ ID NO: 227 or a conservative modification thereof, a CDR2 comprising the amino acid sequence set forth in SEQ ID NO: 228 or a conservative modification thereof, and a CDR3 comprising the amino acid sequence set forth in SEQ ID NO: 229 or a conservative modification thereof. SEQ ID NOS: 227-229 are disclosed 10 in Table 29. In certain embodiments, the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229.

In certain embodiments, the extracellular domain of the TCR comprises a β chain variable region comprising a CDR1 comprising the amino acid sequence set forth in SEQ ID NO: 230 or a conservative modification thereof, a CDR2 comprising the amino acid sequence set forth in SEQ ID NO: 231 or a conservative modification thereof, and a CDR3 comprising the amino acid sequence set forth in SEQ ID NO: 232 or a conservative modification thereof. SEQ ID NOS: 230-232 are disclosed in Table 29. In certain embodiments, the β chain variable region CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232.

In certain embodiments, the α chain variable region comprises a CDR1 comprising the amino acid sequence set forth in SEQ ID NO: 227 or a conservative modification thereof, a CDR2 comprising the amino acid sequence set forth in SEQ ID NO: 228 or a conservative modification thereof, and a CDR3 comprising the amino acid sequence set forth in SEQ ID NO: 229 or a conservative modification thereof; and the β chain variable region comprises a CDR1 comprising the amino acid sequence set forth in SEQ ID NO: 230 or a conservative modification thereof, a CDR2 comprising the amino acid sequence set forth in SEQ ID NO: 231 or a conservative modification thereof, and a CDR3 comprising the amino acid sequence set forth in SEQ ID NO: 232 or a conservative modification thereof. In certain embodiments, the α chain variable region comprises a CDR1 comprising the amino acid sequence set forth in SEQ ID NO: 227, a CDR2 comprising the amino acid sequence set forth in SEQ ID NO: 228, a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 233. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 233. In certain embodiments, the α chain variable region comprises the amino acid sequence set forth in SEQ ID NO: 233. SEQ ID NO: 233 is provided in Table 29.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 234. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 234. In certain embodiments, the β chain variable region comprises the amino acid sequence set forth in SEQ ID NO: 234. SEQ ID NO: 234 is provided in Table 29.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 233; and the β chain variable region comprises an amino acid sequence that is at least about 80% (e.g., at least about 85%, at least about 90%, or at least about 95%) homologous or identical to the amino acid sequence set forth in SEQ ID NO: 234. In certain embodiments, the α chain variable region comprises the amino acid sequence set forth in SEQ ID NO: 233; and the β chain variable region comprises the amino acid sequence set forth in SEQ ID NO: 234.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 235. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 235. In certain embodiments, the α chain comprises the amino acid sequence set forth in SEQ ID NO: 235.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 236. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 236. In certain embodiments, the β chain comprises the amino acid sequence set forth in SEQ ID NO: 236.

In certain embodiments, the α chain comprises an amino acid sequence that is at least about 80% (e.g., at least about 85%, at least about 90%, or at least about 95%) homologous or identical to the amino acid sequence set forth in SEQ ID NO: 235; and the β chain comprises an amino acid sequence that is at least about 80% (e.g., at least about 85%, at least about 90%, or at least about 95%) homologous or identical to the amino acid sequence set forth in SEQ ID NO: 236. In certain embodiments, the α chain comprises the amino acid sequence set forth in SEQ ID NO: 235; and the β chain comprises the amino acid sequence set forth in SEQ ID NO: 236. In certain embodiments, the TCR is designated as “TCR29”. In certain embodiments, the TCR29 binds to a RAS peptide comprising or consisting of the amino acid sequence set forth in SEQ ID NO: 1.

In certain embodiments, the CDRs sequences described above including Table 29 are delineated using the IMGT numbering system.

TABLE 29
(TCR29)

| CDRs | 1 | 2 | 3 |
|---|---|---|---|
| α-chain | NSASQS | VYSSG | VVHDHNAGNMLT |
| | [SEQ ID NO: 227] | [SEQ ID NO: 228] | [SEQ ID NO: 229] |
| ß-chain | LGHDT | YNNKEL | ASSPGVGYGYT |
| | [SEQ ID NO: 230] | [SEQ ID NO: 231] | [SEQ ID NO: 232] |

| | |
|---|---|
| α-chain | MISLRVLLVILWLQLSWVWSQRKEVEQDPGPFNVPEGATVAFNCTYSNSASQSFFW |
| variable | YRQDCRKEPKLLMSVYSSGNEDGRFTAQLNRASQYISLLIRDSKLSDSATYLCVVH |
| | DHNAGNMLTFGGGTRLMVKP |
| | [SEQ ID NO: 233] |
| ß-chain | MGCRLLCCVVFCLLQAGPLDTAVSQTPKYLVTQMGNDKSIKCEQNLGHDTMYWYKQ |
| variable | DSKKFLKIMFSYNNKELIINETVPNRFSPKSPDKAHLNLHINSLELGDSAVYFCAS |
| | SPGVGYGYTFGSGTRLTVV |
| | [SEQ ID NO: 234] |
| Full α- | MISLRVLLVILWLQLSWVWSQRKEVEQDPGPENVPEGATVAFNCTYSNSASQSFFW |
| chain | YRQDCRKEPKLLMSVYSSGNEDGRFTAQLNRASQYISLLIRDSKLSDSATYLCVVH |
| | DHNAGNMLTFGGGTRLMVKPNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 235] |
| Full ß- | MGCRLLCCVVFCLLQAGPLDTAVSQTPKYLVTQMGNDKSIKCEQNLGHDTMYWYKQ |
| chain | DSKKFLKIMFSYNNKELIINETVPNRFSPKSPDKAHLNLHINSLELGDSAVYFCAS |
| | SPGVGYGYTFGSGTRLTVVEDLNKVEPPEVAVFEPSEAEISHTQKATLVCLATGFF |
| | PDHVELSWWVNGKEVHSGVSTDPQPLKEQPALNDSRYCLSSRLRVSATFWQNPRNH |
| | FRCQVQFYGLSENDEWTQDRAKPVTQIVSAEAWGRADCGFTSVSYQQGVLSATILY |
| | EILLGKATLYAVLVSALVLMAMVKRKDF |
| | [SEQ ID NO: 236] |

In certain embodiments, the extracellular domain of the TCR comprises an α chain variable region comprising a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 237 or a conservative modification thereof. SEQ ID NOS: 128, 129, and 237 are disclosed in Table 30. In certain embodiments, the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237.

In certain embodiments, the extracellular domain of the TCR comprises a β chain variable region comprising a CDR1 comprising the amino acid sequence set forth in SEQ ID NO: 238 or a conservative modification thereof, a CDR2 comprising the amino acid sequence set forth in SEQ ID NO: 239 or a conservative modification thereof, and a CDR3 comprising the amino acid sequence set forth in SEQ ID NO: 240 or a conservative modification thereof. SEQ ID NOS: 238-240 are disclosed in Table 30. In certain embodiments, the β chain variable region CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240.

In certain embodiments, the α chain variable region comprises a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 237 or a conservative modification thereof; and the β chain variable region comprises a CDR1 comprising the amino acid sequence set forth in SEQ ID NO: 238 or a conservative modification thereof, a CDR2 comprising the amino acid sequence set forth in SEQ ID NO: 239 or a conservative modification thereof, and a CDR3 comprising the amino acid sequence set forth in SEQ ID NO: 240 or a conservative modification thereof. In certain embodiments, the α chain variable region comprises a CDR1 comprising the amino acid sequence set forth in SEQ ID NO: 128, a CDR2 comprising the amino acid sequence set forth in SEQ ID NO: 129, a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 241. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 241. In certain embodiments, the α chain variable region comprises the amino acid sequence set forth in SEQ ID NO: 241. SEQ ID NO: 241 is provided in Table 30.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 242. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 242. In certain embodiments, the β chain variable region comprises the amino acid sequence set forth in SEQ ID NO: 242. SEQ ID NO: 242 is provided in Table 30.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 241; and the β chain variable region comprises an amino acid sequence that is at least about 80% (e.g., at least about 85%, at least about 90%, or at least about 95%) homologous or identical to the amino acid sequence set forth in SEQ ID NO: 242. In certain embodiments, the α chain variable region comprises the amino acid sequence set forth in SEQ ID NO: 241; and the β chain variable region comprises the amino acid sequence set forth in SEQ ID NO: 242.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 243. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 243. In certain embodiments, the α chain comprises the amino acid sequence set forth in SEQ ID NO: 243.

In certain embodiments, the extracellular domain of the TCR comprises a β chain that comprises a β chain variable region and a β chain constant region. In certain embodiments, the chain comprises an amino acid sequence that is at least about 80% (e.g., at least about 85%, at least about 90%, or at least about 95%) homologous or identical to the amino acid sequence set forth in SEQ ID NO: 244. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 244. In certain embodiments, the β chain comprises the amino acid sequence set forth in SEQ ID NO: 244.

In certain embodiments, the α chain comprises an amino acid sequence that is at least about 80% (e.g., at least about 85%, at least about 90%, or at least about 95%) homologous or identical to the amino acid sequence set forth in SEQ ID NO: 243; and the β chain comprises an amino acid sequence that is at least about 80% (e.g., at least about 85%, at least about 90%, or at least about 95%) homologous or identical to the amino acid sequence set forth in SEQ ID NO: 244. In certain embodiments, the α chain comprises the amino acid sequence set forth in SEQ ID NO: 243; and the β chain comprises the amino acid sequence set forth in SEQ ID NO: 244. In certain embodiments, the TCR is designated as “TCR30”. In certain embodiments, the TCR30 binds to a RAS peptide comprising or consisting of the amino acid sequence set forth in SEQ ID NO: 1.

In certain embodiments, the CDRs sequences described above including Table 30 are delineated using the IMGT numbering system.

TABLE 30
(TCR30)

| CDRs | 1 | 2 | 3 |
|---|---|---|---|
| α-chain | NSMFDY | ISSIKDK | AATAGNNNDMR |
| | [SEQ ID NO: 128] | [SEQ ID NO: 129] | [SEQ ID NO: 237] |
| ß-chain | SGHDT | YYEEEE | ASSLRENYGYT |
| | [SEQ ID NO: 238] | [SEQ ID NO: 239] | [SEQ ID NO: 240] |

| | |
|---|---|
| α-chain | MAMLLGASVLILWLQPDWVNSQQKNDDQQVKQNSPSLSVQEGRISILNCDYTNSMF |
| variable | DYFLWYKKYPAEGPTFLISISSIKDKNEDGRFTVELNKSAKHLSLHIVPSQPGDSA |
| | VYFCAATAGNNNDMRFGAGTRLTVKP |
| | [SEQ ID NO: 241] |
| ß-chain | MGPGLLCWALLCLLGAGLVDAGVTQSPTHLIKTRGQQVTLRCSPKSGHDTVSWYQQ |
| variable | ALGQGPQFIFQYYEEEERQRGNFPDRFSGHQFPNYSSELNVNALLLGDSALYLCAS |
| | SLRENYGYTFGSGTRLTVV |
| | [SEQ ID NO: 242] |
| Full α- | MAMLLGASVLILWLQPDWVNSQQKNDDQQVKQNSPSLSVQEGRISILNCDYTNSMF |
| chain | DYFLWYKKYPAEGPTFLISISSIKDKNEDGRFTVELNKSAKHLSLHIVPSQPGDSA |
| | VYFCAATAGNNNDMRFGAGTRLTVKPNIQNPDPAVYQLRDSKSSDKSVCLFTDFDS |
| | QTNVSQSKDSDVYITDKTVLDMRSMDFKSNSAVAWSNKSDFACANAFNNSIIPEDT |
| | FFPSPESSCDVKLVEKSFETDTNLNFQNLSVIGFRILLLKVAGFNLLMTLRLWSS |
| | [SEQ ID NO: 243] |
| Full ß- | MGPGLLCWALLCLLGAGLVDAGVTQSPTHLIKTRGQQVTLRCSPKSGHDTVSWYQQ |
| chain | ALGQGPQFIFQYYEEEERQRGNFPDRFSGHQFPNYSSELNVNALLLGDSALYLCAS |
| | SLRENYGYTFGSGTRLTVVEDLNKVFPPEVAVFEPSEAEISHTQKATLVCLATGFF |
| | PDHVELSWWVNGKEVHSGVSTDPQPLKEQPALNDSRYCLSSRLRVSATFWQNPRNH |
| | FRCQVQFYGLSENDEWTQDRAKPVTQIVSAEAWGRADCGFTSVSYQQGVLSATILY |
| | EILLGKATLYAVLVSALVLMAMVKRKDF |
| | [SEQ ID NO: 244] |

In certain embodiments, the extracellular domain of the TCR comprises an α chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 245 or a conservative modification thereof. SEQ ID NOS: 72, 73, and 245 are disclosed in Table 31. In certain embodiments, the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245.

In certain embodiments, the extracellular domain of the TCR comprises a β chain variable region comprising a CDR1 comprising the amino acid sequence set forth in SEQ ID NO: 246 or a conservative modification thereof, a CDR2 comprising the amino acid sequence set forth in SEQ ID NO: 247 or a conservative modification thereof, and a CDR3 comprising the amino acid sequence set forth in SEQ ID NO: 248 or a conservative modification thereof. SEQ ID NOS: 246-248 are disclosed in Table 31. In certain embodiments, the β chain variable region CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248.

In certain embodiments, the α chain variable region comprises a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 245 or a conservative modification thereof; and the β chain variable region comprises a CDR1 comprising the amino acid sequence set forth in SEQ ID NO: 246 or a conservative modification thereof, a CDR2 comprising the amino acid sequence set forth in SEQ ID NO: 247 or a conservative modification thereof, and a CDR3 comprising the amino acid sequence set forth in SEQ ID NO: 248 or a conservative modification thereof. In certain embodiments, the α chain variable region comprises a CDR1 comprising the amino acid sequence set forth in SEQ ID NO: 72, a CDR2 comprising the amino acid sequence set forth in SEQ ID NO: 73, a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 249. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 249. In certain embodiments, the α chain variable region comprises the amino acid sequence set forth in SEQ ID NO: 249. SEQ ID NO: 249 is provided in Table 31.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 250. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 250. In certain embodiments, the β chain variable region comprises the amino acid sequence set forth in SEQ ID NO: 250. SEQ ID NO: 250 is provided in Table 31.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 249; and the β chain variable region comprises an amino acid sequence that is at least about 80% (e.g., at least about 85%, at least about 90%, or at least about 95%) homologous or identical to the amino acid sequence set forth in SEQ ID NO: 250. In certain embodiments, the α chain variable region comprises the amino acid sequence set forth in SEQ ID NO: 249; and the β chain variable region comprises the amino acid sequence set forth in SEQ ID NO: 250.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 251. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 251. In certain embodiments, the α chain comprises the amino acid sequence set forth in SEQ ID NO: 251.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 252. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 252. In certain embodiments, the β chain comprises the amino acid sequence set forth in SEQ ID NO: 252.

In certain embodiments, the α chain comprises an amino acid sequence that is at least about 80% (e.g., at least about 85%, at least about 90%, or at least about 95%) homologous or identical to the amino acid sequence set forth in SEQ ID NO: 251; and the β chain comprises an amino acid sequence that is at least about 80% (e.g., at least about 85%, at least about 90%, or at least about 95%) homologous or identical to the amino acid sequence set forth in SEQ ID NO: 252. In certain embodiments, the α chain comprises the amino acid sequence set forth in SEQ ID NO: 251; and the β chain comprises the amino acid sequence set forth in SEQ ID NO: 252. In certain embodiments, the TCR is designated as “TCR31”. In certain embodiments, the TCR31 binds to a RAS peptide comprising or consisting of the amino acid sequence set forth in SEQ ID NO: 1.

In certain embodiments, the CDRs sequences described above including Table 31 are delineated using the IMGT numbering system.

TABLE 31
(TCR31)

| CDRs | 1 | 2 | 3 |
|---|---|---|---|
| α-chain | YGATPY | YFSGDTLV | AVDIGNKLV |
| | [SEQ ID NO: 72] | [SEQ ID NO: 73] | [SEQ ID NO: 245] |
| ß-chain | WSHSY | SAAADI | ASSEGGLAKNIQY |
| | [SEQ ID NO: 246] | [SEQ ID NO: 247] | [SEQ ID NO: 248] |

| | |
|---|---|
| α-chain | MLLELIPLLGIHFVLRTARAQSVTQPDIHITVSEGASLELRCNYSYGATPYLFWYV |
| variable | QSPGQGLQLLLKYFSGDTLVQGIKGFEAEFKRSQSSFNLRKPSVHWSDAAEYFCAV |
| | DIGNKLVFGAGTILRVKS |
| | [SEQ ID NO: 249] |
| ß-chain | MGTRLFFYVALCLLWAGHRDAGITQSPRYKITETGRQVTLMCHQTWSHSYMFWYRQ |
| variable | DLGHGLRLIYYSAAADITDKGEVPDGYVVSRSKTENFPLTLESATRSQTSVYFCAS |
| | SEGGLAKNIQYFGAGTRLSVL |
| | [SEQ ID NO: 250] |
| Full α- | MLLELIPLLGIHFVLRTARAQSVTQPDIHITVSEGASLELRCNYSYGATPYLFWYV |
| chain | QSPGQGLQLLLKYFSGDTLVQGIKGFEAEFKRSQSSFNLRKPSVHWSDAAEYFCAV |
| | DIGNKLVFGAGTILRVKSNIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESS |
| | CDVKLVEKSFETDTNLNFQNLSVIGFRILLLKVAGFNLLMTLRLWSS |
| | [SEQ ID NO: 251] |
| Full ß- | MGTRLFFYVALCLLWAGHRDAGITQSPRYKITETGRQVTLMCHQTWSHSYMFWYRQ |
| chain | DLGHGLRLIYYSAAADITDKGEVPDGYVVSRSKTENEPLTLESATRSQTSVYFCAS |
| | SEGGLAKNIQYFGAGTRLSVLEDLKNVEPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 252] |

In certain embodiments, the extracellular domain of the TCR comprises an α chain variable region comprising a CDR1 comprising the amino acid sequence set forth in SEQ ID NO: 156 or a conservative modification thereof, a CDR2 comprising the amino acid sequence set forth in SEQ ID NO: 253 or a conservative modification thereof, and a CDR3 comprising the amino acid sequence set forth in SEQ ID NO: 254 or a conservative modification thereof. SEQ ID NOS: 156, 253, 254 are disclosed in Table 32. In certain embodiments, the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 255 or a conservative modification thereof. SEQ ID NOS: 37, 38, and 255 are disclosed in Table 32. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255.

In certain embodiments, the α chain variable region comprises a CDR1 comprising the amino acid sequence set forth in SEQ ID NO: 156 or a conservative modification thereof, a CDR2 comprising the amino acid sequence set forth in SEQ ID NO: 253 or a conservative modification thereof, and a CDR3 comprising the amino acid sequence set forth in SEQ ID NO: 254 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 255 or a conservative modification thereof. In certain embodiments, the α chain variable region comprises a CDR1 comprising the amino acid sequence set forth in SEQ ID NO: 156, a CDR2 comprising the amino acid sequence set forth in SEQ ID NO: 253, a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 256. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 256. In certain embodiments, the α chain variable region comprises the amino acid sequence set forth in SEQ ID NO: 256. SEQ ID NO: 256 is provided in Table 32.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 257. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 257. In certain embodiments, the β chain variable region comprises the amino acid sequence set forth in SEQ ID NO: 257. SEQ ID NO: 257 is provided in Table 32.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 256; and the β chain variable region comprises an amino acid sequence that is at least about 80% (e.g., at least about 85%, at least about 90%, or at least about 95%) homologous or identical to the amino acid sequence set forth in SEQ ID NO: 257. In certain embodiments, the α chain variable region comprises the amino acid sequence set forth in SEQ ID NO: 256; and the β chain variable region comprises the amino acid sequence set forth in SEQ ID NO: 257.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 258. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 258. In certain embodiments, the α chain comprises the amino acid sequence set forth in SEQ ID NO: 258.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 259. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 259. In certain embodiments, the β chain comprises the amino acid sequence set forth in SEQ ID NO: 259.

In certain embodiments, the α chain comprises an amino acid sequence that is at least about 80% (e.g., at least about 85%, at least about 90%, or at least about 95%) homologous or identical to the amino acid sequence set forth in SEQ ID NO: 258; and the β chain comprises an amino acid sequence that is at least about 80% (e.g., at least about 85%, at least about 90%, or at least about 95%) homologous or identical to the amino acid sequence set forth in SEQ ID NO: 259. In certain embodiments, the α chain comprises the amino acid sequence set forth in SEQ ID NO: 258; and the β chain comprises the amino acid sequence set forth in SEQ ID NO: 259. In certain embodiments, the TCR is designated as “TCR32”. In certain embodiments, the TCR32 binds to a RAS peptide comprising or consisting of the amino acid sequence set forth in SEQ ID NO: 3.

In certain embodiments, the CDRs sequences described above including Table 32 are delineated using the IMGT numbering system.

TABLE 32
(TCR32)

| CDRs | 1 | 2 | 3 |
|---|---|---|---|
| α-chain | NIATNDY | GYKTK | LALNTGTASKLT |
| | [SEQ ID NO: 156] | [SEQ ID NO: 253] | [SEQ ID NO: 254] |
| ß-chain | SGDLS | YYNGEE | ASSASGRTDTQY |
| | [SEQ ID NO: 37] | [SEQ ID NO: 38] | [SEQ ID NO: 255] |

| | |
|---|---|
| α-chain | MRQVARVIVELTLSTLSLAKTTQPISMDSYEGQEVNITCSHNNIATNDYITWYQQF |
| variable | PSQGPRFIIQGYKTKVTNEVASLFIPADRKSSTLSLPRVSLSDTAVYYCLALNTGT |
| | ASKLTFGTGTRLQVTL |
| | [SEQ ID NO: 256] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SASGRTDTQYFGPGTRLTVL |
| | [SEQ ID NO: 257] |
| Full α- | MRQVARVIVELTLSTLSLAKTTQPISMDSYEGQEVNITCSHNNIATNDYITWYQQF |
| chain | PSQGPRFIIQGYKTKVTNEVASLFIPADRKSSTLSLPRVSLSDTAVYYCLALNTGT |
| | ASKLTFGTGTRLQVTLNIQNPDPAVYQLRDSKSSDKSVCLFTDFDSQTNVSQSKDS |
| | DVYITDKTVLDMRSMDFKSNSAVAWSNKSDFACANAFNNSIIPEDTFFPSPESSCD |
| | VKLVEKSFETDTNLNFQNLSVIGFRILLLKVAGFNLLMTLRLWSS |
| | [SEQ ID NO: 258] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SASGRTDTQYFGPGTRLTVLEDLKNVFPPKVAVFEPSEAEISHTQKATLVCLATGF |
| | YPDHVELSWWVNGKEVHSGVSTDPQPLKEQPALNDSRYCLSSRLRVSATFWQNPRN |
| | HFRCQVQFYGLSENDEWTQDRAKPVTQIVSAEAWGRADCGFTSESYQQGVLSATIL |
| | YEILLGKATLYAVLVSALVLMAMVKRKDSRG |
| | [SEQ ID NO: 259] |

In certain embodiments, the extracellular domain of the TCR comprises an α chain variable region comprising a CDR1 comprising the amino acid sequence set forth in SEQ ID NO: 260 or a conservative modification thereof, a CDR2 comprising the amino acid sequence set forth in SEQ ID NO: 261 or a conservative modification thereof, and a CDR3 comprising the amino acid sequence set forth in SEQ ID NO: 262 or a conservative modification thereof. SEQ ID NOS: 260-262 are disclosed in Table 33. In certain embodiments, the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262.

In certain embodiments, the extracellular domain of the TCR comprises a β chain variable region comprising a CDR1 comprising the amino acid sequence set forth in SEQ ID NO: 263 or a conservative modification thereof, a CDR2 comprising the amino acid sequence set forth in SEQ ID NO: 264 or a conservative modification thereof, and a CDR3 comprising the amino acid sequence set forth in SEQ ID NO: 265 or a conservative modification thereof. SEQ ID NOS: 263-265 are disclosed in Table 33. In certain embodiments, the β chain variable region CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265.

In certain embodiments, the α chain variable region comprises a CDR1 comprising the amino acid sequence set forth in SEQ ID NO: 260 or a conservative modification thereof, a CDR2 comprising the amino acid sequence set forth in SEQ ID NO: 261 or a conservative modification thereof, and a CDR3 comprising the amino acid sequence set forth in SEQ ID NO: 262 or a conservative modification thereof; and the β chain variable region comprises a CDR1 comprising the amino acid sequence set forth in SEQ ID NO: 263 or a conservative modification thereof, a CDR2 comprising the amino acid sequence set forth in SEQ ID NO: 264 or a conservative modification thereof, and a CDR3 comprising the amino acid sequence set forth in SEQ ID NO: 265 or a conservative modification thereof. In certain embodiments, the α chain variable region comprises a CDR1 comprising the amino acid sequence set forth in SEQ ID NO: 260, a CDR2 comprising the amino acid sequence set forth in SEQ ID NO: 261, a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 266. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 266. In certain embodiments, the α chain variable region comprises the amino acid sequence set forth in SEQ ID NO: 266. SEQ ID NO: 266 is provided in Table 33.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 267. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 267. In certain embodiments, the β chain variable region comprises the amino acid sequence set forth in SEQ ID NO: 267. SEQ ID NO: 256 is provided in Table 33.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 266; and the β chain variable region comprises an amino acid sequence that is at least about 80% (e.g., at least about 85%, at least about 90%, or at least about 95%) homologous or identical to the amino acid sequence set forth in SEQ ID NO: 267. In certain embodiments, the α chain variable region comprises the amino acid sequence set forth in SEQ ID NO: 266; and the β chain variable region comprises the amino acid sequence set forth in SEQ ID NO: 267.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 268. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 268. In certain embodiments, the α chain comprises the amino acid sequence set forth in SEQ ID NO: 268.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 269. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 269. In certain embodiments, the β chain comprises the amino acid sequence set forth in SEQ ID NO: 269.

In certain embodiments, the α chain comprises an amino acid sequence that is at least about 80% (e.g., at least about 85%, at least about 90%, or at least about 95%) homologous or identical to the amino acid sequence set forth in SEQ ID NO: 268; and the β chain comprises an amino acid sequence that is at least about 80% (e.g., at least about 85%, at least about 90%, or at least about 95%) homologous or identical to the amino acid sequence set forth in SEQ ID NO: 269. In certain embodiments, the α chain comprises the amino acid sequence set forth in SEQ ID NO: 268; and the β chain comprises the amino acid sequence set forth in SEQ ID NO: 269. In certain embodiments, the TCR is designated as “TCR33”. In certain embodiments, the TCR33 binds to a RAS peptide comprising or consisting of the amino acid sequence set forth in SEQ ID NO: 3.

In certain embodiments, the CDRs sequences described above including Table 33 are delineated using the IMGT numbering system.

TABLE 33
(TCR33)

| CDRs | 1 | 2 | 3 |
|---|---|---|---|
| α-chain | NTAFDY | IRPDVSE | AAGDNYGQNFV |
| | [SEQ ID NO: 260] | [SEQ ID NO: 261] | [SEQ ID NO: 262] |
| ß-chain | SGHTA | FQGNSA | ASSLMSGNSPLH |
| | [SEQ ID NO: 263] | [SEQ ID NO: 264] | [SEQ ID NO: 265] |

| | |
|---|---|
| α-chain | MDKILGASFLVLWLQLCWVSGQQKEKSDQQQVKQSPQSLIVQKGGISIINCAYENT |
| variable | AFDYFPWYQQFPGKGPALLIAIRPDVSEKKEGRFTISENKSAKQFSLHIMDSQPGD |
| | SATYFCAAGDNYGQNFVFGPGTRLSVL |
| | [SEQ ID NO: 266] |
| ß-chain | MGTRLLFWVAFCLLGADHTGAGVSQSPSNKVTEKGKDVELRCDPISGHTALYWYRQ |
| variable | SLGQGLEFLIYFQGNSAPDKSGLPSDRFSAERTGGSVSTLTIQRTQQEDSAVYLCA |
| | SSLMSGNSPLHFGNGTRLTVT |
| | [SEQ ID NO: 267] |
| Full α- | MDKILGASFLVLWLQLCWVSGQQKEKSDQQQVKQSPQSLIVQKGGISIINCAYENT |
| chain | AFDYFPWYQQFPGKGPALLIAIRPDVSEKKEGRFTISENKSAKQFSLHIMDSQPGD |
| | SATYFCAAGDNYGQNFVFGPGTRLSVLNIQNPDPAVYQLRDSKSSDKSVCLFTDFD |
| | SQTNVSQSKDSDVYITDKTVLDMRSMDFKSNSAVAWSNKSDFACANAFNNSIIPED |
| | TFFPSPESSCDVKLVEKSFETDTNLNFQNLSVIGFRILLLKVAGFNLLMTLRLWSS |
| | [SEQ ID NO: 268] |
| Full ß- | MGTRLLFWVAFCLLGADHTGAGVSQSPSNKVTEKGKDVELRCDPISGHTALYWYRQ |
| chain | SLGQGLEFLIYFQGNSAPDKSGLPSDRFSAERTGGSVSTLTIQRTQQEDSAVYLCA |
| | SSLMSGNSPLHFGNGTRLTVTEDLNKVEPPEVAVFEPSEAEISHTQKATLVCLATG |
| | FFPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSVSYQQGVLSATI |
| | LYEILLGKATLYAVLVSALVLMAMVKRKDF |
| | [SEQ ID NO: 269] |

In certain embodiments, the extracellular domain of the TCR comprises an α chain variable region comprising a CDR1 comprising the amino acid sequence set forth in SEQ ID NO: 203 or a conservative modification thereof, a CDR2 comprising the amino acid sequence set forth in SEQ ID NO: 204 or a conservative modification thereof, and a CDR3 comprising the amino acid sequence set forth in SEQ ID NO: 270 or a conservative modification thereof. SEQ ID NOS: 203, 204, and 270 are disclosed in Table 34. In certain embodiments, the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270.

In certain embodiments, the extracellular domain of the TCR comprises a β chain variable region comprising a CDR1 comprising the amino acid sequence set forth in SEQ ID NO: 271 or a conservative modification thereof, a CDR2 comprising the amino acid sequence set forth in SEQ ID NO: 272 or a conservative modification thereof, and a CDR3 comprising the amino acid sequence set forth in SEQ ID NO: 273 or a conservative modification thereof. SEQ ID NOS: 271-273 are disclosed in Table 34. In certain embodiments, the β chain variable region CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273.

In certain embodiments, the α chain variable region comprises a CDR1 comprising the amino acid sequence set forth in SEQ ID NO: 203 or a conservative modification thereof, a CDR2 comprising the amino acid sequence set forth in SEQ ID NO: 204 or a conservative modification thereof, and a CDR3 comprising the amino acid sequence set forth in SEQ ID NO: 270 or a conservative modification thereof; and the β chain variable region comprises a CDR1 comprising the amino acid sequence set forth in SEQ ID NO: 271 or a conservative modification thereof, a CDR2 comprising the amino acid sequence set forth in SEQ ID NO: 272 or a conservative modification thereof, and a CDR3 comprising the amino acid sequence set forth in SEQ ID NO: 273 or a conservative modification thereof. In certain embodiments, the α chain variable region comprises a CDR1 comprising the amino acid sequence set forth in SEQ ID NO: 203, a CDR2 comprising the amino acid sequence set forth in SEQ ID NO: 204, a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 274. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 274. In certain embodiments, the α chain variable region comprises the amino acid sequence set forth in SEQ ID NO: 274. SEQ ID NO: 274 is provided in Table 34.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 275. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 275. In certain embodiments, the β chain variable region comprises the amino acid sequence set forth in SEQ ID NO: 275. SEQ ID NO: 275 is provided in Table 34.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 274; and the β chain variable region comprises an amino acid sequence that is at least about 80% (e.g., at least about 85%, at least about 90%, or at least about 95%) homologous or identical to the amino acid sequence set forth in SEQ ID NO: 275. In certain embodiments, the α chain variable region comprises the amino acid sequence set forth in SEQ ID NO: 274; and the β chain variable region comprises the amino acid sequence set forth in SEQ ID NO: 275.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 276. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 276. In certain embodiments, the α chain comprises the amino acid sequence set forth in SEQ ID NO: 276.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 277. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 277. In certain embodiments, the β chain comprises the amino acid sequence set forth in SEQ ID NO: 277.

In certain embodiments, the α chain comprises an amino acid sequence that is at least about 80% (e.g., at least about 85%, at least about 90%, or at least about 95%) homologous or identical to the amino acid sequence set forth in SEQ ID NO: 276; and the β chain comprises an amino acid sequence that is at least about 80% (e.g., at least about 85%, at least about 90%, or at least about 95%) homologous or identical to the amino acid sequence set forth in SEQ ID NO: 277. In certain embodiments, the α chain comprises the amino acid sequence set forth in SEQ ID NO: 276; and the β chain comprises the amino acid sequence set forth in SEQ ID NO: 277. In certain embodiments, the TCR is designated as “TCR34”. In certain embodiments, the TCR34 binds to a RAS peptide comprising or consisting of the amino acid sequence set forth in SEQ ID NO: 3.

In certain embodiments, the CDRs sequences described above including Table 34 are delineated using the IMGT numbering system.

TABLE 34
(TCR34)

| CDRs | 1 | 2 | 3 |
|---|---|---|---|
| α-chain | DSAIYN | IQSSQRE | AVQTGANNLF |
| | [SEQ ID NO: 203] | [SEQ ID NO: 204] | [SEQ ID NO: 270] |
| ß-chain | KGHSH | LQKENI | ASSPQRGVSSGANVLT |
| | [SEQ ID NO: 271] | [SEQ ID NO: 272] | [SEQ ID NO: 273] |

| | |
|---|---|
| α-chain | METLLGLLILWLQLQWVSSKQEVTQIPAALSVPEGENLVLNCSFTDSAIYNLQWFR |
| variable | QDPGKGLTSLLLIQSSQREQTSGRLNASLDKSSGRSTLYIAASQPGDSATYLCAVQ |
| | TGANNLFFGTGTRLTVIP |
| | [SEQ ID NO: 274] |
| ß-chain | MDTRVLCCAVICLLGAGLSNAGVMQNPRHLVRRRGQEARLRCSPMKGHSHVYWYRQ |
| variable | LPEEGLKEMVYLQKENIIDESGMPKERFSAEFPKEGPSILRIQQVVRGDSAAYFCA |
| | SSPQRGVSSGANVLTFGAGSRLTVL |
| | [SEQ ID NO: 275] |
| Full α- | METLLGLLILWLQLQWVSSKQEVTQIPAALSVPEGENLVLNCSFTDSAIYNLQWFR |
| chain | QDPGKGLTSLLLIQSSQREQTSGRLNASLDKSSGRSTLYIAASQPGDSATYLCAVQ |
| | TGANNLFFGTGTRLTVIPNIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESS |
| | CDVKLVEKSFETDTNLNFQNLSVIGFRILLLKVAGFNLLMTLRLWSS |
| | [SEQ ID NO: 276] |
| Full ß- | MDTRVLCCAVICLLGAGLSNAGVMQNPRHLVRRRGQEARLRCSPMKGHSHVYWYRQ |
| chain | LPEEGLKEMVYLQKENIIDESGMPKERFSAEFPKEGPSILRIQQVVRGDSAAYFCA |
| | SSPQRGVSSGANVLTFGAGSRLTVLEDLKNVFPPKVAVFEPSEAEISHTQKATLVC |
| | LATGFYPDHVELSWWVNGKEVHSGVSTDPQPLKEQPALNDSRYCLSSRLRVSATFW |
| | QNPRNHFRCQVQFYGLSENDEWTQDRAKPVTQIVSAEAWGRADCGFTSESYQQGVL |
| | SATILYEILLGKATLYAVLVSALVLMAMVKRKDSRG |
| | [SEQ ID NO: 277] |

In certain embodiments, the extracellular domain of the TCR comprises an α chain variable region comprising a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 278 or a conservative modification thereof. SEQ ID NOS: 128, 129, and 278 are disclosed in Table 35. In certain embodiments, the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278.

In certain embodiments, the extracellular domain of the TCR comprises a β chain variable region comprising a CDR1 comprising the amino acid sequence set forth in SEQ ID NO: 263 or a conservative modification thereof, a CDR2 comprising the amino acid sequence set forth in SEQ ID NO: 279 or a conservative modification thereof, and a CDR3 comprising the amino acid sequence set forth in SEQ ID NO: 280 or a conservative modification thereof. SEQ ID NOS: 263, 279, and 280 are disclosed in Table 35. In certain embodiments, the β chain variable region CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280.

In certain embodiments, the α chain variable region comprises a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 278 or a conservative modification thereof; and the β chain variable region comprises a CDR1 comprising the amino acid sequence set forth in SEQ ID NO: 263 or a conservative modification thereof, a CDR2 comprising the amino acid sequence set forth in SEQ ID NO: 279 or a conservative modification thereof, and a CDR3 comprising the amino acid sequence set forth in SEQ ID NO: 280 or a conservative modification thereof. In certain embodiments, the α chain variable region comprises a CDR1 comprising the amino acid sequence set forth in SEQ ID NO: 128, a CDR2 comprising the amino acid sequence set forth in SEQ ID NO: 129, a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 281. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 281. In certain embodiments, the α chain variable region comprises the amino acid sequence set forth in SEQ ID NO: 281. SEQ ID NO: 281 is provided in Table 35.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 282. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 282. In certain embodiments, the β chain variable region comprises the amino acid sequence set forth in SEQ ID NO: 282. SEQ ID NO: 282 is provided in Table 35.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 281; and the β chain variable region comprises an amino acid sequence that is at least about 80% (e.g., at least about 85%, at least about 90%, or at least about 95%) homologous or identical to the amino acid sequence set forth in SEQ ID NO: 282. In certain embodiments, the α chain variable region comprises the amino acid sequence set forth in SEQ ID NO: 281; and the β chain variable region comprises the amino acid sequence set forth in SEQ ID NO: 282.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 283. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 283. In certain embodiments, the α chain comprises the amino acid sequence set forth in SEQ ID NO: 283.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 284. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 284. In certain embodiments, the β chain comprises the amino acid sequence set forth in SEQ ID NO: 284.

In certain embodiments, the α chain comprises an amino acid sequence that is at least about 80% (e.g., at least about 85%, at least about 90%, or at least about 95%) homologous or identical to the amino acid sequence set forth in SEQ ID NO: 283; and the β chain comprises an amino acid sequence that is at least about 80% (e.g., at least about 85%, at least about 90%, or at least about 95%) homologous or identical to the amino acid sequence set forth in SEQ ID NO: 284. In certain embodiments, the α chain comprises the amino acid sequence set forth in SEQ ID NO: 283; and the β chain comprises the amino acid sequence set forth in SEQ ID NO: 284. In certain embodiments, the TCR is designated as “TCR35”. In certain embodiments, the TCR35 binds to a RAS peptide comprising or consisting of the amino acid sequence set forth in SEQ ID NO: 3.

In certain embodiments, the CDRs sequences described above including Table 35 are delineated using the IMGT numbering system.

TABLE 35
(TCR35)

| CDRs | 1 | 2 | 3 |
|---|---|---|---|
| α-chain | NSMFDY | ISSIKDK | AASGNAGNNRKLI |
| | [SEQ ID NO: 128] | [SEQ ID NO: 129] | [SEQ ID NO: 278] |
| ß-chain | SGHTA | FQGTGA | ASSLMAGTASGANVLT |
| | [SEQ ID NO: 263] | [SEQ ID NO: 279] | [SEQ ID NO: 280] |

| | |
|---|---|
| α-chain | MAMLLGASVLILWLQPDWVNSQQKNDDQQVKQNSPSLSVQEGRISILNCDYTNSMF |
| variable | DYFLWYKKYPAEGPTFLISISSIKDKNEDGRFTVFLNKSAKHLSLHIVPSQPGDSA |
| | VYFCAASGNAGNNRKLIWGLGTSLAVNP |
| | [SEQ ID NO: 281] |
| ß-chain | MGTRLLCWAALCLLGADHTGAGVSQTPSNKVTEKGKYVELRCDPISGHTALYWYRQ |
| variable | SLGQGPEFLIYFQGTGAADDSGLPNDRFFAVRPEGSVSTLKIQRTERGDSAVYLCA |
| | SSLMAGTASGANVLTFGAGSRLTVL |
| | [SEQ ID NO: 282] |
| Full α- | MAMLLGASVLILWLQPDWVNSQQKNDDQQVKQNSPSLSVQEGRISILNCDYTNSMF |
| chain | DYFLWYKKYPAEGPTFLISISSIKDKNEDGRFTVFLNKSAKHLSLHIVPSQPGDSA |
| | VYFCAASGNAGNNRKLIWGLGTSLAVNPNIQNPDPAVYQLRDSKSSDKSVCLFTDF |
| | DSQTNVSQSKDSDVYITDKTVLDMRSMDFKSNSAVAWSNKSDFACANAFNNSIIPE |
| | DTFFPSPESSCDVKLVEKSFETDTNLNFQNLSVIGFRILLLKVAGFNLLMTLRLWS |
| | S |
| | [SEQ ID NO: 283] |
| Full ß- | MGTRLLCWAALCLLGADHTGAGVSQTPSNKVTEKGKYVELRCDPISGHTALYWYRQ |
| chain | SLGQGPEFLIYFQGTGAADDSGLPNDRFFAVRPEGSVSTLKIQRTERGDSAVYLCA |
| | SSLMAGTASGANVLTFGAGSRLTVLEDLKNVFPPKVAVFEPSEAEISHTQKATLVC |
| | LATGFYPDHVELSWWVNGKEVHSGVSTDPQPLKEQPALNDSRYCLSSRLRVSATFW |
| | QNPRNHFRCQVQFYGLSENDEWTQDRAKPVTQIVSAEAWGRADCGFTSESYQQGVL |
| | SATILYEILLGKATLYAVLVSALVLMAMVKRKDSRG |
| | [SEQ ID NO: 284] |

In certain embodiments, the extracellular domain of the TCR comprises an α chain variable region comprising a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 287 or a conservative modification thereof. SEQ ID NOS: 285-287 are disclosed in Table 36. In certain embodiments, the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287.

In certain embodiments, the extracellular domain of the TCR comprises a β chain variable region comprising a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 290 or a conservative modification thereof. SEQ ID NOS: 288-290 are disclosed in Table 36. In certain embodiments, the β chain variable region CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290.

In certain embodiments, the α chain variable region comprises a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 287 or a conservative modification thereof; and the β chain variable region comprises a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 290 or a conservative modification thereof. In certain embodiments, the α chain variable region comprises a CDR1 comprising the amino acid sequence set forth in SEQ ID NO: 285, a CDR2 comprising the amino acid sequence set forth in SEQ ID NO: 286, a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 291. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 291. In certain embodiments, the α chain variable region comprises the amino acid sequence set forth in SEQ ID NO: 291. SEQ ID NO: 291 is provided in Table 36.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 292. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 292. In certain embodiments, the β chain variable region comprises the amino acid sequence set forth in SEQ ID NO: 292. SEQ ID NO: 292 is provided in Table 36.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 291; and the β chain variable region comprises an amino acid sequence that is at least about 80% (e.g., at least about 85%, at least about 90%, or at least about 95%) homologous or identical to the amino acid sequence set forth in SEQ ID NO: 292. In certain embodiments, the α chain variable region comprises the amino acid sequence set forth in SEQ ID NO: 291; and the β chain variable region comprises the amino acid sequence set forth in SEQ ID NO: 292.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 293. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 293. In certain embodiments, the α chain comprises the amino acid sequence set forth in SEQ ID NO: 293.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 294. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 294. In certain embodiments, the β chain comprises the amino acid sequence set forth in SEQ ID NO: 294.

In certain embodiments, the α chain comprises an amino acid sequence that is at least about 80% (e.g., at least about 85%, at least about 90%, or at least about 95%) homologous or identical to the amino acid sequence set forth in SEQ ID NO: 293; and the β chain comprises an amino acid sequence that is at least about 80% (e.g., at least about 85%, at least about 90%, or at least about 95%) homologous or identical to the amino acid sequence set forth in SEQ ID NO: 294. In certain embodiments, the α chain comprises the amino acid sequence set forth in SEQ ID NO: 293; and the β chain comprises the amino acid sequence set forth in SEQ ID NO: 294. In certain embodiments, the TCR is designated as “TCR36”. In certain embodiments, the TCR36 binds to a RAS peptide comprising or consisting of the amino acid sequence set forth in SEQ ID NO: 1. In certain embodiments, the TCR36 binds to a RAS peptide comprising or consisting of the amino acid sequence set forth in SEQ ID NO: 2. In certain embodiments, the TCR36 binds to a RAS peptide comprising or consisting of the amino acid sequence set forth in SEQ ID NO: 3.

In certain embodiments, the CDRs sequences described above including Table 36 are delineated using the IMGT numbering system.

TABLE 36
(TCR36)

| CDRs | 1 | 2 | 3 |
|---|---|---|---|
| α-chain | TRDTTYY | RNSFDEQN | ALSESVNAGNMLT |
| | [SEQ ID NO: 285] | [SEQ ID NO: 286] | [SEQ ID NO: 287] |
| ß-chain | MNHNS | SASEGT | ASSQNTEAF |
| | [SEQ ID NO: 288] | [SEQ ID NO: 289] | [SEQ ID NO: 290] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSENFTITASQVVDSAVYFC |
| | ALSESVNAGNMLTFGGGTRLMVKP |
| | [SEQ ID NO: 291] |
| ß-chain | MSIGLLCCVAFSLLWASPVNAGVTQTPKFQVLKTGQSMTLQCAQDMNHNSMYWYRQ |
| variable | DPGMGLRLIYYSASEGTTDKGEVPNGYNVSRLNKREFSLRLESAAPSQTSVYFCAS |
| | SQNTEAFFGQGTRLTVV |
| | [SEQ ID NO: 292] |
| Full α- | MLTASLLRAVIASICVVSSMAQKVTQAQTEISVVEKEDVTLDCVYETRDTTYYLFW |
| chain | YKQPPSGELVFLIRRNSFDEQNEISGRYSWNFQKSTSSENFTITASQVVDSAVYFC |
| | ALSESVNAGNMLTFGGGTRLMVKPNIQNPDPAVYQLRDSKSSDKSVCLFTDFDSQT |
| | NVSQSKDSDVYITDKTVLDMRSMDFKSNSAVAWSNKSDFACANAFNNSIIPEDTFF |
| | PSPESSCDVKLVEKSFETDTNLNFQNLSVIGFRILLLKVAGFNLLMTLRLWSS |
| | [SEQ ID NO: 293] |
| Full ß- | MSIGLLCCVAFSLLWASPVNAGVTQTPKFQVLKTGQSMTLQCAQDMNHNSMYWYRQ |
| chain | DPGMGLRLIYYSASEGTTDKGEVPNGYNVSRLNKREFSLRLESAAPSQTSVYFCAS |
| | SQNTEAFFGQGTRLTVVEDLNKVFPPEVAVFEPSEAEISHTQKATLVCLATGFFPD |
| | HVELSWWVNGKEVHSGVSTDPQPLKEQPALNDSRYCLSSRLRVSATFWQNPRNHER |
| | CQVQFYGLSENDEWTQDRAKPVTQIVSAEAWGRADCGFTSVSYQQGVLSATILYEI |
| | LLGKATLYAVLVSALVLMAMVKRKDF |
| | [SEQ ID NO: 294] |

In certain embodiments, the extracellular domain of the TCR comprises an α chain variable region comprising a CDR1 comprising the amino acid sequence set forth in SEQ ID NO: 227 or a conservative modification thereof, a CDR2 comprising the amino acid sequence set forth in SEQ ID NO: 228 or a conservative modification thereof, and a CDR3 comprising the amino acid sequence set forth in SEQ ID NO: 295 or a conservative modification thereof. SEQ ID NOS: 227, 228, and 295 are disclosed in Table 37. In certain embodiments, the α chain variable region comprises a CDR1 comprising the amino acid sequence set forth in SEQ ID NO: 228, a CDR2 comprising the amino acid sequence set forth in SEQ ID NO: 229, and a CDR3 comprising the amino acid sequence set forth in SEQ ID NO: 295.

In certain embodiments, the extracellular domain of the TCR comprises a β chain variable region comprising a CDR1 comprising the amino acid sequence set forth in SEQ ID NO: 296 or a conservative modification thereof, a CDR2 comprising the amino acid sequence set forth in SEQ ID NO: 297 or a conservative modification thereof, and a CDR3 comprising the amino acid sequence set forth in SEQ ID NO: 298 or a conservative modification thereof. SEQ ID NOS: 296-298 are disclosed in Table 37. In certain embodiments, the β chain variable region CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298.

In certain embodiments, the α chain variable region comprises a CDR1 comprising the amino acid sequence set forth in SEQ ID NO: 227 or a conservative modification thereof, a CDR2 comprising the amino acid sequence set forth in SEQ ID NO: 228 or a conservative modification thereof, and a CDR3 comprising the amino acid sequence set forth in SEQ ID NO: 295 or a conservative modification thereof; and the β chain variable region comprises a CDR1 comprising the amino acid sequence set forth in SEQ ID NO: 296 or a conservative modification thereof, a CDR2 comprising the amino acid sequence set forth in SEQ ID NO: 297 or a conservative modification thereof, and a CDR3 comprising the amino acid sequence set forth in SEQ ID NO: 298 or a conservative modification thereof. In certain embodiments, the α chain variable region comprises a CDR1 comprising the amino acid sequence set forth in SEQ ID NO: 227, a CDR2 comprising the amino acid sequence set forth in SEQ ID NO: 228, a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 299. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 299. In certain embodiments, the α chain variable region comprises the amino acid sequence set forth in SEQ ID NO: 299. SEQ ID NO: 299 is provided in Table 37.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 300. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 300. In certain embodiments, the β chain variable region comprises the amino acid sequence set forth in SEQ ID NO: 300. SEQ ID NO: 300 is provided in Table 37.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 299; and the β chain variable region comprises an amino acid sequence that is at least about 80% (e.g., at least about 85%, at least about 90%, or at least about 95%) homologous or identical to the amino acid sequence set forth in SEQ ID NO: 300. In certain embodiments, the α chain variable region comprises the amino acid sequence set forth in SEQ ID NO: 299; and the β chain variable region comprises the amino acid sequence set forth in SEQ ID NO: 300.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 301. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 301. In certain embodiments, the α chain comprises the amino acid sequence set forth in SEQ ID NO: 301.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 302. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 302. In certain embodiments, the β chain comprises the amino acid sequence set forth in SEQ ID NO: 302.

In certain embodiments, the α chain comprises an amino acid sequence that is at least about 80% (e.g., at least about 85%, at least about 90%, or at least about 95%) homologous or identical to the amino acid sequence set forth in SEQ ID NO: 301; and the β chain comprises an amino acid sequence that is at least about 80% (e.g., at least about 85%, at least about 90%, or at least about 95%) homologous or identical to the amino acid sequence set forth in SEQ ID NO: 302. In certain embodiments, the α chain comprises the amino acid sequence set forth in SEQ ID NO: 301; and the β chain comprises the amino acid sequence set forth in SEQ ID NO: 302. In certain embodiments, the TCR is designated as “TCR37”. In certain embodiments, the TCR37 binds to a RAS peptide comprising or consisting of the amino acid sequence set forth in SEQ ID NO: 1. In certain embodiments, the TCR37 binds to a RAS peptide comprising or consisting of the amino acid sequence set forth in SEQ ID NO: 2. In certain embodiments, the TCR37 binds to a RAS peptide comprising or consisting of the amino acid sequence set forth in SEQ ID NO: 3.

In certain embodiments, the CDRs sequences described above including Table 37 are delineated using the IMGT numbering system.

TABLE 37
(TCR37)

| CDRs | 1 | 2 | 3 |
|---|---|---|---|
| α-chain | NSASQS | VYSSG | VVNMPSGQNFV |
| | [SEQ ID NO: 227] | [SEQ ID NO: 228] | [SEQ ID NO: 295] |
| ß-chain | ENHRY | SYGVKD | AISQDRATLAEQY |
| | [SEQ ID NO: 296] | [SEQ ID NO: 297] | [SEQ ID NO: 298] |

| | |
|---|---|
| α-chain | MISLRVLLVILWLQLSWVWSQRKEVEQDPGPENVPEGATVAFNCTYSNSASQSFFW |
| variable | YRQDCRKEPKLLMSVYSSGNEDGRFTAQLNRASQYISLLIRDSKLSDSATYLCVVN |
| | MPSGQNFVFGPGTRLSVLP |
| | [SEQ ID NO: 299] |
| ß-chain | MGTRLFFYVALCLLWTGHVDAGITQSPRHKVTETGTPVTLRCHQTENHRYMYWYRQ |
| variable | DPGHGLRLIHYSYGVKDTDKGEVSDGYSVSRSKTEDFLLTLESATSSQTSVYFCAI |
| | SQDRATLAEQYVGPGTRLTVT |
| | [SEQ ID NO: 300] |
| Full α- | MISLRVLLVILWLQLSWVWSQRKEVEQDPGPFNVPEGATVAFNCTYSNSASQSFFW |
| chain | YRQDCRKEPKLLMSVYSSGNEDGRFTAQLNRASQYISLLIRDSKLSDSATYLCVVN |
| | MPSGQNFVFGPGTRLSVLPNIQNPDPAVYQLRDSKSSDKSVCLFTDFDSQTNVSQS |
| | KDSDVYITDKTVLDMRSMDFKSNSAVAWSNKSDFACANAFNNSIIPEDTFFPSPES |
| | SCDVKLVEKSFETDTNLNFQNLSVIGFRILLLKVAGFNLLMTLRLWSS |
| | [SEQ ID NO: 301] |
| Full ß- | MGTRLFFYVALCLLWTGHVDAGITQSPRHKVTETGTPVTLRCHQTENHRYMYWYRQ |
| chain | DPGHGLRLIHYSYGVKDTDKGEVSDGYSVSRSKTEDELLTLESATSSQTSVYFCAI |
| | SQDRATLAEQYVGPGTRLTVTEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 302] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 3 or a conservative modification thereof, and a CDR3 comprising the amino acid sequence set forth in SEQ ID NO: 303 or a conservative modification thereof. SEQ ID NOS: 5, 6, and 303 are 10 disclosed in Table 38. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303.

In certain embodiments, the extracellular domain of the TCR comprises a β chain variable region comprising a CDR1 comprising the amino acid sequence set forth in SEQ ID NO: 304 or a conservative modification thereof, a CDR2 comprising the amino acid sequence set forth in SEQ ID NO: 305 or a conservative modification thereof, and a CDR3 comprising the amino acid sequence set forth in SEQ ID NO: 306 or a conservative modification thereof. SEQ ID NOS: 304-306 are disclosed in Table 38. In certain embodiments, the β chain variable region CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 303 or a conservative modification thereof; and the β chain variable region comprises a CDR1 comprising the amino acid sequence set forth in SEQ ID NO: 304 or a conservative modification thereof, a CDR2 comprising the amino acid sequence set forth in SEQ ID NO: 305 or a conservative modification thereof, and a CDR3 comprising the amino acid sequence set forth in SEQ ID NO: 306 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 307. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 307. In certain embodiments, the α chain variable region comprises the amino acid sequence set forth in SEQ ID NO: 307. SEQ ID NO: 307 is provided in Table 38.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 308. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 308. In certain embodiments, the β chain variable region comprises the amino acid sequence set forth in SEQ ID NO: 308. SEQ ID NO: 308 is provided in Table 38.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 307; and the β chain variable region comprises an amino acid sequence that is at least about 80% (e.g., at least about 85%, at least about 90%, or at least about 95%) homologous or identical to the amino acid sequence set forth in SEQ ID NO: 308. In certain embodiments, the α chain variable region comprises the amino acid sequence set forth in SEQ ID NO: 307; and the β chain variable region comprises the amino acid sequence set forth in SEQ ID NO: 308.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 309. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 309. In certain embodiments, the α chain comprises the amino acid sequence set forth in SEQ ID NO: 309.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 310. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 310. In certain embodiments, the β chain comprises the amino acid sequence set forth in SEQ ID NO: 310.

In certain embodiments, the α chain comprises an amino acid sequence that is at least about 80% (e.g., at least about 85%, at least about 90%, or at least about 95%) homologous or identical to the amino acid sequence set forth in SEQ ID NO: 309; and the β chain comprises an amino acid sequence that is at least about 80% (e.g., at least about 85%, at least about 90%, or at least about 95%) homologous or identical to the amino acid sequence set forth in SEQ ID NO: 310. In certain embodiments, the α chain comprises the amino acid sequence set forth in SEQ ID NO: 309; and the β chain comprises the amino acid sequence set forth in SEQ ID NO: 310. In certain embodiments, the TCR is designated as “TCR38”. In certain embodiments, the TCR38 binds to a RAS peptide comprising or consisting of the amino acid sequence set forth in SEQ ID NO: 2.

In certain embodiments, the CDRs sequences described above including Table 38 are delineated using the IMGT numbering system.

TABLE 38
(TCR38)

| CDRs | 1 | 2 | 3 |
|---|---|---|---|
| α-chain | SIFNT | LYKAGEL | AGQFTGGTSYGKLT |
| | [SEQ ID NO: 5] | [SEQ ID NO: 6] | [SEQ ID NO: 303] |
| ß-chain | LGHNT | ERNRAP | ATGQGLDTGELF |
| | [SEQ ID NO: 304] | [SEQ ID NO: 305] | [SEQ ID NO: 306] |

| | |
|---|---|
| α-chain | MLLEHLLIILWMQLTWVSGQQLNQSPQSMFIQEGEDVSMNCTSSSIFNTWLWYKQD |
| variable | PGEGPVLLIALYKAGELTSNGRLTAQFGITRKDSELNISASIPSDVGIYFCAGQFT |
| | GGTSYGKLTFGQGTILTVHP |
| | [SEQ ID NO: 307] |
| ß-chain | MATRLLCCVVLCLLGEELIDARVTQTPRDKVTEMGQEVTMRCQPILGHNTVFWYRQ |
| variable | TMMQGLELLAYFRNRAPLDDSGMPKDRFSAEMPDATLATLKIQPSEPRDSAVYFCA |
| | TGQGLDTGELFFGEGSRLTVL |
| | [SEQ ID NO: 308] |
| Full α- | MLLEHLLIILWMQLTWVSGQQLNQSPQSMFIQEGEDVSMNCTSSSIFNTWLWYKQD |
| chain | PGEGPVLLIALYKAGELTSNGRLTAQFGITRKDSFLNISASIPSDVGIYFCAGQFT |
| | GGTSYGKLTFGQGTILTVHPNIQNPDPAVYQLRDSKSSDKSVCLFTDFDSQTNVSQ |
| | SKDSDVYITDKTVLDMRSMDFKSNSAVAWSNKSDFACANAENNSIIPEDTFFPSPE |
| | SSCDVKLVEKSFETDTNLNFQNLSVIGFRILLLKVAGFNLLMTLRLWSS |
| | [SEQ ID NO: 309] |
| Full ß- | MATRLLCCVVLCLLGEELIDARVTQTPRDKVTEMGQEVTMRCQPILGHNTVFWYRQ |
| chain | TMMQGLELLAYERNRAPLDDSGMPKDRFSAEMPDATLATLKIQPSEPRDSAVYFCA |
| | TGQGLDTGELFFGEGSRLTVLEDLKNVEPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 310] |

In certain embodiments, the extracellular domain of the TCR comprises an α chain variable region comprising a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 311 or a conservative modification thereof. SEQ ID NOS: 128, 129, and 311 are disclosed in Table 39. In certain embodiments, the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311.

In certain embodiments, the extracellular domain of the TCR comprises a β chain variable region comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 312 or a conservative modification thereof. SEQ ID NOS: 27, 28, and 312 are disclosed in Table 39. In certain embodiments, the β chain variable region CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312.

In certain embodiments, the α chain variable region comprises a CDR1 comprising the amino acid sequence set forth in SEQ ID NO: 128 or a conservative modification thereof, a CDR2 comprising the amino acid sequence set forth in SEQ ID NO: 129 or a conservative modification thereof, and a CDR3 comprising the amino acid sequence set forth in SEQ ID NO: 311 or a conservative modification thereof; and the β chain variable region comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 312 or a conservative modification thereof. In certain embodiments, the α chain variable region comprises a CDR1 comprising the amino acid sequence set forth in SEQ ID NO: 128, a CDR2 comprising the amino acid sequence set forth in SEQ ID NO: 129, a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 313. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 313. In certain embodiments, the α chain variable region comprises the amino acid sequence set forth in SEQ ID NO: 313. SEQ ID NO: 313 is provided in Table 39.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 314. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 314. In certain embodiments, the β chain variable region comprises the amino acid sequence set forth in SEQ ID NO: 314. SEQ ID NO: 314 is provided in Table 39.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 313; and the β chain variable region comprises an amino acid sequence that is at least about 80% (e.g., at least about 85%, at least about 90%, or at least about 95%) homologous or identical to the amino acid sequence set forth in SEQ ID NO: 314. In certain embodiments, the α chain variable region comprises the amino acid sequence set forth in SEQ ID NO: 313; and the β chain variable region comprises the amino acid sequence set forth in SEQ ID NO: 314.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 315. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 315. In certain embodiments, the α chain comprises the amino acid sequence set forth in SEQ ID NO: 315.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 316. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 316. In certain embodiments, the β chain comprises the amino acid sequence set forth in SEQ ID NO: 316.

In certain embodiments, the α chain comprises an amino acid sequence that is at least about 80% (e.g., at least about 85%, at least about 90%, or at least about 95%) homologous or identical to the amino acid sequence set forth in SEQ ID NO: 315; and the β chain comprises an amino acid sequence that is at least about 80% (e.g., at least about 85%, at least about 90%, or at least about 95%) homologous or identical to the amino acid sequence set forth in SEQ ID NO: 316. In certain embodiments, the α chain comprises the amino acid sequence set forth in SEQ ID NO: 315; and the β chain comprises the amino acid sequence set forth in SEQ ID NO: 316. In certain embodiments, the TCR is designated as “TCR39”. In certain embodiments, the TCR39 binds to a RAS peptide comprising or consisting of the amino acid sequence set forth in SEQ ID NO: 2.

In certain embodiments, the CDRs sequences described above including Table 39 are delineated using the IMGT numbering system.

TABLE 39
(TCR39)

| CDRs | 1 | 2 | 3 |
|---|---|---|---|
| α-chain | NSMFDY | ISSIKDK | AAVDNNNDMR |
| | [SEQ ID NO: 128] | [SEQ ID NO: 129] | [SEQ ID NO: 311] |
| ß-chain | SGHNT | YYREEE | ASSLDRGSSGNTIY |
| | [SEQ ID NO: 27] | [SEQ ID NO: 28] | [SEQ ID NO: 312] |

| | |
|---|---|
| α-chain | MAMLLGASVLILWLQPDWVNSQQKNDDQQVKQNSPSLSVQEGRISILNCDYTNSMF |
| variable | DYFLWYKKYPAEGPTFLISISSIKDKNEDGRFTVELNKSAKHLSLHIVPSQPGDSA |
| | VYFCAAVDNNNDMRFGAGTRLTVKP |
| | [SEQ ID NO: 313] |
| ß-chain | MGPGLLCWVLLCLLGAGSVETGVTQSPTHLIKTRGQQVTLRCSSQSGHNTVSWYQQ |
| variable | ALGQGPQFIFQYYREEENGRGNFPPRFSGLQFPNYSSELNVNALELDDSALYLCAS |
| | SLDRGSSGNTIYFGEGSWLTVV |
| | [SEQ ID NO: 314] |
| Full α- | MAMLLGASVLILWLQPDWVNSQQKNDDQQVKQNSPSLSVQEGRISILNCDYTNSMF |
| chain | DYFLWYKKYPAEGPTFLISISSIKDKNEDGRFTVFLNKSAKHLSLHIVPSQPGDSA |
| | VYFCAAVDNNNDMRFGAGTRLTVKPNIQNPDPAVYQLRDSKSSDKSVCLFTDFDSQ |
| | TNVSQSKDSDVYITDKTVLDMRSMDFKSNSAVAWSNKSDFACANAFNNSIIPEDTF |
| | FPSPESSCDVKLVEKSFETDTNLNFQNLSVIGFRILLLKVAGFNLLMTLRLWSS |
| | [SEQ ID NO: 315] |
| Full ß- | MGPGLLCWVLLCLLGAGSVETGVTQSPTHLIKTRGQQVTLRCSSQSGHNTVSWYQQ |
| chain | ALGQGPQFIFQYYREEENGRGNFPPRFSGLQFPNYSSELNVNALELDDSALYLCAS |
| | SLDRGSSGNTIYFGEGSWLTVVEDLNKVEPPEVAVFEPSEAEISHTQKATLVCLAT |
| | GFFPDHVELSWWVNGKEVHSGVSTDPQPLKEQPALNDSRYCLSSRLRVSATFWQNP |
| | RNHFRCQVQFYGLSENDEWTQDRAKPVTQIVSAEAWGRADCGFTSVSYQQGVLSAT |
| | ILYEILLGKATLYAVLVSALVLMAMVKRKDF |
| | [SEQ ID NO: 316] |

In certain embodiments, the extracellular domain of the TCR comprises an α chain variable region comprising a CDR1 comprising the amino acid sequence set forth in SEQ ID NO: 317 or a conservative modification thereof, a CDR2 comprising the amino acid sequence set forth in SEQ ID NO: 318 or a conservative modification thereof, and a CDR3 comprising the amino acid sequence set forth in SEQ ID NO: 319 or a conservative modification thereof. SEQ ID NOS: 317-318 are disclosed in Table 40. In certain embodiments, the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319.

In certain embodiments, the extracellular domain of the TCR comprises a β chain variable region comprising a CDR1 comprising the amino acid sequence set forth in SEQ ID NO: 263 or a conservative modification thereof, a CDR2 comprising the amino acid sequence set forth in SEQ ID NO: 279 or a conservative modification thereof, and a CDR3 comprising the amino acid sequence set forth in SEQ ID NO: 320 or a conservative modification thereof. SEQ ID NOS: 263, 279, 320 are disclosed in Table 40. In certain embodiments, the β chain variable region CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320.

In certain embodiments, the α chain variable region comprises a CDR1 comprising the amino acid sequence set forth in SEQ ID NO: 317 or a conservative modification thereof, a CDR2 comprising the amino acid sequence set forth in SEQ ID NO: 318 or a conservative modification thereof, and a CDR3 comprising the amino acid sequence set forth in SEQ ID NO: 319 or a conservative modification thereof; and the β chain variable region comprises a CDR1 comprising the amino acid sequence set forth in SEQ ID NO: 263 or a conservative modification thereof, a CDR2 comprising the amino acid sequence set forth in SEQ ID NO: 279 or a conservative modification thereof, and a CDR3 comprising the amino acid sequence set forth in SEQ ID NO: 320 or a conservative modification thereof. In certain embodiments, the α chain variable region comprises a CDR1 comprising the amino acid sequence set forth in SEQ ID NO: 317, a CDR2 comprising the amino acid sequence set forth in SEQ ID NO: 318, a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 321. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 321. In certain embodiments, the α chain variable region comprises the amino acid sequence set forth in SEQ ID NO: 321. SEQ ID NO: 321 is provided in Table 40.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 322. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 322. In certain embodiments, the β chain variable region comprises the amino acid sequence set forth in SEQ ID NO: 322. SEQ ID NO: 322 is provided in Table 40.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 321; and the β chain variable region comprises an amino acid sequence that is at least about 80% (e.g., at least about 85%, at least about 90%, or at least about 95%) homologous or identical to the amino acid sequence set forth in SEQ ID NO: 322. In certain embodiments, the α chain variable region comprises the amino acid sequence set forth in SEQ ID NO: 321; and the β chain variable region comprises the amino acid sequence set forth in SEQ ID NO: 322.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 323. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 323. In certain embodiments, the α chain comprises the amino acid sequence set forth in SEQ ID NO: 323.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 324. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 324. In certain embodiments, the β chain comprises the amino acid sequence set forth in SEQ ID NO: 324.

In certain embodiments, the α chain comprises an amino acid sequence that is at least about 80% (e.g., at least about 85%, at least about 90%, or at least about 95%) homologous or identical to the amino acid sequence set forth in SEQ ID NO: 323; and the β chain comprises an amino acid sequence that is at least about 80% (e.g., at least about 85%, at least about 90%, or at least about 95%) homologous or identical to the amino acid sequence set forth in SEQ ID NO: 324. In certain embodiments, the α chain comprises the amino acid sequence set forth in SEQ ID NO: 323; and the β chain comprises the amino acid sequence set forth in SEQ ID NO: 324. In certain embodiments, the TCR is designated as “TCR40”. In certain embodiments, the TCR40 binds to a RAS peptide comprising or consisting of the amino acid sequence set forth in SEQ ID NO: 2.

In certain embodiments, the CDRs sequences described above including Table 40 are delineated using the IMGT numbering system.

TABLE 40
(TCR40)

| CDRs | 1 | 2 | 3 |
|---|---|---|---|
| α-chain | TSGFNG | NVLDGL | AASFSGGYNKLI |
| | [SEQ ID NO: 317] | [SEQ ID NO: 318] | [SEQ ID NO: 319] |
| ß-chain | SGHTA | FQGTGA | ASSLMRGDTQY |
| | [SEQ ID NO: 263] | [SEQ ID NO: 279] | [SEQ ID NO: 320] |

| | |
|---|---|
| α-chain | MWGVFLLYVSMKMGGTTGQNIDQPTEMTATEGAIVQINCTYQTSGFNGLFWYQQHA |
| variable | GEAPTFLSYNVLDGLEEKGRFSSFLSRSKGYSYLLLKELQMKDSASYLCAASFSGG |
| | YNKLIFGAGTRLAVHP |
| | [SEQ ID NO: 321] |
| ß-chain | MGTRLLCWAALCLLGADHTGAGVSQTPSNKVTEKGKYVELRCDPISGHTALYWYRQ |
| variable | SLGQGPEFLIYFQGTGAADDSGLPNDRFFAVRPEGSVSTLKIQRTERGDSAVYLCA |
| | SSLMRGDTQYFGPGTRLTVL |
| | [SEQ ID NO: 322] |
| Full α- | MWGVFLLYVSMKMGGTTGQNIDQPTEMTATEGAIVQINCTYQTSGFNGLFWYQQHA |
| chain | GEAPTFLSYNVLDGLEEKGRFSSFLSRSKGYSYLLLKELQMKDSASYLCAASFSGG |
| | YNKLIFGAGTRLAVHPNIQNPDPAVYQLRDSKSSDKSVCLFTDFDSQTNVSQSKDS |
| | DVYITDKTVLDMRSMDFKSNSAVAWSNKSDFACANAFNNSIIPEDTFFPSPESSCD |
| | VKLVEKSFETDTNLNFQNLSVIGFRILLLKVAGFNLLMTLRLWSS |
| | [SEQ ID NO: 323] |
| Full ß- | MGTRLLCWAALCLLGADHTGAGVSQTPSNKVTEKGKYVELRCDPISGHTALYWYRQ |
| chain | SLGQGPEFLIYFQGTGAADDSGLPNDRFFAVRPEGSVSTLKIQRTERGDSAVYLCA |
| | SSLMRGDTQYFGPGTRLTVLEDLKNVEPPKVAVFEPSEAEISHTQKATLVCLATGF |
| | YPDHVELSWWVNGKEVHSGVSTDPQPLKEQPALNDSRYCLSSRLRVSATFWQNPRN |
| | HFRCQVQFYGLSENDEWTQDRAKPVTQIVSAEAWGRADCGFTSESYQQGVLSATIL |
| | YEILLGKATLYAVLVSALVLMAMVKRKDSRG |
| | [SEQ ID NO: 324] |

In certain embodiments, the extracellular domain of the TCR comprises an α chain variable region comprising a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 325 or a conservative modification thereof. SEQ ID NOS: 285, 286, and 325 are disclosed in Table 41. In certain embodiments, the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325.

In certain embodiments, the extracellular domain of the TCR comprises a β chain variable region comprising a CDR1 comprising the amino acid sequence set forth in SEQ ID NO: 326 or a conservative modification thereof, a CDR2 comprising the amino acid sequence set forth in SEQ ID NO: 327 or a conservative modification thereof, and a CDR3 comprising the amino acid sequence set forth in SEQ ID NO: 328 or a conservative modification thereof. SEQ ID NOS: 326-328 are disclosed in Table 41. In certain embodiments, the β chain variable region CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328.

In certain embodiments, the α chain variable region comprises a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 325 or a conservative modification thereof; and the β chain variable region comprises a CDR1 comprising the amino acid sequence set forth in SEQ ID NO: 326 or a conservative modification thereof, a CDR2 comprising the amino acid sequence set forth in SEQ ID NO: 327 or a conservative modification thereof, and a CDR3 comprising the amino acid sequence set forth in SEQ ID NO: 328 or a conservative modification thereof. In certain embodiments, the α chain variable region comprises a CDR1 comprising the amino acid sequence set forth in SEQ ID NO: 285, a CDR2 comprising the amino acid sequence set forth in SEQ ID NO: 286, a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 329. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 329. In certain embodiments, the α chain variable region comprises the amino acid sequence set forth in SEQ ID NO: 329. SEQ ID NO: 329 is provided in Table 41.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 330. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 330. In certain embodiments, the β chain variable region comprises the amino acid sequence set forth in SEQ ID NO: 330. SEQ ID NO: 330 is provided in Table 41.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 329; and the β chain variable region comprises an amino acid sequence that is at least about 80% (e.g., at least about 85%, at least about 90%, or at least about 95%) homologous or identical to the amino acid sequence set forth in SEQ ID NO: 330. In certain embodiments, the α chain variable region comprises the amino acid sequence set forth in SEQ ID NO: 329; and the β chain variable region comprises the amino acid sequence set forth in SEQ ID NO: 330.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 331. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 331. In certain embodiments, the α chain comprises the amino acid sequence set forth in SEQ ID NO: 331.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 332. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 332. In certain embodiments, the β chain comprises the amino acid sequence set forth in SEQ ID NO: 332.

In certain embodiments, the α chain comprises an amino acid sequence that is at least about 80% (e.g., at least about 85%, at least about 90%, or at least about 95%) homologous or identical to the amino acid sequence set forth in SEQ ID NO: 331; and the β chain comprises an amino acid sequence that is at least about 80% (e.g., at least about 85%, at least about 90%, or at least about 95%) homologous or identical to the amino acid sequence set forth in SEQ ID NO: 332. In certain embodiments, the α chain comprises the amino acid sequence set forth in SEQ ID NO: 331; and the β chain comprises the amino acid sequence set forth in SEQ ID NO: 332. In certain embodiments, the TCR is designated as “TCR41”. In certain embodiments, the TCR41 binds to a RAS peptide comprising or consisting of the amino acid sequence set forth in SEQ ID NO: 2.

In certain embodiments, the CDRs sequences described above including Table 41 are delineated using the IMGT numbering system.

TABLE 41
(TCR41)

| CDRs | 1 | 2 | 3 |
|---|---|---|---|
| α-chain | TRDTTYY | RNSFDEQN | ALSETYNNAGNMLT |
| | [SEQ ID NO: 285] | [SEQ ID NO: 286] | [SEQ ID NO: 325] |
| ß-chain | MNHNY | SVGAGI | ASTQNTEAF |
| | [SEQ ID NO: 326] | [SEQ ID NO: 327] | [SEQ ID NO: 328] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSFNFTITASQVVDSAVYFC |
| | ALSETYNNAGNMLTFGGGTRLMVKP |
| | [SEQ ID NO: 329] |
| ß-chain | MSISLLCCAAFPLLWAGPVNAGVTQTPKFRILKIGQSMTLQCAQDMNHNYMYWYRQ |
| variable | DPGMGLKLIYYSVGAGITDKGEVPNGYNVSRSTTEDFPLRLELAAPSQTSVYFCAS |
| | TQNTEAFFGQGTRLTVV |
| | [SEQ ID NO: 330] |
| Full α- | MLTASLLRAVIASICVVSSMAQKVTQAQTEISVVEKEDVTLDCVYETRDTTYYLFW |
| chain | YKQPPSGELVFLIRRNSFDEQNEISGRYSWNFQKSTSSFNFTITASQVVDSAVYFC |
| | ALSETYNNAGNMLTFGGGTRLMVKPNIQNPDPAVYQLRDSKSSDKSVCLFTDFDSQ |
| | TNVSQSKDSDVYITDKTVLDMRSMDFKSNSAVAWSNKSDFACANAFNNSIIPEDTF |
| | FPSPESSCDVKLVEKSFETDTNLNFQNLSVIGFRILLLKVAGENLLMTLRLWSS |
| | [SEQ ID NO: 331] |
| Full ß- | MSISLLCCAAFPLLWAGPVNAGVTQTPKFRILKIGQSMTLQCAQDMNHNYMYWYRQ |
| chain | DPGMGLKLIYYSVGAGITDKGEVPNGYNVSRSTTEDEPLRLELAAPSQTSVYFCAS |
| | TQNTEAFFGQGTRLTVVEDLNKVEPPEVAVFEPSEAEISHTQKATLVCLATGFFPD |
| | HVELSWWVNGKEVHSGVSTDPQPLKEQPALNDSRYCLSSRLRVSATFWQNPRNHER |
| | CQVQFYGLSENDEWTQDRAKPVTQIVSAEAWGRADCGFTSVSYQQGVLSATILYEI |
| | LLGKATLYAVLVSALVLMAMVKRKDE |
| | [SEQ ID NO: 332] |

In certain embodiments, the extracellular domain of the TCR comprises an α chain variable region comprising a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 333 or a conservative modification thereof. SEQ ID NOS: 5, 6, and 333 are disclosed in Table 42. In certain embodiments, the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333.

In certain embodiments, the extracellular domain of the TCR comprises a β chain variable region comprising a CDR1 comprising the amino acid sequence set forth in SEQ ID NO: 304 or a conservative modification thereof, a CDR2 comprising the amino acid sequence set forth in SEQ ID NO: 305 or a conservative modification thereof, and a CDR3 comprising the amino acid sequence set forth in SEQ ID NO: 334 or a conservative modification thereof. SEQ ID NOS: 304, 305, and 334 are disclosed in Table 42. In certain embodiments, the β chain variable region CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334.

In certain embodiments, the α chain variable region comprises a CDR1 comprising the amino acid sequence set forth in SEQ ID NO: 5 or a conservative modification thereof, a CDR2 comprising the amino acid sequence set forth in SEQ ID NO: 6 or a conservative modification thereof, and a CDR3 comprising the amino acid sequence set forth in SEQ ID NO: 333 or a conservative modification thereof; and the β chain variable region comprises a CDR1 comprising the amino acid sequence set forth in SEQ ID NO: 304 or a conservative modification thereof, a CDR2 comprising the amino acid sequence set forth in SEQ ID NO: 305 or a conservative modification thereof, and a CDR3 comprising the amino acid sequence set forth in SEQ ID NO: 334 or a conservative modification thereof. In certain embodiments, the α chain variable region comprises a CDR1 comprising the amino acid sequence set forth in SEQ ID NO: 5, a CDR2 comprising the amino acid sequence set forth in SEQ ID NO: 6, a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 335. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 335. In certain embodiments, the α chain variable region comprises the amino acid sequence set forth in SEQ ID NO: 335. SEQ ID NO: 335 is provided in Table 42.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 336. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 336. In certain embodiments, the β chain variable region comprises the amino acid sequence set forth in SEQ ID NO: 336. SEQ ID NO: 336 is provided in Table 42.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 335; and the β chain variable region comprises an amino acid sequence that is at least about 80% (e.g., at least about 85%, at least about 90%, or at least about 95%) homologous or identical to the amino acid sequence set forth in SEQ ID NO: 336. In certain embodiments, the α chain variable region comprises the amino acid sequence set forth in SEQ ID NO: 335; and the β chain variable region comprises the amino acid sequence set forth in SEQ ID NO: 336.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 337. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 337. In certain embodiments, the α chain comprises the amino acid sequence set forth in SEQ ID NO: 337.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 338. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 338. In certain embodiments, the β chain comprises the amino acid sequence set forth in SEQ ID NO: 338.

In certain embodiments, the α chain comprises an amino acid sequence that is at least about 80% (e.g., at least about 85%, at least about 90%, or at least about 95%) homologous or identical to the amino acid sequence set forth in SEQ ID NO: 337; and the β chain comprises an amino acid sequence that is at least about 80% (e.g., at least about 85%, at least about 90%, or at least about 95%) homologous or identical to the amino acid sequence set forth in SEQ ID NO: 338. In certain embodiments, the α chain comprises the amino acid sequence set forth in SEQ ID NO: 337; and the β chain comprises the amino acid sequence set forth in SEQ ID NO: 338. In certain embodiments, the TCR is designated as “TCR42”. In certain embodiments, the TCR42 binds to a RAS peptide comprising or consisting of the amino acid sequence set forth in SEQ ID NO: 2.

In certain embodiments, the CDRs sequences described above including Table 42 are delineated using the IMGT numbering system.

TABLE 42
(TCR42)

| CDRs | 1 | 2 | 3 |
|---|---|---|---|
| α-chain | SIFNT | LYKAGEL | AGQFAGGTSYGKLT |
| | [SEQ ID NO: 5] | [SEQ ID NO: 6] | [SEQ ID NO: 333] |
| ß-chain | LGHNT | FRNRAP | ASGRGLDTGELF |
| | [SEQ ID NO: 304] | [SEQ ID NO: 305] | [SEQ ID NO: 334] |

| | |
|---|---|
| α-chain | MLLEHLLIILWMQLTWVSGQQLNQSPQSMFIQEGEDVSMNCTSSSIFNTWLWYKQD |
| variable | PGEGPVLLIALYKAGELTSNGRLTAQFGITRKDSFLNISASIPSDVGIYFCAGQFA |
| | GGTSYGKLTFGQGTILTVHP |
| | [SEQ ID NO: 335] |
| ß-chain | MATRLLCCVVLCLLGEELIDARVTQTPRHKVTEMGQEVTMRCQPILGHNTVFWYRQ |
| variable | TMMQGLELLAYFRNRAPLDDSGMPKDRFSAEMPDATLATLKIQPSEPRDSAVYFCA |
| | SGRGLDTGELFFGEGSRLTVL |
| | [SEQ ID NO: 336] |
| Full α- | MLLEHLLIILWMQLTWVSGQQLNQSPQSMFIQEGEDVSMNCTSSSIFNTWLWYKQD |
| chain | PGEGPVLLIALYKAGELTSNGRLTAQFGITRKDSFLNISASIPSDVGIYFCAGQFA |
| | GGTSYGKLTFGQGTILTVHPNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 337] |
| Full ß- | MATRLLCCVVLCLLGEELIDARVTQTPRHKVTEMGQEVTMRCQPILGHNTVFWYRQ |
| chain | TMMQGLELLAYFRNRAPLDDSGMPKDRFSAEMPDATLATLKIQPSEPRDSAVYFCA |
| | SGRGLDTGELFFGEGSRLTVLEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 338] |

In certain embodiments, the extracellular domain of the TCR comprises an α chain variable region comprising a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 339 or a conservative modification thereof. SEQ ID NOS: 285, 286, and 339 are disclosed in Table 43. In certain embodiments, the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339.

In certain embodiments, the extracellular domain of the TCR comprises a β chain variable region comprising a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 340 or a conservative modification thereof. SEQ ID NOS: 288, 289, and 340 are disclosed in Table 43. In certain embodiments, the β chain variable region CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340.

In certain embodiments, the α chain variable region comprises a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 339 or a conservative modification thereof; and the β chain variable region comprises a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 340 or a conservative modification thereof. In certain embodiments, the α chain variable region comprises a CDR1 comprising the amino acid sequence set forth in SEQ ID NO: 285, a CDR2 comprising the amino acid sequence set forth in SEQ ID NO: 286, a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 341. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 341. In certain embodiments, the α chain variable region comprises the amino acid sequence set forth in SEQ ID NO: 341. SEQ ID NO: 341 is provided in Table 43.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 342. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 342. In certain embodiments, the β chain variable region comprises the amino acid sequence set forth in SEQ ID NO: 342. SEQ ID NO: 342 is provided in Table 43.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 341; and the β chain variable region comprises an amino acid sequence that is at least about 80% (e.g., at least about 85%, at least about 90%, or at least about 95%) homologous or identical to the amino acid sequence set forth in SEQ ID NO: 342. In certain embodiments, the α chain variable region comprises the amino acid sequence set forth in SEQ ID NO: 341; and the β chain variable region comprises the amino acid sequence set forth in SEQ ID NO: 342.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 343. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 343. In certain embodiments, the α chain comprises the amino acid sequence set forth in SEQ ID NO: 343.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 344. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 344. In certain embodiments, the β chain comprises the amino acid sequence set forth in SEQ ID NO: 344.

In certain embodiments, the α chain comprises an amino acid sequence that is at least about 80% (e.g., at least about 85%, at least about 90%, or at least about 95%) homologous or identical to the amino acid sequence set forth in SEQ ID NO: 343; and the β chain comprises an amino acid sequence that is at least about 80% (e.g., at least about 85%, at least about 90%, or at least about 95%) homologous or identical to the amino acid sequence set forth in SEQ ID NO: 344. In certain embodiments, the α chain comprises the amino acid sequence set forth in SEQ ID NO: 343; and the β chain comprises the amino acid sequence set forth in SEQ ID NO: 344. In certain embodiments, the TCR is designated as “TCR43”. In certain embodiments, the TCR43 binds to a RAS peptide comprising or consisting of the amino acid sequence set forth in SEQ ID NO: 2.

In certain embodiments, the CDRs sequences described above including Table 43 are delineated using the IMGT numbering system.

TABLE 43
(TCR43)

| CDRs | 1 | 2 | 3 |
|---|---|---|---|
| α-chain | TRDTTYY | RNSFDEQN | ALSESGNNAGNMLT |
| | [SEQ ID NO: 285] | [SEQ ID NO: 286] | [SEQ ID NO: 339] |
| ß-chain | MNHNS | SASEGT | ASSENTEAF |
| | [SEQ ID NO: 288] | [SEQ ID NO: 289] | [SEQ ID NO: 340] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSFNFTITASQVVDSAVYFC |
| | ALSESGNNAGNMLTFGGGTRLMVKP |
| | [SEQ ID NO: 341] |
| ß-chain | MSIGLLCCVAFSLLWASPVNAGVTQTPKFQVLKTGQSMTLQCAQDMNHNSMYWYRQ |
| variable | DPGMGLRLIYYSASEGTTDKGEVPNGYNVSRLNKREFSLRLESAAPSQTSVYFCAS |
| | SENTEAFFGQGTRLTVV |
| | [SEQ ID NO: 342] |
| Full α- | MLTASLLRAVIASICVVSSMAQKVTQAQTEISVVEKEDVTLDCVYETRDTTYYLFW |
| chain | YKQPPSGELVFLIRRNSFDEQNEISGRYSWNFQKSTSSFNFTITASQVVDSAVYFC |
| | ALSESGNNAGNMLTFGGGTRLMVKPNIQNPDPAVYQLRDSKSSDKSVCLFTDFDSQ |
| | TNVSQSKDSDVYITDKTVLDMRSMDFKSNSAVAWSNKSDFACANAFNNSIIPEDTF |
| | FPSPESSCDVKLVEKSFETDTNLNFQNLSVIGFRILLLKVAGFNLLMTLRLWSS |
| | [SEQ ID NO: 343] |
| Full ß- | MSIGLLCCVAFSLLWASPVNAGVTQTPKFQVLKTGQSMTLQCAQDMNHNSMYWYRQ |
| chain | DPGMGLRLIYYSASEGTTDKGEVPNGYNVSRLNKREFSLRLESAAPSQTSVYFCAS |
| | SENTEAFFGQGTRLTVVEDLKNVFPPKVAVFEPSEAEISHTQKATLVCLATGFYPD |
| | HVELSWWVNGKEVHSGVSTDPQPLKEQPALNDSRYCLSSRLRVSATFWQNPRNHFR |
| | CQVQFYGLSENDEWTQDRAKPVTQIVSAEAWGRADCGFTSESYQQGVLSATILYEI |
| | LLGKATLYAVLVSALVLMAMVKRKDSRG |
| | [SEQ ID NO: 344] |

In certain embodiments, the extracellular domain of the TCR comprises an α chain variable region comprising a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 345 or a conservative modification thereof. SEQ ID NOS: 285, 286, and 345 are disclosed in Table 44. In certain embodiments, the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345.

In certain embodiments, the extracellular domain of the TCR comprises a β chain variable region comprising a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 340 or a conservative modification thereof. SEQ ID NOS: 288, 289, and 340 are disclosed in Table 44. In certain embodiments, the β chain variable region CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340.

In certain embodiments, the α chain variable region comprises a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 345 or a conservative modification thereof; and the β chain variable region comprises a CDR1 comprising the amino acid sequence set forth in SEQ ID NO: 288 or a conservative modification thereof, a CDR2 comprising the amino acid sequence set forth in SEQ ID NO: 289 or a conservative modification thereof, and a CDR3 comprising the amino acid sequence set forth in SEQ ID NO: 340 or a conservative modification thereof. In certain embodiments, the α chain variable region comprises a CDR1 comprising the amino acid sequence set forth in SEQ ID NO: 285, a CDR2 comprising the amino acid sequence set forth in SEQ ID NO: 286, a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 346. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 346. In certain embodiments, the α chain variable region comprises the amino acid sequence set forth in SEQ ID NO: 346. SEQ ID NO: 346 is provided in Table 44.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 342. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 342. In certain embodiments, the β chain variable region comprises the amino acid sequence set forth in SEQ ID NO: 342. SEQ ID NO: 342 is provided in Table 44.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 346; and the β chain variable region comprises an amino acid sequence that is at least about 80% (e.g., at least about 85%, at least about 90%, or at least about 95%) homologous or identical to the amino acid sequence set forth in SEQ ID NO: 342. In certain embodiments, the α chain variable region comprises the amino acid sequence set forth in SEQ ID NO: 346; and the β chain variable region comprises the amino acid sequence set forth in SEQ ID NO: 342.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 347. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 347. In certain embodiments, the α chain comprises the amino acid sequence set forth in SEQ ID NO: 347.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 348. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 348. In certain embodiments, the β chain comprises the amino acid sequence set forth in SEQ ID NO: 348.

In certain embodiments, the α chain comprises an amino acid sequence that is at least about 80% (e.g., at least about 85%, at least about 90%, or at least about 95%) homologous or identical to the amino acid sequence set forth in SEQ ID NO: 347; and the β chain comprises an amino acid sequence that is at least about 80% (e.g., at least about 85%, at least about 90%, or at least about 95%) homologous or identical to the amino acid sequence set forth in SEQ ID NO: 348. In certain embodiments, the α chain comprises the amino acid sequence set forth in SEQ ID NO: 347; and the β chain comprises the amino acid sequence set forth in SEQ ID NO: 348. In certain embodiments, the TCR is designated as “TCR44”. In certain embodiments, the TCR44 binds to a RAS peptide comprising or consisting of the amino acid sequence set forth in SEQ ID NO: 2.

In certain embodiments, the CDRs sequences described above including Table 44 are delineated using the IMGT numbering system.

TABLE 44
(TCR44)

| CDRs | 1 | 2 | 3 |
|---|---|---|---|
| α-chain | TRDTTYY | RNSFDEQN | ALSESGRNAGNMLT |
| | [SEQ ID NO: 285] | [SEQ ID NO: 286] | [SEQ ID NO: 345] |
| ß-chain | MNHNS | SASEGT | ASSENTEAF |
| | [SEQ ID NO: 288] | [SEQ ID NO: 289] | [SEQ ID NO: 340] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSFNFTITASQVVDSAVYFC |
| | ALSESGRNAGNMLTFGGGTRLMVKP |
| | [SEQ ID NO: 346] |
| ß-chain | MSIGLLCCVAFSLLWASPVNAGVTQTPKFQVLKTGQSMTLQCAQDMNHNSMYWYRQ |
| variable | DPGMGLRLIYYSASEGTTDKGEVPNGYNVSRLNKREFSLRLESAAPSQTSVYFCAS |
| | SENTEAFFGQGTRLTVV |
| | [SEQ ID NO: 342] |
| Full α- | MLTASLLRAVIASICVVSSMAQKVTQAQTEISVVEKEDVTLDCVYETRDTTYYLFW |
| chain | YKQPPSGELVFLIRRNSFDEQNEISGRYSWNFQKSTSSFNFTITASQVVDSAVYFC |
| | ALSESGRNAGNMLTFGGGTRLMVKPNIQNPDPAVYQLRDSKSSDKSVCLFTDFDSQ |
| | TNVSQSKDSDVYITDKTVLDMRSMDFKSNSAVAWSNKSDFACANAENNSIIPEDTF |
| | FPSPESSCDVKLVEKSFETDTNLNFQNLSVIGFRILLLKVAGFNLLMTLRLWSS |
| | [SEQ ID NO: 347] |
| Full ß- | MSIGLLCCVAFSLLWASPVNAGVTQTPKFQVLKTGQSMTLQCAQDMNHNSMYWYRQ |
| chain | DPGMGLRLIYYSASEGTTDKGEVPNGYNVSRLNKREFSLRLESAAPSQTSVYFCAS |
| | SENTEAFFGQGTRLTVVEDLNKVEPPEVAVFEPSEAEISHTQKATLVCLATGFFPD |
| | HVELSWWVNGKEVHSGVSTDPQPLKEQPALNDSRYCLSSRLRVSATFWQNPRNHFR |
| | CQVQFYGLSENDEWTQDRAKPVTQIVSAEAWGRADCGFTSVSYQQGVLSATILYEI |
| | LLGKATLYAVLVSALVLMAMVKRKDF |
| | [SEQ ID NO: 348] |

In certain embodiments, the extracellular domain of the TCR comprises an α chain variable region comprising a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 349 or a conservative modification thereof. SEQ ID NOS: 93, 94, and 349 are disclosed in Table 45. In certain embodiments, the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349.

In certain embodiments, the extracellular domain of the TCR comprises a β chain variable region comprising a CDR1 comprising the amino acid sequence set forth in SEQ ID NO: 182 or a conservative modification thereof, a CDR2 comprising the amino acid sequence set forth in SEQ ID NO: 350 or a conservative modification thereof, and a CDR3 comprising the amino acid sequence set forth in SEQ ID NO: 351 or a conservative modification thereof. SEQ ID NOS: 182, 350, and 351 are disclosed in Table 45. In certain embodiments, the β chain variable region CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351.

In certain embodiments, the α chain variable region comprises a CDR1 comprising the amino acid sequence set forth in SEQ ID NO: 93 or a conservative modification thereof, a CDR2 comprising the amino acid sequence set forth in SEQ ID NO: 94 or a conservative modification thereof, and a CDR3 comprising the amino acid sequence set forth in SEQ ID NO: 349 or a conservative modification thereof; and the β chain variable region comprises a CDR1 comprising the amino acid sequence set forth in SEQ ID NO: 182 or a conservative modification thereof, a CDR2 comprising the amino acid sequence set forth in SEQ ID NO: 350 or a conservative modification thereof, and a CDR3 comprising the amino acid sequence set forth in SEQ ID NO: 351 or a conservative modification thereof. In certain embodiments, the α chain variable region comprises a CDR1 comprising the amino acid sequence set forth in SEQ ID NO: 93, a CDR2 comprising the amino acid sequence set forth in SEQ ID NO: 94, a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 352. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 352. In certain embodiments, the α chain variable region comprises the amino acid sequence set forth in SEQ ID NO: 352. SEQ ID NO: 352 is provided in Table 45.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 353. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 353. In certain embodiments, the β chain variable region comprises the amino acid sequence set forth in SEQ ID NO: 353. SEQ ID NO: 353 is provided in Table 45.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 352; and the β chain variable region comprises an amino acid sequence that is at least about 80% (e.g., at least about 85%, at least about 90%, or at least about 95%) homologous or identical to the amino acid sequence set forth in SEQ ID NO: 353. In certain embodiments, the α chain variable region comprises the amino acid sequence set forth in SEQ ID NO: 352; and the β chain variable region comprises the amino acid sequence set forth in SEQ ID NO: 353.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 354. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 354. In certain embodiments, the α chain comprises the amino acid sequence set forth in SEQ ID NO: 354.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 355. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 355. In certain embodiments, the β chain comprises the amino acid sequence set forth in SEQ ID NO: 355.

In certain embodiments, the α chain comprises an amino acid sequence that is at least about 80% (e.g., at least about 85%, at least about 90%, or at least about 95%) homologous or identical to the amino acid sequence set forth in SEQ ID NO: 354; and the β chain comprises an amino acid sequence that is at least about 80% (e.g., at least about 85%, at least about 90%, or at least about 95%) homologous or identical to the amino acid sequence set forth in SEQ ID NO: 355. In certain embodiments, the α chain comprises the amino acid sequence set forth in SEQ ID NO: 354; and the β chain comprises the amino acid sequence set forth in SEQ ID NO: 355. In certain embodiments, the TCR is designated as “TCR45”. In certain embodiments, the TCR45 binds to a RAS peptide comprising or consisting of the amino acid sequence set forth in SEQ ID NO: 2.

In certain embodiments, the CDRs sequences described above including Table 45 are delineated using the IMGT numbering system.

TABLE 45
(TCR45)

| CDRs | 1 | 2 | 3 |
|---|---|---|---|
| α-chain | ATGYPS | ATKADDK | ALRLRNTGNQFY |
| | [SEQ ID NO: 93] | [SEQ ID NO: 94] | [SEQ ID NO: 349] |
| ß-chain | MNHEY | SVGEGT | ASSGTGRPTTMETQY |
| | [SEQ ID NO: 182] | [SEQ ID NO: 350] | [SEQ ID NO: 351] |

| | |
|---|---|
| α-chain | MNYSPGLVSLILLLLGRTRGNSVTQMEGPVTLSEEAFLTINCTYTATGYPSLFWYV |
| variable | QYPGEGLQLLLKATKADDKGSNKGFEATYRKETTSFHLEKGSVQVSDSAVYFCALR |
| | LRNTGNQFYFGTGTSLTVIP |
| | [SEQ ID NO: 352] |
| ß-chain | MSLGLLCCGAFSLLWAGPVNAGVTQTPKFRVLKTGQSMTLLCAQDMNHEYMYWYRQ |
| variable | DPGMGLRLIHYSVGEGTTAKGEVPDGYNVSRLKKQNELLGLESAAPSQTSVYFCAS |
| | SGTGRPTTMETQYFGPGTRLLVL |
| | [SEQ ID NO: 353] |
| Full α- | MNYSPGLVSLILLLLGRTRGNSVTQMEGPVTLSEEAFLTINCTYTATGYPSLFWYV |
| chain | QYPGEGLQLLLKATKADDKGSNKGFEATYRKETTSFHLEKGSVQVSDSAVYFCALR |
| | LRNTGNQFYFGTGTSLTVIPNIQNPDPAVYQLRDSKSSDKSVCLFTDFDSQTNVSQ |
| | SKDSDVYITDKTVLDMRSMDFKSNSAVAWSNKSDFACANAFNNSIIPEDTFFPSPE |
| | SSCDVKLVEKSFETDTNLNFQNLSVIGFRILLLKVAGFNLLMTLRLWSS |
| | [SEQ ID NO: 354] |
| Full ß- | MSLGLLCCGAFSLLWAGPVNAGVTQTPKFRVLKTGQSMTLLCAQDMNHEYMYWYRQ |
| chain | DPGMGLRLIHYSVGEGTTAKGEVPDGYNVSRLKKQNFLLGLESAAPSQTSVYFCAS |
| | SGTGRPTTMETQYFGPGTRLLVLEDLKNVEPPKVAVFEPSEAEISHTQKATLVCLA |
| | TGFYPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSESYQQGVLSA |
| | TILYEILLGKATLYAVLVSALVLMAMVKRKDSRG |
| | [SEQ ID NO: 355] |

In certain embodiments, the extracellular domain of the TCR comprises an α chain variable region comprising a CDR1 comprising the amino acid sequence set forth in SEQ ID NO: 195 or a conservative modification thereof, a CDR2 comprising the amino acid sequence set forth in SEQ ID NO: 196 or a conservative modification thereof, and a CDR3 comprising the amino acid sequence set forth in SEQ ID NO: 356 or a conservative modification thereof. SEQ ID NOS: 195, 196, and 356 are disclosed in Table 46. In certain embodiments, the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356.

In certain embodiments, the extracellular domain of the TCR comprises a β chain variable region comprising a CDR1 comprising the amino acid sequence set forth in SEQ ID NO: 182 or a conservative modification thereof, a CDR2 comprising the amino acid sequence set forth in SEQ ID NO: 350 or a conservative modification thereof, and a CDR3 comprising the amino acid sequence set forth in SEQ ID NO: 351 or a conservative modification thereof. SEQ ID NOS: 182, 350, and 351 are disclosed in Table 46. In certain embodiments, the β chain variable region CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351.

In certain embodiments, the α chain variable region comprises a CDR1 comprising the amino acid sequence set forth in SEQ ID NO: 195 or a conservative modification thereof, a CDR2 comprising the amino acid sequence set forth in SEQ ID NO: 196 or a conservative modification thereof, and a CDR3 comprising the amino acid sequence set forth in SEQ ID NO: 356 or a conservative modification thereof; and the β chain variable region comprises a CDR1 comprising the amino acid sequence set forth in SEQ ID NO: 182 or a conservative modification thereof, a CDR2 comprising the amino acid sequence set forth in SEQ ID NO: 350 or a conservative modification thereof, and a CDR3 comprising the amino acid sequence set forth in SEQ ID NO: 351 or a conservative modification thereof. In certain embodiments, the α chain variable region comprises a CDR1 comprising the amino acid sequence set forth in SEQ ID NO: 195, a CDR2 comprising the amino acid sequence set forth in SEQ ID NO: 196, a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 357. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 357. In certain embodiments, the α chain variable region comprises the amino acid sequence set forth in SEQ ID NO: 357. SEQ ID NO: 357 is provided in Table 46.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 353. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 353. In certain embodiments, the β chain variable region comprises the amino acid sequence set forth in SEQ ID NO: 353. SEQ ID NO: 353 is provided in Table 46.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 357; and the β chain variable region comprises an amino acid sequence that is at least about 80% (e.g., at least about 85%, at least about 90%, or at least about 95%) homologous or identical to the amino acid sequence set forth in SEQ ID NO: 353. In certain embodiments, the α chain variable region comprises the amino acid sequence set forth in SEQ ID NO: 357; and the β chain variable region comprises the amino acid sequence set forth in SEQ ID NO: 353.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 358. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 358. In certain embodiments, the α chain comprises the amino acid sequence set forth in SEQ ID NO: 358.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 359. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 359. In certain embodiments, the β chain comprises the amino acid sequence set forth in SEQ ID NO: 359.

In certain embodiments, the α chain comprises an amino acid sequence that is at least about 80% (e.g., at least about 85%, at least about 90%, or at least about 95%) homologous or identical to the amino acid sequence set forth in SEQ ID NO: 358; and the β chain comprises an amino acid sequence that is at least about 80% (e.g., at least about 85%, at least about 90%, or at least about 95%) homologous or identical to the amino acid sequence set forth in SEQ ID NO: 359. In certain embodiments, the α chain comprises the amino acid sequence set forth in SEQ ID NO: 358; and the β chain comprises the amino acid sequence set forth in SEQ ID NO: 359. In certain embodiments, the TCR is designated as “TCR46”. In certain embodiments, the TCR46 binds to a RAS peptide comprising or consisting of the amino acid sequence set forth in SEQ ID NO: 2.

In certain embodiments, the CDRs sequences described above including Table 46 are delineated using the IMGT numbering system.

TABLE 46
(TCR46)

| CDRs | 1 | 2 | 3 |
|---|---|---|---|
| α-chain | TTLSN | LVKSGEV | AVSTWPNDYKLS |
| | [SEQ ID NO: 195] | [SEQ ID NO: 196] | [SEQ ID NO: 356] |
| ß-chain | MNHEY | SVGEGT | ASSGTGRPTTMETQY |
| | [SEQ ID NO: 182] | [SEQ ID NO: 350] | [SEQ ID NO: 351] |

| | |
|---|---|
| α-chain | MLLITSMLVLWMQLSQVNGQQVMQIPQYQHVQEGEDFTTYCNSSTTLSNIQWYKQR |
| variable | PGGHPVFLIQLVKSGEVKKQKRLTFQFGEAKKNSSLHITATQTTDVGTYFCAVSTW |
| | PNDYKLSFGAGTTVTVRA |
| | [SEQ ID NO: 357] |
| ß-chain | MSLGLLCCGAFSLLWAGPVNAGVTQTPKFRVLKTGQSMTLLCAQDMNHEYMYWYRQ |
| variable | DPGMGLRLIHYSVGEGTTAKGEVPDGYNVSRLKKQNELLGLESAAPSQTSVYFCAS |
| | SGTGRPTTMETQYFGPGTRLLVL |
| | [SEQ ID NO: 353] |
| Full α- | MLLITSMLVLWMQLSQVNGQQVMQIPQYQHVQEGEDFTTYCNSSTTLSNIQWYKQR |
| chain | PGGHPVFLIQLVKSGEVKKQKRLTFQFGEAKKNSSLHITATQTTDVGTYFCAVSTW |
| | PNDYKLSFGAGTTVTVRANIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESS |
| | CDVKLVEKSFETDTNLNFQNLSVIGFRILLLKVAGFNLLMTLRLWSS |
| | [SEQ ID NO: 358] |
| Full ß- | MSLGLLCCGAFSLLWAGPVNAGVTQTPKFRVLKTGQSMTLLCAQDMNHEYMYWYRQ |
| chain | DPGMGLRLIHYSVGEGTTAKGEVPDGYNVSRLKKQNELLGLESAAPSQTSVYFCAS |
| | SGTGRPTTMETQYFGPGTRLLVLEDLKNVEPPKVAVFEPSEAEISHTQKATLVCLA |
| | TGFYPDHVELSWWVNGKEVHSGVSTDPQPLKEQPALNDSRYCLSSRLRVSATFWQN |
| | PRNHFRCQVQFYGLSENDEWTQDRAKPVTQIVSAEAWGRADCGFTSESYQQGVLSA |
| | TILYEILLGKATLYAVLVSALVLMAMVKRKDSRG |
| | [SEQ ID NO: 359] |

In certain embodiments, the extracellular domain of the TCR comprises an α chain variable region comprising a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 360 or a conservative modification thereof. SEQ ID NOS: 179, 180, and 360 are disclosed in Table 47. In certain embodiments, the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 361 or a conservative modification thereof. SEQ ID NOS: 37, 38, and 361 are disclosed in Table 47. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361.

In certain embodiments, the α chain variable region comprises a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 360 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 361 or a conservative modification thereof. In certain embodiments, the α chain variable region comprises a CDR1 comprising the amino acid sequence set forth in SEQ ID NO: 179, a CDR2 comprising the amino acid sequence set forth in SEQ ID NO: 180, a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 362. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 362. In certain embodiments, the α chain variable region comprises the amino acid sequence set forth in SEQ ID NO: 362. SEQ ID NO: 362 is provided in Table 47.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 363. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 363. In certain embodiments, the β chain variable region comprises the amino acid sequence set forth in SEQ ID NO: 363. SEQ ID NO: 363 is provided in Table 47.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 362; and the β chain variable region comprises an amino acid sequence that is at least about 80% (e.g., at least about 85%, at least about 90%, or at least about 95%) homologous or identical to the amino acid sequence set forth in SEQ ID NO: 363. In certain embodiments, the α chain variable region comprises the amino acid sequence set forth in SEQ ID NO: 362; and the β chain variable region comprises the amino acid sequence set forth in SEQ ID NO: 363.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 364. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 364. In certain embodiments, the α chain comprises the amino acid sequence set forth in SEQ ID NO: 364.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 365. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 365. In certain embodiments, the β chain comprises the amino acid sequence set forth in SEQ ID NO: 365.

In certain embodiments, the α chain comprises an amino acid sequence that is at least about 80% (e.g., at least about 85%, at least about 90%, or at least about 95%) homologous or identical to the amino acid sequence set forth in SEQ ID NO: 364; and the β chain comprises an amino acid sequence that is at least about 80% (e.g., at least about 85%, at least about 90%, or at least about 95%) homologous or identical to the amino acid sequence set forth in SEQ ID NO: 365. In certain embodiments, the α chain comprises the amino acid sequence set forth in SEQ ID NO: 364; and the β chain comprises the amino acid sequence set forth in SEQ ID NO: 365. In certain embodiments, the TCR is designated as “TCR47”. In certain embodiments, the TCR47 binds to a RAS peptide comprising or consisting of the amino acid sequence set forth in SEQ ID NO: 2.

In certain embodiments, the CDRs sequences described above including Table 47 are delineated using the IMGT numbering system.

TABLE 47
(TCR47)

| CDRs | 1 | 2 | 3 |
|---|---|---|---|
| α-chain | DRGSQS | IYSNGD | AVNYGQNFV |
| | [SEQ ID NO: 179] | [SEQ ID NO: 180] | [SEQ ID NO: 360] |
| ß-chain | SGDLS | YYNGEE | ASTSGSDRDYEQY |
| | [SEQ ID NO: 37] | [SEQ ID NO: 38] | [SEQ ID NO: 361] |

| | |
|---|---|
| α-chain | MKSLRVLLVILWLQLSWVWSQQKEVEQNSGPLSVPEGAIASLNCTYSDRGSQSFFW |
| variable | YRQYSGKSPELIMSIYSNGDKEDGRFTAQLNKASQYVSLLIRDSQPSDSATYLCAV |
| | NYGQNFVFGPGTRLSVLP |
| | [SEQ ID NO: 362] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | TSGSDRDYEQYFGPGTRLTVT |
| | [SEQ ID NO: 363] |
| Full α- | MKSLRVLLVILWLQLSWVWSQQKEVEQNSGPLSVPEGAIASLNCTYSDRGSQSFFW |
| chain | YRQYSGKSPELIMSIYSNGDKEDGRFTAQLNKASQYVSLLIRDSQPSDSATYLCAV |
| | NYGQNFVFGPGTRLSVLPNIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESS |
| | CDVKLVEKSFETDTNLNFQNLSVIGFRILLLKVAGFNLLMTLRLWSS |
| | [SEQ ID NO: 364] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | TSGSDRDYEQYFGPGTRLTVTEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 365] |

In certain embodiments, the extracellular domain of the TCR comprises an α chain variable region comprising a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 325 or a conservative modification thereof. SEQ ID NOS: 285, 286, and 325 are disclosed in Table 48. In certain embodiments, the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325.

In certain embodiments, the extracellular domain of the TCR comprises a β chain variable region comprising a CDR1 comprising the amino acid sequence set forth in SEQ ID NO: 326 or a conservative modification thereof, a CDR2 comprising the amino acid sequence set forth in SEQ ID NO: 357 or a conservative modification thereof, and a CDR3 comprising the amino acid sequence set forth in SEQ ID NO: 290 or a conservative modification thereof. SEQ ID NOS: 326, 357, and 290 are disclosed in Table 48. In certain embodiments, the β chain variable region CDR1 comprising the amino acid sequence set forth in SEQ ID NO: 326, a CDR2 comprising the amino acid sequence set forth in SEQ ID NO: 357, and a CDR3 comprising the amino acid sequence set forth in SEQ ID NO: 290.

In certain embodiments, the α chain variable region comprises a CDR1 comprising the amino acid sequence set forth in SEQ ID NO: 285 or a conservative modification thereof, a CDR2 comprising the amino acid sequence set forth in SEQ ID NO: 286 or a conservative modification thereof, and a CDR3 comprising the amino acid sequence set forth in SEQ ID NO: 325 or a conservative modification thereof; and the β chain variable region comprises a CDR1 comprising the amino acid sequence set forth in SEQ ID NO: 326 or a conservative modification thereof, a CDR2 comprising the amino acid sequence set forth in SEQ ID NO: 357 or a conservative modification thereof, and a CDR3 comprising the amino acid sequence set forth in SEQ ID NO: 290 or a conservative modification thereof. In certain embodiments, the α chain variable region comprises a CDR1 comprising the amino acid sequence set forth in SEQ ID NO: 285, a CDR2 comprising the amino acid sequence set forth in SEQ ID NO: 286, a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 357, and a CDR3 comprising the amino acid sequence set forth in SEQ ID NO: 290.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 329. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 329. In certain embodiments, the α chain variable region comprises the amino acid sequence set forth in SEQ ID NO: 329. SEQ ID NO: 329 is provided in Table 48.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 366. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 366. In certain embodiments, the β chain variable region comprises the amino acid sequence set forth in SEQ ID NO: 366. SEQ ID NO: 366 is provided in Table 48.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 329; and the β chain variable region comprises an amino acid sequence that is at least about 80% (e.g., at least about 85%, at least about 90%, or at least about 95%) homologous or identical to the amino acid sequence set forth in SEQ ID NO: 366. In certain embodiments, the α chain variable region comprises the amino acid sequence set forth in SEQ ID NO: 329; and the β chain variable region comprises the amino acid sequence set forth in SEQ ID NO: 366.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 367. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 367. In certain embodiments, the α chain comprises the amino acid sequence set forth in SEQ ID NO: 367.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 368. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 368. In certain embodiments, the β chain comprises the amino acid sequence set forth in SEQ ID NO: 368.

In certain embodiments, the α chain comprises an amino acid sequence that is at least about 80% (e.g., at least about 85%, at least about 90%, or at least about 95%) homologous or identical to the amino acid sequence set forth in SEQ ID NO: 367; and the β chain comprises an amino acid sequence that is at least about 80% (e.g., at least about 85%, at least about 90%, or at least about 95%) homologous or identical to the amino acid sequence set forth in SEQ ID NO: 368. In certain embodiments, the α chain comprises the amino acid sequence set forth in SEQ ID NO: 367; and the β chain comprises the amino acid sequence set forth in SEQ ID NO: 368. In certain embodiments, the TCR is designated as “TCR48”. In certain embodiments, the TCR48 binds to a RAS peptide comprising or consisting of the amino acid sequence set forth in SEQ ID NO: 2.

In certain embodiments, the CDRs sequences described above including Table 48 are delineated using the IMGT numbering system.

TABLE 48
(TCR48)

| CDRs | 1 | 2 | 3 |
|---|---|---|---|
| α-chain | TRDTTYY | RNSFDEQN | ALSETYNNAGNMLT |
| | [SEQ ID NO: 285] | [SEQ ID NO: 286] | [SEQ ID NO: 325] |
| ß-chain | MNHNY | SVGAGI | ASSQNTEAF |
| | [SEQ ID NO: 326] | [SEQ ID NO: 327] | [SEQ ID NO: 290] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSFNFTITASQVVDSAVYFC |
| | ALSETYNNAGNMLTFGGGTRLMVKP |
| | [SEQ ID NO: 329] |
| ß-chain | MSISLLCCAAFPLLWAGPVNAGVTQTPKFRILKIGQSMTLQCTQDMNHNYMYWYRQ |
| variable | DPGMGLKLIYYSVGAGITDKGEVPNGYNVSRSTTEDFPLRLELAAPSQTSVYFCAS |
| | SQNTEAFFGQGTRLTVV |
| | [SEQ ID NO: 366] |
| Full α- | MLTASLLRAVIASICVVSSMAQKVTQAQTEISVVEKEDVTLDCVYETRDTTYYLFW |
| chain | YKQPPSGELVFLIRRNSFDEQNEISGRYSWNFQKSTSSFNFTITASQVVDSAVYFC |
| | ALSETYNNAGNMLTFGGGTRLMVKPNIQNPDPAVYQLRDSKSSDKSVCLFTDFDSQ |
| | TNVSQSKDSDVYITDKTVLDMRSMDFKSNSAVAWSNKSDFACANAFNNSIIPEDTF |
| | FPSPESSCDVKLVEKSFETDTNLNFQNLSVIGFRILLLKVAGFNLLMTLRLWSS |
| | [SEQ ID NO: 367] |
| Full ß- | MSISLLCCAAFPLLWAGPVNAGVTQTPKFRILKIGQSMTLQCTQDMNHNYMYWYRQ |
| chain | DPGMGLKLIYYSVGAGITDKGEVPNGYNVSRSTTEDFPLRLELAAPSQTSVYFCAS |
| | SQNTEAFFGQGTRLTVVEDLNKVFPPEVAVFEPSEAEISHTQKATLVCLATGFFPD |
| | HVELSWWVNGKEVHSGVSTDPQPLKEQPALNDSRYCLSSRLRVSATFWQNPRNHFR |
| | CQVQFYGLSENDEWTQDRAKPVTQIVSAEAWGRADCGFTSVSYQQGVLSATILYEI |
| | LLGKATLYAVLVSALVLMAMVKRKDF |
| | [SEQ ID NO: 368] |

In certain embodiments, the extracellular domain of the TCR comprises an α chain variable region comprising a CDR1 comprising the amino acid sequence set forth in SEQ ID NO: 369 or a conservative modification thereof, a CDR2 comprising the amino acid sequence set forth in SEQ ID NO: 370 or a conservative modification thereof, and a CDR3 comprising the amino acid sequence set forth in SEQ ID NO: 371 or a conservative modification thereof. SEQ ID NOS: 369-371 are disclosed in Table 49. In certain embodiments, the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371.

In certain embodiments, the extracellular domain of the TCR comprises a β chain variable region comprising a CDR1 comprising the amino acid sequence set forth in SEQ ID NO: 326 or a conservative modification thereof, a CDR2 comprising the amino acid sequence set forth in SEQ ID NO: 357 or a conservative modification thereof, and a CDR3 comprising the amino acid sequence set forth in SEQ ID NO: 290 or a conservative modification thereof. SEQ ID NOS: 326, 357, and 290 are disclosed in Table 49. In certain embodiments, the β chain variable region CDR1 comprising the amino acid sequence set forth in SEQ ID NO: 326, a CDR2 comprising the amino acid sequence set forth in SEQ ID NO: 357, and a CDR3 comprising the amino acid sequence set forth in SEQ ID NO: 290.

In certain embodiments, the α chain variable region comprises a CDR1 comprising the amino acid sequence set forth in SEQ ID NO: 369 or a conservative modification thereof, a CDR2 comprising the amino acid sequence set forth in SEQ ID NO: 370 or a conservative modification thereof, and a CDR3 comprising the amino acid sequence set forth in SEQ ID NO: 371 or a conservative modification thereof; and the β chain variable region comprises a CDR1 comprising the amino acid sequence set forth in SEQ ID NO: 326 or a conservative modification thereof, a CDR2 comprising the amino acid sequence set forth in SEQ ID NO: 357 or a conservative modification thereof, and a CDR3 comprising the amino acid sequence set forth in SEQ ID NO: 290 or a conservative modification thereof. In certain embodiments, the α chain variable region comprises a CDR1 comprising the amino acid sequence set forth in SEQ ID NO: 369, a CDR2 comprising the amino acid sequence set forth in SEQ ID NO: 370, a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 357, and a CDR3 comprising the amino acid sequence set forth in SEQ ID NO: 290.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 372. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 372. In certain embodiments, the α chain variable region comprises the amino acid sequence set forth in SEQ ID NO: 372. SEQ ID NO: 372 is provided in Table 49.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 366. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 366. In certain embodiments, the β chain variable region comprises the amino acid sequence set forth in SEQ ID NO: 366. SEQ ID NO: 366 is provided in Table 49.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 372; and the β chain variable region comprises an amino acid sequence that is at least about 80% (e.g., at least about 85%, at least about 90%, or at least about 95%) homologous or identical to the amino acid sequence set forth in SEQ ID NO: 366. In certain embodiments, the α chain variable region comprises the amino acid sequence set forth in SEQ ID NO: 372; and the β chain variable region comprises the amino acid sequence set forth in SEQ ID NO: 366.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 373. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 373. In certain embodiments, the α chain comprises the amino acid sequence set forth in SEQ ID NO: 373.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 374. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 374. In certain embodiments, the β chain comprises the amino acid sequence set forth in SEQ ID NO: 374.

In certain embodiments, the α chain comprises an amino acid sequence that is at least about 80% (e.g., at least about 85%, at least about 90%, or at least about 95%) homologous or identical to the amino acid sequence set forth in SEQ ID NO: 373; and the β chain comprises an amino acid sequence that is at least about 80% (e.g., at least about 85%, at least about 90%, or at least about 95%) homologous or identical to the amino acid sequence set forth in SEQ ID NO: 374. In certain embodiments, the α chain comprises the amino acid sequence set forth in SEQ ID NO: 373; and the β chain comprises the amino acid sequence set forth in SEQ ID NO: 374. In certain embodiments, the TCR is designated as “TCR49”. In certain embodiments, the TCR49 binds to a RAS peptide comprising or consisting of the amino acid sequence set forth in SEQ ID NO: 2.

In certain embodiments, the CDRs sequences described above including Table 49 are delineated using the IMGT numbering system.

TABLE 49
(TCR49)

| CDRs | 1 | 2 | 3 |
|---|---|---|---|
| α-chain | NYSPAY | IRENEKE | ARLRDNYGQNFV |
| | [SEQ ID NO: 369] | [SEQ ID NO: 370] | [SEQ ID NO: 371] |
| ß-chain | MNHNY | SVGAGI | ASSQNTEAF |
| | [SEQ ID NO: 326] | [SEQ ID NO: 327] | [SEQ ID NO: 290] |

| | |
|---|---|
| α-chain | MAFWLRSLGLHFRLHLGRRMESFLGGVLLILWLQVDWVKSQKIEQNSEALNIQEGK |
| variable | TATLTCNYTNYSPAYLQWYRQDPGRGPVELLLIRENEKEKRKERLKVTFDTTLKQS |
| | LFHITASQPADSATYLCARLRDNYGQNFVFGPGTRLSVLP |
| | [SEQ ID NO: 372] |
| ß-chain | MSISLLCCAAFPLLWAGPVNAGVTQTPKFRILKIGQSMTLQCTQDMNHNYMYWYRQ |
| variable | DPGMGLKLIYYSVGAGITDKGEVPNGYNVSRSTTEDEPLRLELAAPSQTSVYFCAS |
| | SQNTEAFFGQGTRLTVV |
| | [SEQ ID NO: 366] |
| Full α- | MAFWLRSLGLHFRLHLGRRMESFLGGVLLILWLQVDWVKSQKIEQNSEALNIQEGK |
| chain | TATLTCNYTNYSPAYLQWYRQDPGRGPVELLLIRENEKEKRKERLKVTEDTTLKQS |
| | LFHITASQPADSATYLCARLRDNYGQNFVFGPGTRLSVLPNIQNPDPAVYQLRDSK |
| | SSDKSVCLFTDFDSQTNVSQSKDSDVYITDKTVLDMRSMDFKSNSAVAWSNKSDFA |
| | CANAFNNSIIPEDTFFPSPESSCDVKLVEKSFETDTNLNFQNLSVIGFRILLLKVA |
| | GFNLLMTLRLWSS |
| | [SEQ ID NO: 373] |
| Full ß- | MSISLLCCAAFPLLWAGPVNAGVTQTPKFRILKIGQSMTLQCTQDMNHNYMYWYRQ |
| chain | DPGMGLKLIYYSVGAGITDKGEVPNGYNVSRSTTEDEPLRLELAAPSQTSVYFCAS |
| | SQNTEAFFGQGTRLTVVEDLNKVEPPEVAVFEPSEAEISHTQKATLVCLATGFFPD |
| | HVELSWWVNGKEVHSGVSTDPQPLKEQPALNDSRYCLSSRLRVSATFWQNPRNHFR |
| | CQVQFYGLSENDEWTQDRAKPVTQIVSAEAWGRADCGFTSVSYQQGVLSATILYEI |
| | LLGKATLYAVLVSALVLMAMVKRKDF |
| | [SEQ ID NO: 374] |

In certain embodiments, the extracellular domain of the TCR comprises an α chain variable region comprising a CDR1 comprising the amino acid sequence set forth in SEQ ID NO: 375 or a conservative modification thereof, a CDR2 comprising the amino acid sequence set forth in SEQ ID NO: 376 or a conservative modification thereof, and a CDR3 comprising the amino acid sequence set forth in SEQ ID NO: 377 or a conservative modification thereof. SEQ ID NOS: 375-377 are disclosed in Table 50. In certain embodiments, the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 378 or a conservative modification thereof. SEQ ID NOS: 37, 38, and 378 are disclosed in Table 50. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments, the α chain variable region comprises a CDR1 comprising the amino acid sequence set forth in SEQ ID NO: 375 or a conservative modification thereof, a CDR2 comprising the amino acid sequence set forth in SEQ ID NO: 376 or a conservative modification thereof, and a CDR3 comprising the amino acid sequence set forth in SEQ ID NO: 377 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 378 or a conservative modification thereof. In certain embodiments, the α chain variable region comprises a CDR1 comprising the amino acid sequence set forth in SEQ ID NO: 375, a CDR2 comprising the amino acid sequence set forth in SEQ ID NO: 376, a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 379. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 379. In certain embodiments, the α chain variable region comprises the amino acid sequence set forth in SEQ ID NO: 379. SEQ ID NO: 379 is provided in Table 50.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 380. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 380. In certain embodiments, the β chain variable region comprises the amino acid sequence set forth in SEQ ID NO: 380. SEQ ID NO: 380 is provided in Table 50.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 379; and the β chain variable region comprises an amino acid sequence that is at least about 80% (e.g., at least about 85%, at least about 90%, or at least about 95%) homologous or identical to the amino acid sequence set forth in SEQ ID NO: 380. In certain embodiments, the α chain variable region comprises the amino acid sequence set forth in SEQ ID NO: 379; and the β chain variable region comprises the amino acid sequence set forth in SEQ ID NO: 380.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 381. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 381. In certain embodiments, the α chain comprises the amino acid sequence set forth in SEQ ID NO: 381.

In certain embodiments, the extracellular domain of the TCR comprises a β chain that comprises a β chain variable region and a β chain constant region. In certain embodiments, the chain comprises an amino acid sequence that is at least about 80% (e.g., at least about 85%, at least about 90%, or at least about 95%) homologous or identical to the amino acid sequence set forth in SEQ ID NO: 382. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 382. In certain embodiments, the β chain comprises the amino acid sequence set forth in SEQ ID NO: 382.

In certain embodiments, the α chain comprises an amino acid sequence that is at least about 80% (e.g., at least about 85%, at least about 90%, or at least about 95%) homologous or identical to the amino acid sequence set forth in SEQ ID NO: 381; and the β chain comprises an amino acid sequence that is at least about 80% (e.g., at least about 85%, at least about 90%, or at least about 95%) homologous or identical to the amino acid sequence set forth in SEQ ID NO: 382. In certain embodiments, the α chain comprises the amino acid sequence set forth in SEQ ID NO: 381; and the β chain comprises the amino acid sequence set forth in SEQ ID NO: 382. In certain embodiments, the TCR is designated as “TCR50”. In certain embodiments, the TCR50 binds to a RAS peptide comprising or consisting of the amino acid sequence set forth in SEQ ID NO: 2.

In certain embodiments, the CDRs sequences described above including Table 50 are delineated using the IMGT numbering system.

TABLE 50
(TCR50)

| CDRs | 1 | 2 | 3 |
|---|---|---|---|
| α-chain | VTNFRS | LTSSGIE | AARGTGTSGSRLT |
| | [SEQ ID NO: 375] | [SEQ ID NO: 376] | [SEQ ID NO: 377] |
| ß-chain | SGDLS | YYNGEE | ASSPGGGHSYEQY |
| | [SEQ ID NO: 37] | [SEQ ID NO: 38] | [SEQ ID NO: 378] |

| | |
|---|---|
| α-chain | MMKCPQALLAIFWLLLSWVSSEDKVVQSPLSLVVHEGDTVTLNCSYEVTNFRSLLW |
| variable | YKQEKKAPTFLFMLTSSGIEKKSGRLSSILDKKELSSILNITATQTGDSAIYLCAA |
| | RGTGTSGSRLTFGEGTQLTVNP |
| | [SEQ ID NO: 379] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SPGGGHSYEQYFGPGTRLTVT |
| | [SEQ ID NO: 380] |
| Full α- | MMKCPQALLAIFWLLLSWVSSEDKVVQSPLSLVVHEGDTVTLNCSYEVTNFRSLLW |
| chain | YKQEKKAPTFLEMLTSSGIEKKSGRLSSILDKKELSSILNITATQTGDSAIYLCAA |
| | RGTGTSGSRLTFGEGTQLTVNPNIQNPDPAVYQLRDSKSSDKSVCLFTDFDSQTNV |
| | SQSKDSDVYITDKTVLDMRSMDFKSNSAVAWSNKSDFACANAFNNSIIPEDTFFPS |
| | PESSCDVKLVEKSFETDINLNFQNLSVIGFRILLLKVAGFNLLMTLRLWSS |
| | [SEQ ID NO: 381] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SPGGGHSYEQYFGPGTRLTVTEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 382] |

In certain embodiments, the extracellular domain of the TCR comprises an α chain variable region comprising a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 383 or a conservative modification thereof. SEQ ID NOS: 179, 180, and 383 are disclosed in Table 51. In certain embodiments, the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383.

In certain embodiments, the extracellular domain of the TCR comprises a β chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 378 or a conservative modification thereof. SEQ ID NOS: 37, 38, and 378 are disclosed in Table 51. In certain embodiments, the β chain variable region CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments, the α chain variable region comprises a CDR1 comprising the amino acid sequence set forth in SEQ ID NO: 179 or a conservative modification thereof, a CDR2 comprising the amino acid sequence set forth in SEQ ID NO: 180 or a conservative modification thereof, and a CDR3 comprising the amino acid sequence set forth in SEQ ID NO: 383 or a conservative modification thereof; and the β chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 378 or a conservative modification thereof. In certain embodiments, the α chain variable region comprises a CDR1 comprising the amino acid sequence set forth in SEQ ID NO: 179, a CDR2 comprising the amino acid sequence set forth in SEQ ID NO: 180, a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 384. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 384. In certain embodiments, the α chain variable region comprises the amino acid sequence set forth in SEQ ID NO: 384. SEQ ID NO: 384 is provided in Table 51.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 380. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 380. In certain embodiments, the β chain variable region comprises the amino acid sequence set forth in SEQ ID NO: 380. SEQ ID NO: 380 is provided in Table 51.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 384; and the β chain variable region comprises an amino acid sequence that is at least about 80% (e.g., at least about 85%, at least about 90%, or at least about 95%) homologous or identical to the amino acid sequence set forth in SEQ ID NO: 350. In certain embodiments, the α chain variable region comprises the amino acid sequence set forth in SEQ ID NO: 384; and the β chain variable region comprises the amino acid sequence set forth in SEQ ID NO: 380.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 385. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 385. In certain embodiments, the α chain comprises the amino acid sequence set forth in SEQ ID NO: 385.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 386. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 386. In certain embodiments, the β chain comprises the amino acid sequence set forth in SEQ ID NO: 386.

In certain embodiments, the α chain comprises an amino acid sequence that is at least about 80% (e.g., at least about 85%, at least about 90%, or at least about 95%) homologous or identical to the amino acid sequence set forth in SEQ ID NO: 385; and the β chain comprises an amino acid sequence that is at least about 80% (e.g., at least about 85%, at least about 90%, or at least about 95%) homologous or identical to the amino acid sequence set forth in SEQ ID NO: 386. In certain embodiments, the α chain comprises the amino acid sequence set forth in SEQ ID NO: 385; and the β chain comprises the amino acid sequence set forth in SEQ ID NO: 386. In certain embodiments, the TCR is designated as “TCR51”. In certain embodiments, the TCR51 binds to a RAS peptide comprising or consisting of the amino acid sequence set forth in SEQ ID NO: 2.

In certain embodiments, the CDRs sequences described above including Table 51 are delineated using the IMGT numbering system.

TABLE 51
(TCR51)

| CDRs | 1 | 2 | 3 |
|---|---|---|---|
| α-chain | DRGSQS | IYSNGD | AVNGAQGGSEKLV |
| | [SEQ ID NO: 179] | [SEQ ID NO: 180] | [SEQ ID NO: 383] |
| ß-chain | SGDLS | YYNGEE | ASSPGGGHSYEQY |
| | [SEQ ID NO: 37] | [SEQ ID NO: 38] | [SEQ ID NO: 378] |

| | |
|---|---|
| α-chain | MKSLRVLLVILWLQLSWVWSQQKEVEQNSGPLSVPEGAIASLNCTYSDRGSQSFFW |
| variable | YRQYSGKSPELIMSIYSNGDKEDGRFTAQLNKASQYVSLLIRDSQPSDSATYLCAV |
| | NGAQGGSEKLVFGKGTKLTVNP |
| | [SEQ ID NO: 384] |
| ß-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SPGGGHSYEQYFGPGTRLTVT |
| | [SEQ ID NO: 380] |
| Full α- | MKSLRVLLVILWLQLSWVWSQQKEVEQNSGPLSVPEGAIASLNCTYSDRGSQSFFW |
| chain | YRQYSGKSPELIMSIYSNGDKEDGRFTAQLNKASQYVSLLIRDSQPSDSATYLCAV |
| | NGAQGGSEKLVFGKGTKLTVNPNIQNPDPAVYQLRDSKSSDKSVCLFTDFDSQTNV |
| | SQSKDSDVYITDKTVLDMRSMDFKSNSAVAWSNKSDFACANAFNNSIIPEDTFFPS |
| | PESSCDVKLVEKSFETDTNLNFQNLSVIGFRILLLKVAGFNLLMTLRLWSS |
| | [SEQ ID NO: 385] |
| Full ß- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SPGGGHSYEQYFGPGTRLTVTEDLKNVFPPKVAVFEPSEAEISHTQKATLVCLATG |
| | FYPDHVELSWWVNGKEVHSGVSTDPQPLKEQPALNDSRYCLSSRLRVSATFWQNPR |
| | NHFRCQVQFYGLSENDEWTQDRAKPVTQIVSAEAWGRADCGFTSESYQQGVLSATI |
| | LYEILLGKATLYAVLVSALVLMAMVKRKDSRG |
| | [SEQ ID NO: 386] |

In certain embodiments, the α chain variable region and/or the β chain variable region amino acid sequences have at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NO: 11, SEQ ID NO: 12, SEQ ID NO: 20, SEQ ID NO: 21, SEQ ID NO: 30, SEQ ID NO: 31, SEQ ID NO: 40, SEQ ID NO: 41, SEQ ID NO: 44, SEQ ID NO: 51, SEQ ID NO: 52, SEQ ID NO: 57, SEQ ID NO: 58, SEQ ID NO: 62, SEQ ID NO: 68, SEQ ID NO: 69, SEQ ID NO: 75, SEQ ID NO: 81, SEQ ID NO: 82, SEQ ID NO: 89, SEQ ID NO: 90, SEQ ID NO: 97, SEQ ID NO: 98, SEQ ID NO: 105, SEQ ID NO: 106, SEQ ID NO: 115, SEQ ID NO: 116, SEQ ID NO: 124, SEQ ID NO: 125, SEQ ID NO: 134, SEQ ID NO: 135, SEQ ID NO: 141, SEQ ID NO: 142, SEQ ID NO: 148, SEQ ID NO: 152, SEQ ID NO: 153, SEQ ID NO: 162, SEQ ID NO: 163, SEQ ID NO: 175, SEQ ID NO: 176, SEQ ID NO: 185, SEQ ID NO: 186, SEQ ID NO: 191, SEQ ID NO: 192, SEQ ID NO: 199, SEQ ID NO: 200, SEQ ID NO: 207, SEQ ID NO: 208, SEQ ID NO: 217, SEQ ID NO: 218, SEQ ID NO: 223, SEQ ID NO: 224, SEQ ID NO: 233, SEQ ID NO: 234, SEQ ID NO: 241, SEQ ID NO: 242, SEQ ID NO: 249, SEQ ID NO: 250, SEQ ID NO: 256, SEQ ID NO: 257, SEQ ID NO: 266, SEQ ID NO: 267, SEQ ID NO: 275, SEQ ID NO: 275, SEQ ID NO: 281, SEQ ID NO: 282, SEQ ID NO: 291, SEQ ID NO: 292, SEQ ID NO: 299, SEQ ID NO: 300, SEQ ID NO: 307, SEQ ID NO: 308, SEQ ID NO: 313, SEQ ID NO: 314, SEQ ID NO: 321, SEQ ID NO: 322, SEQ ID NO: 329, SEQ ID NO: 330, SEQ ID NO: 335, SEQ ID NO: 336, SEQ ID NO: 341, SEQ ID NO: 342, SEQ ID NO: 346, SEQ ID NO: 352, SEQ ID NO: 353, SEQ ID NO: 357, SEQ ID NO: 362, SEQ ID NO: 363, SEQ ID NO: 366, SEQ ID NO: 372, SEQ ID NO: 379, SEQ ID NO: 380, and SEQ ID NO: 384) comprise modifications, including, but not limited to, substitutions (e.g., conservative substitutions), insertions, or deletions relative to the specified sequence(s), but retain the ability to bind to a mutant RAS peptide (e.g., a Q61R mutant RAS peptide). In certain embodiments, such modifications are not within the CDR domains of the variable regions.

In certain embodiments, a total of 1 to 10 amino acids are substituted, inserted and/or deleted in SEQ ID NO: 11, SEQ ID NO: 12, SEQ ID NO: 20, SEQ ID NO: 21, SEQ ID NO: 30, SEQ ID NO: 31, SEQ ID NO: 40, SEQ ID NO: 41, SEQ ID NO: 44, SEQ ID NO: 51, SEQ ID NO: 52, SEQ ID NO: 57, SEQ ID NO: 58, SEQ ID NO: 62, SEQ ID NO: 68, SEQ ID NO: 69, SEQ ID NO: 75, SEQ ID NO: 81, SEQ ID NO: 82, SEQ ID NO: 89, SEQ ID NO: 90, SEQ ID NO: 97, SEQ ID NO: 98, SEQ ID NO: 105, SEQ ID NO: 106, SEQ ID NO: 115, SEQ ID NO: 116, SEQ ID NO: 124, SEQ ID NO: 125, SEQ ID NO: 134, SEQ ID NO: 135, SEQ ID NO: 141, SEQ ID NO: 142, SEQ ID NO: 148, SEQ ID NO: 152, SEQ ID NO: 153, SEQ ID NO: 162, SEQ ID NO: 163, SEQ ID NO: 175, SEQ ID NO: 176, SEQ ID NO: 185, SEQ ID NO: 186, SEQ ID NO: 191, SEQ ID NO: 192, SEQ ID NO: 199, SEQ ID NO: 200, SEQ ID NO: 207, SEQ ID NO: 208, SEQ ID NO: 217, SEQ ID NO: 218, SEQ ID NO: 223, SEQ ID NO: 224, SEQ ID NO: 233, SEQ ID NO: 234, SEQ ID NO: 241, SEQ ID NO: 242, SEQ ID NO: 249, SEQ ID NO: 250, SEQ ID NO: 256, SEQ ID NO: 257, SEQ ID NO: 266, SEQ ID NO: 267, SEQ ID NO: 275, SEQ ID NO: 275, SEQ ID NO: 281, SEQ ID NO: 282, SEQ ID NO: 291, SEQ ID NO: 292, SEQ ID NO: 299, SEQ ID NO: 300, SEQ ID NO: 307, SEQ ID NO: 308, SEQ ID NO: 313, SEQ ID NO: 314, SEQ ID NO: 321, SEQ ID NO: 322, SEQ ID NO: 329, SEQ ID NO: 330, SEQ ID NO: 335, SEQ ID NO: 336, SEQ ID NO: 341, SEQ ID NO: 342, SEQ ID NO: 346, SEQ ID NO: 352, SEQ ID NO: 353, SEQ ID NO: 357, SEQ ID NO: 362, SEQ ID NO: 363, SEQ ID NO: 366, SEQ ID NO: 372, SEQ ID NO: 379, SEQ ID NO: 380, or SEQ ID NO: 384. In certain embodiments, substitutions, insertions, or deletions occur in regions outside the CDRs of the extracellular domain. In certain embodiments, the extracellular domain comprises an α chain variable region and/or a β chain variable region sequence selected from the group consisting of SEQ ID NO: 11, SEQ ID NO: 12, SEQ ID NO: 20, SEQ ID NO: 21, SEQ ID NO: 30, SEQ ID NO: 31, SEQ ID NO: 40, SEQ ID NO: 41, SEQ ID NO: 44, SEQ ID NO: 51, SEQ ID NO: 52, SEQ ID NO: 57, SEQ ID NO: 58, SEQ ID NO: 62, SEQ ID NO: 68, SEQ ID NO: 69, SEQ ID NO: 75, SEQ ID NO: 81, SEQ ID NO: 82, SEQ ID NO: 89, SEQ ID NO: 90, SEQ ID NO: 97, SEQ ID NO: 98, SEQ ID NO: 105, SEQ ID NO: 106, SEQ ID NO: 115, SEQ ID NO: 116, SEQ ID NO: 124, SEQ ID NO: 125, SEQ ID NO: 134, SEQ ID NO: 135, SEQ ID NO: 141, SEQ ID NO: 142, SEQ ID NO: 148, SEQ ID NO: 152, SEQ ID NO: 153, SEQ ID NO: 162, SEQ ID NO: 163, SEQ ID NO: 175, SEQ ID NO: 176, SEQ ID NO: 185, SEQ ID NO: 186, SEQ ID NO: 191, SEQ ID NO: 192, SEQ ID NO: 199, SEQ ID NO: 200, SEQ ID NO: 207, SEQ ID NO: 208, SEQ ID NO: 217, SEQ ID NO: 218, SEQ ID NO: 223, SEQ ID NO: 224, SEQ ID NO: 233, SEQ ID NO: 234, SEQ ID NO: 241, SEQ ID NO: 242, SEQ ID NO: 249, SEQ ID NO: 250, SEQ ID NO: 256, SEQ ID NO: 257, SEQ ID NO: 266, SEQ ID NO: 267, SEQ ID NO: 275, SEQ ID NO: 275, SEQ ID NO: 281, SEQ ID NO: 282, SEQ ID NO: 291, SEQ ID NO: 292, SEQ ID NO: 299, SEQ ID NO: 300, SEQ ID NO: 307, SEQ ID NO: 308, SEQ ID NO: 313, SEQ ID NO: 314, SEQ ID NO: 321, SEQ ID NO: 322, SEQ ID NO: 329, SEQ ID NO: 330, SEQ ID NO: 335, SEQ ID NO: 336, SEQ ID NO: 341, SEQ ID NO: 342, SEQ ID NO: 346, SEQ ID NO: 352, SEQ ID NO: 353, SEQ ID NO: 357, SEQ ID NO: 362, SEQ ID NO: 363, SEQ ID NO: 366, SEQ ID NO: 372, SEQ ID NO: 379, SEQ ID NO: 380, or SEQ ID NO: 384 including post-translational modifications of that sequence (SEQ ID NO: 11, SEQ ID NO: 12, SEQ ID NO: 20, SEQ ID NO: 21, SEQ ID NO: 30, SEQ ID NO: 31, SEQ ID NO: 40, SEQ ID NO: 41, SEQ ID NO: 44, SEQ ID NO: 51, SEQ ID NO: 52, SEQ ID NO: 57, SEQ ID NO: 58, SEQ ID NO: 62, SEQ ID NO: 68, SEQ ID NO: 69, SEQ ID NO: 75, SEQ ID NO: 81, SEQ ID NO: 82, SEQ ID NO: 89, SEQ ID NO: 90, SEQ ID NO: 97, SEQ ID NO: 98, SEQ ID NO: 105, SEQ ID NO: 106, SEQ ID NO: 115, SEQ ID NO: 116, SEQ ID NO: 124, SEQ ID NO: 125, SEQ ID NO: 134, SEQ ID NO: 135, SEQ ID NO: 141, SEQ ID NO: 142, SEQ ID NO: 148, SEQ ID NO: 152, SEQ ID NO: 153, SEQ ID NO: 162, SEQ ID NO: 163, SEQ ID NO: 175, SEQ ID NO: 176, SEQ ID NO: 185, SEQ ID NO: 186, SEQ ID NO: 191, SEQ ID NO: 192, SEQ ID NO: 199, SEQ ID NO: 200, SEQ ID NO: 207, SEQ ID NO: 208, SEQ ID NO: 217, SEQ ID NO: 218, SEQ ID NO: 223, SEQ ID NO: 224, SEQ ID NO: 233, SEQ ID NO: 234, SEQ ID NO: 241, SEQ ID NO: 242, SEQ ID NO: 249, SEQ ID NO: 250, SEQ ID NO: 256, SEQ ID NO: 257, SEQ ID NO: 266, SEQ ID NO: 267, SEQ ID NO: 275, SEQ ID NO: 275, SEQ ID NO: 281, SEQ ID NO: 282, SEQ ID NO: 291, SEQ ID NO: 292, SEQ ID NO: 299, SEQ ID NO: 300, SEQ ID NO: 307, SEQ ID NO: 308, SEQ ID NO: 313, SEQ ID NO: 314, SEQ ID NO: 321, SEQ ID NO: 322, SEQ ID NO: 329, SEQ ID NO: 330, SEQ ID NO: 335, SEQ ID NO: 336, SEQ ID NO: 341, SEQ ID NO: 342, SEQ ID NO: 346, SEQ ID NO: 352, SEQ ID NO: 353, SEQ ID NO: 357, SEQ ID NO: 362, SEQ ID NO: 363, SEQ ID NO: 366, SEQ ID NO: 372, SEQ ID NO: 379, SEQ ID NO: 380, or SEQ ID NO: 384).

### 5.3.1.2. Constant Regions

In certain embodiments, the presently disclosed TCR comprises an α chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 166 or SEQ ID NO: 167. In certain embodiments, the α chain constant region comprises the amino acid sequence set forth in SEQ ID NO: 166. In certain embodiments, the α chain constant region comprises the amino acid sequence set forth in SEQ ID NO: 167.

In certain embodiments, the presently disclosed TCR comprises a β chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 168, SEQ ID NO: 169, SEQ ID NO: 170, or SEQ ID NO: 171. In certain embodiments, the β chain constant region comprises the amino acid sequence set forth in SEQ ID NO: 168. In certain embodiments, the β chain constant region comprises the amino acid sequence set forth in SEQ ID NO: 169. In certain embodiments, the β chain constant region comprises the amino acid sequence set forth in SEQ ID NO: 170. In certain embodiments, the β chain constant region comprises the amino acid sequence set forth in SEQ ID NO: 171. SEQ ID NOS: 166-171 are provided below:

Human α chain constant region:
[SEQ ID NO: 166]
NIQNPDPAVYQLRDSKSSDKSVCLFTDFDSQTNVSQSKDSDVYITDKTVLDMRSMDFKSNSAVAWSNKSDFACANAFNNS
IIPEDTFFPSPESSCDVKLVEKSFETDTNLNFQNLSVIGFRILLLKVAGFNLLMTLRLWSS
Mouse α chain constant region (cysteine-modification and LVL modification in
transmembrane domain underlined):
[SEQ ID NO: 167]
NIQNPEPAVYQLKDPRSQDSTLCLFTDFDSQINVPKTMESGTFITDKCVLDMKAMDSKSNGAIAWSNQTSFTCQDIFKET
NATYPSSDVPCDATLTEKSFETDMNLNFQNLLVIVLRILLLKVAGENLLMTLRLWSS
Human ß chain constant region:
[SEQ ID NO: 168]
EDLNKVFPPEVAVFEPSEAEISHTQKATLVCLATGFFPDHVELSWWVNGKEVHSGVSTDPQPLKEQPALNDSRYCLSSRL
RVSATFWQNPRNHFRCQVQFYGLSENDEWTQDRAKPVTQIVSAEAWGRADCGFTSVSYQQGVLSATILYEILLGKATLYA
VLVSALVLMAMVKRKDF
Human ß chain constant region:
[SEQ ID NO: 169]
EDLKNVFPPKVAVFEPSEAEISHTQKATLVCLATGFYPDHVELSWWVNGKEVHSGVSTDPQPLKEQPALNDSRYCLSSRL
RVSATFWQNPRNHFRCQVQFYGLSENDEWTQDRAKPVTQIVSAEAWGRADCGFTSESYQQGVLSATILYEILLGKATLYA
VLVSALVLMAMVKRKDSRG
Human ß chain constant region:
[SEQ ID NO: 170]
EDLKNVFPPEVAVFEPSEAEISHTQKATLVCLATGFYPDHVELSWWVNGKEVHSGVSTDPQPLKEQPALNDSRYCLSSRL
RVSATFWQNPRNHFRCQVQFYGLSENDEWTQDRAKPVTQIVSAEAWGRADCGFTSESYQQGVLSATILYEILLGKATLYA
VLVSALVLMAMVKRKDSRG
Mouse ß chain constant region (cysteine-modification underlined):
[SEQ ID NO: 171]
EDLRNVTPPKVSLFEPSKAEIANKQKATLVCLARGFFPDHVELSWWVNGKEVHSGVCTDPQAYKESNYSYCLSSRLRVSA
TFWHNPRNHFRCQVQFHGLSEEDKWPEGSPKPVTQNISAEAWGRADCGITSASYQQGVLSATILYEILLGKATLYAVLVS
TLVVMAMVKRKNS

5.3.2. TCRs that Bind to the Same RAS Peptide as TCR Clonotypes

The presently disclosed subject matter further provides TCRs that bind to the same RAS peptide (e.g., a Q61R mutant RAS peptide) as a TCR disclosed herein (e.g., a TCR disclosed in Section 5.3.1). In certain embodiments, the TCR binds to the same RAS peptide (e.g., a Q61R mutant RAS peptide) as a reference TCR or a functional fragment thereof comprising the α chain variable region CDR1, CDR2, and CDR3 sequences and the β chain variable region CDR1, CDR2, and CDR3 sequences of, for example, any one of the TCRs disclosed herein (e.g., those disclosed in Section 5.3.1). In certain embodiments, the TCR binds to the same RAS peptide (e.g., a Q61R mutant RAS peptide) as a reference TCR or a functional fragment thereof comprising the α chain variable region and the β chain variable region sequences of, for example, any one of the presently disclosed TCRs (e.g., those disclosed in Section 5.3.1).

### 5.3.3. TCRs Having Specific CDR3 Sequences

It is well known in the art that the CDR3 domain, independently from the CDR1 and/or CDR2 domain(s), alone can determine the binding specificity of a TCR or a functional fragment thereof, for a cognate antigen and that multiple TCRs can predictably be generated having the same binding specificity based on a common CDR3 sequence.

In certain embodiments, the extracellular domain of the TCR comprises an α chain variable region CDR3 comprising the amino acid sequence set forth in SEQ ID NO: 7 or a conservative modification thereof; and a β chain variable region CDR3 comprising the amino acid sequence set forth in SEQ ID NO: 10 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 9 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 8 or a conservative modification thereof.

In certain embodiments, the extracellular domain of the TCR comprises an α chain variable region CDR3 comprising the amino acid sequence set forth in SEQ ID NO: 17 or a conservative modification thereof; and a β chain variable region CDR3 comprising the amino acid sequence set forth in SEQ ID NO: 19 or a conservative modification thereof. In certain embodiments, the extracellular domain of the TCR further comprises an α chain variable region CDR2 comprising the amino acid sequence set forth in SEQ ID NO: 16 or a conservative modification thereof; and a β chain variable region CDR2 comprising the amino acid sequence set forth in SEQ ID NO: 18 or a conservative modification thereof. In certain embodiments, the extracellular domain of the TCR further comprises an α chain variable region CDR1 comprising the amino acid sequence set forth in SEQ ID NO: 15 or a conservative modification thereof; and a β chain variable region CDR1 comprising the amino acid sequence set forth in SEQ ID NO: 8 or a conservative modification thereof.

In certain embodiments, the extracellular domain of the TCR comprises an α chain variable region CDR3 comprising the amino acid sequence set forth in SEQ ID NO: 26 or a conservative modification thereof; and a β chain variable region CDR3 comprising the amino acid sequence set forth in SEQ ID NO: 29 or a conservative modification thereof. In certain embodiments, the extracellular domain of the TCR further comprises an α chain variable region CDR2 comprising the amino acid sequence set forth in SEQ ID NO: 25 or a conservative modification thereof; and a β chain variable region CDR2 comprising the amino acid sequence set forth in SEQ ID NO: 28 or a conservative modification thereof. In certain embodiments, the extracellular domain of the TCR further comprises an α chain variable region CDR1 comprising the amino acid sequence set forth in SEQ ID NO: 24 or a conservative modification thereof; and a β chain variable region CDR1 comprising the amino acid sequence set forth in SEQ ID NO: 27 or a conservative modification thereof.

In certain embodiments, the extracellular domain of the TCR comprises an α chain variable region CDR3 comprising the amino acid sequence set forth in SEQ ID NO: 36 or a conservative modification thereof; and a β chain variable region CDR3 comprising the amino acid sequence set forth in SEQ ID NO: 39 or a conservative modification thereof. In certain embodiments, the extracellular domain of the TCR further comprises an α chain variable region CDR2 comprising the amino acid sequence set forth in SEQ ID NO: 35 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 34 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 44 or a conservative modification thereof; and a β chain variable region CDR3 comprising the amino acid sequence set forth in SEQ ID NO: 39 or a conservative modification thereof. In certain embodiments, the extracellular domain of the TCR further comprises an α chain variable region CDR2 comprising the amino acid sequence set forth in SEQ ID NO: 35 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 34 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 47 or a conservative modification thereof; and a β chain variable region CDR3 comprising the amino acid sequence set forth in SEQ ID NO: 50 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 49 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 48 or a conservative modification thereof.

In certain embodiments, the extracellular domain of the TCR comprises an α chain variable region CDR3 comprising the amino acid sequence set forth in SEQ ID NO: 55 or a conservative modification thereof; and a β chain variable region CDR3 comprising the amino acid sequence set forth in SEQ ID NO: 56 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 55 or a conservative modification thereof; and a β chain variable region CDR3 comprising the amino acid sequence set forth in SEQ ID NO: 61 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 66 or a conservative modification thereof; and a β chain variable region CDR3 comprising the amino acid sequence set forth in SEQ ID NO: 67 or a conservative modification thereof. In certain embodiments, the extracellular domain of the TCR further comprises an α chain variable region CDR2 comprising the amino acid sequence set forth in SEQ ID NO: 65 or a conservative modification thereof; and a β chain variable region CDR2 comprising the amino acid sequence set forth in SEQ ID NO: 49 or a conservative modification thereof. In certain embodiments, the extracellular domain of the TCR further comprises an α chain variable region CDR1 comprising the amino acid sequence set forth in SEQ ID NO: 64 or a conservative modification thereof; and a β chain variable region CDR1 comprising the amino acid sequence set forth in SEQ ID NO: 48 or a conservative modification thereof.

In certain embodiments, the extracellular domain of the TCR comprises an α chain variable region CDR3 comprising the amino acid sequence set forth in SEQ ID NO: 74 or a conservative modification thereof; and a β chain variable region CDR3 comprising the amino acid sequence set forth in SEQ ID NO: 67 or a conservative modification thereof. In certain embodiments, the extracellular domain of the TCR further comprises an α chain variable region CDR2 comprising the amino acid sequence set forth in SEQ ID NO: 73 or a conservative modification thereof; and a β chain variable region CDR2 comprising the amino acid sequence set forth in SEQ ID NO: 49 or a conservative modification thereof. In certain embodiments, the extracellular domain of the TCR further comprises an α chain variable region CDR1 comprising the amino acid sequence set forth in SEQ ID NO: 72 or a conservative modification thereof; and a β chain variable region CDR1 comprising the amino acid sequence set forth in SEQ ID NO: 48 or a conservative modification thereof.

In certain embodiments, the extracellular domain of the TCR comprises an α chain variable region CDR3 comprising the amino acid sequence set forth in SEQ ID NO: 77 or a conservative modification thereof; and a β chain variable region CDR3 comprising the amino acid sequence set forth in SEQ ID NO: 80 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 79 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 78 or a conservative modification thereof.

In certain embodiments, the extracellular domain of the TCR comprises an α chain variable region CDR3 comprising the amino acid sequence set forth in SEQ ID NO: 87 or a conservative modification thereof; and a β chain variable region CDR3 comprising the amino acid sequence set forth in SEQ ID NO: 88 or a conservative modification thereof. In certain embodiments, the extracellular domain of the TCR further comprises an α chain variable region CDR2 comprising the amino acid sequence set forth in SEQ ID NO: 86 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 85 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 95 or a conservative modification thereof; and a β chain variable region CDR3 comprising the amino acid sequence set forth in SEQ ID NO: 96 or a conservative modification thereof. In certain embodiments, the extracellular domain of the TCR further comprises an α chain variable region CDR2 comprising the amino acid sequence set forth in SEQ ID NO: 94 or a conservative modification thereof; and a β chain variable region CDR2 comprising the amino acid sequence set forth in SEQ ID NO: 49 or a conservative modification thereof. In certain embodiments, the extracellular domain of the TCR further comprises an α chain variable region CDR1 comprising the amino acid sequence set forth in SEQ ID NO: 93 or a conservative modification thereof; and a β chain variable region CDR1 comprising the amino acid sequence set forth in SEQ ID NO: 48 or a conservative modification thereof.

In certain embodiments, the extracellular domain of the TCR comprises an α chain variable region CDR3 comprising the amino acid sequence set forth in SEQ ID NO: 101 or a conservative modification thereof; and a β chain variable region CDR3 comprising the amino acid sequence set forth in SEQ ID NO: 104 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 103 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 102 or a conservative modification thereof.

In certain embodiments, the extracellular domain of the TCR comprises an α chain variable region CDR3 comprising the amino acid sequence set forth in SEQ ID NO: 111 or a conservative modification thereof; and a β chain variable region CDR3 comprising the amino acid sequence set forth in SEQ ID NO: 114 or a conservative modification thereof. In certain embodiments, the extracellular domain of the TCR further comprises an α chain variable region CDR2 comprising the amino acid sequence set forth in SEQ ID NO: 110 or a conservative modification thereof; and a β chain variable region CDR2 comprising the amino acid sequence set forth in SEQ ID NO: 113 or a conservative modification thereof. In certain embodiments, the extracellular domain of the TCR further comprises an α chain variable region CDR1 comprising the amino acid sequence set forth in SEQ ID NO: 109 or a conservative modification thereof; and a β chain variable region CDR1 comprising the amino acid sequence set forth in SEQ ID NO: 112 or a conservative modification thereof.

In certain embodiments, the extracellular domain of the TCR comprises an α chain variable region CDR3 comprising the amino acid sequence set forth in SEQ ID NO: 121 or a conservative modification thereof; and a β chain variable region CDR3 comprising the amino acid sequence set forth in SEQ ID NO: 123 or a conservative modification thereof. In certain embodiments, the extracellular domain of the TCR further comprises an α chain variable region CDR2 comprising the amino acid sequence set forth in SEQ ID NO: 120 or a conservative modification thereof; and a β chain variable region CDR2 comprising the amino acid sequence set forth in SEQ ID NO: 113 or a conservative modification thereof. In certain embodiments, the extracellular domain of the TCR further comprises an α chain variable region CDR1 comprising the amino acid sequence set forth in SEQ ID NO: 119 or a conservative modification thereof; and a β chain variable region CDR1 comprising the amino acid sequence set forth in SEQ ID NO: 122 or a conservative modification thereof.

In certain embodiments, the extracellular domain of the TCR comprises an α chain variable region CDR3 comprising the amino acid sequence set forth in SEQ ID NO: 130 or a conservative modification thereof; and a β chain variable region CDR3 comprising the amino acid sequence set forth in SEQ ID NO: 133 or a conservative modification thereof. In certain embodiments, the extracellular domain of the TCR further comprises an α chain variable region CDR2 comprising the amino acid sequence set forth in SEQ ID NO: 129 or a conservative modification thereof; and a β chain variable region CDR2 comprising the amino acid sequence set forth in SEQ ID NO: 132 or a conservative modification thereof. In certain embodiments, the extracellular domain of the TCR further comprises an α chain variable region CDR1 comprising the amino acid sequence set forth in SEQ ID NO: 128 or a conservative modification thereof; and a β chain variable region CDR1 comprising the amino acid sequence set forth in SEQ ID NO: 131 or a conservative modification thereof.

In certain embodiments, the extracellular domain of the TCR comprises an α chain variable region CDR3 comprising the amino acid sequence set forth in SEQ ID NO: 139 or a conservative modification thereof; and a β chain variable region CDR3 comprising the amino acid sequence set forth in SEQ ID NO: 140 or a conservative modification thereof. In certain embodiments, the extracellular domain of the TCR further comprises an α chain variable region CDR2 comprising the amino acid sequence set forth in SEQ ID NO: 35 or a conservative modification thereof; and a β chain variable region CDR2 comprising the amino acid sequence set forth in SEQ ID NO: 49 or a conservative modification thereof. In certain embodiments, the extracellular domain of the TCR further comprises an α chain variable region CDR1 comprising the amino acid sequence set forth in SEQ ID NO: 138 or a conservative modification thereof; and a β chain variable region CDR1 comprising the amino acid sequence set forth in SEQ ID NO: 102 or a conservative modification thereof.

In certain embodiments, the extracellular domain of the TCR comprises an α chain variable region CDR3 comprising the amino acid sequence set forth in SEQ ID NO: 147 or a conservative modification thereof; and a β chain variable region CDR3 comprising the amino acid sequence set forth in SEQ ID NO: 140 or a conservative modification thereof. In certain embodiments, the extracellular domain of the TCR further comprises an α chain variable region CDR2 comprising the amino acid sequence set forth in SEQ ID NO: 146 or a conservative modification thereof; and a β chain variable region CDR2 comprising the amino acid sequence set forth in SEQ ID NO: 49 or a conservative modification thereof. In certain embodiments, the extracellular domain of the TCR further comprises an comprises an α chain variable region CDR1 comprising the amino acid sequence set forth in SEQ ID NO: 145 or a conservative modification thereof; and a β chain variable region CDR1 comprising the amino acid sequence set forth in SEQ ID NO: 102 or a conservative modification thereof.

In certain embodiments, the extracellular domain of the TCR comprises an α chain variable region CDR3 comprising the amino acid sequence set forth in SEQ ID NO: 150 or a conservative modification thereof; and a β chain variable region CDR3 comprising the amino acid sequence set forth in SEQ ID NO: 151 or a conservative modification thereof. In certain embodiments, the extracellular domain of the TCR further comprises an α chain variable region CDR2 comprising the amino acid sequence set forth in SEQ ID NO: 35 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 34 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 158 or a conservative modification thereof; and a β chain variable region CDR3 comprising the amino acid sequence set forth in SEQ ID NO: 161 or a conservative modification thereof. In certain embodiments, the extracellular domain of the TCR further comprises an α chain variable region CDR2 comprising the amino acid sequence set forth in SEQ ID NO: 157 or a conservative modification thereof; and a β chain variable region CDR2 comprising the amino acid sequence set forth in SEQ ID NO: 160 or a conservative modification thereof. In certain embodiments, the extracellular domain of the TCR further comprises an α chain variable region CDR1 comprising the amino acid sequence set forth in SEQ ID NO: 156 or a conservative modification thereof; and a β chain variable region CDR1 comprising the amino acid sequence set forth in SEQ ID NO: 159 or a conservative modification thereof.

In certain embodiments, the extracellular domain of the TCR comprises an α chain variable region CDR3 comprising the amino acid sequence set forth in SEQ ID NO: 173 or a conservative modification thereof; and a β chain variable region CDR3 comprising the amino acid sequence set forth in SEQ ID NO: 174 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 103 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 102 or a conservative modification thereof.

In certain embodiments, the extracellular domain of the TCR comprises an α chain variable region CDR3 comprising the amino acid sequence set forth in SEQ ID NO: 181 or a conservative modification thereof; and a β chain variable region CDR3 comprising the amino acid sequence set forth in SEQ ID NO: 194 or a conservative modification thereof. In certain embodiments, the extracellular domain of the TCR further comprises an α chain variable region CDR2 comprising the amino acid sequence set forth in SEQ ID NO: 180 or a conservative modification thereof; and a β chain variable region CDR2 comprising the amino acid sequence set forth in SEQ ID NO: 183 or a conservative modification thereof. In certain embodiments, the extracellular domain of the TCR further comprises an α chain variable region CDR1 comprising the amino acid sequence set forth in SEQ ID NO: 179 or a conservative modification thereof; and a β chain variable region CDR1 comprising the amino acid sequence set forth in SEQ ID NO: 182 or a conservative modification thereof.

In certain embodiments, the extracellular domain of the TCR comprises an α chain variable region CDR3 comprising the amino acid sequence set forth in SEQ ID NO: 189 or a conservative modification thereof; and a β chain variable region CDR3 comprising the amino acid sequence set forth in SEQ ID NO: 190 or a conservative modification thereof. In certain embodiments, the extracellular domain of the TCR further comprises an α chain variable region CDR2 comprising the amino acid sequence set forth in SEQ ID NO: 120 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 119 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 197 or a conservative modification thereof; and a β chain variable region CDR3 comprising the amino acid sequence set forth in SEQ ID NO: 198 or a conservative modification thereof. In certain embodiments, the extracellular domain of the TCR further comprises an α chain variable region CDR2 comprising the amino acid sequence set forth in SEQ ID NO: 196 or a conservative modification thereof; and a β chain variable region CDR2 comprising the amino acid sequence set forth in SEQ ID NO: 160 or a conservative modification thereof. In certain embodiments, the extracellular domain of the TCR further comprises an α chain variable region CDR1 comprising the amino acid sequence set forth in SEQ ID NO: 195 or a conservative modification thereof; and a β chain variable region CDR1 comprising the amino acid sequence set forth in SEQ ID NO: 159 or a conservative modification thereof.

In certain embodiments, the extracellular domain of the TCR comprises an α chain variable region CDR3 comprising the amino acid sequence set forth in SEQ ID NO: 205 or a conservative modification thereof; and a β chain variable region CDR3 comprising the amino acid sequence set forth in SEQ ID NO: 206 or a conservative modification thereof. In certain embodiments, the extracellular domain of the TCR further comprises an α chain variable region CDR2 comprising the amino acid sequence set forth in SEQ ID NO: 204 or a conservative modification thereof; and a β chain variable region CDR2 comprising the amino acid sequence set forth in SEQ ID NO: 113 or a conservative modification thereof. In certain embodiments, the extracellular domain of the TCR further comprises an α chain variable region CDR1 comprising the amino acid sequence set forth in SEQ ID NO: 203 or a conservative modification thereof; and a β chain variable region CDR1 comprising the amino acid sequence set forth in SEQ ID NO: 122 or a conservative modification thereof.

In certain embodiments, the extracellular domain of the TCR comprises an α chain variable region CDR3 comprising the amino acid sequence set forth in SEQ ID NO: 213 or a conservative modification thereof; and a β chain variable region CDR3 comprising the amino acid sequence set forth in SEQ ID NO: 216 or a conservative modification thereof. In certain embodiments, the extracellular domain of the TCR further comprises an α chain variable region CDR2 comprising the amino acid sequence set forth in SEQ ID NO: 212 or a conservative modification thereof; and a β chain variable region CDR2 comprising the amino acid sequence set forth in SEQ ID NO: 215 or a conservative modification thereof. In certain embodiments, the extracellular domain of the TCR further comprises an α chain variable region CDR1 comprising the amino acid sequence set forth in SEQ ID NO: 211 or a conservative modification thereof; and a β chain variable region CDR1 comprising the amino acid sequence set forth in SEQ ID NO: 214 or a conservative modification thereof.

In certain embodiments, the extracellular domain of the TCR comprises an α chain variable region CDR3 comprising the amino acid sequence set forth in SEQ ID NO: 221 or a conservative modification thereof; and a β chain variable region CDR3 comprising the amino acid sequence set forth in SEQ ID NO: 222 or a conservative modification thereof. In certain embodiments, the extracellular domain of the TCR further comprises an α chain variable region CDR2 comprising the amino acid sequence set forth in SEQ ID NO: 180 or a conservative modification thereof; and a β chain variable region CDR2 comprising the amino acid sequence set forth in SEQ ID NO: 160 or a conservative modification thereof. In certain embodiments, the extracellular domain of the TCR further comprises an α chain variable region CDR1 comprising the amino acid sequence set forth in SEQ ID NO: 179 or a conservative modification thereof; and a β chain variable region CDR1 comprising the amino acid sequence set forth in SEQ ID NO: 159 or a conservative modification thereof.

In certain embodiments, the extracellular domain of the TCR comprises an α chain variable region CDR3 comprising the amino acid sequence set forth in SEQ ID NO: 229 or a conservative modification thereof; and a β chain variable region CDR3 comprising the amino acid sequence set forth in SEQ ID NO: 232 or a conservative modification thereof. In certain embodiments, the extracellular domain of the TCR further comprises an α chain variable region CDR2 comprising the amino acid sequence set forth in SEQ ID NO: 228 or a conservative modification thereof; and a β chain variable region CDR2 comprising the amino acid sequence set forth in SEQ ID NO: 231 or a conservative modification thereof. In certain embodiments, the extracellular domain of the TCR further comprises an α chain variable region CDR1 comprising the amino acid sequence set forth in SEQ ID NO: 227 or a conservative modification thereof; and a β chain variable region CDR1 comprising the amino acid sequence set forth in SEQ ID NO: 230 or a conservative modification thereof.

In certain embodiments, the extracellular domain of the TCR comprises an α chain variable region CDR3 comprising the amino acid sequence set forth in SEQ ID NO: 237 or a conservative modification thereof; and a β chain variable region CDR3 comprising the amino acid sequence set forth in SEQ ID NO: 240 or a conservative modification thereof. In certain embodiments, the extracellular domain of the TCR further comprises an α chain variable region CDR2 comprising the amino acid sequence set forth in SEQ ID NO: 129 or a conservative modification thereof; and a β chain variable region CDR2 comprising the amino acid sequence set forth in SEQ ID NO: 239 or a conservative modification thereof. In certain embodiments, the extracellular domain of the TCR further comprises an α chain variable region CDR1 comprising the amino acid sequence set forth in SEQ ID NO: 128 or a conservative modification thereof; and a β chain variable region CDR1 comprising the amino acid sequence set forth in SEQ ID NO: 238 or a conservative modification thereof.

In certain embodiments, the extracellular domain of the TCR comprises an α chain variable region CDR3 comprising the amino acid sequence set forth in SEQ ID NO: 245 or a conservative modification thereof; and a β chain variable region CDR3 comprising the amino acid sequence set forth in SEQ ID NO: 248 or a conservative modification thereof. In certain embodiments, the extracellular domain of the TCR further comprises an α chain variable region CDR2 comprising the amino acid sequence set forth in SEQ ID NO: 73 or a conservative modification thereof; and a β chain variable region CDR2 comprising the amino acid sequence set forth in SEQ ID NO: 247 or a conservative modification thereof. In certain embodiments, the extracellular domain of the TCR further comprises an α chain variable region CDR1 comprising the amino acid sequence set forth in SEQ ID NO: 72 or a conservative modification thereof; and a β chain variable region CDR1 comprising the amino acid sequence set forth in SEQ ID NO: 246 or a conservative modification thereof.

In certain embodiments, the extracellular domain of the TCR comprises an α chain variable region CDR3 comprising the amino acid sequence set forth in SEQ ID NO: 254 or a conservative modification thereof; and a β chain variable region CDR3 comprising the amino acid sequence set forth in SEQ ID NO: 255 or a conservative modification thereof. In certain embodiments, the extracellular domain of the TCR further comprises an α chain variable region CDR2 comprising the amino acid sequence set forth in SEQ ID NO: 253 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 156 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 262 or a conservative modification thereof; and a β chain variable region CDR3 comprising the amino acid sequence set forth in SEQ ID NO: 265 or a conservative modification thereof. In certain embodiments, the extracellular domain of the TCR further comprises an α chain variable region CDR2 comprising the amino acid sequence set forth in SEQ ID NO: 261 or a conservative modification thereof; and a β chain variable region CDR2 comprising the amino acid sequence set forth in SEQ ID NO: 264 or a conservative modification thereof. In certain embodiments, the extracellular domain of the TCR further comprises an α chain variable region CDR1 comprising the amino acid sequence set forth in SEQ ID NO: 260 or a conservative modification thereof; and a β chain variable region CDR1 comprising the amino acid sequence set forth in SEQ ID NO: 263 or a conservative modification thereof.

In certain embodiments, the extracellular domain of the TCR comprises an α chain variable region CDR3 comprising the amino acid sequence set forth in SEQ ID NO: 270 or a conservative modification thereof; and a β chain variable region CDR3 comprising the amino acid sequence set forth in SEQ ID NO: 273 or a conservative modification thereof. In certain embodiments, the extracellular domain of the TCR further comprises an α chain variable region CDR2 comprising the amino acid sequence set forth in SEQ ID NO: 204 or a conservative modification thereof; and a β chain variable region CDR2 comprising the amino acid sequence set forth in SEQ ID NO: 272 or a conservative modification thereof. In certain embodiments, the extracellular domain of the TCR further comprises an α chain variable region CDR1 comprising the amino acid sequence set forth in SEQ ID NO: 203 or a conservative modification thereof; and a β chain variable region CDR1 comprising the amino acid sequence set forth in SEQ ID NO: 271 or a conservative modification thereof.

In certain embodiments, the extracellular domain of the TCR comprises an α chain variable region CDR3 comprising the amino acid sequence set forth in SEQ ID NO: 278 or a conservative modification thereof; and a β chain variable region CDR3 comprising the amino acid sequence set forth in SEQ ID NO: 280 or a conservative modification thereof. In certain embodiments, the extracellular domain of the TCR further comprises an α chain variable region CDR2 comprising the amino acid sequence set forth in SEQ ID NO: 129 or a conservative modification thereof; and a β chain variable region CDR2 comprising the amino acid sequence set forth in SEQ ID NO: 279 or a conservative modification thereof. In certain embodiments, the extracellular domain of the TCR further comprises an α chain variable region CDR1 comprising the amino acid sequence set forth in SEQ ID NO: 128 or a conservative modification thereof; and a β chain variable region CDR1 comprising the amino acid sequence set forth in SEQ ID NO: 263 or a conservative modification thereof.

In certain embodiments, the extracellular domain of the TCR comprises an α chain variable region CDR3 comprising the amino acid sequence set forth in SEQ ID NO: 287 or a conservative modification thereof; and a β chain variable region CDR3 comprising the amino acid sequence set forth in SEQ ID NO: 290 or a conservative modification thereof. In certain embodiments, the extracellular domain of the TCR further comprises an α chain variable region CDR2 comprising the amino acid sequence set forth in SEQ ID NO: 286 or a conservative modification thereof; and a β chain variable region CDR2 comprising the amino acid sequence set forth in SEQ ID NO: 289 or a conservative modification thereof. In certain embodiments, the extracellular domain of the TCR further comprises an α chain variable region CDR1 comprising the amino acid sequence set forth in SEQ ID NO: 285 or a conservative modification thereof; and a β chain variable region CDR1 comprising the amino acid sequence set forth in SEQ ID NO: 288 or a conservative modification thereof.

In certain embodiments, the extracellular domain of the TCR comprises an α chain variable region CDR3 comprising the amino acid sequence set forth in SEQ ID NO: 295 or a conservative modification thereof; and a β chain variable region CDR3 comprising the amino acid sequence set forth in SEQ ID NO: 298 or a conservative modification thereof. In certain embodiments, the extracellular domain of the TCR further comprises an α chain variable region CDR2 comprising the amino acid sequence set forth in SEQ ID NO: 228 or a conservative modification thereof; and a β chain variable region CDR2 comprising the amino acid sequence set forth in SEQ ID NO: 297 or a conservative modification thereof. In certain embodiments, the extracellular domain of the TCR further comprises an α chain variable region CDR1 comprising the amino acid sequence set forth in SEQ ID NO: 227 or a conservative modification thereof; and a β chain variable region CDR1 comprising the amino acid sequence set forth in SEQ ID NO: 296 or a conservative modification thereof.

In certain embodiments, the extracellular domain of the TCR comprises an α chain variable region CDR3 comprising the amino acid sequence set forth in SEQ ID NO: 303 or a conservative modification thereof; and a β chain variable region CDR3 comprising the amino acid sequence set forth in SEQ ID NO: 306 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 305 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 304 or a conservative modification thereof.

In certain embodiments, the extracellular domain of the TCR comprises an α chain variable region CDR3 comprising the amino acid sequence set forth in SEQ ID NO: 311 or a conservative modification thereof; and a β chain variable region CDR3 comprising the amino acid sequence set forth in SEQ ID NO: 312 or a conservative modification thereof. In certain embodiments, the extracellular domain of the TCR further comprises an α chain variable region CDR2 comprising the amino acid sequence set forth in SEQ ID NO: 129 or a conservative modification thereof; and a β chain variable region CDR2 comprising the amino acid sequence set forth in SEQ ID NO: 28 or a conservative modification thereof. In certain embodiments, the extracellular domain of the TCR further comprises an α chain variable region CDR1 comprising the amino acid sequence set forth in SEQ ID NO: 128 or a conservative modification thereof; and a β chain variable region CDR1 comprising the amino acid sequence set forth in SEQ ID NO: 27 or a conservative modification thereof.

In certain embodiments, the extracellular domain of the TCR comprises an α chain variable region CDR3 comprising the amino acid sequence set forth in SEQ ID NO: 319 or a conservative modification thereof; and a β chain variable region CDR3 comprising the amino acid sequence set forth in SEQ ID NO: 320 or a conservative modification thereof. In certain embodiments, the extracellular domain of the TCR further comprises an α chain variable region CDR2 comprising the amino acid sequence set forth in SEQ ID NO: 318 or a conservative modification thereof; and a β chain variable region CDR2 comprising the amino acid sequence set forth in SEQ ID NO: 279 or a conservative modification thereof. In certain embodiments, the extracellular domain of the TCR further comprises an α chain variable region CDR1 comprising the amino acid sequence set forth in SEQ ID NO: 317 or a conservative modification thereof; and a β chain variable region CDR1 comprising the amino acid sequence set forth in SEQ ID NO: 263 or a conservative modification thereof.

In certain embodiments, the extracellular domain of the TCR comprises an α chain variable region CDR3 comprising the amino acid sequence set forth in SEQ ID NO: 325 or a conservative modification thereof; and a β chain variable region CDR3 comprising the amino acid sequence set forth in SEQ ID NO: 328 or a conservative modification thereof. In certain embodiments, the extracellular domain of the TCR further comprises an α chain variable region CDR2 comprising the amino acid sequence set forth in SEQ ID NO: 286 or a conservative modification thereof; and a β chain variable region CDR2 comprising the amino acid sequence set forth in SEQ ID NO: 327 or a conservative modification thereof. In certain embodiments, the extracellular domain of the TCR further comprises an α chain variable region CDR1 comprising the amino acid sequence set forth in SEQ ID NO: 285 or a conservative modification thereof; and a β chain variable region CDR1 comprising the amino acid sequence set forth in SEQ ID NO: 326 or a conservative modification thereof.

In certain embodiments, the extracellular domain of the TCR comprises an α chain variable region CDR3 comprising the amino acid sequence set forth in SEQ ID NO: 333 or a conservative modification thereof; and a β chain variable region CDR3 comprising the amino acid sequence set forth in SEQ ID NO: 334 or a conservative modification thereof. In certain embodiments, the extracellular domain of the TCR further comprises an α chain variable region CDR2 comprising the amino acid sequence set forth in SEQ ID NO: 6 or a conservative modification thereof; and a β chain variable region CDR2 comprising the amino acid sequence set forth in SEQ ID NO: 305 or a conservative modification thereof. In certain embodiments, the extracellular domain of the TCR further comprises an α chain variable region CDR1 comprising the amino acid sequence set forth in SEQ ID NO: 5 or a conservative modification thereof; and a β chain variable region CDR1 comprising the amino acid sequence set forth in SEQ ID NO: 304 or a conservative modification thereof.

In certain embodiments, the extracellular domain of the TCR comprises an α chain variable region CDR3 comprising the amino acid sequence set forth in SEQ ID NO: 339 or a conservative modification thereof; and a β chain variable region CDR3 comprising the amino acid sequence set forth in SEQ ID NO: 340 or a conservative modification thereof. In certain embodiments, the extracellular domain of the TCR further comprises an α chain variable region CDR2 comprising the amino acid sequence set forth in SEQ ID NO: 286 or a conservative modification thereof; and a β chain variable region CDR2 comprising the amino acid sequence set forth in SEQ ID NO: 289 or a conservative modification thereof. In certain embodiments, the extracellular domain of the TCR further comprises an α chain variable region CDR1 comprising the amino acid sequence set forth in SEQ ID NO: 285 or a conservative modification thereof; and a β chain variable region CDR1 comprising the amino acid sequence set forth in SEQ ID NO: 288 or a conservative modification thereof.

In certain embodiments, the extracellular domain of the TCR comprises an α chain variable region CDR3 comprising the amino acid sequence set forth in SEQ ID NO: 345 or a conservative modification thereof; and a β chain variable region CDR3 comprising the amino acid sequence set forth in SEQ ID NO: 340 or a conservative modification thereof. In certain embodiments, the extracellular domain of the TCR further comprises an α chain variable region CDR2 comprising the amino acid sequence set forth in SEQ ID NO: 286 or a conservative modification thereof; and a β chain variable region CDR2 comprising the amino acid sequence set forth in SEQ ID NO: 289 or a conservative modification thereof. In certain embodiments, the extracellular domain of the TCR further comprises an α chain variable region CDR1 comprising the amino acid sequence set forth in SEQ ID NO: 285 or a conservative modification thereof; and a β chain variable region CDR1 comprising the amino acid sequence set forth in SEQ ID NO: 288 or a conservative modification thereof.

In certain embodiments, the extracellular domain of the TCR comprises an α chain variable region CDR3 comprising the amino acid sequence set forth in SEQ ID NO: 349 or a conservative modification thereof; and a β chain variable region CDR3 comprising the amino acid sequence set forth in SEQ ID NO: 351 or a conservative modification thereof. In certain embodiments, the extracellular domain of the TCR further comprises an α chain variable region CDR2 comprising the amino acid sequence set forth in SEQ ID NO: 94 or a conservative modification thereof; and a β chain variable region CDR2 comprising the amino acid sequence set forth in SEQ ID NO: 350 or a conservative modification thereof. In certain embodiments, the extracellular domain of the TCR further comprises an α chain variable region CDR1 comprising the amino acid sequence set forth in SEQ ID NO: 93 or a conservative modification thereof; and a β chain variable region CDR1 comprising the amino acid sequence set forth in SEQ ID NO: 182 or a conservative modification thereof.

In certain embodiments, the extracellular domain of the TCR comprises an α chain variable region CDR3 comprising the amino acid sequence set forth in SEQ ID NO: 356 or a conservative modification thereof; and a β chain variable region CDR3 comprising the amino acid sequence set forth in SEQ ID NO: 351 or a conservative modification thereof. In certain embodiments, the extracellular domain of the TCR further comprises an α chain variable region CDR2 comprising the amino acid sequence set forth in SEQ ID NO: 196 or a conservative modification thereof; and a β chain variable region CDR2 comprising the amino acid sequence set forth in SEQ ID NO: 350 or a conservative modification thereof. In certain embodiments, the extracellular domain of the TCR further comprises an α chain variable region CDR1 comprising the amino acid sequence set forth in SEQ ID NO: 195 or a conservative modification thereof; and a β chain variable region CDR1 comprising the amino acid sequence set forth in SEQ ID NO: 182 or a conservative modification thereof.

In certain embodiments, the extracellular domain of the TCR comprises an α chain variable region CDR3 comprising the amino acid sequence set forth in SEQ ID NO: 360 or a conservative modification thereof; and a β chain variable region CDR3 comprising the amino acid sequence set forth in SEQ ID NO: 361 or a conservative modification thereof. In certain embodiments, the extracellular domain of the TCR further comprises an α chain variable region CDR2 comprising the amino acid sequence set forth in SEQ ID NO: 180 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 179 or a conservative modification thereof; and a β chain variable region CDR1 comprising the amino acid sequence set forth in SEQ ID NO: 37 or a conservative modification thereof.

In certain embodiments, the extracellular domain of the TCR comprises an α chain variable region CDR3 comprising the amino acid sequence set forth in SEQ ID NO: 325 or a conservative modification thereof; and a β chain variable region CDR3 comprising the amino acid sequence set forth in SEQ ID NO: 290 or a conservative modification thereof. In certain embodiments, the extracellular domain of the TCR further comprises an α chain variable region CDR2 comprising the amino acid sequence set forth in SEQ ID NO: 286 or a conservative modification thereof; and a β chain variable region CDR2 comprising the amino acid sequence set forth in SEQ ID NO: 327 or a conservative modification thereof. In certain embodiments, the extracellular domain of the TCR further comprises an α chain variable region CDR1 comprising the amino acid sequence set forth in SEQ ID NO: 285 or a conservative modification thereof; and a β chain variable region CDR1 comprising the amino acid sequence set forth in SEQ ID NO: 326 or a conservative modification thereof.

In certain embodiments, the extracellular domain of the TCR comprises an α chain variable region CDR3 comprising the amino acid sequence set forth in SEQ ID NO: 371 or a conservative modification thereof; and a β chain variable region CDR3 comprising the amino acid sequence set forth in SEQ ID NO: 290 or a conservative modification thereof. In certain embodiments, the extracellular domain of the TCR further comprises an α chain variable region CDR2 comprising the amino acid sequence set forth in SEQ ID NO: 370 or a conservative modification thereof; and a β chain variable region CDR2 comprising the amino acid sequence set forth in SEQ ID NO: 327 or a conservative modification thereof. In certain embodiments, the extracellular domain of the TCR further comprises an α chain variable region CDR1 comprising the amino acid sequence set forth in SEQ ID NO: 369 or a conservative modification thereof; and a β chain variable region CDR1 comprising the amino acid sequence set forth in SEQ ID NO: 326 or a conservative modification thereof.

In certain embodiments, the extracellular domain of the TCR comprises an α chain variable region CDR3 comprising the amino acid sequence set forth in SEQ ID NO: 377 or a conservative modification thereof; and a β chain variable region CDR3 comprising the amino acid sequence set forth in SEQ ID NO: 378 or a conservative modification thereof. In certain embodiments, the extracellular domain of the TCR further comprises an α chain variable region CDR2 comprising the amino acid sequence set forth in SEQ ID NO: 376 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 375 or a conservative modification thereof; and a β chain variable region CDR1 comprising the amino acid sequence set forth in SEQ ID NO: 37 or a conservative modification thereof.

In certain embodiments, the extracellular domain of the TOR comprises an α chain variable region CDR3 comprising the amino acid sequence set forth in SEQ ID NO: 383 or a conservative modification thereof; and a β chain variable region CDR3 comprising the amino acid sequence set forth in SEQ ID NO: 378 or a conservative modification thereof. In certain embodiments, the extracellular domain of the TOR further comprises an α chain variable region CDR2 comprising the amino acid sequence set forth in SEQ ID NO: 180 or a conservative modification thereof; and a β chain variable region CDR2 comprising the amino acid sequence set forth in SEQ ID NO: 38 or a conservative modification thereof. In certain embodiments, the extracellular domain of the TCR further comprises an α chain variable region CDR1 comprising the amino acid sequence set forth in SEQ ID NO: 179 or a conservative modification thereof; and a β chain variable region CDR1 comprising the amino acid sequence set forth in SEQ ID NO: 37 or a conservative modification thereof.
5.3.4. TCRs with Modifications within CDRs

In certain embodiments, a presently disclosed TCR (or a functional fragment thereof) comprises an α chain variable region comprising CDR1, CDR2 and CDR3 sequences and a β chain variable region comprising CDR1, CDR2 and CDR3 sequences, wherein one or more of these CDR sequences comprise specified amino acid sequences based on the TCRs (or a functional fragments thereof) described herein (see Tables 1-51), or modifications thereof, and wherein the TCRs (or a functional fragments thereof) retain the desired functional properties of the mutant RAS peptide-specific TCRs (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCRs (or a functional fragments thereof) described herein (see Tables 1-51), or modifications thereof, and wherein the TCR (or a functional fragment thereof) retains the desired functional properties of the mutant RAS peptide-specific TCRs (or a functional fragments thereof) of the presently disclosed subject matter.

In certain embodiments, such modifications do not significantly affect or alter the binding characteristics of the TCR comprising the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 52. In certain embodiments, amino acid substitutions may be introduced into a TCR of interest and the products screened for a desired activity, e.g., retained/improved antigen binding, decreased immunogenicity, or improved ADCC or CDC.

TABLE 52

| | Exemplary conservative amino |
|---|---|
| Original Residue | acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006; 66(17):8878-8886; Cohen et al. Enhanced antitumor activity of T cells engineered to express T-cell receptors with a second disulfide bond, Cancer Res. 2007; 67(8):3898-3903; Kuball et al. Facilitating matched pairing and expression of TCR chains introduced into human T cells, Blood 2007; 109(6):2331-2338; Haga-Friedman et al. Incorporation of transmembrane hydrophobic mutations in the TCR enhance its surface expression and T cell functional avidity, Journal of immunology 2012; 188(11):5538-5546, the contents of each of which are incorporated by reference in their entireties).

### 5.3.5. Multispecific Molecules

The presently disclosed subject matter provides multispecific molecules comprising a presently disclosed TCR (or a functional fragment thereof). A presently disclosed TCR or a functional fragment thereof can be derivatized or linked to one or more functional molecules, e.g., one or more peptides or proteins (e.g., one or more antibodies or ligands for a receptor) to generate a multispecific molecule that binds to at least two different binding sites and/or target molecules. To create a multispecific molecule, a presently disclosed TCR or a functional fragment thereof can be functionally linked (e.g., by chemical coupling, genetic fusion, noncovalent association or otherwise) to one or more other binding molecules, such as one or more antibodies, antibody fragments, peptides or binding mimetic.

The presently disclosed subject matter provides multispecific molecules comprising at least a first binding specificity for a mutant RAS peptide and a second binding specificity for a second target peptide region. The second target epitope region can be a second RAS peptide, or a non-RAS peptide, e.g., a different antigen. In certain embodiments, the multi-specific molecule further comprises a third binding specificity. Where a first portion of a multispecific molecule, e.g., antibody, binds to an antigen on a tumor cell for example and a second portion of a multispecific molecule recognizes an antigen on the surface of a human immune effector cell, the multispecific molecule is capable of recruiting the activity of that effector cell by specifically binding to the effector antigen on the human immune effector cell. In certain embodiments, multispecific molecules are able to form a link between effector cells, for example, T cells and tumor cells, thereby enhancing effector function. In certain embodiments, a presently disclosed multispecific molecule comprises at least a first binding to a mutant RAS peptide and at least a second binding to an immune cell or a molecule associated with an immune cell.

The multispecific molecules of the presently disclosed subject matter can be prepared by conjugating the constituent binding specificities using methods known in the art. For example, each binding specificity of the multispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-malcimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multispecific molecule is a monoclonal antibody (mAb) and a mAb, a mAb and a Fab, a Fab and a F(ab′)₂, or a ligand and a Fab fusion protein.

Binding of the multispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography.

### 5.4. Cells

The presently disclosed subject matter provides cells comprising a presently disclosed TCR (e.g., one disclosed in Section 5.3). In certain embodiments, the cell is selected from the group consisting of cells of lymphoid lineage, and stem cells from which cells of lymphoid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, B cells, Natural Killer cells, and stem cells from which lymphoid cells may be differentiated. In certain embodiments, the stem cell is a pluripotent stem cell (e.g., embryonic stem cell, induced pluripotent stem cell (iPSC)).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the T cell is a γδ T cell. In certain embodiments, the T cell is a Natural killer T cell. In certain embodiments, the TCR-expressing T cells express Foxp3 to achieve and maintain a T regulatory phenotype.

In certain embodiments, the cell is a Natural Killer cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadclain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

In certain embodiments, cell further comprises at least one recombinant or exogenous co-stimulatory ligand. For example, a presently disclosed cell can be further transduced with at least one co-stimulatory ligand, such that the cell co-expresses or is induced to co-express the presently disclosed TCR and the at least one co-stimulatory ligand. The interaction between the presently disclosed TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type 11 transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNF-β)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins-they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the cell comprises one recombinant co-stimulatory ligand that is 4-1BBL. In certain embodiments, the cell comprises two recombinant co-stimulatory ligands that are 4-1BBL and CD80.

In certain embodiments, a presently disclosed cell further comprises at least one exogenous cytokine. For example, a presently disclosed cell can be further transduced with at least one cytokine, such that the cell secretes the at least one cytokine as well as expresses the presently disclosed TCR. In certain embodiments, the at least one cytokine is selected from the group consisting of IL-2, IL-3, IL-6, IL-7, IL-11, IL-12, IL-15, IL-17, IL-18, and IL-21. In certain embodiments, the cytokine is IL-12.

In certain embodiments, a presently disclosed cell further comprises at least one exogenous coreceptor. For example, a presently disclosed cell can be further transduced with at least one coreceptor, such that the cell co-expresses or is induced to co-express the presently disclosed TCR and the at least one coreceptor. In certain embodiments, the at least one coreceptors is selected from the group consisting of CD45, CD8, and CD4. In certain embodiments, the at least one coreceptor comprises CD8.

In certain embodiments, a presently disclosed cell further comprises at least one exogenous integrin. For example, a presently disclosed cell can be further transduced with at least one integrin, such that the cell co-expresses or is induced to co-express the presently disclosed TCR and the at least one integrin. In certain embodiments, the at least one integrins is selected from the group consisting of LFA-1 and VLA-4. In certain embodiments, the at least one integrin comprises LFA-1.

### 5.5. Nucleic Acids and Genetic Modifications of Cells

The present discloses subject matter provides a nucleic acid encoding a presently disclosed TCR (e.g., one disclosed in Section 5.3). Further provided are cells comprising such nucleic acids. In certain embodiments, a promoter is operably linked to the presently disclosed TCR.

In certain embodiments, the promoter is endogenous or exogenous. In certain embodiments, the exogenous promoter is selected from the group consisting of a long terminal repeat (LTR) promoter, an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiment, the exogenous promoter is a LTR promoter. In certain embodiments, the promoter is an inducible promoter. In certain embodiment, the inducible promoter is selected from the group consisting of a NFAT transcriptional response element (TRE) promoter, a CD69 promoter, a CD25 promoter, and an IL-2 promoter.

In certain embodiments, the nucleic acid encodes both the α chain and the β chain of a presently disclosed TCR. In certain embodiments, the α chain and the β chain are separated by a self-cleavage peptide, e.g., a 2A-peptide. In certain embodiments, the α chain and the β chain are separated by a furin-2A-peptide. In certain embodiments, the peptide comprises the amino acid sequence set forth in SEQ ID NO: 172.
RAKRSGSGATNFSLLKQAGDVEENPGP [SEQ ID NO: 172]

In certain embodiments, the nucleic acid encodes a functional portion/fragment of a presently disclosed TCR. As used herein, the term “functional portion” or “functional fragment” refers to any portion, part or fragment of a presently disclosed TCR, which portion, part or fragment retains the biological activity of the TCR (the parent TCR). For example, functional portions encompass the portions, parts or fragments of a presently disclosed TCR that retains the ability to recognize the RAS peptide (e.g., a RAS peptide comprising a Q61R mutation) to a similar, same, or even a higher extent as the parent TCR. In certain embodiments, the nucleic acid encoding a functional portion of a presently disclosed TCR encodes a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Genetic modification of a cell (e.g., a T cell) can be accomplished by transducing a substantially homogeneous cell composition with a recombinant DNA or RNA construct. In certain embodiments, a retroviral vector (e.g., gamma-retroviral vector or lentiviral vector) is employed for the introduction of the DNA or RNA construct into the cell. For example, a polynucleotide encoding a presently disclosed TCR can be cloned into a retroviral vector and expression can be driven from its endogenous promoter, from the retroviral long terminal repeat, or from an alternative internal promoter, or from a promoter specific for a target cell type of interest. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used. For initial genetic modification of a cell to include a presently disclosed TCR, a retroviral vector can be employed for transduction, however any other suitable viral vector or non-viral delivery system can be used. The TCR can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

In certain embodiments, a presently disclosed TCR can be integrated into a selected locus of the genome of a cell. Any targeted genome editing methods can also be used to deliver a presently disclosed TCR to a cell or a subject. In certain embodiments, a CRISPR system is used to deliver a presently disclosed TCR. In certain embodiments, zinc-finger nucleases are used to deliver presently disclosed TCR. In certain embodiments, a TALEN system is used to deliver a presently disclosed TCR.

In certain embodiments, a presently disclosed TCR can be integrated at a locus encoding a T cell receptor. Non-limiting examples of the loci include a TRAC locus, a TRBC locus, a TRDC locus, and a TRGC locus. In certain embodiments, the locus is a TRAC locus or a TRBC locus. Methods of targeting a TCR to a site within the genome of T cell can be found in WO2017180989 and Eyquem et al., Nature. (2017 Mar. 2); 543(7643): 113-117, both of which are incorporated by reference in their entireties. In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of a cell. In certain embodiments, the CRISPR system uses a DNA donor-template guided homology directed repair at a defined genetic locus, e.g., a TRAC locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. In certain embodiments, CRISPR/Cas9 is a recombinant ribonucleoprotein complex that is transfected into target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of a cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR) In certain embodiments, the modification is introduced downstream of the protein coding region of the integrated TCR.

### 5.6. Formulations and Administration

The presently disclosed subject matter also provides compositions comprising the presently disclosed cells (e.g., those disclosed in Section 5.4). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a tumor. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹¹, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, at least about 1×10⁵ cells can be administered, eventually reaching about 1×10¹⁰ or more. In certain embodiments, at least about 1×10⁶ cells can be administered. In certain embodiments, from about 10⁴ to about 10¹¹, from about 10⁵ to about 10⁹, or from about 10⁶ to about 10⁸ the presently disclosed cells are administered to a subject. More effective cells may be administered in even smaller numbers. In certain embodiments, at least about 1×10⁸, about 2×10⁸, about 3×10⁸, about 4×10⁸, and about 5× 10⁸ the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosscous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor.

In certain embodiments, each of the above-noted methods comprises administering the presently disclosed cells or a composition (e.g., a pharmaceutical composition) comprising thereof to achieve the desired effect, e.g., palliation of an existing condition or prevention of recurrence of tumor. For treatment, the amount administered is an amount effective in producing the desired effect. An effective amount can be provided in one or a series of administrations. An effective amount can be provided in a bolus or by continuous perfusion.

In certain embodiments, the tumor is associated with RAS. In certain embodiments, the tumor is associated with a RAS mutation or a RAS mutant. In certain embodiments, the RAS mutation is a Q61 mutation. In certain embodiments, the RAS mutation is a Q61R mutation. In certain embodiments, the RAS mutation is a Q61K mutation. In certain embodiments, the RAS mutation is a Q61L mutation.

In certain embodiments, the tumor is a cancer. In certain embodiments, the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer (also known as “stomach cancer”), neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma. In certain embodiments, the tumor is colorectal cancer. In certain embodiments, the tumor is thyroid cancer. In certain embodiments, the tumor is cutaneous melanoma.

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

### 5.8. Diagnostic and Prognostic Methods

The presently disclosed TCR, multi-specific molecules, and nucleic acids encode thereof can be used for diagnostic and prognostic applications as well as use as research tools for detection of RAS in a biological sample, in a cell, a tissue, or a blood sample. The presently disclosed subject matter provides methods for detecting RAS in a cell, a tissue, or a blood sample. In certain embodiments, the method comprises: contacting a cell, a tissue, or a blood sample with presently disclosed TCR (or a functional fragment thereof) or multi-specific molecule disclosed herein, wherein the TCR or multi-specific molecule comprises a detectable label; and determining the amount of the labeled TCR or multi-specific molecule bound to the cell, tissue, or blood sample by measuring the amount of detectable label associated with the cell or tissue, wherein the amount of bound TCR or multi-specific molecule indicates the amount of RAS in the cell, tissue, or a blood sample. The cell or tissue can be any cell or tissue, including any normal, healthy, or cancerous cells and tissues. In certain embodiments, the blood sample is a peripheral blood sample.

The presently disclosed TCR (or a functional fragment thereof) can be used in methods known in the art relating to the localization and/or quantitation of RAS polypeptides (e.g., for use in measuring levels of the RAS protein within appropriate physiological samples, for use in diagnostic methods, for use in imaging the polypeptide, and the like). The presently disclosed TCR (or a functional fragment thereof) can be used to isolate a cell including a RAS polypeptide by standard techniques, such as affinity chromatography or immunoprecipitation. Moreover, the presently disclosed TCR (or a functional fragment thereof) can be used to detect an immunoreactive RAS protein (e.g., in plasma, a cellular lysate or cell supernatant) in order to evaluate the abundance and pattern of expression of the immunoreactive polypeptide. the presently disclosed TCR (or a functional fragment thereof) can be used diagnostically to monitor immunoreactive RAS protein levels in tissue as part of a clinical testing procedure, e.g., to determine the efficacy of a given treatment regimen. As noted above, the detection can be facilitated by coupling (i.e., physically linking) the presently disclosed TCR (or a functional fragment thereof) to a detectable substance.

An exemplary method for detecting the presence or absence of an immunoreactive RAS protein in a biological sample comprises contacting a biological sample from a subject with the presently disclosed TCR (or a functional fragment thereof), wherein the presence of an immunoreactive RAS protein is detected in the biological sample. Detection may be accomplished by means of a detectable label attached to the antibody.

The term “labeled” with regard to the presently disclosed TCR (or a functional fragment thereof) is intended to encompass direct labeling of the TCR by coupling (i.e., physically linking) a detectable substance to the antibody, as well as indirect labeling of the antibody by reactivity with another compound that is directly labeled, such as a secondary antibody.

In certain embodiments, the presently disclosed TCR (or a functional fragment thereof) is conjugated to one or more detectable labels. For such uses, the presently disclosed TCR (or a functional fragment thereof) may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive RAS protein in a biological sample in vitro as well as in vivo. Non-limiting examples of in vitro techniques for detection of an immunoreactive RAS protein include enzyme linked immunosorbent assays (ELISAs), Western blots, immunoprecipitations, radioimmunoassay, and immunofluorescence. Furthermore, in vivo techniques for detection of an immunoreactive RAS protein include introducing into a subject a labeled TCR (or a functional fragment thereof). For example, the presently disclosed TCR (or a functional fragment thereof) can be labeled with a radioactive marker whose presence and location in a subject can be detected by standard imaging techniques. In certain embodiments, the biological sample comprises RAS protein molecules from the test subject.

The presently disclosed TCR (or a functional fragment thereof) can be used to assay immunoreactive RAS protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive RAS protein levels in a biological sample, the presently disclosed TCR (or a functional fragment thereof) may be used for in vivo imaging of RAS. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the presently disclosed TCR (or a functional fragment thereof).

The presently disclosed TCR (or a functional fragment thereof), which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries. The labeled TCR (or a functional fragment thereof) then accumulates at the location of cells which contain the specific target polypeptide. For example, the presently disclosed TCR (or a functional fragment thereof) accumulate within the subject in cells and tissues in which the RAS protein has localized.

Thus, the presently disclosed subject matter provides diagnostic methods of a medical condition. In certain embodiments, the method comprises: (a) assaying the expression of immunoreactive RAS protein by measuring binding of a presently disclosed TCR (or a functional fragment thereof) in cells or body fluid of an individual; and (b) comparing the amount of immunoreactive RAS protein present in the sample with a standard reference, wherein an increase or decrease in immunoreactive RAS protein levels compared to the standard is indicative of a medical condition.

Furthermore, the presently disclosed TCR (or a functional fragment thereof) may be used to purify cells including a RAS protein from a sample. In certain embodiments, the TCRs are immobilized on a solid support. Non-limiting examples of such solid supports include plastics such as polycarbonate, complex carbohydrates such as agarose and sepharose, acrylic resins and such as polyacrylamide and latex beads. Techniques for coupling TCRs to such solid supports are well known in the art.

A TCR or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (Sec, e.g., Wong (1991), supra; and Hermanson (1996), supra).

A TCR or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. A TCR or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed TCR (or a functional fragment thereof) is useful in diagnostic methods. As such, the presently disclosed subject matter provides methods using the presently disclosed TCR (or a functional fragment thereof) in diagnosis of RAS activity in a subject. The presently disclosed TCR (or a functional fragment thereof) may be selected such that they have any level of epitope binding specificity and high binding affinity to a RAS polypeptide.

The presently disclosed TCR (or a functional fragment thereof) can be used to detect an immunoreactive RAS protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of RAS protein in a sample obtained from the subject. In certain embodiments, the sample is selected from the group consisting of urine, blood, serum, plasma, saliva, amniotic fluid, cerebrospinal fluid (CSF), and biopsied body tissue.

In certain embodiments, the presently disclosed TCR (or a functional fragment thereof) is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed TCR (or a functional fragment thereof) using standard chemistries. The chelate is normally linked to the antibody by a group which enables formation of a bond to the molecule with minimal loss of immunoreactivity and minimal aggregation and/or internal cross-linking.

### 5.9. Kits

The presently disclosed subject matter provides kits for treatment or ameliorating a disease or disorder associated with RAS (e.g., a metastatic cancer cell), and/or detecting RAS. In certain embodiments, the kit comprises the presently disclosed TCR (or a functional fragment thereof), the cells, the multi-specific molecule, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the presently disclosed TCR (or a functional fragment thereof), the cells, the multi-specific molecule, or the composition disclosed herein to a subject in need the treatment. The instructions can generally include information about the use of the presently disclosed TCR (or a functional fragment thereof), the cells, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides a T cell receptor (TCR) that binds to a RAS peptide, wherein the RAS peptide comprises a Q61 mutation.

A2. The foregoing TCR of A1, wherein the RAS peptide comprises a Q61R mutation, a Q61K mutation, or a Q61L mutation.

A3. The foregoing TCR of A1 or A2, wherein the RAS peptide comprises or consists of the amino acid sequence set forth in SEQ ID NO: 1, SEQ ID NO: 2, or SEQ ID NO: 3.

A4. The foregoing TCR of any one of A1-A3, wherein the RAS peptide comprises or consists of the amino acid sequence set forth in SEQ ID NO: 1.

A5. The foregoing TCR of any one of A1-A3, wherein the RAS peptide comprises or consists of the amino acid sequence set forth in SEQ ID NO: 2.

A6. The foregoing TCR of any one of A1-A3, wherein the RAS peptide comprises or consists of the amino acid sequence set forth in SEQ ID NO: 3.

A7. The foregoing TCR of any one of A1-A6, wherein the RAS peptide is associated with an HLA class I complex.

A8. The foregoing TCR of A7, wherein the HLA class I complex is selected from an HLA-A, an HLA-B, and an HLA-C.

A9. The foregoing TCR of A7 or A8, wherein the HLA class I complex is an HLA-A.

A10. The foregoing TCR of A7 or A8, wherein the HLA-A is an HLA-A*01 superfamily member.

All. The foregoing TCR of A10, wherein the HLA-A*01 superfamily member is selected from the group consisting of HLA-A*01, HLA-A*25, HLA-A*26, HLA-A*30, HLA-A*31, HLA-A*32, HLA-A*36, HLA-A*74:10, and HLA-A*80.

A12. The foregoing TCR of A10 or A11, wherein the HLA-A*01 superfamily member is HLA-A*01:01.

A13. The foregoing TCR of any one of A1-A12, wherein the TCR comprises an extracellular domain that binds to the RAS peptide, wherein the extracellular domain comprises an α chain and a β chain, wherein the α chain comprises an α chain variable region and α chain constant region, and the β chain comprises a β chain variable region and a β chain constant region.

A14. The foregoing TCR of A13, wherein:
a) the α chain variable region comprises a CDR3 comprising the amino acid sequence set forth in SEQ ID NO: 7 or a conservative modification thereof, and the β chain variable region comprises a CDR3 comprising the amino acid sequence set forth in SEQ ID NO: 10 or a conservative modification thereof;
b) the α chain variable region comprises a CDR3 comprising the amino acid sequence set forth in SEQ ID NO: 17 or a conservative modification thereof, and the β chain variable region comprises a CDR3 comprising the amino acid sequence set forth in SEQ ID NO: 19 or a conservative modification thereof;
c) the α chain variable region comprises a CDR3 comprising the amino acid sequence set forth in SEQ ID NO: 26 or a conservative modification thereof, and the β chain variable region comprises a CDR3 comprising the amino acid sequence set forth in SEQ ID NO: 29 or a conservative modification thereof;
d) the α chain variable region comprises a CDR3 comprising the amino acid sequence set forth in SEQ ID NO: 36 or a conservative modification thereof, and the β chain variable region comprises a CDR3 comprising the amino acid sequence set forth in SEQ ID NO: 39 or a conservative modification thereof;
e) the α chain variable region comprises a CDR3 comprising the amino acid sequence set forth in SEQ ID NO: 44 or a conservative modification thereof, and the β chain variable region comprises a CDR3 comprising the amino acid sequence set forth in SEQ ID NO: 39 or a conservative modification thereof;
f) the α chain variable region comprises a CDR3 comprising the amino acid sequence set forth in SEQ ID NO: 47 or a conservative modification thereof, and the β chain variable region comprises a CDR3 comprising the amino acid sequence set forth in SEQ ID NO: 50 or a conservative modification thereof;
g) the α chain variable region comprises a CDR3 comprising the amino acid sequence set forth in SEQ ID NO: 55 or a conservative modification thereof, and the β chain variable region comprises a CDR3 comprising the amino acid sequence set forth in SEQ ID NO: 56 or a conservative modification thereof;
h) the α chain variable region comprises a CDR3 comprising the amino acid sequence set forth in SEQ ID NO: 55 or a conservative modification thereof, and the β chain variable region comprises a CDR3 comprising the amino acid sequence set forth in SEQ ID NO: 61 or a conservative modification thereof;
i) the α chain variable region comprises a CDR3 comprising the amino acid sequence set forth in SEQ ID NO: 66 or a conservative modification thereof, and the β chain variable region comprises a CDR3 comprising the amino acid sequence set forth in SEQ ID NO: 67 or a conservative modification thereof;
j) the α chain variable region comprises a CDR3 comprising the amino acid sequence set forth in SEQ ID NO: 74 or a conservative modification thereof, and the β chain variable region comprises a CDR3 comprising the amino acid sequence set forth in SEQ ID NO: 67 or a conservative modification thereof;
k) the α chain variable region comprises a CDR3 comprising the amino acid sequence set forth in SEQ ID NO: 77 or a conservative modification thereof, and the β chain variable region comprises a CDR3 comprising the amino acid sequence set forth in SEQ ID NO: 80 or a conservative modification thereof;
l) the α chain variable region comprises a CDR3 comprising the amino acid sequence set forth in SEQ ID NO: 87 or a conservative modification thereof, and the β chain variable region comprises a CDR3 comprising the amino acid sequence set forth in SEQ ID NO: 88 or a conservative modification thereof;
m) the α chain variable region comprises a CDR3 comprising the amino acid sequence set forth in SEQ ID NO: 95 or a conservative modification thereof, and the β chain variable region comprises a CDR3 comprising the amino acid sequence set forth in SEQ ID NO: 96 or a conservative modification thereof;
n) the α chain variable region comprises a CDR3 comprising the amino acid sequence set forth in SEQ ID NO: 101 or a conservative modification thereof, and the β chain variable region comprises a CDR3 comprising the amino acid sequence set forth in SEQ ID NO: 104 or a conservative modification thereof;
o) the α chain variable region comprises a CDR3 comprising the amino acid sequence set forth in SEQ ID NO: 111 or a conservative modification thereof, and the β chain variable region comprises a CDR3 comprising the amino acid sequence set forth in SEQ ID NO: 114 or a conservative modification thereof;
p) the α chain variable region comprises a CDR3 comprising the amino acid sequence set forth in SEQ ID NO: 121 or a conservative modification thereof, and the β chain variable region comprises a CDR3 comprising the amino acid sequence set forth in SEQ ID NO: 123 or a conservative modification thereof;
q) the α chain variable region comprises a CDR3 comprising the amino acid sequence set forth in SEQ ID NO: 130 or a conservative modification thereof, and the β chain variable region comprises a CDR3 comprising the amino acid sequence set forth in SEQ ID NO: 133 or a conservative modification thereof;
r) the α chain variable region comprises a CDR3 comprising the amino acid sequence set forth in SEQ ID NO: 139 or a conservative modification thereof, and the β chain variable region comprises a CDR3 comprising the amino acid sequence set forth in SEQ ID NO: 140 or a conservative modification thereof;
s) the α chain variable region comprises a CDR3 comprising the amino acid sequence set forth in SEQ ID NO: 147 or a conservative modification thereof, and the β chain variable region comprises a CDR3 comprising the amino acid sequence set forth in SEQ ID NO: 140 or a conservative modification thereof;
t) the α chain variable region comprises a CDR3 comprising the amino acid sequence set forth in SEQ ID NO: 150 or a conservative modification thereof, and the β chain variable region comprises a CDR3 comprising the amino acid sequence set forth in SEQ ID NO: 151 or a conservative modification thereof;
u) the α chain variable region comprises a CDR3 comprising the amino acid sequence set forth in SEQ ID NO: 158 or a conservative modification thereof, and the β chain variable region comprises a CDR3 comprising the amino acid sequence set forth in SEQ ID NO: 161 or a conservative modification thereof;
v) the α chain variable region comprises a CDR3 comprising the amino acid sequence set forth in SEQ ID NO: 173 or a conservative modification thereof, and the β chain variable region comprises a CDR3 comprising the amino acid sequence set forth in SEQ ID NO: 174 or a conservative modification thereof;
w) the α chain variable region comprises a CDR3 comprising the amino acid sequence set forth in SEQ ID NO: 181 or a conservative modification thereof, and the β chain variable region comprises a CDR3 comprising the amino acid sequence set forth in SEQ ID NO: 184 or a conservative modification thereof;
x) the α chain variable region comprises a CDR3 comprising the amino acid sequence set forth in SEQ ID NO: 189 or a conservative modification thereof, and the β chain variable region comprises a CDR3 comprising the amino acid sequence set forth in SEQ ID NO: 190 or a conservative modification thereof;
y) the α chain variable region comprises a CDR3 comprising the amino acid sequence set forth in SEQ ID NO: 197 or a conservative modification thereof, and the β chain variable region comprises a CDR3 comprising the amino acid sequence set forth in SEQ ID NO: 198 or a conservative modification thereof;
z) the α chain variable region comprises a CDR3 comprising the amino acid sequence set forth in SEQ ID NO: 205 or a conservative modification thereof, and the β chain variable region comprises a CDR3 comprising the amino acid sequence set forth in SEQ ID NO: 206 or a conservative modification thereof;
aa) the α chain variable region comprises a CDR3 comprising the amino acid sequence set forth in SEQ ID NO: 213 or a conservative modification thereof, and the β chain variable region comprises a CDR3 comprising the amino acid sequence set forth in SEQ ID NO: 216 or a conservative modification thereof;
ab) the α chain variable region comprises a CDR3 comprising the amino acid sequence set forth in SEQ ID NO: 221 or a conservative modification thereof, and the β chain variable region comprises a CDR3 comprising the amino acid sequence set forth in SEQ ID NO: 222 or a conservative modification thereof;
ac) the α chain variable region comprises a CDR3 comprising the amino acid sequence set forth in SEQ ID NO: 229 or a conservative modification thereof, and the β chain variable region comprises a CDR3 comprising the amino acid sequence set forth in SEQ ID NO: 232 or a conservative modification thereof;
ad) the α chain variable region comprises a CDR3 comprising the amino acid sequence set forth in SEQ ID NO: 237 or a conservative modification thereof, and the β chain variable region comprises a CDR3 comprising the amino acid sequence set forth in SEQ ID NO: 240 or a conservative modification thereof;
ae) the α chain variable region comprises a CDR3 comprising the amino acid sequence set forth in SEQ ID NO: 245 or a conservative modification thereof, and the β chain variable region comprises a CDR3 comprising the amino acid sequence set forth in SEQ ID NO: 248 or a conservative modification thereof;
af) the α chain variable region comprises a CDR3 comprising the amino acid sequence set forth in SEQ ID NO: 254 or a conservative modification thereof, and the β chain variable region comprises a CDR3 comprising the amino acid sequence set forth in SEQ ID NO: 255 or a conservative modification thereof;
ag) the α chain variable region comprises a CDR3 comprising the amino acid sequence set forth in SEQ ID NO: 262 or a conservative modification thereof, and the β chain variable region comprises a CDR3 comprising the amino acid sequence set forth in SEQ ID NO: 265 or a conservative modification thereof;
ah) the α chain variable region comprises a CDR3 comprising the amino acid sequence set forth in SEQ ID NO: 270 or a conservative modification thereof, and the β chain variable region comprises a CDR3 comprising the amino acid sequence set forth in SEQ ID NO: 273 or a conservative modification thereof;
ai) the α chain variable region comprises a CDR3 comprising the amino acid sequence set forth in SEQ ID NO: 278 or a conservative modification thereof, and the β chain variable region comprises a CDR3 comprising the amino acid sequence set forth in SEQ ID NO: 280 or a conservative modification thereof;
aj) the α chain variable region comprises a CDR3 comprising the amino acid sequence set forth in SEQ ID NO: 287 or a conservative modification thereof, and the β chain variable region comprises a CDR3 comprising the amino acid sequence set forth in SEQ ID NO: 290 or a conservative modification thereof;
ak) the α chain variable region comprises a CDR3 comprising the amino acid sequence set forth in SEQ ID NO: 295 or a conservative modification thereof, and the β chain variable region comprises a CDR3 comprising the amino acid sequence set forth in SEQ ID NO: 298 or a conservative modification thereof;
al) the α chain variable region comprises a CDR3 comprising the amino acid sequence set forth in SEQ ID NO: 303 or a conservative modification thereof, and the β chain variable region comprises a CDR3 comprising the amino acid sequence set forth in SEQ ID NO: 306 or a conservative modification thereof;
am) the α chain variable region comprises a CDR3 comprising the amino acid sequence set forth in SEQ ID NO: 311 or a conservative modification thereof, and the β chain variable region comprises a CDR3 comprising the amino acid sequence set forth in SEQ ID NO: 312 or a conservative modification thereof;
an) the α chain variable region comprises a CDR3 comprising the amino acid sequence set forth in SEQ ID NO: 319 or a conservative modification thereof, and the β chain variable region comprises a CDR3 comprising the amino acid sequence set forth in SEQ ID NO: 320 or a conservative modification thereof;
ao) the α chain variable region comprises a CDR3 comprising the amino acid sequence set forth in SEQ ID NO: 325 or a conservative modification thereof, and the β chain variable region comprises a CDR3 comprising the amino acid sequence set forth in SEQ ID NO: 328 or a conservative modification thereof;
ap) the α chain variable region comprises a CDR3 comprising the amino acid sequence set forth in SEQ ID NO: 333 or a conservative modification thereof, and the β chain variable region comprises a CDR3 comprising the amino acid sequence set forth in SEQ ID NO: 334 or a conservative modification thereof;
aq) the α chain variable region comprises a CDR3 comprising the amino acid sequence set forth in SEQ ID NO: 339 or a conservative modification thereof, and the β chain variable region comprises a CDR3 comprising the amino acid sequence set forth in SEQ ID NO: 340 or a conservative modification thereof;
ar) the α chain variable region comprises a CDR3 comprising the amino acid sequence set forth in SEQ ID NO: 345 or a conservative modification thereof, and the β chain variable region comprises a CDR3 comprising the amino acid sequence set forth in SEQ ID NO: 340 or a conservative modification thereof;
as) the α chain variable region comprises a CDR3 comprising the amino acid sequence set forth in SEQ ID NO: 349 or a conservative modification thereof, and the β chain variable region comprises a CDR3 comprising the amino acid sequence set forth in SEQ ID NO: 351 or a conservative modification thereof;
at) the α chain variable region comprises a CDR3 comprising the amino acid sequence set forth in SEQ ID NO: 356 or a conservative modification thereof, and the β chain variable region comprises a CDR3 comprising the amino acid sequence set forth in SEQ ID NO: 351 or a conservative modification thereof;
au) the α chain variable region comprises a CDR3 comprising the amino acid sequence set forth in SEQ ID NO: 360 or a conservative modification thereof, and the β chain variable region comprises a CDR3 comprising the amino acid sequence set forth in SEQ ID NO: 361 or a conservative modification thereof;
av) the α chain variable region comprises a CDR3 comprising the amino acid sequence set forth in SEQ ID NO: 325 or a conservative modification thereof, and the β chain variable region comprises a CDR3 comprising the amino acid sequence set forth in SEQ ID NO: 290 or a conservative modification thereof;
aw) the α chain variable region comprises a CDR3 comprising the amino acid sequence set forth in SEQ ID NO: 371 or a conservative modification thereof, and the β chain variable region comprises a CDR3 comprising the amino acid sequence set forth in SEQ ID NO: 290 or a conservative modification thereof;
ax) the α chain variable region comprises a CDR3 comprising the amino acid sequence set forth in SEQ ID NO: 377 or a conservative modification thereof, and the β chain variable region comprises a CDR3 comprising the amino acid sequence set forth in SEQ ID NO: 378 or a conservative modification thereof; or
ay) the α chain variable region comprises a CDR3 comprising the amino acid sequence set forth in SEQ ID NO: 383 or a conservative modification thereof, and the β chain variable region comprises a CDR3 comprising the amino acid sequence set forth in SEQ ID NO: 378 or a conservative modification thereof.

A15. The foregoing TCR of A13 or A14, wherein:
a) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 9 or a conservative modification thereof;
b) the α chain variable region comprises a CDR2 comprising the amino acid sequence set forth in SEQ ID NO: 16 or a conservative modification thereof, and the β chain variable region comprises a CDR2 comprising the amino acid sequence set forth in SEQ ID NO: 18 or a conservative modification thereof;
c) the α chain variable region comprises a CDR2 comprising the amino acid sequence set forth in SEQ ID NO: 25 or a conservative modification thereof, and the β chain variable region comprises a CDR2 comprising the amino acid sequence set forth in SEQ ID NO: 28 or a conservative modification thereof;
d) the α chain variable region comprises a CDR2 comprising the amino acid sequence set forth in SEQ ID NO: 35 or a conservative modification thereof, and the β chain variable region comprises a CDR2 comprising the amino acid sequence set forth in SEQ ID NO: 38 or a conservative modification thereof;
e) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 49 or a conservative modification thereof;
f) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 38 or a conservative modification thereof;
g) the α chain variable region comprises a CDR2 comprising the amino acid sequence set forth in SEQ ID NO: 65 or a conservative modification thereof, and the β chain variable region comprises a CDR2 comprising the amino acid sequence set forth in SEQ ID NO: 49 or a conservative modification thereof;
h) the α chain variable region comprises a CDR2 comprising the amino acid sequence set forth in SEQ ID NO: 73 or a conservative modification thereof, and the β chain variable region comprises a CDR2 comprising the amino acid sequence set forth in SEQ ID NO: 49 or a conservative modification thereof;
i) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 79 or a conservative modification thereof;
j) the α chain variable region comprises a CDR2 comprising the amino acid sequence set forth in SEQ ID NO: 86 or a conservative modification thereof, and the β chain variable region comprises a CDR2 comprising the amino acid sequence set forth in SEQ ID NO: 38 or a conservative modification thereof;
k) the α chain variable region comprises a CDR2 comprising the amino acid sequence set forth in SEQ ID NO: 94 or a conservative modification thereof, and the β chain variable region comprises a CDR2 comprising the amino acid sequence set forth in SEQ ID NO: 49 or a conservative modification thereof;
l) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 103 or a conservative modification thereof;
m) the α chain variable region comprises a CDR2 comprising the amino acid sequence set forth in SEQ ID NO: 110 or a conservative modification thereof, and the β chain variable region comprises a CDR2 comprising the amino acid sequence set forth in SEQ ID NO: 113 or a conservative modification thereof;
n) the α chain variable region comprises a CDR2 comprising the amino acid sequence set forth in SEQ ID NO: 120 or a conservative modification thereof, and the β chain variable region comprises a CDR2 comprising the amino acid sequence set forth in SEQ ID NO: 113 or a conservative modification thereof;
o) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 132 or a conservative modification thereof;
p) the α chain variable region comprises a CDR2 comprising the amino acid sequence set forth in SEQ ID NO: 35 or a conservative modification thereof, and the β chain variable region comprises a CDR2 comprising the amino acid sequence set forth in SEQ ID NO: 49 or a conservative modification thereof;
q) the α chain variable region comprises a CDR2 comprising the amino acid sequence set forth in SEQ ID NO: 146 or a conservative modification thereof, and the β chain variable region comprises a CDR2 comprising the amino acid sequence set forth in SEQ ID NO: 49 or a conservative modification thereof;
r) the α chain variable region comprises a CDR2 comprising the amino acid sequence set forth in SEQ ID NO: 157 or a conservative modification thereof, and the β chain variable region comprises a CDR2 comprising the amino acid sequence set forth in SEQ ID NO: 160 or a conservative modification thereof;
s) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 183 or a conservative modification thereof;
t) the α chain variable region comprises a CDR2 comprising the amino acid sequence set forth in SEQ ID NO: 120 or a conservative modification thereof, and the β chain variable region comprises a CDR2 comprising the amino acid sequence set forth in SEQ ID NO: 38 or a conservative modification thereof;
u) the α chain variable region comprises a CDR2 comprising the amino acid sequence set forth in SEQ ID NO: 196 or a conservative modification thereof, and the β chain variable region comprises a CDR2 comprising the amino acid sequence set forth in SEQ ID NO: 160 or a conservative modification thereof;
v) the α chain variable region comprises a CDR2 comprising the amino acid sequence set forth in SEQ ID NO: 204 or a conservative modification thereof, and the β chain variable region comprises a CDR2 comprising the amino acid sequence set forth in SEQ ID NO: 113 or a conservative modification thereof;
w) the α chain variable region comprises a CDR2 comprising the amino acid sequence set forth in SEQ ID NO: 212 or a conservative modification thereof, and the β chain variable region comprises a CDR2 comprising the amino acid sequence set forth in SEQ ID NO: 215 or a conservative modification thereof;
x) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 160 or a conservative modification thereof;
y) the α chain variable region comprises a CDR2 comprising the amino acid sequence set forth in SEQ ID NO: 228 or a conservative modification thereof, and the β chain variable region comprises a CDR2 comprising the amino acid sequence set forth in SEQ ID NO: 231 or a conservative modification thereof;
z) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 239 or a conservative modification thereof;
aa) the α chain variable region comprises a CDR2 comprising the amino acid sequence set forth in SEQ ID NO: 73 or a conservative modification thereof, and the β chain variable region comprises a CDR2 comprising the amino acid sequence set forth in SEQ ID NO: 247 or a conservative modification thereof;
ab) the α chain variable region comprises a CDR2 comprising the amino acid sequence set forth in SEQ ID NO: 253 or a conservative modification thereof, and the β chain variable region comprises a CDR2 comprising the amino acid sequence set forth in SEQ ID NO: 248 or a conservative modification thereof;
ac) the α chain variable region comprises a CDR2 comprising the amino acid sequence set forth in SEQ ID NO: 254 or a conservative modification thereof, and the β chain variable region comprises a CDR2 comprising the amino acid sequence set forth in SEQ ID NO: 38 or a conservative modification thereof;
ad) the α chain variable region comprises a CDR2 comprising the amino acid sequence set forth in SEQ ID NO: 261 or a conservative modification thereof, and the β chain variable region comprises a CDR2 comprising the amino acid sequence set forth in SEQ ID NO: 264 or a conservative modification thereof;
ae) the α chain variable region comprises a CDR2 comprising the amino acid sequence set forth in SEQ ID NO: 204 or a conservative modification thereof, and the β chain variable region comprises a CDR2 comprising the amino acid sequence set forth in SEQ ID NO: 272 or a conservative modification thereof;
af) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 279 or a conservative modification thereof;
ag) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 289 or a conservative modification thereof;
ah) the α chain variable region comprises a CDR2 comprising the amino acid sequence set forth in SEQ ID NO: 228 or a conservative modification thereof, and the β chain variable region comprises a CDR2 comprising the amino acid sequence set forth in SEQ ID NO: 297 or a conservative modification thereof;
ai) the α chain variable region comprises a CDR2 comprising the amino acid sequence set forth in SEQ ID NO: 6 or a conservative modification thereof, and the β chain variable region comprises a CDR2 comprising the amino acid sequence set forth in SEQ ID NO: 305 or a conservative modification thereof;
aj) the α chain variable region comprises a CDR2 comprising the amino acid sequence set forth in SEQ ID NO: 129 or a conservative modification thereof, and the β chain variable region comprises a CDR2 comprising the amino acid sequence set forth in SEQ ID NO: 28 or a conservative modification thereof;
ak) the α chain variable region comprises a CDR2 comprising the amino acid sequence set forth in SEQ ID NO: 318 or a conservative modification thereof, and the β chain variable region comprises a CDR2 comprising the amino acid sequence set forth in SEQ ID NO: 279 or a conservative modification thereof;
al) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 327 or a conservative modification thereof;
am) the α chain variable region comprises a CDR2 comprising the amino acid sequence set forth in SEQ ID NO: 94 or a conservative modification thereof, and the β chain variable region comprises a CDR2 comprising the amino acid sequence set forth in SEQ ID NO: 350 or a conservative modification thereof;
an) the α chain variable region comprises a CDR2 comprising the amino acid sequence set forth in SEQ ID NO: 196 or a conservative modification thereof, and the β chain variable region comprises a CDR2 comprising the amino acid sequence set forth in SEQ ID NO: 350 or a conservative modification thereof;
ao) the α chain variable region comprises a CDR2 comprising the amino acid sequence set forth in SEQ ID NO: 180 or a conservative modification thereof, and the β chain variable region comprises a CDR2 comprising the amino acid sequence set forth in SEQ ID NO: 38 or a conservative modification thereof;
ap) the α chain variable region comprises a CDR2 comprising the amino acid sequence set forth in SEQ ID NO: 286 or a conservative modification thereof, and the β chain variable region comprises a CDR2 comprising the amino acid sequence set forth in SEQ ID NO: 327 or a conservative modification thereof;
aq) the α chain variable region comprises a CDR2 comprising the amino acid sequence set forth in SEQ ID NO: 370 or a conservative modification thereof, and the β chain variable region comprises a CDR2 comprising the amino acid sequence set forth in SEQ ID NO: 327 or a conservative modification thereof; or
ar) the α chain variable region comprises a CDR2 comprising the amino acid sequence set forth in SEQ ID NO: 376 or a conservative modification thereof, and the β chain variable region comprises a CDR2 comprising the amino acid sequence set forth in SEQ ID NO: 38 or a conservative modification thereof.

A16. The foregoing composition of any one of A13-A15, wherein:
a) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 8 or a conservative modification thereof;
b) the α chain variable region comprises a CDR1 comprising the amino acid sequence set forth in SEQ ID NO: 15 or a conservative modification thereof, and the β chain variable region comprises a CDR1 comprising the amino acid sequence set forth in SEQ ID NO: 8 or a conservative modification thereof;
c) the α chain variable region comprises a CDR1 comprising the amino acid sequence set forth in SEQ ID NO: 24 or a conservative modification thereof, and the β chain variable region comprises a CDR1 comprising the amino acid sequence set forth in SEQ ID NO: 27 or a conservative modification thereof;
d) the α chain variable region comprises a CDR1 comprising the amino acid sequence set forth in SEQ ID NO: 34 or a conservative modification thereof, and the β chain variable region comprises a CDR1 comprising the amino acid sequence set forth in SEQ ID NO: 37 or a conservative modification thereof;
e) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 48 or a conservative modification thereof;
f) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 37 or a conservative modification thereof;
g) the α chain variable region comprises a CDR1 comprising the amino acid sequence set forth in SEQ ID NO: 64 or a conservative modification thereof, and the β chain variable region comprises a CDR1 comprising the amino acid sequence set forth in SEQ ID NO: 48 or a conservative modification thereof;
h) the α chain variable region comprises a CDR1 comprising the amino acid sequence set forth in SEQ ID NO: 72 or a conservative modification thereof, and the β chain variable region comprises a CDR1 comprising the amino acid sequence set forth in SEQ ID NO: 48 or a conservative modification thereof;
i) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 78 or a conservative modification thereof;
j) the α chain variable region comprises a CDR1 comprising the amino acid sequence set forth in SEQ ID NO: 85 or a conservative modification thereof, and the β chain variable region comprises a CDR1 comprising the amino acid sequence set forth in SEQ ID NO: 37 or a conservative modification thereof;
k) the α chain variable region comprises a CDR1 comprising the amino acid sequence set forth in SEQ ID NO: 93 or a conservative modification thereof, and the β chain variable region comprises a CDR1 comprising the amino acid sequence set forth in SEQ ID NO: 48 or a conservative modification thereof;
l) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 102 or a conservative modification thereof;
m) the α chain variable region comprises a CDR1 comprising the amino acid sequence set forth in SEQ ID NO: 109 or a conservative modification thereof, and the β chain variable region comprises a CDR1 comprising the amino acid sequence set forth in SEQ ID NO: 112 or a conservative modification thereof;
n) the α chain variable region comprises a CDR1 comprising the amino acid sequence set forth in SEQ ID NO: 119 or a conservative modification thereof, and the β chain variable region comprises a CDR1 comprising the amino acid sequence set forth in SEQ ID NO: 122 or a conservative modification thereof;
o) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 131 or a conservative modification thereof;
p) the α chain variable region comprises a CDR1 comprising the amino acid sequence set forth in SEQ ID NO: 138 or a conservative modification thereof, and the β chain variable region comprises a CDR1 comprising the amino acid sequence set forth in SEQ ID NO: 102 or a conservative modification thereof;
q) the α chain variable region comprises a CDR1 comprising the amino acid sequence set forth in SEQ ID NO: 145 or a conservative modification thereof, and the β chain variable region comprises a CDR1 comprising the amino acid sequence set forth in SEQ ID NO: 102 or a conservative modification thereof;
r) the α chain variable region comprises a CDR1 comprising the amino acid sequence set forth in SEQ ID NO: 156 or a conservative modification thereof, and the β chain variable region comprises a CDR1 comprising the amino acid sequence set forth in SEQ ID NO: 159 or a conservative modification thereof;
s) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 182 or a conservative modification thereof;
t) the α chain variable region comprises a CDR1 comprising the amino acid sequence set forth in SEQ ID NO: 119 or a conservative modification thereof, and the β chain variable region comprises a CDR1 comprising the amino acid sequence set forth in SEQ ID NO: 37 or a conservative modification thereof;
u) the α chain variable region comprises a CDR1 comprising the amino acid sequence set forth in SEQ ID NO: 195 or a conservative modification thereof, and the β chain variable region comprises a CDR1 comprising the amino acid sequence set forth in SEQ ID NO: 159 or a conservative modification thereof;
v) the α chain variable region comprises a CDR1 comprising the amino acid sequence set forth in SEQ ID NO: 203 or a conservative modification thereof, and the β chain variable region comprises a CDR1 comprising the amino acid sequence set forth in SEQ ID NO: 122 or a conservative modification thereof;
w) the α chain variable region comprises a CDR1 comprising the amino acid sequence set forth in SEQ ID NO: 211 or a conservative modification thereof, and the β chain variable region comprises a CDR1 comprising the amino acid sequence set forth in SEQ ID NO: 214 or a conservative modification thereof;
x) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 159 or a conservative modification thereof;
y) the α chain variable region comprises a CDR1 comprising the amino acid sequence set forth in SEQ ID NO: 227 or a conservative modification thereof, and the β chain variable region comprises a CDR1 comprising the amino acid sequence set forth in SEQ ID NO: 230 or a conservative modification thereof;
z) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 238 or a conservative modification thereof;
aa) the α chain variable region comprises a CDR1 comprising the amino acid sequence set forth in SEQ ID NO: 72 or a conservative modification thereof, and the β chain variable region comprises a CDR1 comprising the amino acid sequence set forth in SEQ ID NO: 246 or a conservative modification thereof;
ab) the α chain variable region comprises a CDR1 comprising the amino acid sequence set forth in SEQ ID NO: 156 or a conservative modification thereof, and the β chain variable region comprises a CDR1 comprising the amino acid sequence set forth in SEQ ID NO: 37 or a conservative modification thereof;
ac) the α chain variable region comprises a CDR1 comprising the amino acid sequence set forth in SEQ ID NO: 260 or a conservative modification thereof, and the β chain variable region comprises a CDR1 comprising the amino acid sequence set forth in SEQ ID NO: 263 or a conservative modification thereof;
ad) the α chain variable region comprises a CDR1 comprising the amino acid sequence set forth in SEQ ID NO: 203 or a conservative modification thereof, and the β chain variable region comprises a CDR1 comprising the amino acid sequence set forth in SEQ ID NO: 271 or a conservative modification thereof;
ae) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 263 or a conservative modification thereof;
af) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 288 or a conservative modification thereof;
ag) the α chain variable region comprises a CDR1 comprising the amino acid sequence set forth in SEQ ID NO: 227 or a conservative modification thereof, and the β chain variable region comprises a CDR1 comprising the amino acid sequence set forth in SEQ ID NO: 296 or a conservative modification thereof;
ah) the α chain variable region comprises a CDR1 comprising the amino acid sequence set forth in SEQ ID NO: 5 or a conservative modification thereof, and the β chain variable region comprises a CDR1 comprising the amino acid sequence set forth in SEQ ID NO: 304 or a conservative modification thereof;
ai) the α chain variable region comprises a CDR1 comprising the amino acid sequence set forth in SEQ ID NO: 128 or a conservative modification thereof, and the β chain variable region comprises a CDR1 comprising the amino acid sequence set forth in SEQ ID NO: 27 or a conservative modification thereof;
aj) the α chain variable region comprises a CDR1 comprising the amino acid sequence set forth in SEQ ID NO: 317 or a conservative modification thereof, and the β chain variable region comprises a CDR1 comprising the amino acid sequence set forth in SEQ ID NO: 263 or a conservative modification thereof;
ak) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 326 or a conservative modification thereof;
al) the α chain variable region comprises a CDR1 comprising the amino acid sequence set forth in SEQ ID NO: 285 or a conservative modification thereof, and the β chain variable region comprises a CDR1 comprising the amino acid sequence set forth in SEQ ID NO: 288 or a conservative modification thereof;
am) the α chain variable region comprises a CDR1 comprising the amino acid sequence set forth in SEQ ID NO: 93 or a conservative modification thereof, and the β chain variable region comprises a CDR1 comprising the amino acid sequence set forth in SEQ ID NO: 182 or a conservative modification thereof;
an) the α chain variable region comprises a CDR1 comprising the amino acid sequence set forth in SEQ ID NO: 195 or a conservative modification thereof, and the β chain variable region comprises a CDR1 comprising the amino acid sequence set forth in SEQ ID NO: 182 or a conservative modification thereof;
ao) the α chain variable region comprises a CDR1 comprising the amino acid sequence set forth in SEQ ID NO: 179 or a conservative modification thereof, and the β chain variable region comprises a CDR1 comprising the amino acid sequence set forth in SEQ ID NO: 37 or a conservative modification thereof;
ap) the α chain variable region comprises a CDR1 comprising the amino acid sequence set forth in SEQ ID NO: 369 or a conservative modification thereof, and the β chain variable region comprises a CDR1 comprising the amino acid sequence set forth in SEQ ID NO: 326 or a conservative modification thereof; or
aq) the α chain variable region comprises a CDR1 comprising the amino acid sequence set forth in SEQ ID NO: 375 or a conservative modification thereof, and the β chain variable region comprises a CDR1 comprising the amino acid sequence set forth in SEQ ID NO: 37 or a conservative modification thereof.

A17. The foregoing TCR of any one of A13-A16, wherein:
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55;
h) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66;
i) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
j) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77;
k) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87;
l) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95;
m) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101;
n) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111;
o) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121;
p) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130;
q) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139;
r) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147;
s) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150;
t) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158;
u) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173;
v) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181;
w) the α chain variable region comprises a CDR1 comprising the amino acid sequence set forth in SEQ ID NO: 119; a CDR2 comprising the amino acid sequence set forth in SEQ ID NO: 120;
and a CDR3 comprising the amino acid sequence set forth in SEQ ID NO: 189;
x) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197;
y) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205;
z) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213;
aa) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221;
ab) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229;
ac) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237;
ad) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245;
ae) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254;
af) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262;
ag) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270;
ah) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278;
ai) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287;
aj) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295;
ak) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303;
al) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311;
am) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319;
an) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325;
ao) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333;
ap) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345;
ar) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349;
as) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356;
at) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360;
au) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371;
av) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; or
aw) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383.

A18. The foregoing TCR of any one of A13-A17, wherein:
a) the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
f) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 55;
g) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
h) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
i) the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
j) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
k) the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
l) the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
m) the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
n) the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
o) the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
p) the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
q) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
r) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
s) the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
t) the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
u) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
v) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
w) the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
x) the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
y) the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
z) the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
aa) the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ab) the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
ac) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ad) the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ae) the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
af) the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
ag) the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ah) the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
ai) the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
aj) the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
ak) the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
al) the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
am) the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
an) the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ao) the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
ap) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
aq) the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290; or
ar) the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

A19. The foregoing TCR of any one of A13-A18, wherein:
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56;
h) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
i) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
j) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
k) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
l) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
m) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the β chain variable comprising the amino acid sequence set forth in SEQ ID NO: 49, and a CDR3 comprising the amino acid sequence set forth in SEQ ID NO: 96;
n) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
o) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
p) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
q) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
r) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
s) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
t) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
u) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
v) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
w) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
x) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
y) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
z) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
aa) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
ab) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
ac) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
ad) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ae) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
af) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ag) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ah) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
ai) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
aj) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ak) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
al) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
am) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
an) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
ao) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
ap) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ar) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
as) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
at) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
au) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
av) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
aw) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
ax) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378; or
ay) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

A20. The foregoing TCR of any one of A13-A19, wherein
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; or
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19.

A21. The foregoing TCR of any one of A13-A20, wherein the α chain variable region comprises an amino acid sequence that is at least about 80% homologous or identical to the amino acid sequence set forth in SEQ ID NO: 11, SEQ ID NO: 20, SEQ ID NO: 30, SEQ ID NO: 40, SEQ ID NO: 45, SEQ ID NO: 51, SEQ ID NO: 57, SEQ ID NO: 68, SEQ ID NO: 75, SEQ ID NO: 81, SEQ ID NO: 89, SEQ ID NO: 97, SEQ ID NO: 105, SEQ ID NO: 115, SEQ ID NO: 124, SEQ ID NO: 134, SEQ ID NO: 141, SEQ ID NO: 148, SEQ ID NO: 152, SEQ ID NO: 162, SEQ ID NO: 175, SEQ ID NO: 185, SEQ ID NO: 191, SEQ ID NO: 199, SEQ ID NO: 207, SEQ ID NO: 217, SEQ ID NO: 223, SEQ ID NO: 233, SEQ ID NO: 241, SEQ ID NO: 249, SEQ ID NO: 256, SEQ ID NO: 266, SEQ ID NO: 274, SEQ ID NO: 281, SEQ ID NO: 291, SEQ ID NO: 299, SEQ ID NO: 307, SEQ ID NO: 313, SEQ ID NO: 321, SEQ ID NO: 329, SEQ ID NO: 335, SEQ ID NO: 341, SEQ ID NO: 346, SEQ ID NO: 352, SEQ ID NO: 357, SEQ ID NO: 362, SEQ ID NO: 375, SEQ ID NO: 379, or SEQ ID NO: 384.

A22. The foregoing TCR of A21, wherein the α chain variable region comprises the amino acid sequence set forth in SEQ ID NO: 11, SEQ ID NO: 20, SEQ ID NO: 30, SEQ ID NO: 40, SEQ ID NO: 45, SEQ ID NO: 51, SEQ ID NO: 57, SEQ ID NO: 68, SEQ ID NO: 75, SEQ ID NO: 81, SEQ ID NO: 89, SEQ ID NO: 97, SEQ ID NO: 105, SEQ ID NO: 115, SEQ ID NO: 124, SEQ ID NO: 134, SEQ ID NO: 141, SEQ ID NO: 148, SEQ ID NO: 152, SEQ ID NO: 162, SEQ ID NO: 175, SEQ ID NO: 185, SEQ ID NO: 191, SEQ ID NO: 199, SEQ ID NO: 207, SEQ ID NO: 217, SEQ ID NO: 223, SEQ ID NO: 233, SEQ ID NO: 241, SEQ ID NO: 249, SEQ ID NO: 256, SEQ ID NO: 266, SEQ ID NO: 274, SEQ ID NO: 281, SEQ ID NO: 291, SEQ ID NO: 299, SEQ ID NO: 307, SEQ ID NO: 313, SEQ ID NO: 321, SEQ ID NO: 329, SEQ ID NO: 335, SEQ ID NO: 341, SEQ ID NO: 346, SEQ ID NO: 352, SEQ ID NO: 357, SEQ ID NO: 362, SEQ ID NO: 375, SEQ ID NO: 379, or SEQ ID NO: 384.

A23. The foregoing TCR of A22, wherein the α chain variable region comprises the amino acid sequence set forth in SEQ ID NO: 11 or SEQ ID NO: 20.

A24. The foregoing TCR of any one of A13-A23, wherein the β chain variable region comprises an amino acid sequence that is at least about 80% homologous or identical to the amino acid sequence set forth in SEQ ID NO: 12, SEQ ID NO: 21, SEQ ID NO: 31, SEQ ID NO: 41, SEQ ID NO: 52, SEQ ID NO: 58, SEQ ID NO: 62, SEQ ID NO: 69, SEQ ID NO: 82, SEQ ID NO: 90, SEQ ID NO: 98, SEQ ID NO: 106, SEQ ID NO: 125, SEQ ID NO: 135, SEQ ID NO: 142, SEQ ID NO: 153, SEQ ID NO: 162, SEQ ID NO: 176, SEQ ID NO: 186, SEQ ID NO: 192, SEQ ID NO: 200, SEQ ID NO: 208, SEQ ID NO: 218, SEQ ID NO: 224, SEQ ID NO: 234, SEQ ID NO: 242, SEQ ID NO: 250, SEQ ID NO: 257, SEQ ID NO: 267, SEQ ID NO: 275, SEQ ID NO: 282, SEQ ID NO: 292, SEQ ID NO: 300, SEQ ID NO: 308, SEQ ID NO: 314, SEQ ID NO: 322, SEQ ID NO: 330, SEQ ID NO: 336, SEQ ID NO: 342, SEQ ID NO: 353, SEQ ID NO: 363, SEQ ID NO: 366, or SEQ ID NO: 380.

A25. The foregoing TCR of A24, wherein the β chain variable region comprises the amino acid sequence set forth in SEQ ID NO: 12, SEQ ID NO: 21, SEQ ID NO: 31, SEQ ID NO: 41, SEQ ID NO: 52, SEQ ID NO: 58, SEQ ID NO: 62, SEQ ID NO: 69, SEQ ID NO: 82, SEQ ID NO: 90, SEQ ID NO: 98, SEQ ID NO: 106, SEQ ID NO: 125, SEQ ID NO: 135, SEQ ID NO: 142, SEQ ID NO: 153, SEQ ID NO: 162, SEQ ID NO: 176, SEQ ID NO: 186, SEQ ID NO: 192, SEQ ID NO: 200, SEQ ID NO: 208, SEQ ID NO: 218, SEQ ID NO: 224, SEQ ID NO: 234, SEQ ID NO: 242, SEQ ID NO: 250, SEQ ID NO: 257, SEQ ID NO: 267, SEQ ID NO: 275, SEQ ID NO: 282, SEQ ID NO: 292, SEQ ID NO: 300, SEQ ID NO: 308, SEQ ID NO: 314, SEQ ID NO: 322, SEQ ID NO: 330, SEQ ID NO: 336, SEQ ID NO: 342, SEQ ID NO: 353, SEQ ID NO: 363, SEQ ID NO: 366, or SEQ ID NO: 380.

A26. The foregoing TCR of A25, wherein the β chain variable region comprises the amino acid sequence set forth in SEQ ID NO: 12 or SEQ ID NO: 21.

A27. The foregoing TCR of any one of A13-A26, wherein:
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 20, and the β chain variable region comprises an amino acid sequence that is at least about 80% homologous or identical to the amino acid sequence set forth in SEQ ID NO: 21;
c) the α chain variable region comprises an amino acid sequence that is at least about 80% homologous or identical to the amino acid sequence set forth in SEQ ID NO: 30, and the β chain variable region comprises an amino acid sequence that is at least about 80% homologous or identical to the amino acid sequence set forth in SEQ ID NO: 31;
d) the α chain variable region comprises an amino acid sequence that is at least about 80% homologous or identical to the amino acid sequence set forth in SEQ ID NO: 40, and the β chain variable region comprises an amino acid sequence that is at least about 80% homologous or identical to the amino acid sequence set forth in SEQ ID NO: 41;
e) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 41;
f) the α chain variable region comprises an amino acid sequence that is at least about 80% homologous or identical to the amino acid sequence set forth in SEQ ID NO: 51, and the β chain variable region comprises an amino acid sequence that is at least about 80% homologous or identical to the amino acid sequence set forth in SEQ ID NO: 52;
g) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 58;
h) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 62;
i) the α chain variable region comprises an amino acid sequence that is at least about 80% homologous or identical to the amino acid sequence set forth in SEQ ID NO: 68, and the β chain variable region comprises an amino acid sequence that is at least about 80% homologous or identical to the amino acid sequence set forth in SEQ ID NO: 69;
j) the α chain variable region comprises an amino acid sequence that is at least about 80% homologous or identical to the amino acid sequence set forth in SEQ ID NO: 75, and the β chain variable region comprises an amino acid sequence that is at least about 80% homologous or identical to the amino acid sequence set forth in SEQ ID NO: 69;
k) the α chain variable region comprises an amino acid sequence that is at least about 80% homologous or identical to the amino acid sequence set forth in SEQ ID NO: 81, and the β chain variable region comprises an amino acid sequence that is at least about 80% homologous or identical to the amino acid sequence set forth in SEQ ID NO: 82;
l) the α chain variable region comprises an amino acid sequence that is at least about 80% homologous or identical to the amino acid sequence set forth in SEQ ID NO: 89, and the β chain variable region comprises an amino acid sequence that is at least about 80% homologous or identical to the amino acid sequence set forth in SEQ ID NO: 90;
m) the α chain variable region comprises an amino acid sequence that is at least about 80% homologous or identical to the amino acid sequence set forth in SEQ ID NO: 97, and the β chain variable region comprises an amino acid sequence that is at least about 80% homologous or identical to the amino acid sequence set forth in SEQ ID NO: 98;
n) the α chain variable region comprises an amino acid sequence that is at least about 80% homologous or identical to the amino acid sequence set forth in SEQ ID NO: 105, and the β chain variable region comprises an amino acid sequence that is at least about 80% homologous or identical to the amino acid sequence set forth in SEQ ID NO: 106;
o) the α chain variable region comprises an amino acid sequence that is at least about 80% homologous or identical to the amino acid sequence set forth in SEQ ID NO: 115, and the β chain variable region comprises an amino acid sequence that is at least about 80% homologous or identical to the amino acid sequence set forth in SEQ ID NO: 116;
p) the α chain variable region comprises an amino acid sequence that is at least about 80% homologous or identical to the amino acid sequence set forth in SEQ ID NO: 124, and the β chain variable region comprises an amino acid sequence that is at least about 80% homologous or identical to the amino acid sequence set forth in SEQ ID NO: 125;
q) the α chain variable region comprises an amino acid sequence that is at least about 80% homologous or identical to the amino acid sequence set forth in SEQ ID NO: 134, and the β chain variable region comprises an amino acid sequence that is at least about 80% homologous or identical to the amino acid sequence set forth in SEQ ID NO: 135;
r) the α chain variable region comprises an amino acid sequence that is at least about 80% homologous or identical to the amino acid sequence set forth in SEQ ID NO: 141, and the β chain variable region comprises an amino acid sequence that is at least about 80% homologous or identical to the amino acid sequence set forth in SEQ ID NO: 142;
s) the α chain variable region comprises an amino acid sequence that is at least about 80% homologous or identical to the amino acid sequence set forth in SEQ ID NO: 148, and the β chain variable region comprises an amino acid sequence that is at least about 80% homologous or identical to the amino acid sequence set forth in SEQ ID NO: 142;
t) the α chain variable region comprises an amino acid sequence that is at least about 80% homologous or identical to the amino acid sequence set forth in SEQ ID NO: 152, and the β chain variable region comprises an amino acid sequence that is at least about 80% homologous or identical to the amino acid sequence set forth in SEQ ID NO: 153;
u) the α chain variable region comprises an amino acid sequence that is at least about 80% homologous or identical to the amino acid sequence set forth in SEQ ID NO: 162, and the β chain variable region comprises an amino acid sequence that is at least about 80% homologous or identical to the amino acid sequence set forth in SEQ ID NO: 163;
v) the α chain variable region comprises an amino acid sequence that is at least about 80% homologous or identical to the amino acid sequence set forth in SEQ ID NO: 175, and the β chain variable region comprises an amino acid sequence that is at least about 80% homologous or identical to the amino acid sequence set forth in SEQ ID NO: 176;
w) the α chain variable region comprises an amino acid sequence that is at least about 80% homologous or identical to the amino acid sequence set forth in SEQ ID NO: 185, and the β chain variable region comprises an amino acid sequence that is at least about 80% homologous or identical to the amino acid sequence set forth in SEQ ID NO: 186;
x) the α chain variable region comprises an amino acid sequence that is at least about 80% homologous or identical to the amino acid sequence set forth in SEQ ID NO: 191, and the β chain variable region comprises an amino acid sequence that is at least about 80% homologous or identical to the amino acid sequence set forth in SEQ ID NO: 192;
y) the α chain variable region comprises an amino acid sequence that is at least about 80% homologous or identical to the amino acid sequence set forth in SEQ ID NO: 199, and the β chain variable region comprises an amino acid sequence that is at least about 80% homologous or identical to the amino acid sequence set forth in SEQ ID NO: 200;
z) the α chain variable region comprises an amino acid sequence that is at least about 80% homologous or identical to the amino acid sequence set forth in SEQ ID NO: 207, and the β chain variable region comprises an amino acid sequence that is at least about 80% homologous or identical to the amino acid sequence set forth in SEQ ID NO: 208;
aa) the α chain variable region comprises an amino acid sequence that is at least about 80% homologous or identical to the amino acid sequence set forth in SEQ ID NO: 217, and the β chain variable region comprises an amino acid sequence that is at least about 80% homologous or identical to the amino acid sequence set forth in SEQ ID NO: 218;
ab) the α chain variable region comprises an amino acid sequence that is at least about 80% homologous or identical to the amino acid sequence set forth in SEQ ID NO: 223, and the β chain variable region comprises an amino acid sequence that is at least about 80% homologous or identical to the amino acid sequence set forth in SEQ ID NO: 224;
ac) the α chain variable region comprises an amino acid sequence that is at least about 80% homologous or identical to the amino acid sequence set forth in SEQ ID NO: 233, and the β chain variable region comprises an amino acid sequence that is at least about 80% homologous or identical to the amino acid sequence set forth in SEQ ID NO: 234;
ad) the α chain variable region comprises an amino acid sequence that is at least about 80% homologous or identical to the amino acid sequence set forth in SEQ ID NO: 241, and the β chain variable region comprises an amino acid sequence that is at least about 80% homologous or identical to the amino acid sequence set forth in SEQ ID NO: 242;
ae) the α chain variable region comprises an amino acid sequence that is at least about 80% homologous or identical to the amino acid sequence set forth in SEQ ID NO: 249, and the β chain variable region comprises an amino acid sequence that is at least about 80% homologous or identical to the amino acid sequence set forth in SEQ ID NO: 250;
af) the α chain variable region comprises an amino acid sequence that is at least about 80% homologous or identical to the amino acid sequence set forth in SEQ ID NO: 256, and the β chain variable region comprises an amino acid sequence that is at least about 80% homologous or identical to the amino acid sequence set forth in SEQ ID NO: 257;
ag) the α chain variable region comprises an amino acid sequence that is at least about 80% homologous or identical to the amino acid sequence set forth in SEQ ID NO: 266, and the β chain variable region comprises an amino acid sequence that is at least about 80% homologous or identical to the amino acid sequence set forth in SEQ ID NO: 267;
ah) the α chain variable region comprises an amino acid sequence that is at least about 80% homologous or identical to the amino acid sequence set forth in SEQ ID NO: 274, and the β chain variable region comprises an amino acid sequence that is at least about 80% homologous or identical to the amino acid sequence set forth in SEQ ID NO: 275;
ai) the α chain variable region comprises an amino acid sequence that is at least about 80% homologous or identical to the amino acid sequence set forth in SEQ ID NO: 281, and the β chain variable region comprises an amino acid sequence that is at least about 80% homologous or identical to the amino acid sequence set forth in SEQ ID NO: 282;
aj) the α chain variable region comprises an amino acid sequence that is at least about 80% homologous or identical to the amino acid sequence set forth in SEQ ID NO: 291, and the β chain variable region comprises an amino acid sequence that is at least about 80% homologous or identical to the amino acid sequence set forth in SEQ ID NO: 292;
ak) the α chain variable region comprises an amino acid sequence that is at least about 80% homologous or identical to the amino acid sequence set forth in SEQ ID NO: 299, and the β chain variable region comprises an amino acid sequence that is at least about 80% homologous or identical to the amino acid sequence set forth in SEQ ID NO: 300;
al) the α chain variable region comprises an amino acid sequence that is at least about 80% homologous or identical to the amino acid sequence set forth in SEQ ID NO: 307, and the β chain variable region comprises an amino acid sequence that is at least about 80% homologous or identical to the amino acid sequence set forth in SEQ ID NO: 308;
am) the α chain variable region comprises an amino acid sequence that is at least about 80% homologous or identical to the amino acid sequence set forth in SEQ ID NO: 313, and the β chain variable region comprises an amino acid sequence that is at least about 80% homologous or identical to the amino acid sequence set forth in SEQ ID NO: 314;
an) the α chain variable region comprises an amino acid sequence that is at least about 80% homologous or identical to the amino acid sequence set forth in SEQ ID NO: 321, and the β chain variable region comprises an amino acid sequence that is at least about 80% homologous or identical to the amino acid sequence set forth in SEQ ID NO: 322;
ao) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 330;
ap) the α chain variable region comprises an amino acid sequence that is at least about 80% homologous or identical to the amino acid sequence set forth in SEQ ID NO: 335, and the β chain variable region comprises an amino acid sequence that is at least about 80% homologous or identical to the amino acid sequence set forth in SEQ ID NO: 336;
aq) the α chain variable region comprises an amino acid sequence that is at least about 80% homologous or identical to the amino acid sequence set forth in SEQ ID NO: 341, and the β chain variable region comprises an amino acid sequence that is at least about 80% homologous or identical to the amino acid sequence set forth in SEQ ID NO: 342;
ar) the α chain variable region comprises an amino acid sequence that is at least about 80% homologous or identical to the amino acid sequence set forth in SEQ ID NO: 346, and the β chain variable region comprises an amino acid sequence that is at least about 80% homologous or identical to the amino acid sequence set forth in SEQ ID NO: 342;
as) the α chain variable region comprises an amino acid sequence that is at least about 80% homologous or identical to the amino acid sequence set forth in SEQ ID NO: 352, and the β chain variable region comprises an amino acid sequence that is at least about 80% homologous or identical to the amino acid sequence set forth in SEQ ID NO: 353;
at) the α chain variable region comprises an amino acid sequence that is at least about 80% homologous or identical to the amino acid sequence set forth in SEQ ID NO: 357, and the β chain variable region comprises an amino acid sequence that is at least about 80% homologous or identical to the amino acid sequence set forth in SEQ ID NO: 353;
au) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
av) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
aw) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 366;
ax) the α chain variable region comprises an amino acid sequence that is at least about 80% homologous or identical to the amino acid sequence set forth in SEQ ID NO: 372, and the β chain variable region comprises an amino acid sequence that is at least about 80% homologous or identical to the amino acid sequence set forth in SEQ ID NO: 366;
ay) the α chain variable region comprises an amino acid sequence that is at least about 80% homologous or identical to the amino acid sequence set forth in SEQ ID NO: 379, and the β chain variable region comprises an amino acid sequence that is at least about 80% homologous or identical to the amino acid sequence set forth in SEQ ID NO: 380; or az) the α chain variable region comprises an amino acid sequence that is at least about 80% homologous or identical to the amino acid sequence set forth in SEQ ID NO: 384, and the β chain variable region comprises an amino acid sequence that is at least about 80% homologous or identical to the amino acid sequence set forth in SEQ ID NO: 380.

A28. The foregoing TCR of any one of A13-A27, wherein:
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 20, and the β chain variable region comprises the amino acid sequence set forth in SEQ ID NO: 21;
c) the α chain variable region comprises the amino acid sequence set forth in SEQ ID NO: 30, and the β chain variable region comprises the amino acid sequence set forth in SEQ ID NO: 31;
d) the α chain variable region comprises the amino acid sequence set forth in SEQ ID NO: 40, and the β chain variable region comprises the amino acid sequence set forth in SEQ ID NO: 41;
e) the α chain variable region comprises the amino acid sequence set forth in SEQ ID NO: 45, and the β chain variable region comprises the amino acid sequence set forth in SEQ ID NO: 41;
f) the α chain variable region comprises the amino acid sequence set forth in SEQ ID NO: 51, and the β chain variable region comprises the amino acid sequence set forth in SEQ ID NO: 52;
g) the α chain variable region comprises the amino acid sequence set forth in SEQ ID NO: 57, and the β chain variable region comprises the amino acid sequence set forth in SEQ ID NO: 58;
h) the α chain variable region comprises the amino acid sequence set forth in SEQ ID NO: 57, and the β chain variable region comprises the amino acid sequence set forth in SEQ ID NO: 62;
i) the α chain variable region comprises the amino acid sequence set forth in SEQ ID NO: 68, and the β chain variable region comprises the amino acid sequence set forth in SEQ ID NO: 69;
j) the α chain variable region comprises the amino acid sequence set forth in SEQ ID NO: 75, and the β chain variable region comprises the amino acid sequence set forth in SEQ ID NO: 69;
k) the α chain variable region comprises the amino acid sequence set forth in SEQ ID NO: 81, and the β chain variable region comprises the amino acid sequence set forth in SEQ ID NO: 82;
l) the α chain variable region comprises the amino acid sequence set forth in SEQ ID NO: 89, and the β chain variable region comprises the amino acid sequence set forth in SEQ ID NO: 90;
m) the α chain variable region comprises the amino acid sequence set forth in SEQ ID NO: 97, and the β chain variable region comprises the amino acid sequence set forth in SEQ ID NO: 98;
n) the α chain variable region comprises the amino acid sequence set forth in SEQ ID NO: 105, and the β chain variable region comprises the amino acid sequence set forth in SEQ ID NO: 106;
o) the α chain variable region comprises the amino acid sequence set forth in SEQ ID NO: 115, and the β chain variable region comprises the amino acid sequence set forth in SEQ ID NO: 116;
p) the α chain variable region comprises the amino acid sequence set forth in SEQ ID NO: 124, and the β chain variable region comprises the amino acid sequence set forth in SEQ ID NO: 125;
q) the α chain variable region comprises the amino acid sequence set forth in SEQ ID NO: 134, and the β chain variable region comprises the amino acid sequence set forth in SEQ ID NO: 135;
r) the α chain variable region comprises the amino acid sequence set forth in SEQ ID NO: 141, and the β chain variable region comprises the amino acid sequence set forth in SEQ ID NO: 142;
s) the α chain variable region comprises the amino acid sequence set forth in SEQ ID NO: 148, and the β chain variable region comprises the amino acid sequence set forth in SEQ ID NO: 142;
t) the α chain variable region comprises the amino acid sequence set forth in SEQ ID NO: 152, and the β chain variable region comprises the amino acid sequence set forth in SEQ ID NO: 153;
u) the α chain variable region comprises the amino acid sequence set forth in SEQ ID NO: 162, and the β chain variable region comprises the amino acid sequence set forth in SEQ ID NO: 163;
v) the α chain variable region comprises the amino acid sequence set forth in SEQ ID NO: 175, and the β chain variable region comprises the amino acid sequence set forth in SEQ ID NO: 176;
w) the α chain variable region comprises the amino acid sequence set forth in SEQ ID NO: 185, and the β chain variable region comprises the amino acid sequence set forth in SEQ ID NO: 186;
x) the α chain variable region comprises the amino acid sequence set forth in SEQ ID NO: 191, and the β chain variable region comprises the amino acid sequence set forth in SEQ ID NO: 192;
y) the α chain variable region comprises the amino acid sequence set forth in SEQ ID NO: 199, and the β chain variable region comprises the amino acid sequence set forth in SEQ ID NO: 200;
z) the α chain variable region comprises the amino acid sequence set forth in SEQ ID NO: 207, and the β chain variable region comprises the amino acid sequence set forth in SEQ ID NO: 208;
aa) the α chain variable region comprises the amino acid sequence set forth in SEQ ID NO: 217, and the β chain variable region comprises the amino acid sequence set forth in SEQ ID NO: 218;
ab) the α chain variable region comprises the amino acid sequence set forth in SEQ ID NO: 223, and the β chain variable region comprises the amino acid sequence set forth in SEQ ID NO: 224;
ac) the α chain variable region comprises the amino acid sequence set forth in SEQ ID NO: 233, and the β chain variable region comprises the amino acid sequence set forth in SEQ ID NO: 234;
ad) the α chain variable region comprises the amino acid sequence set forth in SEQ ID NO: 241, and the β chain variable region comprises the amino acid sequence set forth in SEQ ID NO: 242;
ae) the α chain variable region comprises the amino acid sequence set forth in SEQ ID NO: 249, and the β chain variable region comprises the amino acid sequence set forth in SEQ ID NO: 250;
af) the α chain variable region comprises the amino acid sequence set forth in SEQ ID NO: 256, and the β chain variable region comprises the amino acid sequence set forth in SEQ ID NO: 257;
ag) the α chain variable region comprises the amino acid sequence set forth in SEQ ID NO: 266, and the β chain variable region comprises the amino acid sequence set forth in SEQ ID NO: 267;
ah) the α chain variable region comprises the amino acid sequence set forth in SEQ ID NO: 274, and the β chain variable region comprises the amino acid sequence set forth in SEQ ID NO: 275;
ai) the α chain variable region comprises the amino acid sequence set forth in SEQ ID NO: 281, and the β chain variable region comprises the amino acid sequence set forth in SEQ ID NO: 282;
aj) the α chain variable region comprises the amino acid sequence set forth in SEQ ID NO: 291, and the β chain variable region comprises the amino acid sequence set forth in SEQ ID NO: 292;
ak) the α chain variable region comprises the amino acid sequence set forth in SEQ ID NO: 299, and the β chain variable region comprises the amino acid sequence set forth in SEQ ID NO: 300;
al) the α chain variable region comprises the amino acid sequence set forth in SEQ ID NO: 307, and the β chain variable region comprises the amino acid sequence set forth in SEQ ID NO: 308;
am) the α chain variable region comprises the amino acid sequence set forth in SEQ ID NO: 313, and the β chain variable region comprises the amino acid sequence set forth in SEQ ID NO: 314;
an) the α chain variable region comprises the amino acid sequence set forth in SEQ ID NO: 321, and the β chain variable region comprises the amino acid sequence set forth in SEQ ID NO: 322;
ao) the α chain variable region comprises the amino acid sequence set forth in SEQ ID NO: 329, and the β chain variable region comprises the amino acid sequence set forth in SEQ ID NO: 330;
ap) the α chain variable region comprises the amino acid sequence set forth in SEQ ID NO: 335, and the β chain variable region comprises the amino acid sequence set forth in SEQ ID NO: 336;
aq) the α chain variable region comprises the amino acid sequence set forth in SEQ ID NO: 341, and the β chain variable region comprises the amino acid sequence set forth in SEQ ID NO: 342;
ar) the α chain variable region comprises the amino acid sequence set forth in SEQ ID NO: 346, and the β chain variable region comprises the amino acid sequence set forth in SEQ ID NO: 342;
as) the α chain variable region comprises the amino acid sequence set forth in SEQ ID NO: 352, and the β chain variable region comprises the amino acid sequence set forth in SEQ ID NO: 353;
at) the α chain variable region comprises the amino acid sequence set forth in SEQ ID NO: 357, and the β chain variable region comprises the amino acid sequence set forth in SEQ ID NO: 353;
au) the α chain variable region comprises the amino acid sequence set forth in SEQ ID NO: 362, and the β chain variable region comprises the amino acid sequence set forth in SEQ ID NO: 363;
av) the α chain variable region comprises the amino acid sequence set forth in SEQ ID NO: 362, and the β chain variable region comprises the amino acid sequence set forth in SEQ ID NO: 363;
aw) the α chain variable region comprises the amino acid sequence set forth in SEQ ID NO: 329, and the β chain variable region comprises the amino acid sequence set forth in SEQ ID NO: 366;
ax) the α chain variable region comprises the amino acid sequence set forth in SEQ ID NO: 372, and the β chain variable region comprises the amino acid sequence set forth in SEQ ID NO: 366;
ay) the α chain variable region comprises the amino acid sequence set forth in SEQ ID NO: 379, and the β chain variable region comprises the amino acid sequence set forth in SEQ ID NO: 380; or
az) the α chain variable region comprises the amino acid sequence set forth in SEQ ID NO: 384, and the β chain variable region comprises the amino acid sequence set forth in SEQ ID NO: 380.

A29. The foregoing TCR of A28, wherein
a) the α chain variable region comprises the amino acid sequence set forth in SEQ ID NO: 11, and the β chain variable region comprises the amino acid sequence set forth in SEQ ID NO: 12; or
b) the α chain variable region comprises the amino acid sequence set forth in SEQ ID NO: 20, and the β chain variable region comprises the amino acid sequence set forth in SEQ ID NO: 21.

A30. The foregoing TCR of any one of A13-A29, wherein:
a) the α chain comprises the amino acid sequence set forth in SEQ ID NO: 13, and the β chain comprises the amino acid sequence set forth in SEQ ID NO: 14;
b) the α chain comprises the amino acid sequence set forth in SEQ ID NO: 22, and the β chain comprises the amino acid sequence set forth in SEQ ID NO: 23;
c) the α chain comprises the amino acid sequence set forth in SEQ ID NO: 32, and the β chain comprises the amino acid sequence set forth in SEQ ID NO: 33;
d) the α chain comprises the amino acid sequence set forth in SEQ ID NO: 42, and the β chain comprising the amino acid sequence set forth in SEQ ID NO: 43;
e) the α chain comprises the amino acid sequence set forth in SEQ ID NO: 46, and the β chain comprises the amino acid sequence set forth in SEQ ID NO: 43;
f) the α chain comprises the amino acid sequence set forth in SEQ ID NO: 53, and the β chain comprises the amino acid sequence set forth in SEQ ID NO: 54;
g) the α chain comprises the amino acid sequence set forth in SEQ ID NO: 59, and the β chain comprises the amino acid sequence set forth in SEQ ID NO: 60;
h) the α chain comprises the amino acid sequence set forth in SEQ ID NO: 59, and the β chain comprises the amino acid sequence set forth in SEQ ID NO: 63;
i) the α chain comprises the amino acid sequence set forth in SEQ ID NO: 70, and the β chain comprises the amino acid sequence set forth in SEQ ID NO: 71;
j) the α chain comprises the amino acid sequence set forth in SEQ ID NO: 76, and the β chain comprises the amino acid sequence set forth in SEQ ID NO: 71;
k) the α chain comprises the amino acid sequence set forth in SEQ ID NO: 83, and the β chain comprises the amino acid sequence set forth in SEQ ID NO: 84;
l) the α chain comprises the amino acid sequence set forth in SEQ ID NO: 91, and the β chain comprises the amino acid sequence set forth in SEQ ID NO: 92;
m) the α chain comprises the amino acid sequence set forth in SEQ ID NO: 99, and the β chain comprises the amino acid sequence set forth in SEQ ID NO: 100;
n) the α chain comprises the amino acid sequence set forth in SEQ ID NO: 107, and the β chain comprises the amino acid sequence set forth in SEQ ID NO: 108;
o) the α chain comprises the amino acid sequence set forth in SEQ ID NO: 117, and the β chain comprises the amino acid sequence set forth in SEQ ID NO: 118;
p) the α chain comprises the amino acid sequence set forth in SEQ ID NO: 126, and the β chain comprises the amino acid sequence set forth in SEQ ID NO: 127;
q) the α chain comprises the amino acid sequence set forth in SEQ ID NO: 136, and the β chain comprises the amino acid sequence set forth in SEQ ID NO: 137;
r) the α chain comprises the amino acid sequence set forth in SEQ ID NO: 143, and the β chain comprises the amino acid sequence set forth in SEQ ID NO: 144;
s) the α chain comprises the amino acid sequence set forth in SEQ ID NO: 149, and the β chain comprises the amino acid sequence set forth in SEQ ID NO: 144;
t) the α chain comprises the amino acid sequence set forth in SEQ ID NO: 154, and the β chain comprises the amino acid sequence set forth in SEQ ID NO: 155;
u) the α chain comprises the amino acid sequence set forth in SEQ ID NO: 164, and the β chain comprises the amino acid sequence set forth in SEQ ID NO: 165;
v) the α chain comprises the amino acid sequence set forth in SEQ ID NO: 177, and the β chain comprises the amino acid sequence set forth in SEQ ID NO: 178;
w) the α chain comprises the amino acid sequence set forth in SEQ ID NO: 187, and the β chain comprises the amino acid sequence set forth in SEQ ID NO: 188;
x) the α chain comprises the amino acid sequence set forth in SEQ ID NO: 193, and the β chain comprises the amino acid sequence set forth in SEQ ID NO: 194;
y) the α chain comprises the amino acid sequence set forth in SEQ ID NO: 201, and the β chain comprising the amino acid sequence set forth in SEQ ID NO: 202;
z) the α chain comprises the amino acid sequence set forth in SEQ ID NO: 209, and the β chain comprises the amino acid sequence set forth in SEQ ID NO: 210;
aa) the α chain comprises the amino acid sequence set forth in SEQ ID NO: 219, and the β chain comprises the amino acid sequence set forth in SEQ ID NO: 220;
ab) the α chain comprises the amino acid sequence set forth in SEQ ID NO: 225, and the β chain comprises the amino acid sequence set forth in SEQ ID NO: 226;
ac) the α chain comprises the amino acid sequence set forth in SEQ ID NO: 235, and the β chain comprises the amino acid sequence set forth in SEQ ID NO: 236;
ad) the α chain comprises the amino acid sequence set forth in SEQ ID NO: 243, and the β chain comprises the amino acid sequence set forth in SEQ ID NO: 244;
ae) the α chain comprises the amino acid sequence set forth in SEQ ID NO: 251, and the β chain comprises the amino acid sequence set forth in SEQ ID NO: 252;
af) the α chain comprises the amino acid sequence set forth in SEQ ID NO: 258, and the β chain comprises the amino acid sequence set forth in SEQ ID NO: 259;
ag) the α chain comprises the amino acid sequence set forth in SEQ ID NO: 268, and the β chain comprises the amino acid sequence set forth in SEQ ID NO: 269;
ah) the α chain comprises the amino acid sequence set forth in SEQ ID NO: 276, and the β chain comprises the amino acid sequence set forth in SEQ ID NO: 277;
ai) the α chain comprises the amino acid sequence set forth in SEQ ID NO: 283, and the β chain comprises the amino acid sequence set forth in SEQ ID NO: 284;
aj) the α chain comprises the amino acid sequence set forth in SEQ ID NO: 293, and the β chain comprises the amino acid sequence set forth in SEQ ID NO: 294;
ak) the α chain comprises the amino acid sequence set forth in SEQ ID NO: 301, and the β chain comprises the amino acid sequence set forth in SEQ ID NO: 302;
al) the α chain comprises the amino acid sequence set forth in SEQ ID NO: 309, and the β chain comprises the amino acid sequence set forth in SEQ ID NO: 310;
am) the α chain comprises the amino acid sequence set forth in SEQ ID NO: 315, and the β chain comprises the amino acid sequence set forth in SEQ ID NO: 316;
an) the α chain comprises the amino acid sequence set forth in SEQ ID NO: 323, and the β chain comprises the amino acid sequence set forth in SEQ ID NO: 324;
ao) the α chain comprises the amino acid sequence set forth in SEQ ID NO: 331, and the β chain comprises the amino acid sequence set forth in SEQ ID NO: 332;
ap) the α chain comprises the amino acid sequence set forth in SEQ ID NO: 337, and the β chain comprises the amino acid sequence set forth in SEQ ID NO: 338;
aq) the α chain comprises the amino acid sequence set forth in SEQ ID NO: 343, and the β chain comprises the amino acid sequence set forth in SEQ ID NO: 344;
ar) the α chain comprises the amino acid sequence set forth in SEQ ID NO: 347, and the β chain comprising the amino acid sequence set forth in SEQ ID NO: 348;
as) the α chain comprises the amino acid sequence set forth in SEQ ID NO: 354, and the β chain comprises the amino acid sequence set forth in SEQ ID NO: 355;
at) the α chain comprises the amino acid sequence set forth in SEQ ID NO: 358, and the β chain comprises the amino acid sequence set forth in SEQ ID NO: 359;
au) the α chain comprises the amino acid sequence set forth in SEQ ID NO: 364, and the β chain comprises the amino acid sequence set forth in SEQ ID NO: 365;
av) the α chain comprises the amino acid sequence set forth in SEQ ID NO: 367, and the β chain comprises the amino acid sequence set forth in SEQ ID NO: 368;
aw) the α chain comprises the amino acid sequence set forth in SEQ ID NO: 373, and the β chain comprises the amino acid sequence set forth in SEQ ID NO: 374;
ax) the α chain comprises the amino acid sequence set forth in SEQ ID NO: 381, and the β chain comprises the amino acid sequence set forth in SEQ ID NO: 382; or
ay) the α chain comprises the amino acid sequence set forth in SEQ ID NO: 385, and the β chain comprises the amino acid sequence set forth in SEQ ID NO: 386.

A31. The foregoing TCR of A30, wherein
a) the α chain comprises the amino acid sequence set forth in SEQ ID NO: 13, and the β chain comprises the amino acid sequence set forth in SEQ ID NO: 14;
b) the α chain comprises the amino acid sequence set forth in SEQ ID NO: 22, and the β chain comprises the amino acid sequence set forth in SEQ ID NO: 23.

A32. The foregoing TCR of any one of A13-A31, wherein the extracellular domain binds to the same RAS peptide as a reference TCR or a functional fragment thereof, wherein the reference TCR or functional fragment thereof comprises an α chain variable region and a β chain variable region, wherein:
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56;
h) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
i) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
j) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
k) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
l) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
m) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
n) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
o) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
p) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
q) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
r) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
s) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
t) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
u) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
v) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
w) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
x) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
y) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
z) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
aa) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
ab) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
ac) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
ad) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ae) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
af) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ag) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ah) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
ai) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
aj) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ak) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
al) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
am) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
an) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
ao) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
ap) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ar) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
as) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
at) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
au) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
av) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
aw) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
ax) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378; or
ay) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

A33. The foregoing TCR of any one of A1-A32, wherein the TCR is recombinantly expressed, and/or expressed from a vector.

A34. The foregoing TCR of any one of A1-A33, wherein the TCR does not bind to a RAS peptide comprising or consisting of the amino acid sequence set forth in SEQ ID NO: 4.

A35. The foregoing TCR of any one of A13-A34, wherein the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 166 or SEQ ID NO: 167.

A36. The foregoing TCR of A35, wherein the α chain constant region comprises the amino acid sequence set forth in SEQ ID NO: 166 or SEQ ID NO: 167.

A37. The foregoing TCR of A13-A36, wherein the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 168, SEQ ID NO: 169, SEQ ID NO: 170, or SEQ ID NO: 171.

A38. The foregoing TCR of A37, wherein the β chain constant region comprises the amino acid sequence set forth in SEQ ID NO: 168, SEQ ID NO: 169, SEQ ID NO: 170, or SEQ ID NO: 171.

B1. In certain non-limiting embodiments, the presently disclosed subject matter provides a nucleic acid encoding the T cell receptor of any one of A1-A38.

C1. In certain non-limiting embodiments, the presently disclosed subject matter provides a cell comprising the TCR of any one of A1-A38 or the nucleic acid of B1.

C2. The foregoing cell of C1, wherein the cell is transduced with the TCR.

C3. The foregoing cell of C1 or C2, wherein the TCR is constitutively expressed on the surface of the cell.

C4. The foregoing cell of any one of C1-C3, wherein the cell is an immunoresponsive cell.

C5. The foregoing cell of any one of C1-C4, wherein the cell is selected from the group consisting of a T cell, a Natural Killer (NK) cell, and a pluripotent stem cell from which a lymphoid cell may be differentiated.

C6. The foregoing cell of C5, wherein the cell is a T cell.

C7. The foregoing cell of C6, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

C8. The foregoing cell of C7, wherein the T cell is a γδ T cell.

C9. The foregoing cell of C7, wherein the T cell is a NK-T cell.

C10. The foregoing cell of any one of C1-C9, wherein the TCR or the nucleic acid is integrated at a locus within the genome of the cell.

C11. The foregoing cell of C10, wherein the locus is selected from the group consisting of a TRAC locus, a TRBC locus, a TRDC locus, and a TRGC locus.

C12. The foregoing cell of C10 or C11, wherein the locus is a TRAC locus or a TRBC locus.

C13. The foregoing cell of any one of C1-C5, wherein the cell is a Natural Killer (NK) cell.

D1. In certain non-limiting embodiments, the presently disclosed subject matter provides a composition comprising the cell of any one of C1-C13.

D2. The foregoing composition of D1, which is a pharmaceutical composition further comprising a pharmaceutically acceptable carrier.

E1. In certain non-limiting embodiments, the presently disclosed subject matter provides a vector comprising the nucleic acid of B1.

E2. The foregoing vector of E1, wherein the vector is a γ-retroviral vector.

F1. In certain non-limiting embodiments, the presently disclosed subject matter provides a method for producing a cell that binds to a RAS peptide that comprises a Q61 mutation, comprising introducing into the cell the nucleic acid of B1 or the vector of E1 or E2.

G1. In certain non-limiting embodiments, the presently disclosed subject matter provides a polypeptide comprising a TCR or functional fragment thereof of any one of A1-A38 and a first binding molecule.

G2. The foregoing polypeptide of G1, wherein the first binding molecule is an antibody, an antibody fragment, a peptide, or a binding mimetic.

G3. The foregoing polypeptide of G1 or G2, wherein the polypeptide further comprises a second binding molecule.

G4. The foregoing polypeptide of G3, wherein the second binding molecule is an antibody, an antibody fragment, a peptide, or a binding mimetic.

H1. In certain non-limiting embodiments, the presently disclosed subject matter provides a method of treating and/or preventing a tumor associated with RAS in a subject, comprising administering to the subject the cell of any one of C1-C13, the composition of D1 or D2, or the polypeptide of any one of G1-G4.

H2. The foregoing method of H1, wherein the tumor is associated with a RAS mutation.

H3. The foregoing method of H2, wherein the RAS mutation is a Q61R mutation, a Q61K mutation, or a Q61L mutation.

H4. The foregoing method of any one of H1-H3, wherein the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

H5. The foregoing method of H4, wherein the tumor is colorectal cancer.

H6. The foregoing method of H4, wherein the tumor is thyroid cancer.

H7. The foregoing method of H4, wherein the tumor is cutaneous melanoma.

H8. The foregoing method of any of H1-H7, wherein the subject is a human.

I1. In certain non-limiting embodiments, the presently disclosed subject matter provides the cell of any one of C1-C13, the composition of D1 or D2, or the polypeptide of any one of G1-G4 for use in treating and/or preventing a tumor associated with RAS in a subject.

I2. The foregoing cell, composition, or polypeptide for use of I1, wherein the tumor is associated with a RAS mutation.

I3. The foregoing cell, composition, or polypeptide for use of I2, wherein the RAS mutation is a Q61R mutation, a Q61K mutation, or a Q61L mutation.

I4. The foregoing cell, composition, or polypeptide for use of any one of I1-I3, wherein the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

I5. The foregoing cell, composition, or polypeptide for of I4, wherein the tumor is colorectal cancer, thyroid cancer, or cutaneous melanoma.

I6. The foregoing cell, composition, or polypeptide for use of any one of 11-15, wherein the subject is human.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

The present example shows that neopeptides derived from the three most common NRAS(Q61) hotspot substitutions (R, K, and L) are naturally processed and presented in the context of HLA-A*01:01. This HLA allele is expressed by about 25% of the North American population and a comparably high frequency in western Europe.

Initial experiments were performed to identify T cells from melanoma patients that specifically recognize mutant NRAS (Q61) peptides but not the corresponding wild type (WT) sequence. Using single-cell sequencing, two TCR gene sequences from these patients were retrieved and functionally validated. These TCR gene sequences conferred recognition to NRAS(Q61R)+/HLA-A*01+ tumor cell lines when transduced into non-specific T cells but not NRAS(WT) or HLA mismatched controls. One of these TCRs (TCR1) was capable of cross-protecting against both NRAS(Q61R and Q61K)+ cell lines.

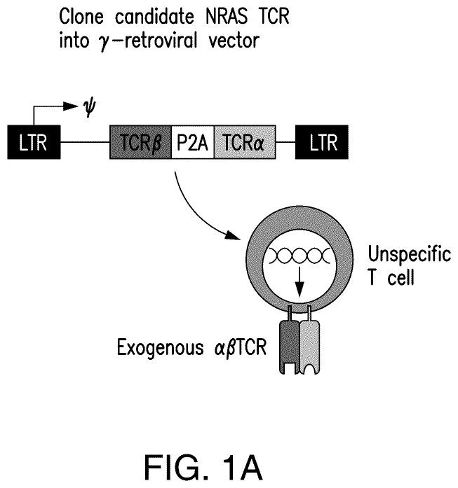

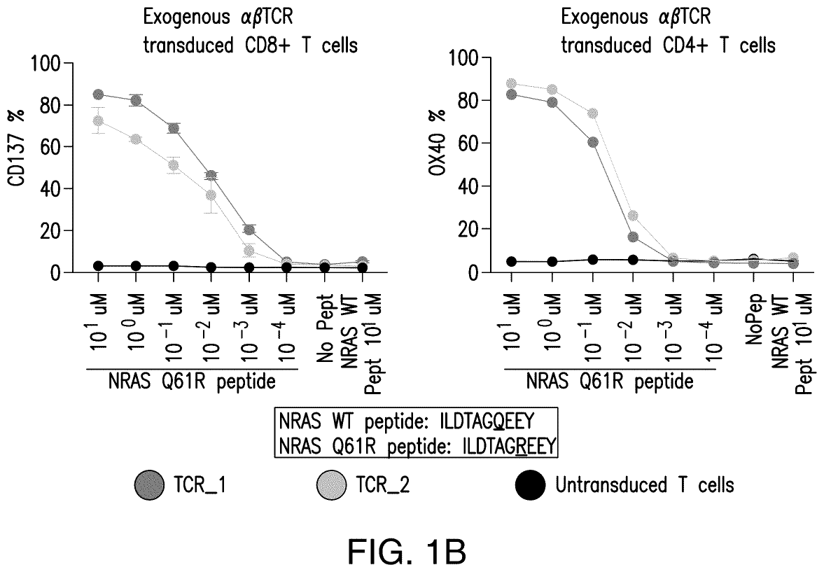

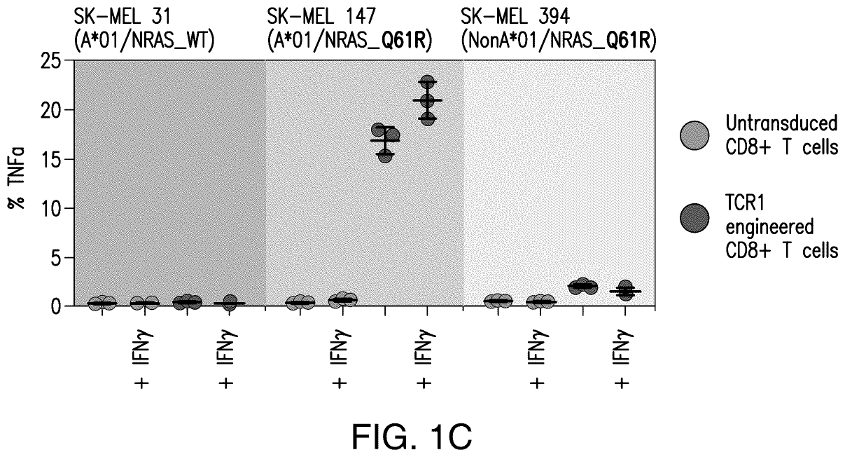

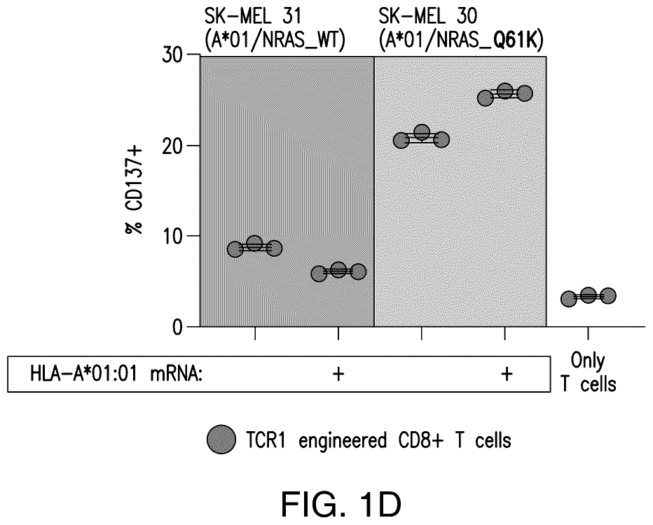

To identify T cell receptors recognizing processed and presented epitope(s) resulting from NRAS, COS-7 cells were used as an artificial antigen presenting cells (aAPC). COS-7 cells were co-electroporated with mRNA encoding HLA-A*01:01 and pulsed with either NRAS(Q61R), NRAS(Q61K), or Wild type (WT) NRAS peptides. Next, studies were conducted to functionally validate and measure co-receptor dependency of healthy donor (HD) and patient-derived TCRs specific for a RAS(Q61R) public neoantigen. Open repertoire (non-specific) T cells were retrovirally transduced with an individual retrieved TCR gene sequence. See FIG. 1A. The function of TCR transduced T cells was measured by coculturing with HLA-A*01:01⁺ target cells pulsed with either wild type (WT) NRAS or NRAS(Q61R) peptide. As shown in FIG. 1B, CD8⁺ and CD4⁺ T cells individually transduced with TCRs 1 and 2 showed mutation-specific recognition of titrating amounts of the NRAS(Q61R) peptide. TCR 1 and 2 expressing T cells did not recognize the WT NRAS peptide.

Next, studies were conducted to evaluate the recognition of endogenous levels of NRAS(Q61R) in two tumor lines by RAS-specific TCR library members, one of which was HLA-A*01:01⁺. Open-repertoire T cells were retrovirally transduced with an individual retrieved TCR gene sequence and cocultured with melanoma cell lines in the presence or absence of IFNγ. SK-MEL 31 is a melanoma line HLA-A*01:01⁺ and expressing NRAS(WT); SK-MEL 147 is a melanoma line HLA-A*01:01⁺ and expressing NRAS(Q61R); and SK-MEL 394 is a melanoma line non-HLA-A*01:01⁺ and expressing NRAS(Q61R). Intracellular TNF-α production was determined in CD8⁺ TCR⁺ T cells and is shown in FIG. 1C. TCR members were capable of recognizing endogenously processed and presented NRAS(Q61R) levels in a class-I restricted manner.

To test if TCR1 engineered T cells could afford cross-protection against alternative mutant RAS proteins, TCR-transduced T cells were co-cultured with HLA-A*01:01⁺ targets co-expressing individual NRAS(Q61K). WT RAS isoforms were used as specificity controls. Intracellular TNF-α production was determined in CD8⁺ T cells expressing the transduced TCR. As shown in FIG. 1D, cross-protective function of TCR1 engineered CD8⁺ T cells was observed.

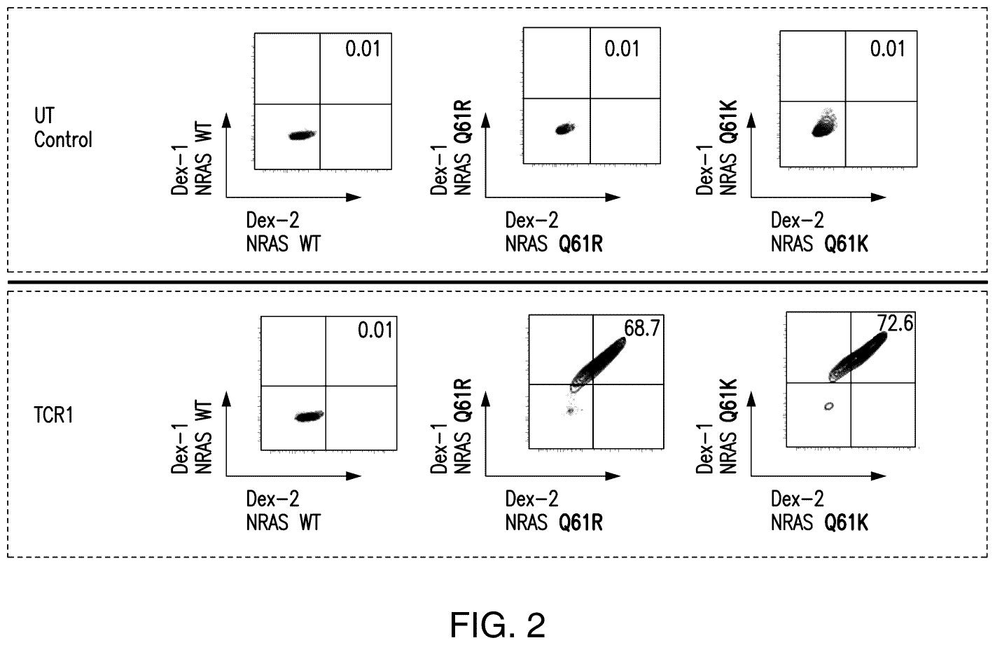

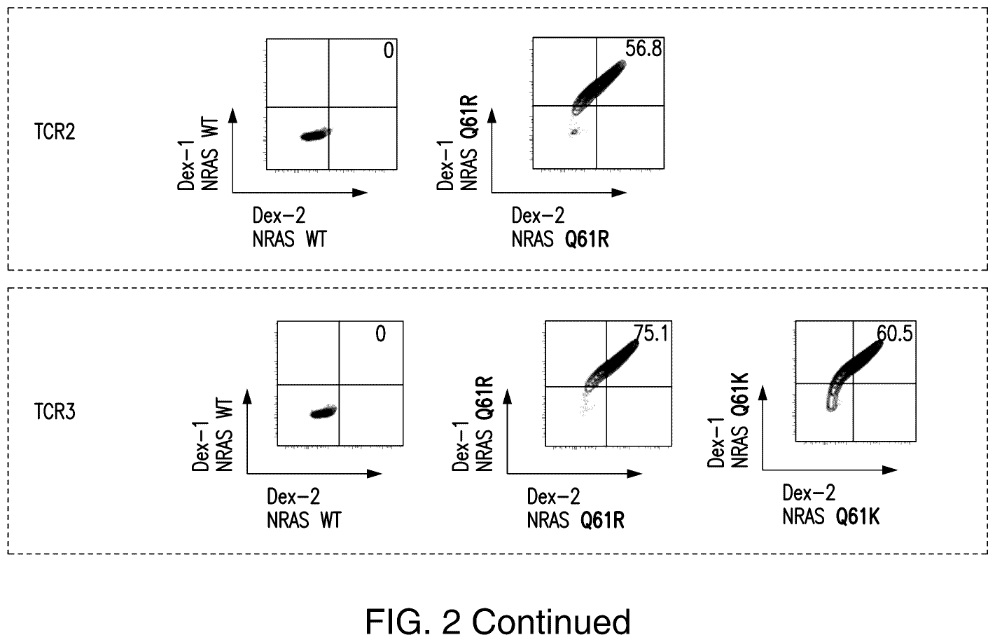

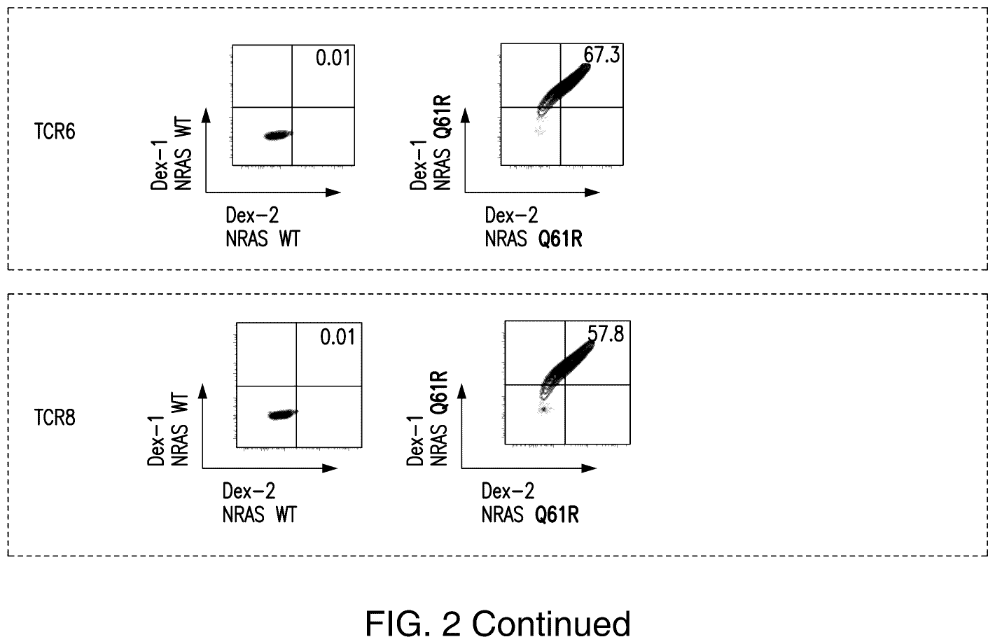

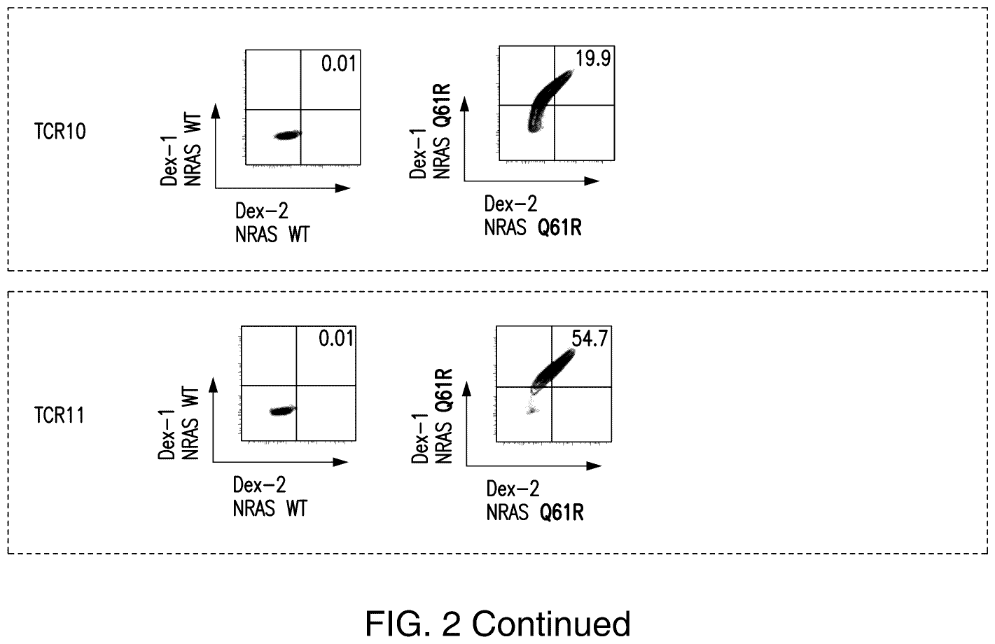

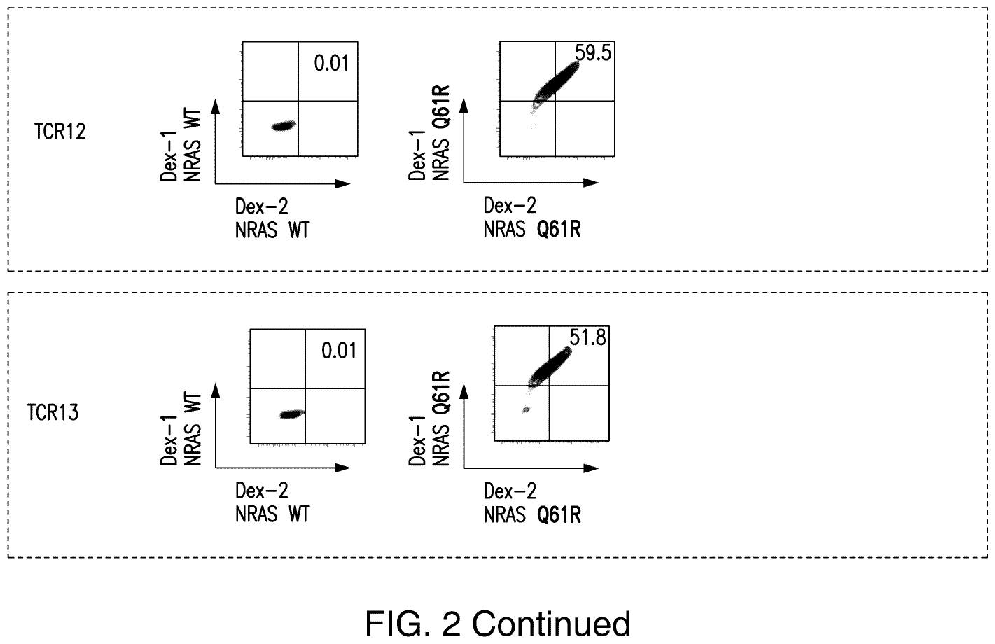

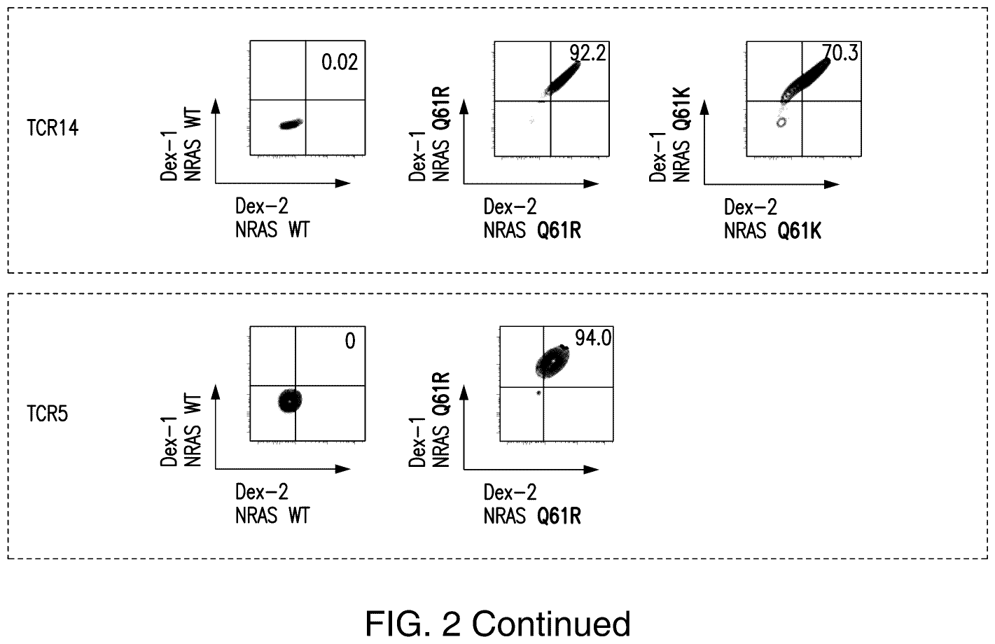

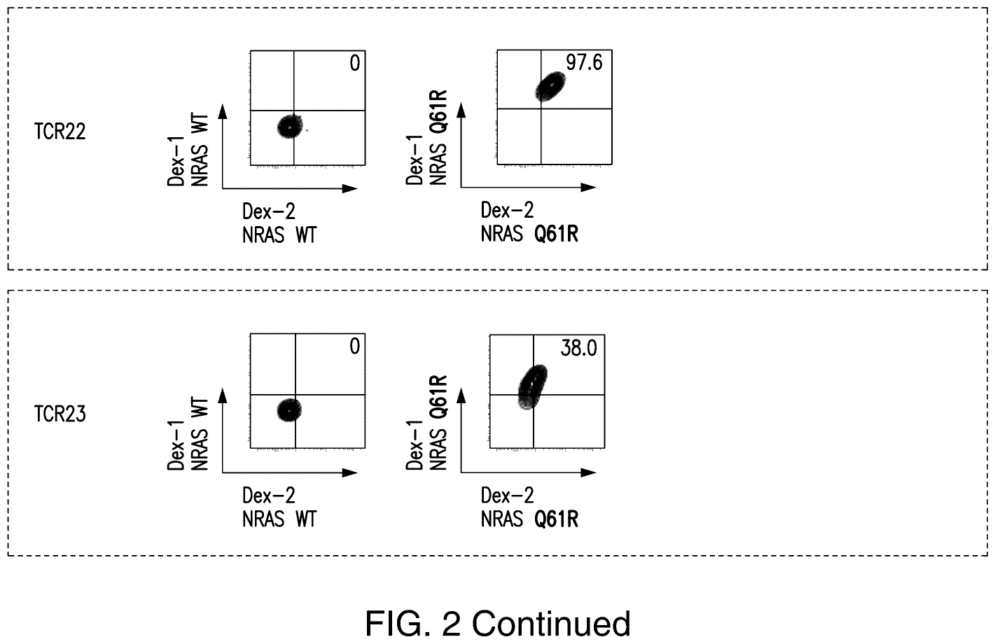

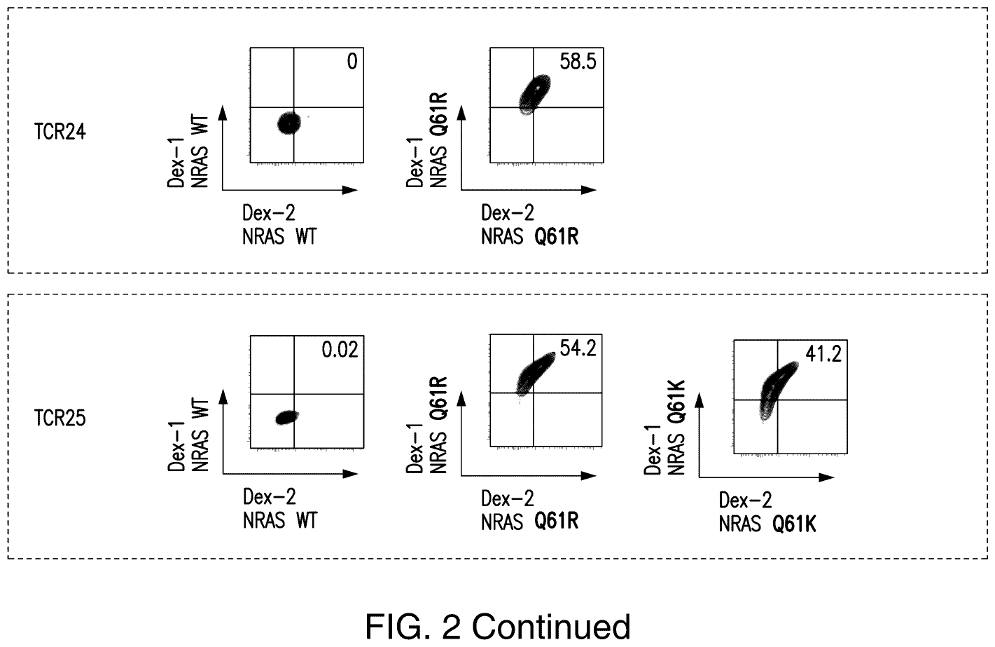

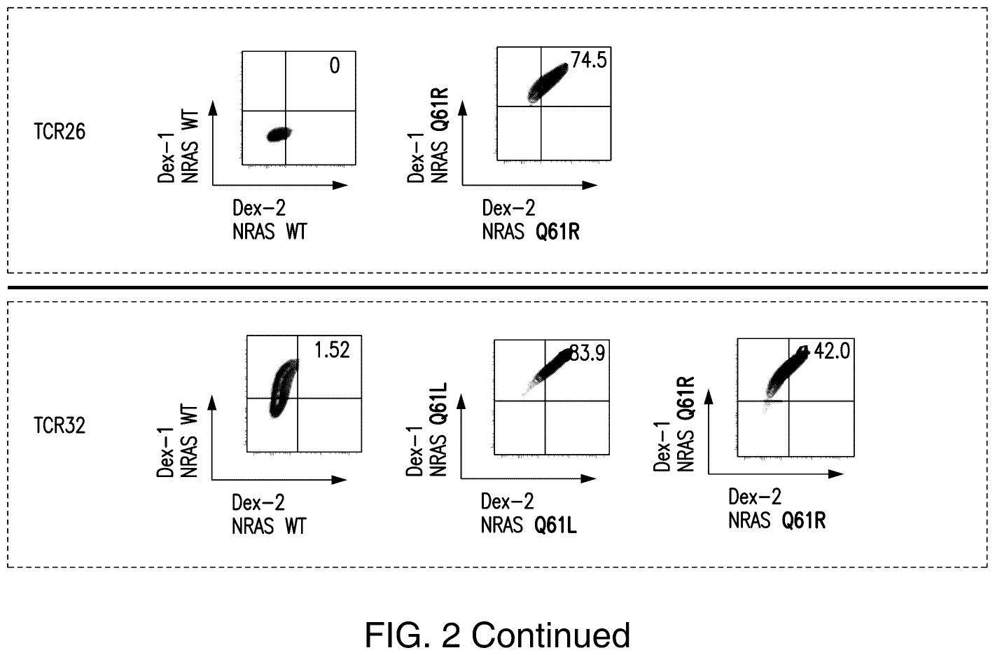

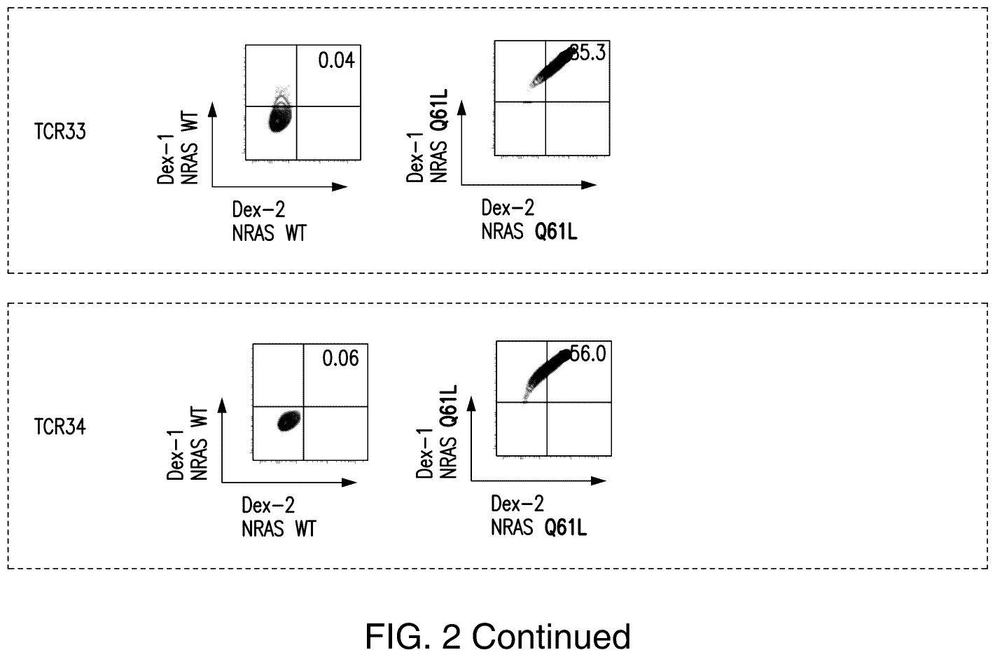

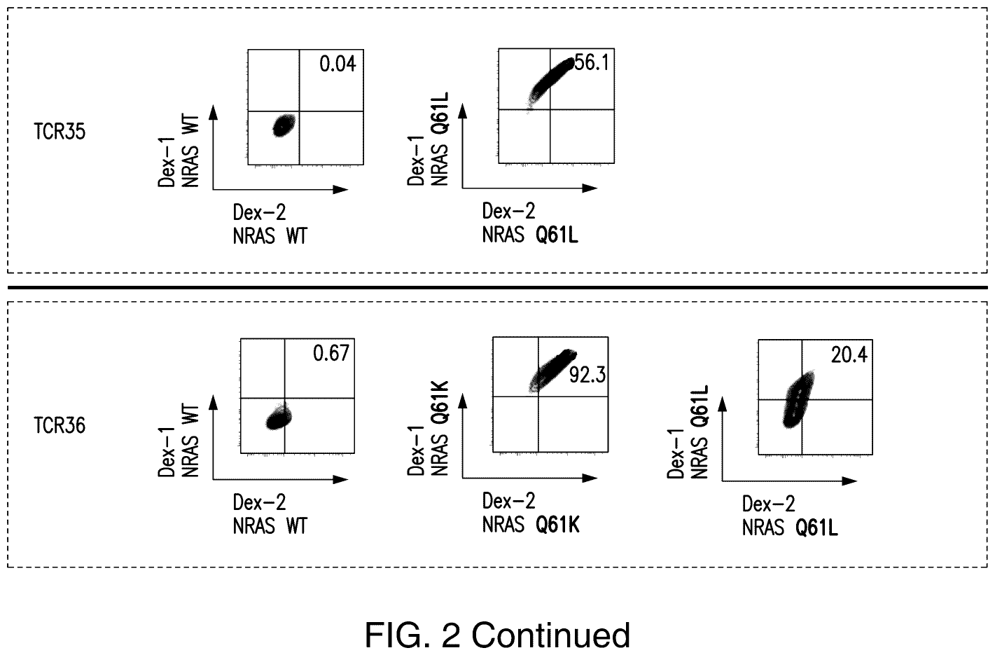

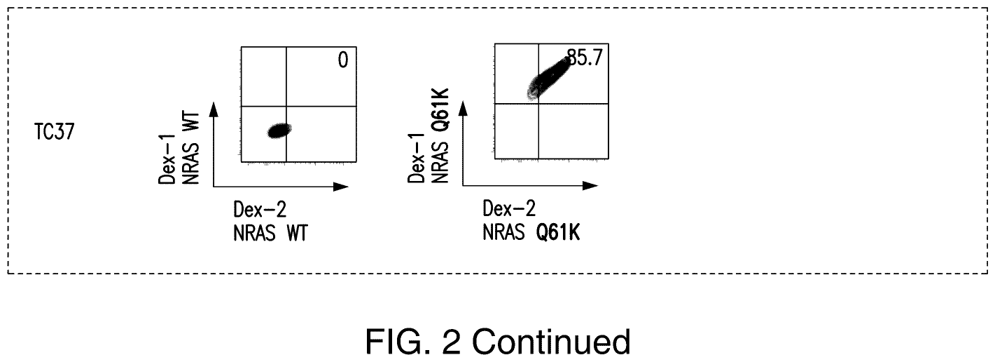

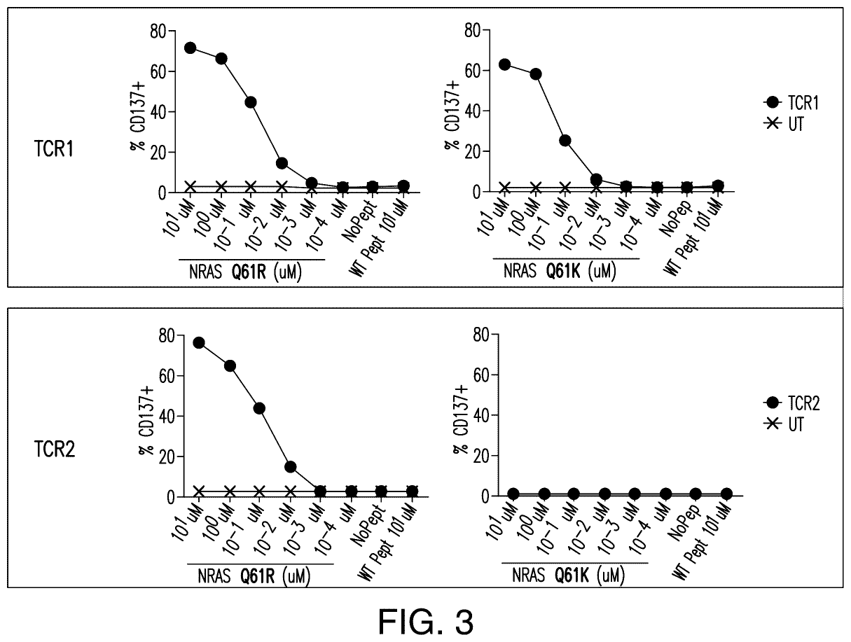

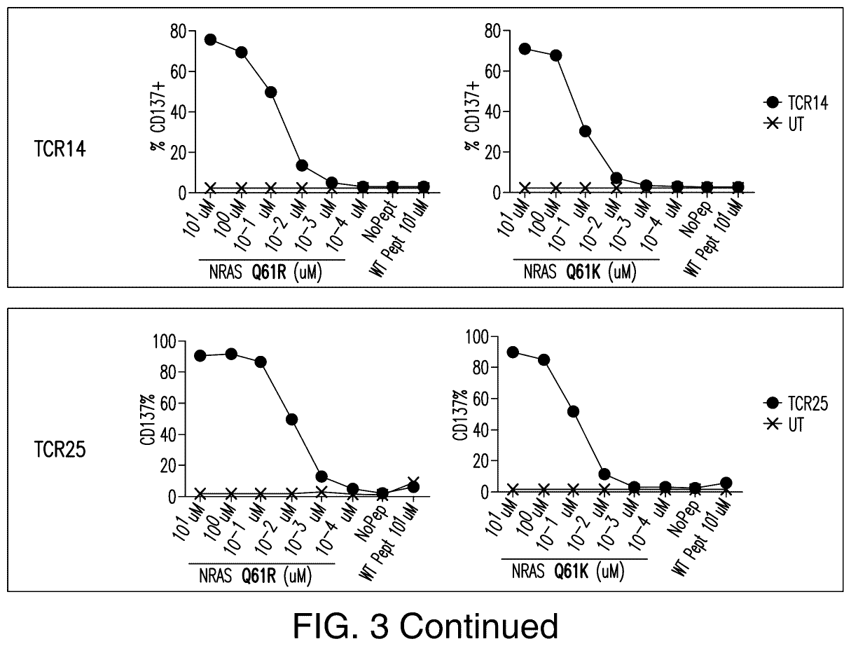

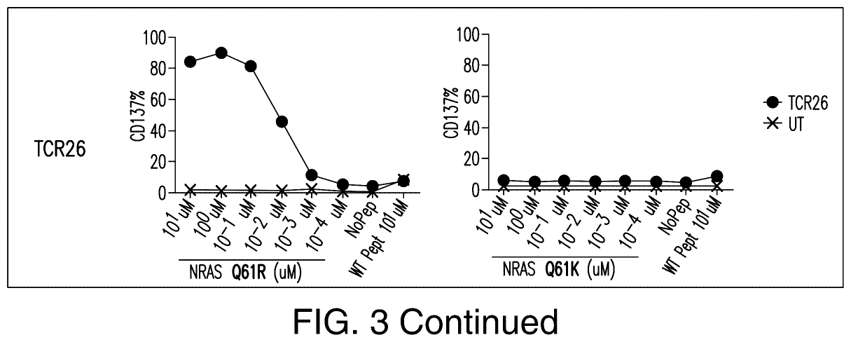

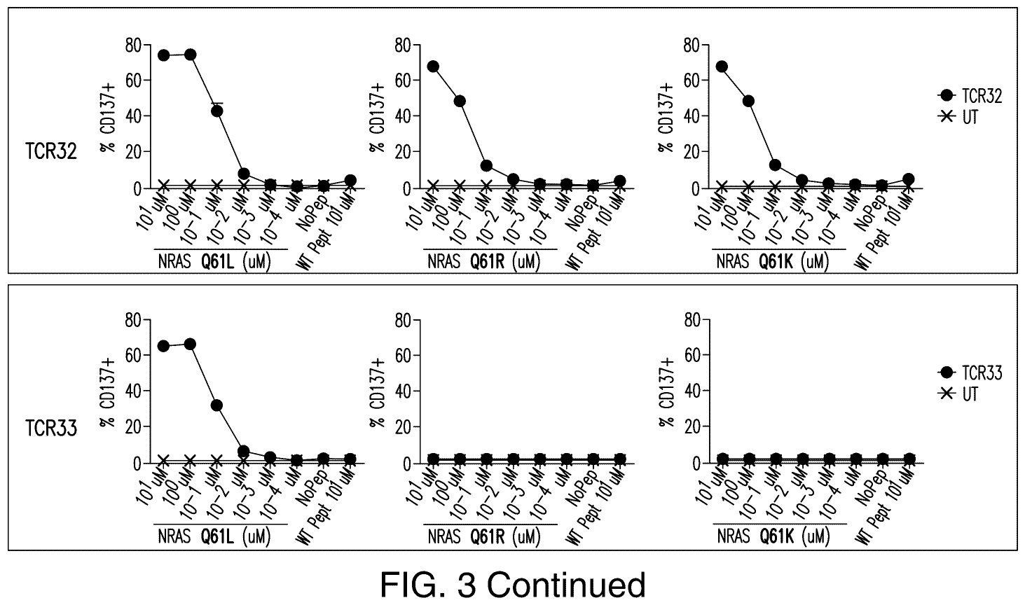

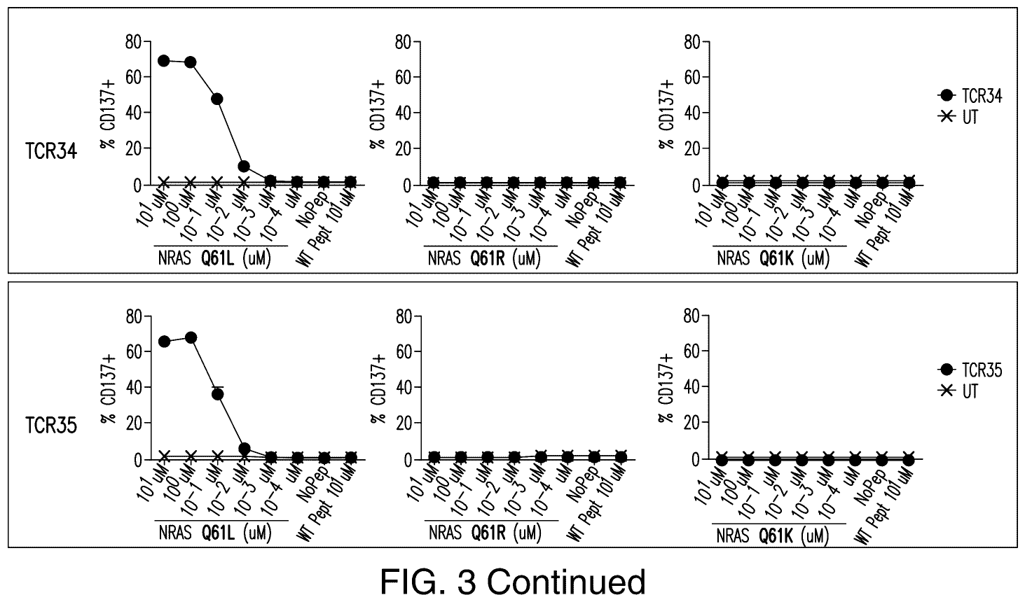

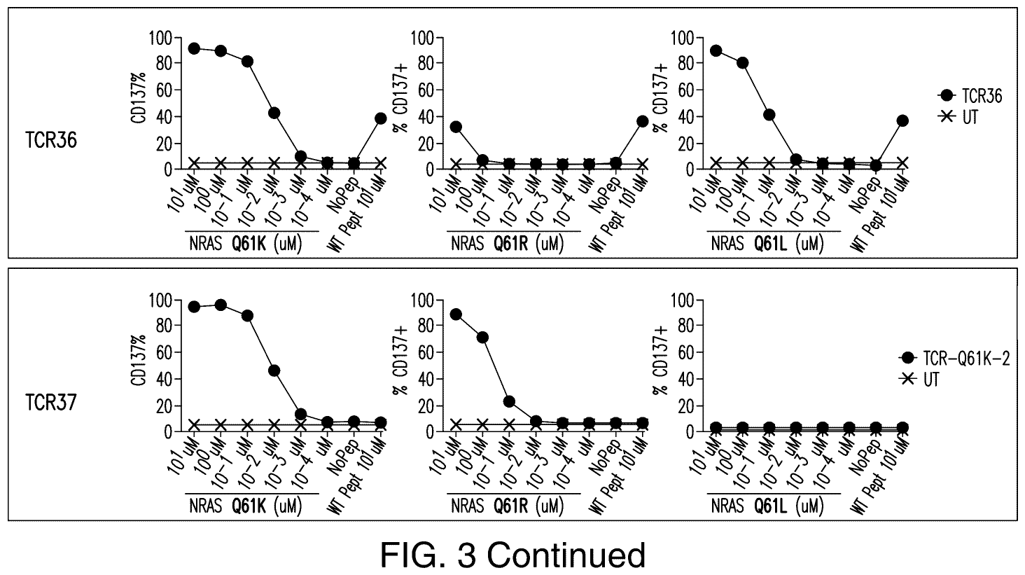

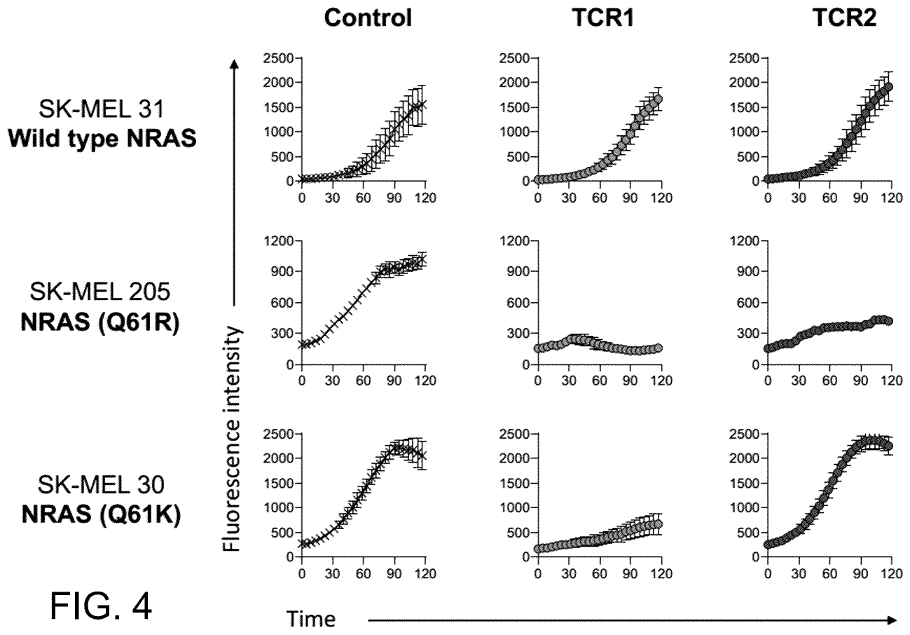

To further validate the recognition of RAS Q61mut public neoantigens, T cells expressing the indicated TCR were labeled with dual fluorophore-conjugated dextramers including HLA-A multimers bound to RAS wild-type or Q61mut peptide. As shown in FIGS. 2 and 3, T cells individually transduced with the indicated TCR showed mutation-specific recognition dextramers including the Q61mut peptide as well as titrating amounts of the same. Notably, the tested TCRs did not recognize the wild-type (WT) NRAS peptides. Finally, Finally, the capacity of + T cells transduced with the presently disclosed TCRs to mediate specific cell killing was measured. All tested TCRs mediated cytolysis of primary melanoma cells expressing a RAS Q61mut peptide (FIG. 4). Together, these findings establish the immunogenicity and therapeutic potential of a mutant RAS-derived public NeoAg and the recognition of these NeoAg by the presently disclosed TCRs.

From the foregoing description, it will be apparent that variations and modifications may be made to the invention described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A recombinant T cell receptor (TCR) that binds to a RAS peptide, wherein the TCR comprises an extracellular domain comprising an α chain and a β chain,
wherein the α chain comprises an α chain variable region and α chain constant region, and the β chain comprises a β chain variable region and a β chain constant region, and wherein
a) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7; and the β chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
b) the α chain variable region comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; and the β chain variable region comprises a CDR1 comprising the amino acid sequence set forth in SEQ ID NO: 8, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
d) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
e) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 44; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39;
f) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 47; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 50;
g) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 56;
h) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 55; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 61;
i) the α chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 67;
j) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and the β chain variable comprising the amino acid sequence set forth in SEQ ID NO: 49, and a CDR3 comprising the amino acid sequence set forth in SEQ ID NO: 67;
k) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 77; and the β chain variable region comprises a CDR1 comprising the amino acid sequence set forth in SEQ ID NO: 78, a CDR2 comprising the amino acid sequence set forth in SEQ ID NO: 79, and a CDR3 comprising the amino acid sequence set forth in SEQ ID NO: 80;
l) the α chain variable region comprises a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 88;
m) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 95; and the β chain variable region comprises a CDR1 comprising the amino acid sequence set forth in SEQ ID NO: 48, a CDR2 comprising the amino acid sequence set forth in SEQ ID NO: 49, and a CDR3 comprising the amino acid sequence set forth in SEQ ID NO: 96;
n) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 101; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 104;
o) the α chain variable region comprises a CDR1 comprising the amino acid sequence set forth in SEQ ID NO: 109, a CDR2 comprising the amino acid sequence set forth in SEQ ID NO: 110, and a CDR3 comprising the amino acid sequence set forth in SEQ ID NO: 111; and the β chain variable region comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114;
p) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 123;
q) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 130; and the β chain variable region comprises a CDR1 comprising the amino acid sequence set forth in SEQ ID NO: 131, a CDR2 comprising the amino acid sequence set forth in SEQ ID NO: 132, and a CDR3 comprising the amino acid sequence set forth in SEQ ID NO: 133;
r) the α chain variable region comprises a CDR1 comprising the amino acid sequence set forth in SEQ ID NO: 138, a CDR2 comprising the amino acid sequence set forth in SEQ ID NO: 35, and a CDR3 comprising the amino acid sequence set forth in SEQ ID NO: 139; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
s) the α chain variable region comprises a CDR1 comprising the amino acid sequence set forth in SEQ ID NO: 145, a CDR2 comprising the amino acid sequence set forth in SEQ ID NO: 146, and a CDR3 comprising the amino acid sequence set forth in SEQ ID NO: 147; and the β chain variable region comprises a CDR1 comprising the amino acid sequence set forth in SEQ ID NO: 102, a CDR2 comprising the amino acid sequence set forth in SEQ ID NO: 49, and a CDR3 comprising the amino acid sequence set forth in SEQ ID NO: 140;
t) the α chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 150; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 151;
u) the α chain variable region comprises a CDR1 comprising the amino acid sequence set forth in SEQ ID NO: 156, a CDR2 comprising the amino acid sequence set forth in SEQ ID NO: 157, and a CDR3 comprising the amino acid sequence set forth in SEQ ID NO: 158; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 161;
v) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 173; and the β chain variable region comprises a CDR1 comprising the amino acid sequence set forth in SEQ ID NO: 102, a CDR2 comprising the amino acid sequence set forth in SEQ ID NO: 103, and a CDR3 comprising the amino acid sequence set forth in SEQ ID NO: 174;
w) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 181; and the β chain variable region comprises a CDR1 comprising the amino acid sequence set forth in SEQ ID NO: 182, a CDR2 comprising the amino acid sequence set forth in SEQ ID NO: 183, and a CDR3 comprising the amino acid sequence set forth in SEQ ID NO: 184;
x) the α chain variable region comprises a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 189; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 190;
y) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 197; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 198;
z) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 205; and the β chain variable region comprises a CDR1 comprising the amino acid sequence set forth in SEQ ID NO: 122, a CDR2 comprising the amino acid sequence set forth in SEQ ID NO: 113, and a CDR3 comprising the amino acid sequence set forth in SEQ ID NO: 206;
aa) the α chain variable region comprises a CDR1 comprising the amino acid sequence set forth in SEQ ID NO: 211, a CDR2 comprising the amino acid sequence set forth in SEQ ID NO: 212, and a CDR3 comprising the amino acid sequence set forth in SEQ ID NO: 213; and the β chain variable region comprises a CDR1 comprising the amino acid sequence set forth in SEQ ID NO: 214, a CDR2 comprising the amino acid sequence set forth in SEQ ID NO: 215, and a CDR3 comprising the amino acid sequence set forth in SEQ ID NO: 216;
ab) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 221; and the β chain variable region comprises a CDR1 comprising the amino acid sequence set forth in SEQ ID NO: 159, a CDR2 comprising the amino acid sequence set forth in SEQ ID NO: 160, and a CDR3 comprising the amino acid sequence set forth in SEQ ID NO: 222;
ac) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 229; and the β chain variable region comprises a CDR1 comprising the amino acid sequence set forth in SEQ ID NO: 230, a CDR2 comprising the amino acid sequence set forth in SEQ ID NO: 231, and a CDR3 comprising the amino acid sequence set forth in SEQ ID NO: 232;
ad) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 237; and the β chain variable region comprises a CDR1 comprising the amino acid sequence set forth in SEQ ID NO: 238, a CDR2 comprising the amino acid sequence set forth in SEQ ID NO: 239, and a CDR3 comprising the amino acid sequence set forth in SEQ ID NO: 240;
ae) the α chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 245; and the β chain variable region comprises a CDR1 comprising the amino acid sequence set forth in SEQ ID NO: 246, a CDR2 comprising the amino acid sequence set forth in SEQ ID NO: 247, and a CDR3 comprising the amino acid sequence set forth in SEQ ID NO: 248;
af) the α chain variable region comprises a CDR1 comprising the amino acid sequence set forth in SEQ ID NO: 156, a CDR2 comprising the amino acid sequence set forth in SEQ ID NO: 253, and a CDR3 comprising the amino acid sequence set forth in SEQ ID NO: 254; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 255;
ag) the α chain variable region comprises a CDR1 comprising the amino acid sequence set forth in SEQ ID NO: 260, a CDR2 comprising the amino acid sequence set forth in SEQ ID NO: 261, and a CDR3 comprising the amino acid sequence set forth in SEQ ID NO: 262; and the β chain variable region comprises a CDR1 comprising the amino acid sequence set forth in SEQ ID NO: 263, a CDR2 comprising the amino acid sequence set forth in SEQ ID NO: 264, and a CDR3 comprising the amino acid sequence set forth in SEQ ID NO: 265;
ah) the α chain variable region comprises a CDR1 comprising the amino acid sequence set forth in SEQ ID NO: 203, a CDR2 comprising the amino acid sequence set forth in SEQ ID NO: 204, and a CDR3 comprising the amino acid sequence set forth in SEQ ID NO: 270; and the β chain variable region comprises a CDR1 comprising the amino acid sequence set forth in SEQ ID NO: 271, a CDR2 comprising the amino acid sequence set forth in SEQ ID NO: 272, and a CDR3 comprising the amino acid sequence set forth in SEQ ID NO: 273;
ai) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 278; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 280;
aj) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 287; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 290;
ak) the α chain variable region comprises a CDR1 comprising the amino acid sequence set forth in SEQ ID NO: 227, a CDR2 comprising the amino acid sequence set forth in SEQ ID NO: 228, and a CDR3 comprising the amino acid sequence set forth in SEQ ID NO: 295; and the β chain variable region comprises a CDR1 comprising the amino acid sequence set forth in SEQ ID NO: 296, a CDR2 comprising the amino acid sequence set forth in SEQ ID NO: 297, and a CDR3 comprising the amino acid sequence set forth in SEQ ID NO: 298;
al) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 303; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 306;
am) the α chain variable region comprises a CDR1 comprising the amino acid sequence set forth in SEQ ID NO: 128, a CDR2 comprising the amino acid sequence set forth in SEQ ID NO: 129, and a CDR3 comprising the amino acid sequence set forth in SEQ ID NO: 311; and the β chain variable region comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 312;
an) the α chain variable region comprises a CDR1 comprising the amino acid sequence set forth in SEQ ID NO: 317, a CDR2 comprising the amino acid sequence set forth in SEQ ID NO: 318, and a CDR3 comprising the amino acid sequence set forth in SEQ ID NO: 319; and the β chain variable region comprises a CDR1 comprising the amino acid sequence set forth in SEQ ID NO: 263, a CDR2 comprising the amino acid sequence set forth in SEQ ID NO: 279, and a CDR3 comprising the amino acid sequence set forth in SEQ ID NO: 320;
ao) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 328;
ap) the α chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 333; and the β chain variable region comprises a CDR1 comprising the amino acid sequence set forth in SEQ ID NO: 304, a CDR2 comprising the amino acid sequence set forth in SEQ ID NO: 305, and a CDR3 comprising the amino acid sequence set forth in SEQ ID NO: 334;
aq) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 339; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
ar) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 345; and the β chain variable region comprises a CDR1 comprising the amino acid sequence set forth in SEQ ID NO: 288, a CDR2 comprising the amino acid sequence set forth in SEQ ID NO: 289, and a CDR3 comprising the amino acid sequence set forth in SEQ ID NO: 340;
as) the α chain variable region comprises a CDR1 comprising the amino acid sequence set forth in SEQ ID NO: 93, a CDR2 comprising the amino acid sequence set forth in SEQ ID NO: 94, and a CDR3 comprising the amino acid sequence set forth in SEQ ID NO: 349; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
at) the α chain variable region comprises a CDR1 comprising the amino acid sequence set forth in SEQ ID NO: 195, a CDR2 comprising the amino acid sequence set forth in SEQ ID NO: 196, and a CDR3 comprising the amino acid sequence set forth in SEQ ID NO: 356; and the β chain variable region comprises a CDR1 comprising the amino acid sequence set forth in SEQ ID NO: 182, a CDR2 comprising the amino acid sequence set forth in SEQ ID NO: 350, and a CDR3 comprising the amino acid sequence set forth in SEQ ID NO: 351;
au) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 360; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 361;
av) the α chain variable region comprises a CDR1 comprising the amino acid sequence set forth in SEQ ID NO: 285, a CDR2 comprising the amino acid sequence set forth in SEQ ID NO: 286, and a CDR3 comprising the amino acid sequence set forth in SEQ ID NO: 325; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
aw) the α chain variable region comprises a CDR1 comprising the amino acid sequence set forth in SEQ ID NO: 369, a CDR2 comprising the amino acid sequence set forth in SEQ ID NO: 370, and a CDR3 comprising the amino acid sequence set forth in SEQ ID NO: 371; and the β chain variable region comprises a CDR1 comprising the amino acid sequence set forth in SEQ ID NO: 326, a CDR2 comprising the amino acid sequence set forth in SEQ ID NO: 327, and a CDR3 comprising the amino acid sequence set forth in SEQ ID NO: 290;
ax) the α chain variable region comprises a CDR1 comprising the amino acid sequence set forth in SEQ ID NO: 375, a CDR2 comprising the amino acid sequence set forth in SEQ ID NO: 376, and a CDR3 comprising the amino acid sequence set forth in SEQ ID NO: 377; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378; or
ay) the α chain variable region comprises a CDR1 comprising the amino acid sequence set forth in SEQ ID NO: 179, a CDR2 comprising the amino acid sequence set forth in SEQ ID NO: 180, and a CDR3 comprising the amino acid sequence set forth in SEQ ID NO: 383; and the β chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 378.

2. The recombinant TCR of claim 1, wherein the RAS peptide comprises a Q61R mutation, a Q61K mutation, or a Q61L mutation.

3. The recombinant TCR of claim 1, wherein the RAS peptide comprises or consists of the amino acid sequence set forth in SEQ ID NO: 1, SEQ ID NO: 2, or SEQ ID NO: 3.

4. The recombinant TCR of claim 1, wherein the RAS peptide is associated with an HLA class I complex.

5. The recombinant TCR of claim 4, wherein the HLA class I complex is selected from an HLA-A, an HLA-B, and an HLA-C.

6. The recombinant TCR of claim 4, wherein the HLA class I complex is an HLA-A.

7. The recombinant TCR of claim 4, wherein the HLA-A is an HLA-A*01 superfamily member.

8. The recombinant TCR of claim 7, wherein the HLA-A*01 superfamily member is selected from the group consisting of HLA-A*01, HLA-A*25, HLA-A*26, HLA-A*30, HLA-A*31, HLA-A*32, HLA-A*36, HLA-A*74:10, and HLA-A*80.

9. The recombinant TCR of claim 7, wherein the HLA-A*01 superfamily member is HLA-A*01:01.

10. The recombinant TCR of claim 1, wherein:
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 20, and the β chain variable region comprises an amino acid sequence that is at least about 80% homologous or identical to the amino acid sequence set forth in SEQ ID NO: 21;
c) the α chain variable region comprises an amino acid sequence that is at least about 80% homologous or identical to the amino acid sequence set forth in SEQ ID NO: 30, and the β chain variable region comprises an amino acid sequence that is at least about 80% homologous or identical to the amino acid sequence set forth in SEQ ID NO: 31;
d) the α chain variable region comprises an amino acid sequence that is at least about 80% homologous or identical to the amino acid sequence set forth in SEQ ID NO: 40, and the β chain variable region comprises an amino acid sequence that is at least about 80% homologous or identical to the amino acid sequence set forth in SEQ ID NO: 41;
e) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 41;
f) the α chain variable region comprises an amino acid sequence that is at least about 80% homologous or identical to the amino acid sequence set forth in SEQ ID NO: 51, and the β chain variable region comprises an amino acid sequence that is at least about 80% homologous or identical to the amino acid sequence set forth in SEQ ID NO: 52;
g) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 58;
h) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 62;
i) the α chain variable region comprises an amino acid sequence that is at least about 80% homologous or identical to the amino acid sequence set forth in SEQ ID NO: 68, and the β chain variable region comprises an amino acid sequence that is at least about 80% homologous or identical to the amino acid sequence set forth in SEQ ID NO: 69;
j) the α chain variable region comprises an amino acid sequence that is at least about 80% homologous or identical to the amino acid sequence set forth in SEQ ID NO: 75, and the β chain variable region comprises an amino acid sequence that is at least about 80% homologous or identical to the amino acid sequence set forth in SEQ ID NO: 69;
k) the α chain variable region comprises an amino acid sequence that is at least about 80% homologous or identical to the amino acid sequence set forth in SEQ ID NO: 81, and the β chain variable region comprises an amino acid sequence that is at least about 80% homologous or identical to the amino acid sequence set forth in SEQ ID NO: 82;
l) the α chain variable region comprises an amino acid sequence that is at least about 80% homologous or identical to the amino acid sequence set forth in SEQ ID NO: 89, and the β chain variable region comprises an amino acid sequence that is at least about 80% homologous or identical to the amino acid sequence set forth in SEQ ID NO: 90;
m) the α chain variable region comprises an amino acid sequence that is at least about 80% homologous or identical to the amino acid sequence set forth in SEQ ID NO: 97, and the β chain variable region comprises an amino acid sequence that is at least about 80% homologous or identical to the amino acid sequence set forth in SEQ ID NO: 98;
n) the α chain variable region comprises an amino acid sequence that is at least about 80% homologous or identical to the amino acid sequence set forth in SEQ ID NO: 105, and the β chain variable region comprises an amino acid sequence that is at least about 80% homologous or identical to the amino acid sequence set forth in SEQ ID NO: 106;
o) the α chain variable region comprises an amino acid sequence that is at least about 80% homologous or identical to the amino acid sequence set forth in SEQ ID NO: 115, and the β chain variable region comprises an amino acid sequence that is at least about 80% homologous or identical to the amino acid sequence set forth in SEQ ID NO: 116;
p) the α chain variable region comprises an amino acid sequence that is at least about 80% homologous or identical to the amino acid sequence set forth in SEQ ID NO: 124, and the β chain variable region comprises an amino acid sequence that is at least about 80% homologous or identical to the amino acid sequence set forth in SEQ ID NO: 125;
q) the α chain variable region comprises an amino acid sequence that is at least about 80% homologous or identical to the amino acid sequence set forth in SEQ ID NO: 134, and the β chain variable region comprises an amino acid sequence that is at least about 80% homologous or identical to the amino acid sequence set forth in SEQ ID NO: 135;
r) the α chain variable region comprises an amino acid sequence that is at least about 80% homologous or identical to the amino acid sequence set forth in SEQ ID NO: 141, and the β chain variable region comprises an amino acid sequence that is at least about 80% homologous or identical to the amino acid sequence set forth in SEQ ID NO: 142;
s) the α chain variable region comprises an amino acid sequence that is at least about 80% homologous or identical to the amino acid sequence set forth in SEQ ID NO: 148, and the β chain variable region comprises an amino acid sequence that is at least about 80% homologous or identical to the amino acid sequence set forth in SEQ ID NO: 142;
t) the α chain variable region comprises an amino acid sequence that is at least about 80% homologous or identical to the amino acid sequence set forth in SEQ ID NO: 152, and the β chain variable region comprises an amino acid sequence that is at least about 80% homologous or identical to the amino acid sequence set forth in SEQ ID NO: 153;
u) the α chain variable region comprises an amino acid sequence that is at least about 80% homologous or identical to the amino acid sequence set forth in SEQ ID NO: 162, and the β chain variable region comprises an amino acid sequence that is at least about 80% homologous or identical to the amino acid sequence set forth in SEQ ID NO: 163;
v) the α chain variable region comprises an amino acid sequence that is at least about 80% homologous or identical to the amino acid sequence set forth in SEQ ID NO: 175, and the β chain variable region comprises an amino acid sequence that is at least about 80% homologous or identical to the amino acid sequence set forth in SEQ ID NO: 176;
w) the α chain variable region comprises an amino acid sequence that is at least about 80% homologous or identical to the amino acid sequence set forth in SEQ ID NO: 185, and the β chain variable region comprises an amino acid sequence that is at least about 80% homologous or identical to the amino acid sequence set forth in SEQ ID NO: 186;
x) the α chain variable region comprises an amino acid sequence that is at least about 80% homologous or identical to the amino acid sequence set forth in SEQ ID NO: 191, and the β chain variable region comprises an amino acid sequence that is at least about 80% homologous or identical to the amino acid sequence set forth in SEQ ID NO: 192;
y) the α chain variable region comprises an amino acid sequence that is at least about 80% homologous or identical to the amino acid sequence set forth in SEQ ID NO: 199, and the β chain variable region comprises an amino acid sequence that is at least about 80% homologous or identical to the amino acid sequence set forth in SEQ ID NO: 200;
z) the α chain variable region comprises an amino acid sequence that is at least about 80% homologous or identical to the amino acid sequence set forth in SEQ ID NO: 207, and the β chain variable region comprises an amino acid sequence that is at least about 80% homologous or identical to the amino acid sequence set forth in SEQ ID NO: 208;
aa) the α chain variable region comprises an amino acid sequence that is at least about 80% homologous or identical to the amino acid sequence set forth in SEQ ID NO: 217, and the β chain variable region comprises an amino acid sequence that is at least about 80% homologous or identical to the amino acid sequence set forth in SEQ ID NO: 218;
ab) the α chain variable region comprises an amino acid sequence that is at least about 80% homologous or identical to the amino acid sequence set forth in SEQ ID NO: 223, and the β chain variable region comprises an amino acid sequence that is at least about 80% homologous or identical to the amino acid sequence set forth in SEQ ID NO: 224;
ac) the α chain variable region comprises an amino acid sequence that is at least about 80% homologous or identical to the amino acid sequence set forth in SEQ ID NO: 233, and the β chain variable region comprises an amino acid sequence that is at least about 80% homologous or identical to the amino acid sequence set forth in SEQ ID NO: 234;
ad) the α chain variable region comprises an amino acid sequence that is at least about 80% homologous or identical to the amino acid sequence set forth in SEQ ID NO: 241, and the β chain variable region comprises an amino acid sequence that is at least about 80% homologous or identical to the amino acid sequence set forth in SEQ ID NO: 242;
ae) the α chain variable region comprises an amino acid sequence that is at least about 80% homologous or identical to the amino acid sequence set forth in SEQ ID NO: 249, and the β chain variable region comprises an amino acid sequence that is at least about 80% homologous or identical to the amino acid sequence set forth in SEQ ID NO: 250;
af) the α chain variable region comprises an amino acid sequence that is at least about 80% homologous or identical to the amino acid sequence set forth in SEQ ID NO: 256, and the β chain variable region comprises an amino acid sequence that is at least about 80% homologous or identical to the amino acid sequence set forth in SEQ ID NO: 257;
ag) the α chain variable region comprises an amino acid sequence that is at least about 80% homologous or identical to the amino acid sequence set forth in SEQ ID NO: 266, and the β chain variable region comprises an amino acid sequence that is at least about 80% homologous or identical to the amino acid sequence set forth in SEQ ID NO: 267;
ah) the α chain variable region comprises an amino acid sequence that is at least about 80% homologous or identical to the amino acid sequence set forth in SEQ ID NO: 274, and the β chain variable region comprises an amino acid sequence that is at least about 80% homologous or identical to the amino acid sequence set forth in SEQ ID NO: 275;
ai) the α chain variable region comprises an amino acid sequence that is at least about 80% homologous or identical to the amino acid sequence set forth in SEQ ID NO: 281, and the β chain variable region comprises an amino acid sequence that is at least about 80% homologous or identical to the amino acid sequence set forth in SEQ ID NO: 282;
aj) the α chain variable region comprises an amino acid sequence that is at least about 80% homologous or identical to the amino acid sequence set forth in SEQ ID NO: 291, and the β chain variable region comprises an amino acid sequence that is at least about 80% homologous or identical to the amino acid sequence set forth in SEQ ID NO: 292;
ak) the α chain variable region comprises an amino acid sequence that is at least about 80% homologous or identical to the amino acid sequence set forth in SEQ ID NO: 299, and the β chain variable region comprises an amino acid sequence that is at least about 80% homologous or identical to the amino acid sequence set forth in SEQ ID NO: 300;
al) the α chain variable region comprises an amino acid sequence that is at least about 80% homologous or identical to the amino acid sequence set forth in SEQ ID NO: 307, and the β chain variable region comprises an amino acid sequence that is at least about 80% homologous or identical to the amino acid sequence set forth in SEQ ID NO: 308;
am) the α chain variable region comprises an amino acid sequence that is at least about 80% homologous or identical to the amino acid sequence set forth in SEQ ID NO: 313, and the β chain variable region comprises an amino acid sequence that is at least about 80% homologous or identical to the amino acid sequence set forth in SEQ ID NO: 314;
an) the α chain variable region comprises an amino acid sequence that is at least about 80% homologous or identical to the amino acid sequence set forth in SEQ ID NO: 321, and the β chain variable region comprises an amino acid sequence that is at least about 80% homologous or identical to the amino acid sequence set forth in SEQ ID NO: 322;
ao) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 330;
ap) the α chain variable region comprises an amino acid sequence that is at least about 80% homologous or identical to the amino acid sequence set forth in SEQ ID NO: 335, and the β chain variable region comprises an amino acid sequence that is at least about 80% homologous or identical to the amino acid sequence set forth in SEQ ID NO: 336;
aq) the α chain variable region comprises an amino acid sequence that is at least about 80% homologous or identical to the amino acid sequence set forth in SEQ ID NO: 341, and the β chain variable region comprises an amino acid sequence that is at least about 80% homologous or identical to the amino acid sequence set forth in SEQ ID NO: 342;
ar) the α chain variable region comprises an amino acid sequence that is at least about 80% homologous or identical to the amino acid sequence set forth in SEQ ID NO: 346, and the β chain variable region comprises an amino acid sequence that is at least about 80% homologous or identical to the amino acid sequence set forth in SEQ ID NO: 342;
as) the α chain variable region comprises an amino acid sequence that is at least about 80% homologous or identical to the amino acid sequence set forth in SEQ ID NO: 352, and the β chain variable region comprises an amino acid sequence that is at least about 80% homologous or identical to the amino acid sequence set forth in SEQ ID NO: 353;
at) the α chain variable region comprises an amino acid sequence that is at least about 80% homologous or identical to the amino acid sequence set forth in SEQ ID NO: 357, and the β chain variable region comprises an amino acid sequence that is at least about 80% homologous or identical to the amino acid sequence set forth in SEQ ID NO: 353;
au) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
av) the α chain variable region comprises an amino acid sequence that is at least about 80% homologous or identical to the amino acid sequence set forth in SEQ ID NO: 362, and the β chain variable region comprises an amino acid sequence that is at least about 80% homologous or identical to the amino acid sequence set forth in SEQ ID NO: 363;
aw) the α chain variable region comprises an amino acid sequence that is at least about 80% homologous or identical to the amino acid sequence set forth in SEQ ID NO: 329, and the β chain variable region comprises an amino acid sequence that is at least about 80% homologous or identical to the amino acid sequence set forth in SEQ ID NO: 366;
ax) the α chain variable region comprises an amino acid sequence that is at least about 80% homologous or identical to the amino acid sequence set forth in SEQ ID NO: 372, and the β chain variable region comprises an amino acid sequence that is at least about 80% homologous or identical to the amino acid sequence set forth in SEQ ID NO: 366;
ay) the α chain variable region comprises an amino acid sequence that is at least about 80% homologous or identical to the amino acid sequence set forth in SEQ ID NO: 379, and the β chain variable region comprises an amino acid sequence that is at least about 80% homologous or identical to the amino acid sequence set forth in SEQ ID NO: 380; or
az) the α chain variable region comprises an amino acid sequence that is at least about 80% homologous or identical to the amino acid sequence set forth in SEQ ID NO: 384, and the β chain variable region comprises an amino acid sequence that is at least about 80% homologous or identical to the amino acid sequence set forth in SEQ ID NO: 380.

11. The recombinant TCR of claim 1, wherein:
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 20, and the β chain variable region comprises the amino acid sequence set forth in SEQ ID NO: 21;
c) the α chain variable region comprises the amino acid sequence set forth in SEQ ID NO: 30, and the β chain variable region comprises the amino acid sequence set forth in SEQ ID NO: 31;
d) the α chain variable region comprises the amino acid sequence set forth in SEQ ID NO: 40, and the β chain variable region comprises the amino acid sequence set forth in SEQ ID NO: 41;
e) the α chain variable region comprises the amino acid sequence set forth in SEQ ID NO: 45, and the β chain variable region comprises the amino acid sequence set forth in SEQ ID NO: 41;
f) the α chain variable region comprises the amino acid sequence set forth in SEQ ID NO: 51, and the β chain variable region comprises the amino acid sequence set forth in SEQ ID NO: 52;
g) the α chain variable region comprises the amino acid sequence set forth in SEQ ID NO: 57, and the β chain variable region comprises the amino acid sequence set forth in SEQ ID NO: 58;
h) the α chain variable region comprises the amino acid sequence set forth in SEQ ID NO: 57, and the β chain variable region comprises the amino acid sequence set forth in SEQ ID NO: 62;
i) the α chain variable region comprises the amino acid sequence set forth in SEQ ID NO: 68, and the β chain variable region comprises the amino acid sequence set forth in SEQ ID NO: 69;
j) the α chain variable region comprises the amino acid sequence set forth in SEQ ID NO: 75, and the β chain variable region comprises the amino acid sequence set forth in SEQ ID NO: 69;
k) the α chain variable region comprises the amino acid sequence set forth in SEQ ID NO: 81, and the β chain variable region comprises the amino acid sequence set forth in SEQ ID NO: 82;
l) the α chain variable region comprises the amino acid sequence set forth in SEQ ID NO: 89, and the β chain variable region comprises the amino acid sequence set forth in SEQ ID NO: 90;
m) the α chain variable region comprises the amino acid sequence set forth in SEQ ID NO: 97, and the β chain variable region comprises the amino acid sequence set forth in SEQ ID NO: 98;
n) the α chain variable region comprises the amino acid sequence set forth in SEQ ID NO: 105, and the β chain variable region comprises the amino acid sequence set forth in SEQ ID NO: 106;
o) the α chain variable region comprises the amino acid sequence set forth in SEQ ID NO: 115, and the β chain variable region comprises the amino acid sequence set forth in SEQ ID NO: 116;
p) the α chain variable region comprises the amino acid sequence set forth in SEQ ID NO: 124, and the β chain variable region comprises the amino acid sequence set forth in SEQ ID NO: 125;
q) the α chain variable region comprises the amino acid sequence set forth in SEQ ID NO: 134, and the β chain variable region comprises the amino acid sequence set forth in SEQ ID NO: 135;
r) the α chain variable region comprises the amino acid sequence set forth in SEQ ID NO: 141, and the β chain variable region comprises the amino acid sequence set forth in SEQ ID NO: 142;
s) the α chain variable region comprises the amino acid sequence set forth in SEQ ID NO: 148, and the β chain variable region comprises the amino acid sequence set forth in SEQ ID NO: 142;
t) the α chain variable region comprises the amino acid sequence set forth in SEQ ID NO: 152, and the β chain variable region comprises the amino acid sequence set forth in SEQ ID NO: 153;
u) the α chain variable region comprises the amino acid sequence set forth in SEQ ID NO: 162, and the β chain variable region comprises the amino acid sequence set forth in SEQ ID NO: 163;
v) the α chain variable region comprises the amino acid sequence set forth in SEQ ID NO: 175, and the β chain variable region comprises the amino acid sequence set forth in SEQ ID NO: 176;
w) the α chain variable region comprises the amino acid sequence set forth in SEQ ID NO: 185, and the β chain variable region comprises the amino acid sequence set forth in SEQ ID NO: 186;
x) the α chain variable region comprises the amino acid sequence set forth in SEQ ID NO: 191, and the β chain variable region comprises the amino acid sequence set forth in SEQ ID NO: 192;
y) the α chain variable region comprises the amino acid sequence set forth in SEQ ID NO: 199, and the β chain variable region comprises the amino acid sequence set forth in SEQ ID NO: 200;
z) the α chain variable region comprises the amino acid sequence set forth in SEQ ID NO: 207, and the β chain variable region comprises the amino acid sequence set forth in SEQ ID NO: 208;
aa) the α chain variable region comprises the amino acid sequence set forth in SEQ ID NO: 217, and the β chain variable region comprises the amino acid sequence set forth in SEQ ID NO: 218;
ab) the α chain variable region comprises the amino acid sequence set forth in SEQ ID NO: 223, and the β chain variable region comprises the amino acid sequence set forth in SEQ ID NO: 224;
ac) the α chain variable region comprises the amino acid sequence set forth in SEQ ID NO: 233, and the β chain variable region comprises the amino acid sequence set forth in SEQ ID NO: 234;
ad) the α chain variable region comprises the amino acid sequence set forth in SEQ ID NO: 241, and the β chain variable region comprises the amino acid sequence set forth in SEQ ID NO: 242;
ae) the α chain variable region comprises the amino acid sequence set forth in SEQ ID NO: 249, and the β chain variable region comprises the amino acid sequence set forth in SEQ ID NO: 250;
af) the α chain variable region comprises the amino acid sequence set forth in SEQ ID NO: 256, and the β chain variable region comprises the amino acid sequence set forth in SEQ ID NO: 257;
ag) the α chain variable region comprises the amino acid sequence set forth in SEQ ID NO: 266, and the β chain variable region comprises the amino acid sequence set forth in SEQ ID NO: 267;
ah) the α chain variable region comprises the amino acid sequence set forth in SEQ ID NO: 274, and the β chain variable region comprises the amino acid sequence set forth in SEQ ID NO: 275;
ai) the α chain variable region comprises the amino acid sequence set forth in SEQ ID NO: 281, and the β chain variable region comprises the amino acid sequence set forth in SEQ ID NO: 282;
aj) the α chain variable region comprises the amino acid sequence set forth in SEQ ID NO: 291, and the β chain variable region comprises the amino acid sequence set forth in SEQ ID NO: 292;
ak) the α chain variable region comprises the amino acid sequence set forth in SEQ ID NO: 299, and the β chain variable region comprises the amino acid sequence set forth in SEQ ID NO: 300;
al) the α chain variable region comprises the amino acid sequence set forth in SEQ ID NO: 307, and the β chain variable region comprises the amino acid sequence set forth in SEQ ID NO: 308;
am) the α chain variable region comprises the amino acid sequence set forth in SEQ ID NO: 313, and the β chain variable region comprises the amino acid sequence set forth in SEQ ID NO: 314;
an) the α chain variable region comprises the amino acid sequence set forth in SEQ ID NO: 321, and the β chain variable region comprises the amino acid sequence set forth in SEQ ID NO: 322;
ao) the α chain variable region comprises the amino acid sequence set forth in SEQ ID NO: 329, and the β chain variable region comprises the amino acid sequence set forth in SEQ ID NO: 330;
ap) the α chain variable region comprises the amino acid sequence set forth in SEQ ID NO: 335, and the β chain variable region comprises the amino acid sequence set forth in SEQ ID NO: 336;
aq) the α chain variable region comprises the amino acid sequence set forth in SEQ ID NO: 341, and the β chain variable region comprises the amino acid sequence set forth in SEQ ID NO: 342;
ar) the α chain variable region comprises the amino acid sequence set forth in SEQ ID NO: 346, and the β chain variable region comprises the amino acid sequence set forth in SEQ ID NO: 342;
as) the α chain variable region comprises the amino acid sequence set forth in SEQ ID NO: 352, and the β chain variable region comprises the amino acid sequence set forth in SEQ ID NO: 353;
at) the α chain variable region comprises the amino acid sequence set forth in SEQ ID NO: 357, and the β chain variable region comprises the amino acid sequence set forth in SEQ ID NO: 353;
au) the α chain variable region comprises the amino acid sequence set forth in SEQ ID NO: 362, and the β chain variable region comprises the amino acid sequence set forth in SEQ ID NO: 363;
av) the α chain variable region comprises the amino acid sequence set forth in SEQ ID NO: 362, and the β chain variable region comprises the amino acid sequence set forth in SEQ ID NO: 363;
aw) the α chain variable region comprises the amino acid sequence set forth in SEQ ID NO: 329, and the β chain variable region comprises the amino acid sequence set forth in SEQ ID NO: 366;
ax) the α chain variable region comprises the amino acid sequence set forth in SEQ ID NO: 372, and the β chain variable region comprises the amino acid sequence set forth in SEQ ID NO: 366;
ay) the α chain variable region comprises the amino acid sequence set forth in SEQ ID NO: 379, and the β chain variable region comprises the amino acid sequence set forth in SEQ ID NO: 380; or
az) the α chain variable region comprises the amino acid sequence set forth in SEQ ID NO: 384, and the β chain variable region comprises the amino acid sequence set forth in SEQ ID NO: 380.

12. The recombinant TCR of claim 1, wherein:
a) the α chain comprises the amino acid sequence set forth in SEQ ID NO: 13, and the β chain comprises the amino acid sequence set forth in SEQ ID NO: 14;
b) the α chain comprises the amino acid sequence set forth in SEQ ID NO: 22, and the β chain comprises the amino acid sequence set forth in SEQ ID NO: 23;
c) the α chain comprises the amino acid sequence set forth in SEQ ID NO: 32, and the β chain comprises the amino acid sequence set forth in SEQ ID NO: 33;
d) the α chain comprises the amino acid sequence set forth in SEQ ID NO: 42, and the β chain comprising the amino acid sequence set forth in SEQ ID NO: 43;
e) the α chain comprises the amino acid sequence set forth in SEQ ID NO: 46, and the β chain comprises the amino acid sequence set forth in SEQ ID NO: 43;
f) the α chain comprises the amino acid sequence set forth in SEQ ID NO: 53, and the β chain comprises the amino acid sequence set forth in SEQ ID NO: 54;
g) the α chain comprises the amino acid sequence set forth in SEQ ID NO: 59, and the β chain comprises the amino acid sequence set forth in SEQ ID NO: 60;
h) the α chain comprises the amino acid sequence set forth in SEQ ID NO: 59, and the β chain comprises the amino acid sequence set forth in SEQ ID NO: 63;
i) the α chain comprises the amino acid sequence set forth in SEQ ID NO: 70, and the β chain comprises the amino acid sequence set forth in SEQ ID NO: 71;
j) the α chain comprises the amino acid sequence set forth in SEQ ID NO: 76, and the β chain comprises the amino acid sequence set forth in SEQ ID NO: 71;
k) the α chain comprises the amino acid sequence set forth in SEQ ID NO: 83, and the β chain comprises the amino acid sequence set forth in SEQ ID NO: 84;
l) the α chain comprises the amino acid sequence set forth in SEQ ID NO: 91, and the β chain comprises the amino acid sequence set forth in SEQ ID NO: 92;
m) the α chain comprises the amino acid sequence set forth in SEQ ID NO: 99, and the β chain comprises the amino acid sequence set forth in SEQ ID NO: 100;
n) the α chain comprises the amino acid sequence set forth in SEQ ID NO: 107, and the β chain comprises the amino acid sequence set forth in SEQ ID NO: 108;
o) the α chain comprises the amino acid sequence set forth in SEQ ID NO: 117, and the β chain comprises the amino acid sequence set forth in SEQ ID NO: 118;
p) the α chain comprises the amino acid sequence set forth in SEQ ID NO: 126, and the β chain comprises the amino acid sequence set forth in SEQ ID NO: 127;
q) the α chain comprises the amino acid sequence set forth in SEQ ID NO: 136, and the β chain comprises the amino acid sequence set forth in SEQ ID NO: 137;
r) the α chain comprises the amino acid sequence set forth in SEQ ID NO: 143, and the β chain comprises the amino acid sequence set forth in SEQ ID NO: 144;
s) the α chain comprises the amino acid sequence set forth in SEQ ID NO: 149, and the β chain comprises the amino acid sequence set forth in SEQ ID NO: 144;
t) the α chain comprises the amino acid sequence set forth in SEQ ID NO: 154, and the β chain comprises the amino acid sequence set forth in SEQ ID NO: 155;
u) the α chain comprises the amino acid sequence set forth in SEQ ID NO: 164, and the β chain comprises the amino acid sequence set forth in SEQ ID NO: 165;
v) the α chain comprises the amino acid sequence set forth in SEQ ID NO: 177, and the β chain comprises the amino acid sequence set forth in SEQ ID NO: 178;
w) the α chain comprises the amino acid sequence set forth in SEQ ID NO: 187, and the β chain comprises the amino acid sequence set forth in SEQ ID NO: 188;
x) the α chain comprises the amino acid sequence set forth in SEQ ID NO: 193, and the β chain comprises the amino acid sequence set forth in SEQ ID NO: 194;
y) the α chain comprises the amino acid sequence set forth in SEQ ID NO: 201, and the β chain comprising the amino acid sequence set forth in SEQ ID NO: 202;
z) the α chain comprises the amino acid sequence set forth in SEQ ID NO: 209, and the β chain comprises the amino acid sequence set forth in SEQ ID NO: 210;
aa) the α chain comprises the amino acid sequence set forth in SEQ ID NO: 219, and the β chain comprises the amino acid sequence set forth in SEQ ID NO: 220;
ab) the α chain comprises the amino acid sequence set forth in SEQ ID NO: 225, and the β chain comprises the amino acid sequence set forth in SEQ ID NO: 226;
ac) the α chain comprises the amino acid sequence set forth in SEQ ID NO: 235, and the β chain comprises the amino acid sequence set forth in SEQ ID NO: 236;
ad) the α chain comprises the amino acid sequence set forth in SEQ ID NO: 243, and the β chain comprises the amino acid sequence set forth in SEQ ID NO: 244;
ae) the α chain comprises the amino acid sequence set forth in SEQ ID NO: 251, and the β chain comprises the amino acid sequence set forth in SEQ ID NO: 252;
af) the α chain comprises the amino acid sequence set forth in SEQ ID NO: 258, and the β chain comprises the amino acid sequence set forth in SEQ ID NO: 259;
ag) the α chain comprises the amino acid sequence set forth in SEQ ID NO: 268, and the β chain comprises the amino acid sequence set forth in SEQ ID NO: 269;
ah) the α chain comprises the amino acid sequence set forth in SEQ ID NO: 276, and the β chain comprises the amino acid sequence set forth in SEQ ID NO: 277;
ai) the α chain comprises the amino acid sequence set forth in SEQ ID NO: 283, and the β chain comprises the amino acid sequence set forth in SEQ ID NO: 284;
aj) the α chain comprises the amino acid sequence set forth in SEQ ID NO: 293, and the β chain comprises the amino acid sequence set forth in SEQ ID NO: 294;
ak) the α chain comprises the amino acid sequence set forth in SEQ ID NO: 301, and the β chain comprises the amino acid sequence set forth in SEQ ID NO: 302;
al) the α chain comprises the amino acid sequence set forth in SEQ ID NO: 309, and the β chain comprises the amino acid sequence set forth in SEQ ID NO: 310;
am) the α chain comprises the amino acid sequence set forth in SEQ ID NO: 315, and the β chain comprises the amino acid sequence set forth in SEQ ID NO: 316;
an) the α chain comprises the amino acid sequence set forth in SEQ ID NO: 323, and the β chain comprises the amino acid sequence set forth in SEQ ID NO: 324;
ao) the α chain comprises the amino acid sequence set forth in SEQ ID NO: 331, and the β chain comprises the amino acid sequence set forth in SEQ ID NO: 332;
ap) the α chain comprises the amino acid sequence set forth in SEQ ID NO: 337, and the β chain comprises the amino acid sequence set forth in SEQ ID NO: 338;
aq) the α chain comprises the amino acid sequence set forth in SEQ ID NO: 343, and the β chain comprises the amino acid sequence set forth in SEQ ID NO: 344;
ar) the α chain comprises the amino acid sequence set forth in SEQ ID NO: 347, and the β chain comprising the amino acid sequence set forth in SEQ ID NO: 348;
as) the α chain comprises the amino acid sequence set forth in SEQ ID NO: 354, and the β chain comprises the amino acid sequence set forth in SEQ ID NO: 355;
at) the α chain comprises the amino acid sequence set forth in SEQ ID NO: 358, and the β chain comprises the amino acid sequence set forth in SEQ ID NO: 359;
au) the α chain comprises the amino acid sequence set forth in SEQ ID NO: 364, and the β chain comprises the amino acid sequence set forth in SEQ ID NO: 365;
av) the α chain comprises the amino acid sequence set forth in SEQ ID NO: 367, and the β chain comprises the amino acid sequence set forth in SEQ ID NO: 368;
aw) the α chain comprises the amino acid sequence set forth in SEQ ID NO: 373, and the β chain comprises the amino acid sequence set forth in SEQ ID NO: 374;
ax) the α chain comprises the amino acid sequence set forth in SEQ ID NO: 381, and the β chain comprises the amino acid sequence set forth in SEQ ID NO: 382; or
ay) the α chain comprises the amino acid sequence set forth in SEQ ID NO: 385, and the β chain comprises the amino acid sequence set forth in SEQ ID NO: 386.

13. The recombinant TCR of claim 1, wherein the TCR does not bind to a RAS peptide comprising the amino acid sequence set forth in SEQ ID NO: 4.

14. The recombinant TCR of claim 1, wherein
a) the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence set forth in SEQ ID NO: 166 or SEQ ID NO: 167; and/or
b) the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 168, SEQ ID NO: 169, SEQ ID NO: 170, or SEQ ID NO: 171.

15. The recombinant TCR of claim 14, wherein (a) the α chain constant region comprises the amino acid sequence set forth in SEQ ID NO: 166 or SEQ ID NO: 167; and/or (b) the β chain constant region comprises the amino acid sequence set forth in SEQ ID NO: 168, SEQ ID NO: 169, SEQ ID NO: 170, or SEQ ID NO: 171.

16. A nucleic acid encoding the T cell receptor (TCR) of claim 1.

17. A cell comprising the recombinant TCR of claim 1.

18. The cell of claim 17, wherein the TCR is constitutively expressed on the surface of the cell.

19. The cell of claim 17, wherein the cell is an immunoresponsive cell.

20. The immunoresponsive cell of claim 17, wherein the cell is selected from the group consisting of a T cell, a Natural Killer (NK) cell, and a pluripotent stem cell from which a lymphoid cell may be differentiated.

21. The cell of claim 20, wherein the cell is a T cell.

22. The cell of claim 21, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

23. The cell of claim 22, wherein the T cell is a γδ T cell or a NK-T cell.

24. The cell of claim 17, wherein the TCR or the nucleic acid is integrated at a locus within the genome of the cell.

25. The cell of claim 17, wherein the cell is a Natural Killer (NK) cell.

26. A composition comprising the cell of claim 17.

27. A vector comprising the nucleic acid of claim 16.

28. A method for producing a cell that binds to a RAS peptide that comprises a Q61 mutation, comprising introducing into the cell the nucleic acid of claim 16.

29. A polypeptide comprising a TCR or functional fragment thereof of claim 1 and a first binding molecule.

30. A method of treating and/or preventing a tumor associated with RAS in a subject, comprising administering to the subject the cell of claim 17.

31. The method of claim 30, wherein the tumor is associated with a RAS mutation.

32. The method of claim 31, wherein the RAS mutation is a Q61R mutation, a Q61K mutation, or a Q61L mutation.

33. The method of claim 30, wherein the tumor is selected from the group consisting of colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

34. The method of claim 33, wherein the tumor is colorectal cancer.

35. The method of claim 33, wherein the tumor is thyroid cancer.

36. The method of claim 33, wherein the tumor is cutaneous melanoma.

37. The method of claim 30, wherein the subject is a human.

38. A recombinant T cell receptor (TCR) that binds to a RAS peptide, wherein the TCR comprises an extracellular domain comprising an α chain and a β chain, wherein the α chain comprises an α chain variable region and α chain constant region, and the β chain comprises a β chain variable region and a β chain constant region, and wherein
a) the α chain variable region comprises a CDR1, a CDR2, and a CDR3 of the α chain variable region comprising the amino acid sequence set forth in SEQ ID NO: 11, and the β chain variable region comprises a CDR1, a CDR2, and a CDR3 of the β chain variable region comprising the amino acid sequence set forth in SEQ ID NO: 12;
b) the α chain variable region comprises a CDR1, a CDR2, and a CDR3 of the α chain variable region comprising the amino acid sequence set forth in SEQ ID NO: 20, and the β chain variable region comprises a CDR1, a CDR2, and a CDR3 of the β chain variable region comprising the amino acid sequence set forth in SEQ ID NO: 21;
c) the α chain variable region comprises a CDR1, a CDR2, and a CDR3 of the α chain variable region comprising the amino acid sequence set forth in SEQ ID NO: 30, and the β chain variable region comprises a CDR1, a CDR2, and a CDR3 of the β chain variable region comprising the amino acid sequence set forth in SEQ ID NO: 31;
d) the α chain variable region comprises a CDR1, a CDR2, and a CDR3 of the α chain variable region comprising the amino acid sequence set forth in SEQ ID NO: 40, and the β chain variable region comprises a CDR1, a CDR2, and a CDR3 of the β chain variable region comprising the amino acid sequence set forth in SEQ ID NO: 41;
e) the α chain variable region comprises a CDR1, a CDR2, and a CDR3 of the α chain variable region comprising the amino acid sequence set forth in SEQ ID NO: 45, and the β chain variable region comprises a CDR1, a CDR2, and a CDR3 of the β chain variable region comprising the amino acid sequence set forth in SEQ ID NO: 41;
f) the α chain variable region comprises a CDR1, a CDR2, and a CDR3 of the α chain variable region comprising the amino acid sequence set forth in SEQ ID NO: 51, and the β chain variable region comprises a CDR1, a CDR2, and a CDR3 of the β chain variable region comprising the amino acid sequence set forth in SEQ ID NO: 52;
g) the α chain variable region comprises a CDR1, a CDR2, and a CDR3 of the α chain variable region comprising the amino acid sequence set forth in SEQ ID NO: 57, and the β chain variable region comprises a CDR1, a CDR2, and a CDR3 of the β chain variable region comprising the amino acid sequence set forth in SEQ ID NO: 58;
h) the α chain variable region comprises a CDR1, a CDR2, and a CDR3 of the α chain variable region comprising the amino acid sequence set forth in SEQ ID NO: 57, and the β chain variable region comprises a CDR1, a CDR2, and a CDR3 of the β chain variable region comprising the amino acid sequence set forth in SEQ ID NO: 62;
i) the α chain variable region comprises a CDR1, a CDR2, and a CDR3 of the α chain variable region comprising the amino acid sequence set forth in SEQ ID NO: 68, and the β chain variable region comprises a CDR1, a CDR2, and a CDR3 of the β chain variable region comprising the amino acid sequence set forth in SEQ ID NO: 69;
j) the α chain variable region comprises a CDR1, a CDR2, and a CDR3 of the α chain variable region comprising the amino acid sequence set forth in SEQ ID NO: 75, and the β chain variable region comprises a CDR1, a CDR2, and a CDR3 of the β chain variable region comprising the amino acid sequence set forth in SEQ ID NO: 69;
k) the α chain variable region comprises a CDR1, a CDR2, and a CDR3 of the α chain variable region comprising the amino acid sequence set forth in SEQ ID NO: 81, and the β chain variable region comprises a CDR1, a CDR2, and a CDR3 of the β chain variable region comprising the amino acid sequence set forth in SEQ ID NO: 82;
l) the α chain variable region comprises a CDR1, a CDR2, and a CDR3 of the α chain variable region comprising the amino acid sequence set forth in SEQ ID NO: 89, and the β chain variable region comprises a CDR1, a CDR2, and a CDR3 of the β chain variable region comprising the amino acid sequence set forth in SEQ ID NO: 90;
m) the α chain variable region comprises a CDR1, a CDR2, and a CDR3 of the α chain variable region comprising the amino acid sequence set forth in SEQ ID NO: 97, and the β chain variable region comprises a CDR1, a CDR2, and a CDR3 of the β chain variable region comprising the amino acid sequence set forth in SEQ ID NO: 98;
n) the α chain variable region comprises a CDR1, a CDR2, and a CDR3 of the α chain variable region comprising the amino acid sequence set forth in SEQ ID NO: 105, and the β chain variable region comprises a CDR1, a CDR2, and a CDR3 of the β chain variable region comprising the amino acid sequence set forth in SEQ ID NO: 106;
o) the α chain variable region comprises a CDR1, a CDR2, and a CDR3 of the α chain variable region comprising the amino acid sequence set forth in SEQ ID NO: 115, and the β chain variable region comprises a CDR1, a CDR2, and a CDR3 of the β chain variable region comprising the amino acid sequence set forth in SEQ ID NO: 116;
p) the α chain variable region comprises a CDR1, a CDR2, and a CDR3 of the α chain variable region comprising the amino acid sequence set forth in SEQ ID NO: 124, and the β chain variable region comprises a CDR1, a CDR2, and a CDR3 of the β chain variable region comprising the amino acid sequence set forth in SEQ ID NO: 125;
q) the α chain variable region comprises a CDR1, a CDR2, and a CDR3 of the α chain variable region comprising the amino acid sequence set forth in SEQ ID NO: 134, and the β chain variable region comprises a CDR1, a CDR2, and a CDR3 of the β chain variable region comprising the amino acid sequence set forth in SEQ ID NO: 135;
r) the α chain variable region comprises a CDR1, a CDR2, and a CDR3 of the α chain variable region comprising the amino acid sequence set forth in SEQ ID NO: 141, and the β chain variable region comprises a CDR1, a CDR2, and a CDR3 of the β chain variable region comprising the amino acid sequence set forth in SEQ ID NO: 142;
s) the α chain variable region comprises a CDR1, a CDR2, and a CDR3 of the α chain variable region comprising the amino acid sequence set forth in SEQ ID NO: 148, and the β chain variable region comprises a CDR1, a CDR2, and a CDR3 of the β chain variable region comprising the amino acid sequence set forth in SEQ ID NO: 142;
t) the α chain variable region comprises a CDR1, a CDR2, and a CDR3 of the α chain variable region comprising the amino acid sequence set forth in SEQ ID NO: 152, and the β chain variable region comprises a CDR1, a CDR2, and a CDR3 of the β chain variable region comprising the amino acid sequence set forth in SEQ ID NO: 153;
u) the α chain variable region comprises a CDR1, a CDR2, and a CDR3 of the α chain variable region comprising the amino acid sequence set forth in SEQ ID NO: 162, and the β chain variable region comprises a CDR1, a CDR2, and a CDR3 of the β chain variable region comprising the amino acid sequence set forth in SEQ ID NO: 163;
v) the α chain variable region comprises a CDR1, a CDR2, and a CDR3 of the α chain variable region comprising the amino acid sequence set forth in SEQ ID NO: 175, and the β chain variable region comprises a CDR1, a CDR2, and a CDR3 of the β chain variable region comprising the amino acid sequence set forth in SEQ ID NO: 176;
w) the α chain variable region comprises a CDR1, a CDR2, and a CDR3 of the α chain variable region comprising the amino acid sequence set forth in SEQ ID NO: 185, and the β chain variable region comprises a CDR1, a CDR2, and a CDR3 of the β chain variable region comprising the amino acid sequence set forth in SEQ ID NO: 186;
x) the α chain variable region comprises a CDR1, a CDR2, and a CDR3 of the α chain variable region comprising the amino acid sequence set forth in SEQ ID NO: 191, and the β chain variable region comprises a CDR1, a CDR2, and a CDR3 of the β chain variable region comprising the amino acid sequence set forth in SEQ ID NO: 192;
y) the α chain variable region comprises a CDR1, a CDR2, and a CDR3 of the α chain variable region comprising the amino acid sequence set forth in SEQ ID NO: 199, and the β chain variable region comprises a CDR1, a CDR2, and a CDR3 of the β chain variable region comprising the amino acid sequence set forth in SEQ ID NO: 200;
z) the α chain variable region comprises a CDR1, a CDR2, and a CDR3 of the α chain variable region comprising the amino acid sequence set forth in SEQ ID NO: 207, and the β chain variable region comprises a CDR1, a CDR2, and a CDR3 of the β chain variable region comprising the amino acid sequence set forth in SEQ ID NO: 208;
aa) the α chain variable region comprises a CDR1, a CDR2, and a CDR3 of the α chain variable region comprising the amino acid sequence set forth in SEQ ID NO: 217, and the β chain variable region comprises a CDR1, a CDR2, and a CDR3 of the β chain variable region comprising the amino acid sequence set forth in SEQ ID NO: 218;
ab) the α chain variable region comprises a CDR1, a CDR2, and a CDR3 of the α chain variable region comprising the amino acid sequence set forth in SEQ ID NO: 223, and the β chain variable region comprises a CDR1, a CDR2, and a CDR3 of the β chain variable region comprising the amino acid sequence set forth in SEQ ID NO: 224;
ac) the α chain variable region comprises a CDR1, a CDR2, and a CDR3 of the α chain variable region comprising the amino acid sequence set forth in SEQ ID NO: 233, and the β chain variable region comprises a CDR1, a CDR2, and a CDR3 of the β chain variable region comprising the amino acid sequence set forth in SEQ ID NO: 234;
ad) the α chain variable region comprises a CDR1, a CDR2, and a CDR3 of the α chain variable region comprising the amino acid sequence set forth in SEQ ID NO: 241, and the β chain variable region comprises a CDR1, a CDR2, and a CDR3 of the β chain variable region comprising the amino acid sequence set forth in SEQ ID NO: 242;
ae) the α chain variable region comprises a CDR1, a CDR2, and a CDR3 of the α chain variable region comprising the amino acid sequence set forth in SEQ ID NO: 249, and the β chain variable region comprises a CDR1, a CDR2, and a CDR3 of the β chain variable region comprising the amino acid sequence set forth in SEQ ID NO: 250;
af) the α chain variable region comprises a CDR1, a CDR2, and a CDR3 of the α chain variable region comprising the amino acid sequence set forth in SEQ ID NO: 256, and the β chain variable region comprises a CDR1, a CDR2, and a CDR3 of the β chain variable region comprising the amino acid sequence set forth in SEQ ID NO: 257;
ag) the α chain variable region comprises a CDR1, a CDR2, and a CDR3 of the α chain variable region comprising the amino acid sequence set forth in SEQ ID NO: 266, and the β chain variable region comprises a CDR1, a CDR2, and a CDR3 of the β chain variable region comprising the amino acid sequence set forth in SEQ ID NO: 267;
ah) the α chain variable region comprises a CDR1, a CDR2, and a CDR3 of the α chain variable region comprising the amino acid sequence set forth in SEQ ID NO: 274, and the β chain variable region comprises a CDR1, a CDR2, and a CDR3 of the β chain variable region comprising the amino acid sequence set forth in SEQ ID NO: 275;
ai) the α chain variable region comprises a CDR1, a CDR2, and a CDR3 of the α chain variable region comprising the amino acid sequence set forth in SEQ ID NO: 281, and the β chain variable region comprises a CDR1, a CDR2, and a CDR3 of the β chain variable region comprising the amino acid sequence set forth in SEQ ID NO: 282;
aj) the α chain variable region comprises a CDR1, a CDR2, and a CDR3 of the α chain variable region comprising the amino acid sequence set forth in SEQ ID NO: 291, and the β chain variable region comprises a CDR1, a CDR2, and a CDR3 of the β chain variable region comprising the amino acid sequence set forth in SEQ ID NO: 292;
ak) the α chain variable region comprises a CDR1, a CDR2, and a CDR3 of the α chain variable region comprising the amino acid sequence set forth in SEQ ID NO: 299, and the β chain variable region comprises a CDR1, a CDR2, and a CDR3 of the β chain variable region comprising the amino acid sequence set forth in SEQ ID NO: 300;
al) the α chain variable region comprises a CDR1, a CDR2, and a CDR3 of the α chain variable region comprising the amino acid sequence set forth in SEQ ID NO: 307, and the β chain variable region comprises a CDR1, a CDR2, and a CDR3 of the β chain variable region comprising the amino acid sequence set forth in SEQ ID NO: 308;
am) the α chain variable region comprises a CDR1, a CDR2, and a CDR3 of the α chain variable region comprising the amino acid sequence set forth in SEQ ID NO: 313, and the β chain variable region comprises a CDR1, a CDR2, and a CDR3 of the β chain variable region comprising the amino acid sequence set forth in SEQ ID NO: 314;
an) the α chain variable region comprises a CDR1, a CDR2, and a CDR3 of the α chain variable region comprising the amino acid sequence set forth in SEQ ID NO: 321, and the β chain variable region comprises a CDR1, a CDR2, and a CDR3 of the β chain variable region comprising the amino acid sequence set forth in SEQ ID NO: 322;
ao) the α chain variable region comprises a CDR1, a CDR2, and a CDR3 of the α chain variable region comprising the amino acid sequence set forth in SEQ ID NO: 329, and the β chain variable region comprises a CDR1, a CDR2, and a CDR3 of the β chain variable region comprising the amino acid sequence set forth in SEQ ID NO: 330;
ap) the α chain variable region comprises a CDR1, a CDR2, and a CDR3 of the α chain variable region comprising the amino acid sequence set forth in SEQ ID NO: 335, and the β chain variable region comprises a CDR1, a CDR2, and a CDR3 of the β chain variable region comprising the amino acid sequence set forth in SEQ ID NO: 336;
aq) the α chain variable region comprises a CDR1, a CDR2, and a CDR3 of the α chain variable region comprising the amino acid sequence set forth in SEQ ID NO: 341, and the β chain variable region comprises a CDR1, a CDR2, and a CDR3 of the β chain variable region comprising the amino acid sequence set forth in SEQ ID NO: 342;
ar) the α chain variable region comprises a CDR1, a CDR2, and a CDR3 of the α chain variable region comprising the amino acid sequence set forth in SEQ ID NO: 346, and the β chain variable region comprises a CDR1, a CDR2, and a CDR3 of the β chain variable region comprising the amino acid sequence set forth in SEQ ID NO: 342;
as) the α chain variable region comprises a CDR1, a CDR2, and a CDR3 of the α chain variable region comprising the amino acid sequence set forth in SEQ ID NO: 352, and the β chain variable region comprises a CDR1, a CDR2, and a CDR3 of the β chain variable region comprising the amino acid sequence set forth in SEQ ID NO: 353;
at) the α chain variable region comprises a CDR1, a CDR2, and a CDR3 of the α chain variable region comprising the amino acid sequence set forth in SEQ ID NO: 357, and the β chain variable region comprises a CDR1, a CDR2, and a CDR3 of the β chain variable region comprising the amino acid sequence set forth in SEQ ID NO: 353;
au) the α chain variable region comprises a CDR1, a CDR2, and a CDR3 of the α chain variable region comprising the amino acid sequence set forth in SEQ ID NO: 362, and the β chain variable region comprises a CDR1, a CDR2, and a CDR3 of the β chain variable region comprising the amino acid sequence set forth in SEQ ID NO: 363;
av) the α chain variable region comprises a CDR1, a CDR2, and a CDR3 of the α chain variable region comprising the amino acid sequence set forth in SEQ ID NO: 362, and the β chain variable region comprises a CDR1, a CDR2, and a CDR3 of the β chain variable region comprising the amino acid sequence set forth in SEQ ID NO: 363;
aw) the α chain variable region comprises a CDR1, a CDR2, and a CDR3 of the α chain variable region comprising the amino acid sequence set forth in SEQ ID NO: 329, and the β chain variable region comprises a CDR1, a CDR2, and a CDR3 of the β chain variable region comprising the amino acid sequence set forth in SEQ ID NO: 366;
ax) the α chain variable region comprises a CDR1, a CDR2, and a CDR3 of the α chain variable region comprising the amino acid sequence set forth in SEQ ID NO: 372, and the β chain variable region comprises a CDR1, a CDR2, and a CDR3 of the β chain variable region comprising the amino acid sequence set forth in SEQ ID NO: 366;
ay) the α chain variable region comprises a CDR1, a CDR2, and a CDR3 of the α chain variable region comprising the amino acid sequence set forth in SEQ ID NO: 379, and the β chain variable region comprises a CDR1, a CDR2, and a CDR3 of the β chain variable region comprising the amino acid sequence set forth in SEQ ID NO: 380; or
az) the α chain variable region comprises a CDR1, a CDR2, and a CDR3 of the α chain variable region comprising the amino acid sequence set forth in SEQ ID NO: 384, and the β chain variable region comprises a CDR1, a CDR2, and a CDR3 of the β chain variable region comprising the amino acid sequence set forth in SEQ ID NO: 380.


SEQUENCE LISTING (386 sequences)

SEQ ID NO: 1 - protein, 10 aa, synthetic construct
ILDTAGREEY

SEQ ID NO: 2 - protein, 10 aa, synthetic construct
ILDTAGKEEY

SEQ ID NO: 3 - protein, 10 aa, synthetic construct
ILDTAGLEEY

SEQ ID NO: 4 - protein, 10 aa, synthetic construct
ILDTAGQEEY

SEQ ID NO: 5 - protein, 5 aa, synthetic construct
SIFNT

SEQ ID NO: 6 - protein, 7 aa, synthetic construct
LYKAGEL

SEQ ID NO: 7 - protein, 13 aa, synthetic construct
CAGRDDAGNMLTF

SEQ ID NO: 8 - protein, 6 aa, synthetic construct
DFQATT

SEQ ID NO: 9 - protein, 7 aa, synthetic construct
SNEGSKA

SEQ ID NO: 10 - protein, 13 aa, synthetic construct
CSASVRMNTEAFF

SEQ ID NO: 11 - protein, 129 aa, synthetic construct
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGRDDAGNMLTFGGGTRLMVKP

SEQ ID NO: 12 - protein, 129 aa, synthetic construct
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASVRMNTEAFFGQGTRLTVV

SEQ ID NO: 13 - protein, 270 aa, synthetic construct
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGRDDAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 306 aa, synthetic construct
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASVRMNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 15 - protein, 6 aa, synthetic construct
NSASDY

SEQ ID NO: 16 - protein, 7 aa, synthetic construct
IRSNMDK

SEQ ID NO: 17 - protein, 15 aa, synthetic construct
CAENIRLTGANNLFF

SEQ ID NO: 18 - protein, 8 aa, synthetic construct
SNEGSKAT

SEQ ID NO: 19 - protein, 12 aa, synthetic construct
CSARLLSYEQYF

SEQ ID NO: 20 - protein, 134 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENIRLTGANNLFFGTGTRLTVIP

SEQ ID NO: 21 - protein, 128 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARLLSYEQYFGPGTRLTVT

SEQ ID NO: 22 - protein, 275 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENIRLTGANNLFFGTGTRLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 307 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARLLSYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 5 aa, synthetic construct
SVFSS

SEQ ID NO: 25 - protein, 7 aa, synthetic construct
VVTGGEV

SEQ ID NO: 26 - protein, 17 aa, synthetic construct
CAGGWLISNFGNEKLTF

SEQ ID NO: 27 - protein, 5 aa, synthetic construct
SGHNT

SEQ ID NO: 28 - protein, 6 aa, synthetic construct
YYREEE

SEQ ID NO: 29 - protein, 15 aa, synthetic construct
CASSLGTAGNEKLFF

SEQ ID NO: 30 - protein, 133 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGGWLISNFGNEKLTFGTGTRLTIIP

SEQ ID NO: 31 - protein, 133 aa, synthetic construct
MGPGLLCWALLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSLGTAGNEKLFFGSGTQLSVL

SEQ ID NO: 32 - protein, 274 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGGWLISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 133 aa, synthetic construct; identical to SEQ ID NO: 31

SEQ ID NO: 34 - protein, 7 aa, synthetic construct
TSESDYY

SEQ ID NO: 35 - protein, 8 aa, synthetic construct
QEAYKQQN

SEQ ID NO: 36 - protein, 12 aa, synthetic construct
CAYREYGNKLVF

SEQ ID NO: 37 - protein, 5 aa, synthetic construct
SGDLS

SEQ ID NO: 38 - protein, 6 aa, synthetic construct
YYNGEE

SEQ ID NO: 39 - protein, 15 aa, synthetic construct
CASSAGRGGFHEQYF

SEQ ID NO: 40 - protein, 133 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYREYGNKLVFGAGTILRVKS

SEQ ID NO: 41 - protein, 133 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAGRGGFHEQYFGPGTRLTVT

SEQ ID NO: 42 - protein, 274 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYREYGNKLVFGAGTILRVKSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - protein, 310 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAGRGGFHEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 44 - protein, 14 aa, synthetic construct
CAYSNDNYGQNFVF

SEQ ID NO: 45 - protein, 135 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYSNDNYGQNFVFGPGTRLSVLP

SEQ ID NO: 46 - protein, 276 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYSNDNYGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 47 - protein, 14 aa, synthetic construct
CAGHEVSDGQKLLF

SEQ ID NO: 48 - protein, 5 aa, synthetic construct
LNHNV

SEQ ID NO: 49 - protein, 6 aa, synthetic construct
YYDKDF

SEQ ID NO: 50 - protein, 13 aa, synthetic construct
CATSRTHSYEQYF

SEQ ID NO: 51 - protein, 130 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHEVSDGQKLLFARGTMLKVDL

SEQ ID NO: 52 - protein, 131 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRTHSYEQYFGPGTRLTVT

SEQ ID NO: 53 - protein, 271 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHEVSDGQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 308 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRTHSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 55 - protein, 13 aa, synthetic construct
CAGYTGANSKLTF

SEQ ID NO: 56 - protein, 14 aa, synthetic construct
CASSVESGDYEQYF

SEQ ID NO: 57 - protein, 129 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGYTGANSKLTFGKGITLSVRP

SEQ ID NO: 58 - protein, 132 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESGDYEQYFGPGTRLTVT

SEQ ID NO: 59 - protein, 270 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGYTGANSKLTFGKGITLSVRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 309 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESGDYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 13 aa, synthetic construct
CASSVGATYEQYF

SEQ ID NO: 62 - protein, 131 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGATYEQYFGPGTRLTVT

SEQ ID NO: 63 - protein, 308 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGATYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 64 - protein, 6 aa, synthetic construct
VSNAYN

SEQ ID NO: 65 - protein, 4 aa, synthetic construct
GSKP

SEQ ID NO: 66 - protein, 13 aa, synthetic construct
CAAPASSASKIIF

SEQ ID NO: 67 - protein, 17 aa, synthetic construct
CATSRLGGPTPNEKLFF

SEQ ID NO: 68 - protein, 131 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAAPASSASKIIFGSGTRLSIRP

SEQ ID NO: 69 - protein, 135 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRLGGPTPNEKLFFGSGTQLSVL

SEQ ID NO: 70 - protein, 272 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAAPASSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 71 - protein, 314 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRLGGPTPNEKLFFGSGTQLSVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 6 aa, synthetic construct
YGATPY

SEQ ID NO: 73 - protein, 8 aa, synthetic construct
YFSGDTLV

SEQ ID NO: 74 - protein, 14 aa, synthetic construct
CAVGAAGGSYIPTF

SEQ ID NO: 75 - protein, 133 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAAGGSYIPTFGRGTSLIVHP

SEQ ID NO: 76 - protein, 274 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAAGGSYIPTFGRGTSLIVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 77 - protein, 15 aa, synthetic construct
CAGRRTTDSWGKLQF

SEQ ID NO: 78 - protein, 5 aa, synthetic construct
MDHEN

SEQ ID NO: 79 - protein, 6 aa, synthetic construct
SYDVKM

SEQ ID NO: 80 - protein, 16 aa, synthetic construct
CASSLFGGSNTGELFF

SEQ ID NO: 81 - protein, 131 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRRTTDSWGKLQFGAGTQVVVTP

SEQ ID NO: 82 - protein, 134 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLFGGSNTGELFFGEGSRLTVL

SEQ ID NO: 83 - protein, 272 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRRTTDSWGKLQFGAGTQVVVTPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 311 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLFGGSNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 85 - protein, 6 aa, synthetic construct
SSNFYA

SEQ ID NO: 86 - protein, 7 aa, synthetic construct
MTLNGDE

SEQ ID NO: 87 - protein, 12 aa, synthetic construct
CASPDNNARLMF

SEQ ID NO: 88 - protein, 13 aa, synthetic construct
CASSPNLSWELFF

SEQ ID NO: 89 - protein, 133 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASPDNNARLMFGDGTQLVVKP

SEQ ID NO: 90 - protein, 131 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPNLSWELFFGEGSRLTVL

SEQ ID NO: 91 - protein, 274 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASPDNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 308 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPNLSWELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 93 - protein, 6 aa, synthetic construct
ATGYPS

SEQ ID NO: 94 - protein, 7 aa, synthetic construct
ATKADDK

SEQ ID NO: 95 - protein, 12 aa, synthetic construct
CALLNNNNDMRF

SEQ ID NO: 96 - protein, 17 aa, synthetic construct
CATSRDKGTEAHYGYTF

SEQ ID NO: 97 - protein, 130 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALLNNNNDMRFGAGTRLTVKP

SEQ ID NO: 98 - protein, 135 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRDKGTEAHYGYTFGSGTRLTVV

SEQ ID NO: 99 - protein, 271 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALLNNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 100 - protein, 314 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRDKGTEAHYGYTFGSGTRLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 101 - protein, 15 aa, synthetic construct
CAGHRGDNYGQNFVF

SEQ ID NO: 102 - protein, 5 aa, synthetic construct
MGHRA

SEQ ID NO: 103 - protein, 6 aa, synthetic construct
YSYEKL

SEQ ID NO: 104 - protein, 15 aa, synthetic construct
CASSQDRLTYNEQFF

SEQ ID NO: 105 - protein, 131 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHRGDNYGQNFVFGPGTRLSVLP

SEQ ID NO: 106 - protein, 133 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDRLTYNEQFFGPGTRLTVL

SEQ ID NO: 107 - protein, 272 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHRGDNYGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 108 - protein, 310 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDRLTYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 109 - protein, 6 aa, synthetic construct
TSGFYG

SEQ ID NO: 110 - protein, 6 aa, synthetic construct
NALDGL

SEQ ID NO: 111 - protein, 11 aa, synthetic construct
CAVHSWGKLQF

SEQ ID NO: 112 - protein, 5 aa, synthetic construct
SEHNR

SEQ ID NO: 113 - protein, 6 aa, synthetic construct
FQNEAQ

SEQ ID NO: 114 - protein, 14 aa, synthetic construct
CASSPLGGSYEQYF

SEQ ID NO: 115 - protein, 125 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVHSWGKLQFGAGTQVVVTP

SEQ ID NO: 116 - protein, 133 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLGGSYEQYFGPGTRLTVT

SEQ ID NO: 117 - protein, 266 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVHSWGKLQFGAGTQVVVTPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 118 - protein, 310 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLGGSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 119 - protein, 5 aa, synthetic construct
TSINN

SEQ ID NO: 120 - protein, 7 aa, synthetic construct
IRSNERE

SEQ ID NO: 121 - protein, 13 aa, synthetic construct
CATDAGGGNKLVF

SEQ ID NO: 122 - protein, 5 aa, synthetic construct
SGHVS

SEQ ID NO: 123 - protein, 15 aa, synthetic construct
CASSFLGGSNEKLFF

SEQ ID NO: 124 - protein, 131 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAGGGNKLVFGAGTILRVKS

SEQ ID NO: 125 - protein, 134 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFLGGSNEKLFFGSGTQLSVL

SEQ ID NO: 126 - protein, 272 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAGGGNKLVFGAGTILRVKSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 127 - protein, 313 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFLGGSNEKLFFGSGTQLSVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 128 - protein, 6 aa, synthetic construct
NSMFDY

SEQ ID NO: 129 - protein, 7 aa, synthetic construct
ISSIKDK

SEQ ID NO: 130 - protein, 13 aa, synthetic construct
CAASDQAGTALIF

SEQ ID NO: 131 - protein, 5 aa, synthetic construct
KGHSY

SEQ ID NO: 132 - protein, 6 aa, synthetic construct
FQNENV

SEQ ID NO: 133 - protein, 15 aa, synthetic construct
CASSPPGQGTNTIYF

SEQ ID NO: 134 - protein, 138 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASDQAGTALIFGKGTTLSVSS

SEQ ID NO: 135 - protein, 134 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSPPGQGTNTIYFGEGSWLTVV

SEQ ID NO: 136 - protein, 279 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASDQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 137 - protein, 313 aa, synthetic construct
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSPPGQGTNTIYFGEGSWLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 138 - protein, 7 aa, synthetic construct
TSENNYY

SEQ ID NO: 139 - protein, 10 aa, synthetic construct
CAFLNARLMF

SEQ ID NO: 140 - protein, 16 aa, synthetic construct
CASSQDPQGSYNEQFF

SEQ ID NO: 141 - protein, 131 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFLNARLMFGDGTQLVVKP

SEQ ID NO: 142 - protein, 134 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDPQGSYNEQFFGPGTRLTVL

SEQ ID NO: 143 - protein, 272 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFLNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 144 - protein, 311 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDPQGSYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 145 - protein, 7 aa, synthetic construct
TISGTDY

SEQ ID NO: 146 - protein, 5 aa, synthetic construct
GLTSN

SEQ ID NO: 147 - protein, 12 aa, synthetic construct
CILRGGSEKLVF

SEQ ID NO: 148 - protein, 126 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRGGSEKLVFGKGTKLTVNP

SEQ ID NO: 149 - protein, 267 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRGGSEKLVFGKGTKLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 150 - protein, 12 aa, synthetic construct
CAYRSPNTPLVF

SEQ ID NO: 151 - protein, 13 aa, synthetic construct
CASSAWTSGELFF

SEQ ID NO: 152 - protein, 133 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSPNTPLVFGKGTRLSVIA

SEQ ID NO: 153 - protein, 131 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAWTSGELFFGEGSRLTVL

SEQ ID NO: 154 - protein, 274 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSPNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 155 - protein, 308 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAWTSGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 156 - protein, 7 aa, synthetic construct
NIATNDY

SEQ ID NO: 157 - protein, 5 aa, synthetic construct
GYKTK

SEQ ID NO: 158 - protein, 17 aa, synthetic construct
CLVGDSLANFGNEKLTF

SEQ ID NO: 159 - protein, 5 aa, synthetic construct
SNHLY

SEQ ID NO: 160 - protein, 6 aa, synthetic construct
FYNNEI

SEQ ID NO: 161 - protein, 14 aa, synthetic construct
CASSSRTAQDGYTF

SEQ ID NO: 162 - protein, 131 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDSLANFGNEKLTFGTGTRLTIIP

SEQ ID NO: 163 - protein, 133 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSSRTAQDGYTFGSGTRLTVV

SEQ ID NO: 164 - protein, 272 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDSLANFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 165 - protein, 312 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSSRTAQDGYTFGSGTRLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 166 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 167 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 168 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 169 - protein, 179 aa, synthetic construct
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 170 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 171 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 172 - protein, 27 aa, synthetic construct
RAKRSGSGATNFSLLKQAGDVEENPGP

SEQ ID NO: 173 - protein, 13 aa, synthetic construct
AGHSGDNYGQNFV

SEQ ID NO: 174 - protein, 13 aa, synthetic construct
ASSPDRLTYNEQF

SEQ ID NO: 175 - protein, 131 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHSGDNYGQNFVFGPGTRLSVLP

SEQ ID NO: 176 - protein, 133 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSPDRLTYNEQFFGPGTRLTVL

SEQ ID NO: 177 - protein, 272 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHSGDNYGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 178 - protein, 312 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSPDRLTYNEQFFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 179 - protein, 6 aa, synthetic construct
DRGSQS

SEQ ID NO: 180 - protein, 6 aa, synthetic construct
IYSNGD

SEQ ID NO: 181 - protein, 11 aa, synthetic construct
AVNRGFGNVLH

SEQ ID NO: 182 - protein, 5 aa, synthetic construct
MNHEY

SEQ ID NO: 183 - protein, 6 aa, synthetic construct
SMNVEV

SEQ ID NO: 184 - protein, 13 aa, synthetic construct
ASSLGGILVTEAF

SEQ ID NO: 185 - protein, 132 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNRGFGNVLHCGSGTQVIVLP

SEQ ID NO: 186 - protein, 133 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGGILVTEAFFGQGTRLTVV

SEQ ID NO: 187 - protein, 273 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNRGFGNVLHCGSGTQVIVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 188 - protein, 310 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGGILVTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 189 - protein, 11 aa, synthetic construct
ATEPDSNYQLI

SEQ ID NO: 190 - protein, 10 aa, synthetic construct
ASSGDRGKLF

SEQ ID NO: 191 - protein, 131 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATEPDSNYQLIWGAGTKLIIKP

SEQ ID NO: 192 - protein, 130 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGDRGKLFFGSGTQLSVL

SEQ ID NO: 193 - protein, 272 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATEPDSNYQLIWGAGTKLIIKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 194 - protein, 307 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGDRGKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 195 - protein, 5 aa, synthetic construct
TTLSN

SEQ ID NO: 196 - protein, 7 aa, synthetic construct
LVKSGEV

SEQ ID NO: 197 - protein, 9 aa, synthetic construct
GNQAGTALI

SEQ ID NO: 198 - protein, 14 aa, synthetic construct
ASSETFRDSIDGYT

SEQ ID NO: 199 - protein, 127 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCGNQAGTALIFGKGTTLSVSS

SEQ ID NO: 200 - protein, 135 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSETFRDSIDGYTFGSGTRLTVV

SEQ ID NO: 201 - protein, 268 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCGNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 202 - protein, 312 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSETFRDSIDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 203 - protein, 6 aa, synthetic construct
DSAIYN

SEQ ID NO: 204 - protein, 7 aa, synthetic construct
IQSSQRE

SEQ ID NO: 205 - protein, 12 aa, synthetic construct
AVMAMDSSYKLI

SEQ ID NO: 206 - protein, 12 aa, synthetic construct
ASAGGLDPDTQY

SEQ ID NO: 207 - protein, 132 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVMAMDSSYKLIFGSGTRLLVRP

SEQ ID NO: 208 - protein, 133 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASAGGLDPDTQYFGPGTRLTVL

SEQ ID NO: 209 - protein, 273 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVMAMDSSYKLIFGSGTRLLVRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 210 - protein, 312 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASAGGLDPDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 211 - protein, 6 aa, synthetic construct
SSVSVY

SEQ ID NO: 212 - protein, 8 aa, synthetic construct
YLSGSTLV

SEQ ID NO: 213 - protein, 13 aa, synthetic construct
AVSSRGGNTDKLI

SEQ ID NO: 214 - protein, 6 aa, synthetic construct
GTSNPN

SEQ ID NO: 215 - protein, 5 aa, synthetic construct
SVGIG

SEQ ID NO: 216 - protein, 12 aa, synthetic construct
AWSRQGLGQPQH

SEQ ID NO: 217 - protein, 134 aa, synthetic construct
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSSRGGNTDKLIFGTGTRLQVFP

SEQ ID NO: 218 - protein, 130 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSRQGLGQPQHFGDGTRLSIL

SEQ ID NO: 219 - protein, 275 aa, synthetic construct
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSSRGGNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 220 - protein, 307 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSRQGLGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 221 - protein, 10 aa, synthetic construct
AVPSGNTPLV

SEQ ID NO: 222 - protein, 9 aa, synthetic construct
ASESDEKLF

SEQ ID NO: 223 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVPSGNTPLVFGKGTRLSVIAN

SEQ ID NO: 224 - protein, 130 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASESDEKLFFGSGTQLSVL

SEQ ID NO: 225 - protein, 273 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVPSGNTPLVFGKGTRLSVIANNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 226 - protein, 307 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASESDEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 227 - protein, 6 aa, synthetic construct
NSASQS

SEQ ID NO: 228 - protein, 5 aa, synthetic construct
VYSSG

SEQ ID NO: 229 - protein, 12 aa, synthetic construct
VVHDHNAGNMLT

SEQ ID NO: 230 - protein, 5 aa, synthetic construct
LGHDT

SEQ ID NO: 231 - protein, 6 aa, synthetic construct
YNNKEL

SEQ ID NO: 232 - protein, 11 aa, synthetic construct
ASSPGVGYGYT

SEQ ID NO: 233 - protein, 132 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVHDHNAGNMLTFGGGTRLMVKP

SEQ ID NO: 234 - protein, 131 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSPGVGYGYTFGSGTRLTVV

SEQ ID NO: 235 - protein, 273 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVHDHNAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 236 - protein, 308 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSPGVGYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 237 - protein, 11 aa, synthetic construct
AATAGNNNDMR

SEQ ID NO: 238 - protein, 5 aa, synthetic construct
SGHDT

SEQ ID NO: 239 - protein, 6 aa, synthetic construct
YYEEEE

SEQ ID NO: 240 - protein, 11 aa, synthetic construct
ASSLRENYGYT

SEQ ID NO: 241 - protein, 138 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATAGNNNDMRFGAGTRLTVKP

SEQ ID NO: 242 - protein, 131 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLRENYGYTFGSGTRLTVV

SEQ ID NO: 243 - protein, 279 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATAGNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 244 - protein, 308 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLRENYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 245 - protein, 9 aa, synthetic construct
AVDIGNKLV

SEQ ID NO: 246 - protein, 5 aa, synthetic construct
WSHSY

SEQ ID NO: 247 - protein, 6 aa, synthetic construct
SAAADI

SEQ ID NO: 248 - protein, 13 aa, synthetic construct
ASSEGGLAKNIQY

SEQ ID NO: 249 - protein, 130 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVDIGNKLVFGAGTILRVKS

SEQ ID NO: 250 - protein, 133 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSEGGLAKNIQYFGAGTRLSVL

SEQ ID NO: 251 - protein, 271 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVDIGNKLVFGAGTILRVKSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 252 - protein, 312 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSEGGLAKNIQYFGAGTRLSVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 253 - protein, 5 aa, synthetic construct
GYKTK

SEQ ID NO: 254 - protein, 12 aa, synthetic construct
LALNTGTASKLT

SEQ ID NO: 255 - protein, 12 aa, synthetic construct
ASSASGRTDTQY

SEQ ID NO: 256 - protein, 128 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLALNTGTASKLTFGTGTRLQVTL

SEQ ID NO: 257 - protein, 132 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSASGRTDTQYFGPGTRLTVL

SEQ ID NO: 258 - protein, 269 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLALNTGTASKLTFGTGTRLQVTLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 259 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSASGRTDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 260 - protein, 6 aa, synthetic construct
NTAFDY

SEQ ID NO: 261 - protein, 7 aa, synthetic construct
IRPDVSE

SEQ ID NO: 262 - protein, 11 aa, synthetic construct
AAGDNYGQNFV

SEQ ID NO: 263 - protein, 5 aa, synthetic construct
SGHTA

SEQ ID NO: 264 - protein, 6 aa, synthetic construct
FQGNSA

SEQ ID NO: 265 - protein, 12 aa, synthetic construct
ASSLMSGNSPLH

SEQ ID NO: 266 - protein, 139 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAAGDNYGQNFVFGPGTRLSVL

SEQ ID NO: 267 - protein, 133 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLMSGNSPLHFGNGTRLTVT

SEQ ID NO: 268 - protein, 280 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAAGDNYGQNFVFGPGTRLSVLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLMSGNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 270 - protein, 10 aa, synthetic construct
AVQTGANNLF

SEQ ID NO: 271 - protein, 5 aa, synthetic construct
KGHSH

SEQ ID NO: 272 - protein, 6 aa, synthetic construct
LQKENI

SEQ ID NO: 273 - protein, 16 aa, synthetic construct
ASSPQRGVSSGANVLT

SEQ ID NO: 274 - protein, 130 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVQTGANNLFFGTGTRLTVIP

SEQ ID NO: 275 - protein, 137 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQRGVSSGANVLTFGAGSRLTVL

SEQ ID NO: 276 - protein, 271 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVQTGANNLFFGTGTRLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - protein, 316 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQRGVSSGANVLTFGAGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 278 - protein, 13 aa, synthetic construct
AASGNAGNNRKLI

SEQ ID NO: 279 - protein, 6 aa, synthetic construct
FQGTGA

SEQ ID NO: 280 - protein, 16 aa, synthetic construct
ASSLMAGTASGANVLT

SEQ ID NO: 281 - protein, 140 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGNAGNNRKLIWGLGTSLAVNP

SEQ ID NO: 282 - protein, 137 aa, synthetic construct
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLMAGTASGANVLTFGAGSRLTVL

SEQ ID NO: 283 - protein, 281 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGNAGNNRKLIWGLGTSLAVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 284 - protein, 316 aa, synthetic construct
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLMAGTASGANVLTFGAGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 285 - protein, 7 aa, synthetic construct
TRDTTYY

SEQ ID NO: 286 - protein, 8 aa, synthetic construct
RNSFDEQN

SEQ ID NO: 287 - protein, 13 aa, synthetic construct
ALSESVNAGNMLT

SEQ ID NO: 288 - protein, 5 aa, synthetic construct
MNHNS

SEQ ID NO: 289 - protein, 6 aa, synthetic construct
SASEGT

SEQ ID NO: 290 - protein, 9 aa, synthetic construct
ASSQNTEAF

SEQ ID NO: 291 - protein, 135 aa, synthetic construct
LTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESVNAGNMLTFGGGTRLMVKP

SEQ ID NO: 292 - protein, 129 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSQNTEAFFGQGTRLTVV

SEQ ID NO: 293 - protein, 277 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESVNAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 294 - protein, 306 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSQNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 295 - protein, 11 aa, synthetic construct
VVNMPSGQNFV

SEQ ID NO: 296 - protein, 5 aa, synthetic construct
ENHRY

SEQ ID NO: 297 - protein, 6 aa, synthetic construct
SYGVKD

SEQ ID NO: 298 - protein, 13 aa, synthetic construct
AISQDRATLAEQY

SEQ ID NO: 299 - protein, 131 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNMPSGQNFVFGPGTRLSVLP

SEQ ID NO: 300 - protein, 133 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISQDRATLAEQYVGPGTRLTVT

SEQ ID NO: 301 - protein, 272 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNMPSGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 302 - protein, 312 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISQDRATLAEQYVGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 303 - protein, 14 aa, synthetic construct
AGQFTGGTSYGKLT

SEQ ID NO: 304 - protein, 5 aa, synthetic construct
LGHNT

SEQ ID NO: 305 - protein, 6 aa, synthetic construct
FRNRAP

SEQ ID NO: 306 - protein, 12 aa, synthetic construct
ATGQGLDTGELF

SEQ ID NO: 307 - protein, 132 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQFTGGTSYGKLTFGQGTILTVHP

SEQ ID NO: 308 - protein, 133 aa, synthetic construct
MATRLLCCVVLCLLGEELIDARVTQTPRDKVTEMGQEVTMRCQPILGHNTVFWYRQTMMQGLELLAYFRNRAPLDDSGMPKDRFSAEMPDATLATLKIQPSEPRDSAVYFCATGQGLDTGELFFGEGSRLTVL

SEQ ID NO: 309 - protein, 273 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQFTGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 310 - protein, 312 aa, synthetic construct
MATRLLCCVVLCLLGEELIDARVTQTPRDKVTEMGQEVTMRCQPILGHNTVFWYRQTMMQGLELLAYFRNRAPLDDSGMPKDRFSAEMPDATLATLKIQPSEPRDSAVYFCATGQGLDTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 311 - protein, 10 aa, synthetic construct
AAVDNNNDMR

SEQ ID NO: 312 - protein, 14 aa, synthetic construct
ASSLDRGSSGNTIY

SEQ ID NO: 313 - protein, 137 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAVDNNNDMRFGAGTRLTVKP

SEQ ID NO: 314 - protein, 134 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSLDRGSSGNTIYFGEGSWLTVV

SEQ ID NO: 315 - protein, 278 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAVDNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 316 - protein, 311 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSLDRGSSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 317 - protein, 6 aa, synthetic construct
TSGFNG

SEQ ID NO: 318 - protein, 6 aa, synthetic construct
NVLDGL

SEQ ID NO: 319 - protein, 12 aa, synthetic construct
AASFSGGYNKLI

SEQ ID NO: 320 - protein, 11 aa, synthetic construct
ASSLMRGDTQY

SEQ ID NO: 321 - protein, 128 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAASFSGGYNKLIFGAGTRLAVHP

SEQ ID NO: 322 - protein, 132 aa, synthetic construct
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLMRGDTQYFGPGTRLTVL

SEQ ID NO: 323 - protein, 269 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAASFSGGYNKLIFGAGTRLAVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 324 - protein, 311 aa, synthetic construct
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLMRGDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 325 - protein, 14 aa, synthetic construct
ALSETYNNAGNMLT

SEQ ID NO: 326 - protein, 5 aa, synthetic construct
MNHNY

SEQ ID NO: 327 - protein, 6 aa, synthetic construct
SVGAGI

SEQ ID NO: 328 - protein, 9 aa, synthetic construct
ASTQNTEAF

SEQ ID NO: 329 - protein, 137 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSETYNNAGNMLTFGGGTRLMVKP

SEQ ID NO: 330 - protein, 129 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASTQNTEAFFGQGTRLTVV

SEQ ID NO: 331 - protein, 278 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSETYNNAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 332 - protein, 306 aa, synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASTQNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 333 - protein, 14 aa, synthetic construct
AGQFAGGTSYGKLT

SEQ ID NO: 334 - protein, 12 aa, synthetic construct
ASGRGLDTGELF

SEQ ID NO: 335 - protein, 132 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQFAGGTSYGKLTFGQGTILTVHP

SEQ ID NO: 336 - protein, 133 aa, synthetic construct
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGRGLDTGELFFGEGSRLTVL

SEQ ID NO: 337 - protein, 273 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQFAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 338 - protein, 312 aa, synthetic construct
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGRGLDTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 339 - protein, 14 aa, synthetic construct
ALSESGNNAGNMLT

SEQ ID NO: 340 - protein, 9 aa, synthetic construct
ASSENTEAF

SEQ ID NO: 341 - protein, 137 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESGNNAGNMLTFGGGTRLMVKP

SEQ ID NO: 342 - protein, 129 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSENTEAFFGQGTRLTVV

SEQ ID NO: 343 - protein, 278 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESGNNAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 344 - protein, 308 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSENTEAFFGQGTRLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 345 - protein, 14 aa, synthetic construct
ALSESGRNAGNMLT

SEQ ID NO: 346 - protein, 137 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESGRNAGNMLTFGGGTRLMVKP

SEQ ID NO: 347 - protein, 278 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESGRNAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 348 - protein, 306 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSENTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 349 - protein, 12 aa, synthetic construct
ALRLRNTGNQFY

SEQ ID NO: 350 - protein, 6 aa, synthetic construct
SVGEGT

SEQ ID NO: 351 - protein, 15 aa, synthetic construct
ASSGTGRPTTMETQY

SEQ ID NO: 352 - protein, 132 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRLRNTGNQFYFGTGTSLTVIP

SEQ ID NO: 353 - protein, 135 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSGTGRPTTMETQYFGPGTRLLVL

SEQ ID NO: 354 - protein, 273 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRLRNTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 355 - protein, 314 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSGTGRPTTMETQYFGPGTRLLVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 356 - protein, 12 aa, synthetic construct
AVSTWPNDYKLS

SEQ ID NO: 357 - protein, 130 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAVSTWPNDYKLSFGAGTTVTVRA

SEQ ID NO: 358 - protein, 271 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAVSTWPNDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 359 - protein, 314 aa, synthetic construct; identical to SEQ ID NO: 355

SEQ ID NO: 360 - protein, 9 aa, synthetic construct
AVNYGQNFV

SEQ ID NO: 361 - protein, 13 aa, synthetic construct
ASTSGSDRDYEQY

SEQ ID NO: 362 - protein, 130 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNYGQNFVFGPGTRLSVLP

SEQ ID NO: 363 - protein, 133 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASTSGSDRDYEQYFGPGTRLTVT

SEQ ID NO: 364 - protein, 271 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNYGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 365 - protein, 312 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASTSGSDRDYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 366 - protein, 129 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSQNTEAFFGQGTRLTVV

SEQ ID NO: 367 - protein, 278 aa, synthetic construct; identical to SEQ ID NO: 331

SEQ ID NO: 368 - protein, 306 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSQNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 369 - protein, 6 aa, synthetic construct
NYSPAY

SEQ ID NO: 370 - protein, 7 aa, synthetic construct
IRENEKE

SEQ ID NO: 371 - protein, 12 aa, synthetic construct
ARLRDNYGQNFV

SEQ ID NO: 372 - protein, 152 aa, synthetic construct
MAFWLRSLGLHFRLHLGRRMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCARLRDNYGQNFVFGPGTRLSVLP

SEQ ID NO: 373 - protein, 293 aa, synthetic construct
MAFWLRSLGLHFRLHLGRRMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCARLRDNYGQNFVFGPGTRLSVLPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 374 - protein, 306 aa, synthetic construct; identical to SEQ ID NO: 368

SEQ ID NO: 375-377 - skipped (3 entries)

SEQ ID NO: 378 - protein, 13 aa, synthetic construct
ASSPGGGHSYEQY

SEQ ID NO: 379 - skipped

SEQ ID NO: 380 - protein, 133 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGGHSYEQYFGPGTRLTVT

SEQ ID NO: 381-382 - skipped (2 entries)

SEQ ID NO: 383 - protein, 13 aa, synthetic construct
AVNGAQGGSEKLV

SEQ ID NO: 384 - protein, 134 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNGAQGGSEKLVFGKGTKLTVNP

SEQ ID NO: 385 - protein, 275 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNGAQGGSEKLVFGKGTKLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 386 - protein, 312 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGGHSYEQYFGPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG